(19) United States
(12) Patent Application Publication
(10) Pub. No.: US 2023/0117167 A1
(43) Pub. Date: Apr. 20, 2023

(54) DEOPTIMIZED SARS-CoV-2 AND METHODS AND USES THEREOF

(71) Applicants: CODAGENIX INC., Farmingdale, NY (US); SERUM INSTITUTE OF INDIA PRIVATE LIMITED, Hadapsar, Pune (IN)

(72) Inventors: Steffen Mueller, Great Neck, NY (US); John Robert Coleman, Blauvelt, NY (US); Ying Wang, South Setauket, NY (US); Chen Yang, Plainview, NY (US)

(73) Assignees: CODAGENIX INC., Farmingdale, NY (US); SERUM INSTITUTE OF INDIA PRIVATE LIMITED, Hadapsar, Pune (IN)

(21) Appl. No.: 17/794,862

(22) PCT Filed: Jan. 27, 2021
(86) PCT No.: PCT/US21/15246

§ 371 Date: Jul. 22, 2022

Related U.S. Application Data
(60) Provisional application No. 62/966,750, filed on Jan. 28, 2020.
(60) Provisional application No. 63/048,942, filed on Jul. 7, 2020.
(60) Provisional application No. 63/079,337, filed on Sep. 16, 2020.
(60) Provisional application No. 63/079,853, filed on Sep. 17, 2020.

(51) Int. Cl. C07K 14/005; A61K 39/215; A61P 31/14

(52) U.S. Cl. CPC C07K 14/005; A61K 39/215; A61P 31/14; C12N 2770/20022; C12N 2770/20071; C12N 2770/20034; A61K 2039/5254

(57) ABSTRACT

Described herein are modified SARS-CoV-2 coronaviruses. These viruses have been recoded, for example, codon deoptimized or codon pair bias deoptimized and are useful for reducing the likelihood or severity of a SARS-CoV-2 coronavirus infection, preventing a SARS-CoV-2 coronavirus infection, eliciting and immune response, or treating a SARS-CoV-2 coronavirus infection.

### CROSS-REFERENCE TO RELATED APPLICATIONS

This application includes a claim of priority under 35 U.S.C. § 119(e) to U.S. provisional patent application No. 62/966,750, filed Jan. 28, 2020, No. 63/048,942, filed Jul. 7, 2020, No. 63/079,337, filed Sep. 16, 2020, and No. 63/079,853, filed Sep. 17, 2020, the entirety of which are all hereby incorporated by reference.

### FIELD OF INVENTION

This invention relates to modified SARS-CoV-2 coronaviruses, compositions for eliciting an immune response and vaccines for providing protective immunity, prevention and treatment.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

An outbreak of a novel coronavirus was identified during mid-December 2019 in the city of Wuhan in central China. A new strain of coronavirus—previously designated as 2019-nCoV (and also previously known as Wuhan Coronavirus), now designated as SARS-CoV-2—was identified. The deadly coronavirus has been declared by the WHO as pandemic. The public health crisis of this virus rapidly grew from claiming the lives of dozens of people and infecting over a thousand as of the end of January 2020, to claiming the lives of over 900,000 people and infecting over 28 million people as of the beginning of September 2020, and to claiming the lives of over 2 million people and infecting over 100 million people as of the last week of January 2021. SARS-CoV-2 viruses are particularly dangerous for the elderly and those with underlying medical conditions such as chronic kidney disease, chronic obstructive pulmonary disease, being immunocompromised from a solid organ transplant, obesity, serious heart conditions, sickle cell disease and type 2 diabetes mellitus. Accordingly, prophylactic and therapeutic treatments are exceedingly and urgently needed.

### SUMMARY OF THE INVENTION

The following embodiments and aspects thereof are described and illustrated in conjunction with compositions and methods which are meant to be exemplary and illustrative, not limiting in scope.

Various embodiments of the present invention provide for a polynucleotide encoding one or more viral proteins or one or more fragments thereof of a parent SARS-CoV-2 coronavirus: wherein the polynucleotide is recoded compared to its parent SARS-CoV-2 coronavirus polynucleotide, and wherein the amino acid sequence of the one or more viral proteins, or one or more fragments thereof of the parent SARS-CoV-2 coronavirus encoded by the polynucleotide remains the same, or wherein the amino acid sequence of the one or more viral proteins or one or more fragments thereof of the parent SARS-CoV-2 coronavirus encoded by the polynucleotide comprises up to 20 amino acid substitutions, additions, or deletions.

In various embodiments, the parent SARS-CoV-2 coronavirus can be a wild-type SARS-CoV-2. In various embodiments, the parent SARS-CoV-2 coronavirus can be a natural isolate SARS-CoV-2. In various embodiments, the parent SARS-CoV-2 coronavirus can be Washington isolate of SARS-CoV-2 coronavirus having a nucleic acid sequence of GenBank accession no. MN985325.1. In various embodiments, the parent SARS-CoV-2 coronavirus can be BetaCoV/Wuhan/IVDC-HB-01/2019 isolate of SARS-CoV-2 coronavirus (SEQ ID NO: 1). In various embodiments, the parent SARS-CoV-2 coronavirus can be a SARS-CoV-2 variant. In various embodiments, the parent SARS-CoV-2 coronavirus can be a SARS-CoV-2 variant selected from the group consisting of U.K. variant, South Africa variant, and Brazil variant.

In various embodiments, the polynucleotide can be recoded by reducing codon-pair bias (CPB) or reducing codon usage bias compared to its parent SARS-CoV-2 coronavirus polynucleotide. In various embodiments, the polynucleotide can be recoded by increasing the number of CpG or UpA di-nucleotides compared to its parent SARS-CoV-2 coronavirus polynucleotide. In various embodiments, each of the recoded one or more viral proteins, or each of the recoded one or more fragments thereof can have a codon pair bias less than, −0.05, less than −0.1, less than −0.2, less than −0.3, or less than −0.4. In various embodiments, the polynucleotide can be CPB deoptimized compared to its parent SARS-CoV-2 coronavirus polynucleotide. In various embodiments, the polynucleotide can be codon deoptimized compared to its parent SARS-CoV-2 coronavirus polynucleotide.

In various embodiments, the codon-deoptimized or CPB deoptimized can be based on frequently used codons or CPB in humans. In various embodiments, the codon-deoptimized or CPB deoptimized can be based on frequently used codons or CPB in a coronavirus. In various embodiments, the codon-deoptimized or CPB deoptimized can be based on frequently used codons or CPB in a SARS-CoV-2 coronavirus. In various embodiments, the codon-deoptimized or CPB deoptimized can be based on frequently used codons or CPB in a wild-type SARS-CoV-2 coronavirus.

In various embodiments, the recoded nucleotide sequence can be selected from RNA-dependent RNA polymerase (RdRP), a fragment of RdRP, a spike protein, a fragment of spike protein, and combinations thereof.

In various embodiments, the polynucleotide can comprise at least one CPB deoptimized region can be selected from bp 11294-12709, bp 14641-15903, bp 21656-22306, bp 22505-23905, and bp 24110-25381 of SEQ ID NO:1 or SEQ ID NO:2.

In various embodiments, the polynucleotide can comprise a recoded spike protein or a fragment of spike protein wherein the furin cleavage site can be eliminated.

In various embodiments, the polynucleotide can comprise the nucleotide sequence of SEQ ID NO:4, nucleotides 1-29,834 of SEQ ID NO:4, SEQ ID NO:7, or nucleotides 1-29,834 of SEQ ID NO:7. In various embodiments, the polynucleotide can further comprise one or more consecutive adenines on the 3′ end.

In various embodiments, the polynucleotide can comprise the nucleotide sequence of SEQ ID NO:3.

Various embodiments of the present invention provide for a bacterial artificial chromosome (BAC) comprising any one of the recoded polynucleotides of the present invention.

Various embodiments of the present invention provide for a vector comprising any one of the recoded polynucleotides of the present invention.

Various embodiments of the present invention provide for a cell comprising any one of the recoded polynucleotides of the present invention, any one of the BAC of the present invention, or any one of the vectors of the present invention. In various embodiments, the cell can be Vero cell or baby hamster kidney (BHK) cell.

Various embodiments of the present invention provide for a polypeptide encoded by any one of the recoded polynucleotides of the present invention.

Various embodiments of the present invention provide for a modified SARS-CoV-2 coronavirus comprising any one of the recoded polynucleotides of the present invention.

Various embodiments of the present invention provide for a modified SARS-CoV-2 coronavirus comprising any one of the polypeptides of the present invention encoded by any one of the recoded polynucleotides of the present invention.

In various embodiments, wherein expression of one or more viral proteins in any one of the modified SARS-CoV-2 coronavirus of the present invention can be reduced compared to its parent SARS-CoV-2 coronavirus.

In various embodiments, the reduction in the expression of one or more of its viral proteins can be reduced as the result of recoding a region selected from RdRP, spike protein and combinations thereof.

In various embodiments, the modified SARS-CoV-2 coronavirus can comprise a polynucleotide having SEQ ID NO:4, or nucleotides 1-29,834 of SEQ ID NO:4, or nucleotides 1-29,834 of SEQ ID NO:4 and one or more consecutive adenines on the 3′ end.

In various embodiments, the modified SARS-CoV-2 coronavirus can comprise a polypeptide encoded by a polynucleotide having SEQ ID NO:4, or nucleotides 1-29,834 of SEQ ID NO:4, or nucleotides 1-29,834 of SEQ ID NO:4 and one or more consecutive adenines on the 3′ end.

Various embodiments of the present invention provide for a vaccine composition for inducing a protective an immune response in a subject, comprising: any one of the modified SARS-CoV-2 coronavirus of the present invention. In various embodiments, the vaccine composition can further comprise a pharmaceutically acceptable carrier or excipient.

Various embodiments of the present invention provide for an immune composition for eliciting an immune response in a subject, comprising: any one of the modified SARS-CoV-2 coronavirus of the present invention. In various embodiments, the immune composition can further comprise a pharmaceutically acceptable carrier or excipient.

Various embodiments of the present invention provide for a method of eliciting an immune response in a subject, comprising: administering to the subject a dose of: any one of the modified SARS-CoV-2 coronaviruses of the present invention, or any one of the vaccine compositions the present invention, or any one of the immune compositions of the present invention.

Various embodiments of the present invention provide for a method of eliciting an immune response in a subject, comprising: administering to the subject a prime dose of any one of the modified SARS-CoV-2 coronaviruses of the present invention, or any one of the vaccine compositions of the present invention, or any one of the immune compositions of the present invention; and administering to the subject one or more boost doses of any one of the modified SARS-CoV-2 coronaviruses of the present invention, or any one of the vaccine compositions of the present invention, or any one of the immune compositions of the present invention.

In various embodiments, the immune response is a protective immune response.

In various embodiments, the dose can be a prophylactically effective or therapeutically effective dose. In various embodiments, the dose can be about 10⁴-10⁶ PFU, or the prime dose can be about 10⁴-10⁶ PFU and the one or more boost dose can be about 10⁴-10⁶ PFU.

In various embodiments, administering can be via a nasal route. In various embodiments, administering can be via nasal drop. In various embodiments, administering can be via nasal spray.

Various embodiments of the present invention provide for a modified SARS-CoV-2 coronavirus of the present invention, a vaccine composition of the present invention, or an immune composition of the present invention for use in eliciting an immune response, or for therapeutic or prophylactic treatment of COVID-19.

Various embodiments of the present invention provide for a modified SARS-CoV-2 coronavirus of the present invention, a vaccine composition of the present invention, or an immune composition of the present invention for use in eliciting an immune response, or for therapeutic or prophylactic treatment of COVID-19, wherein the use comprises a prime dose of the modified SARS-CoV-2 coronavirus of the present invention, or the vaccine composition of the present invention, or the immune composition of the present invention, and one or more boost doses of the modified SARS-CoV-2 coronavirus of the present invention, or the vaccine composition of the present invention, or the immune composition of the present invention.

Various embodiments of the present invention provide for a use of modified SARS-CoV-2 coronavirus of the present invention, a vaccine composition of the present invention, or an immune composition of the present invention in the manufacture of a medicament for eliciting an immune response, or for therapeutic or prophylactic treatment of COVID-19.

Various embodiments of the present invention provide for a use of modified SARS-CoV-2 coronavirus of the present invention, a vaccine composition of the present invention, or an immune composition of the present invention in the manufacture of a medicament for use in eliciting an immune response, or for therapeutic or prophylactic treatment of COVID-19, wherein the medicament comprises a prime dose of the modified SARS-CoV-2 coronavirus of the present invention, or the vaccine composition of the present invention, or the immune composition of the present invention, and one or more boost doses of the modified SARS-CoV-2 coronavirus of the present invention, or the vaccine composition of the present invention, or the immune composition of the present invention.

The modified SARS-CoV-2 coronavirus of the present invention is any one of the modified SARS-CoV-2 coronavirus discussed herein. The vaccine composition of the present invention is any one of the vaccine compositions discussed herein. The immune composition of the present invention is any one of the immune compositions discussed herein. In various embodiments, the immune response is a protective immune response.

Various embodiments of the present invention provide for a method of making a modified SARS-CoV-2 coronavirus, comprising: obtaining a nucleotide sequence encoding one or more proteins of a parent SARS-CoV-2 coronavirus or one or more fragments thereof, recoding the nucleotide sequence to reduce protein expression of the one or more proteins, or the one or more fragments thereof, and substituting a nucleic acid having the recoded nucleotide sequence into the parent SARS-CoV-2 coronavirus genome to make the modified SARS-CoV-2 coronavirus genome, wherein expression of the recoded nucleotide sequence is reduced compared to the parent virus.

In various embodiments, the parent SARS-CoV-2 coronavirus sequence can be a wild-type (wt) viral nucleic acid, or a natural isolate.

In various embodiments, the modified SARS-CoV-2 coronavirus is any one of the modified SARS-CoV-2 coronavirus of the present invention.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 shows exemplary CoV Attenuation and Synthesis Strategy BAC Cloning/DNA Transfection in accordance with various embodiments of the present invention.

FIG. 2 shows exemplary CoV Attenuation and Synthesis Strategy In Vitro Ligation/RNA Transfection in accordance with various embodiments of the present invention.

FIG. 3 depicts plaque phenotype of wild-type (left) and CDX-005 (right) strains of SARS-CoV-2 on Vero E6 cells. CDX-005 produces smaller plaques and grows to 40% lower titers on Vero E6 cells as compared to wild-type virus.

FIG. 4 depicts body weight changes after dosing of wild-type SARS-COV-2 and CDX-005 in Syrian Gold hamsters.

FIG. 5 depicts Growth of wt WA1 and CDX-005 in Vero cells. Vero cells were infected with the 0.01 MOI of wt WA1 or CDX-005 and cultured for up to 96 hrs at 33° C. or 37° C. Supernatants were collected to recover virus. Titers were determined by plaque forming assays and reported as log of PFU/ml culture medium.

FIGS. 6a-6d depict in vivo attenuation of CDX-005 in hamsters. Hamsters were inoculated with 5×10⁴ or 5×10³ PFU/ml of wt WA1, 5×10⁴ PFU/ml CDX-005. Viral RNA was measured by qPCR at Days 2 and 4 PI in the 6a) olfactory bulb, 6b) brain, and 6c) lungs. (N=3/group; Bars=SEM). 6d) Infectious viral load in left lung tissue of inoculated hamsters was assessed by TCID₅₀ assay and expressed as log₁₀ of TCID₅₀/ml. Differences between CDX-005 and wt WA1 treated groups were significant (N=3/group; P<0.001; Bars=SEM). Horizontal lines indicate LOD.

FIGS. 7a-7c depict in vivo attenuation of CDX-005 in hamsters. Hamsters inoculated with 5×10⁴ or 5×10³ PFU/ml of wt WA1 or 5×10⁴ PFU/ml CDX-005. 7a) The weight of hamsters was measured daily for nine days. Weight changes were significantly different between CDX-005 and wt WA1 treated groups (N=10-40/group for CDX-005 and wt WA1 5×10⁴; N=3-12/group wt WA1 5×10³; P<0.001; Bars=SEM). 7b & 7c) Hematoxylin and eosin stained lung sections were examined on Days 2, 4, and 6 PI and scored for cell infiltration. (N=3/group)

FIGS. 8a-8d depicts efficacy in Hamsters. 8a) A Spike-S1 ELISA was performed with naïve hamster control serum or with serum collected from hamsters on Day 16 post-inoculation with wt WA1 or 5×10⁴ PFU COVI-VAC (CDX-005). Spike S1 IgG in COVI-VAC (CDX-005) inoculated hamsters was also measured on Day 18 (two days post WA1 challenge). The endpoint IgG titers are shown as the log of the dilution that was 5× above the background. (N=3/group; Bars=SEM) 8b) Plaque Reduction Neutralization Titers (PRNT) against SARS-CoV-2 WA1 were tested in serum of hamsters 16 days after inoculation with 5×10⁴ or 5×10³ PFU of wt WA1 or 5×10⁴ PFU COVI-VAC (CDX-005). The PRNT is the reciprocal of the last serum dilution that reduced plaque numbers 50, 80, or 90 percent relative to those in wells containing naïve hamster serum. (N=3/group; Bars=SD); 8c) CDX-005 vaccinated hamsters on Day 16 post-vaccination and naïve animals were challenged with 5×10⁴ PFU wt SARS-CoV-2. Lungs were harvested on Day 2 post-challenge and viral loads were measured by qPCR and expressed as log₁₀ of qPCR genomes/ml of tissue. (N=3/group; Bars=SD). 8d) Hamsters vaccinated with vehicle, 5×10⁴ PFU of wt WA1 or 5×10⁴ COVI-VAC (CDX-005) and challenged with 5×10⁴ PFU/ml wt WA1 intranasally 27 days post-inoculation. Weights were recorded on the day of challenge and daily for 4 days thereafter. (N=5-6 days 0-2, N=3 Days 3-4, Bars=SEM). The results in a) and b) are from two separate hamster studies.

FIG. 9 depicts attenuation in African Green Monkeys. Tracheal lavage fluid was collected from monkeys at Day 4 and Day 6 post-inoculation with 10⁶ PFU wt WA1 or CDX-005. Lavage fluid was subjected to RT-qPCR to detect virus. N=3/group (Day 4) or N=2/group (Day 6).

FIG. 10 depicts wt SARS-COV2 v. CDX-005 intranasal dose of 10⁶ in African Green Monkeys.

FIG. 11 depicts crude bulk titers of CDX-005 harvested from Vero cells. Vero WHO “10-87” cells were inoculated with 1.8×10⁴ PFU of CDX-005 (˜0.01 MOI) then grown for 48 hr. Virus was harvested using the different schemes shown.

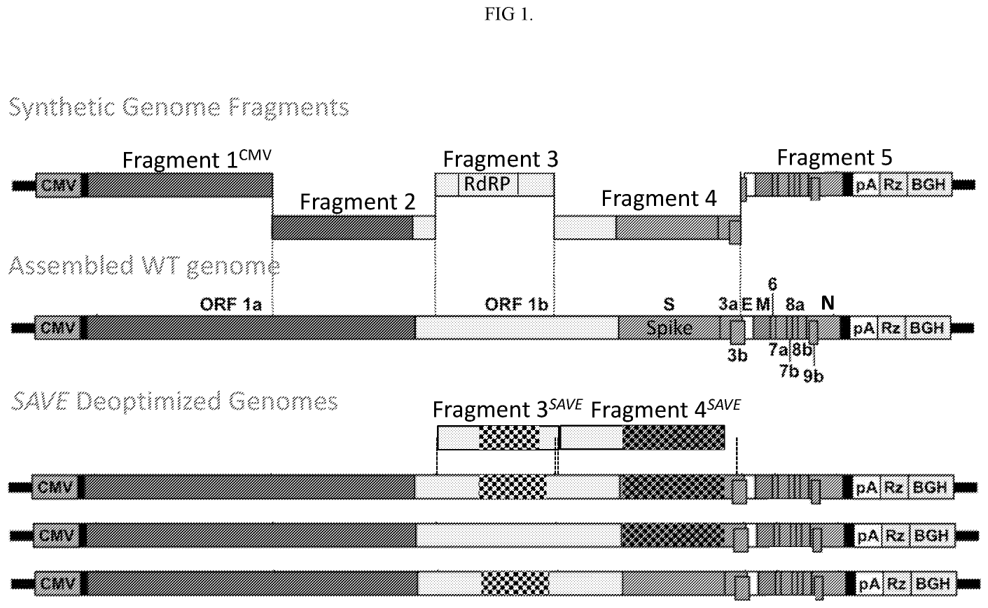

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, N.Y. 2006); March, Advanced Organic Chemistry Reactions, Mechanisms and Structure 7^{th} ed., J. Wiley & Sons (New York, N.Y. 2013); and Sambrook and Russel, Molecular Cloning: A Laboratory Manual 4^{th} ed., Cold Spring Harbor Laboratory Press (Cold Spring Harbor, N.Y. 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, or 0.5% of that referenced numeric indication, if specifically provided for in the claims.

“Parent virus” as used herein refer to a reference virus to which a recoded nucleotide sequence is compared for encoding the same or similar amino acid sequence.

“Wuhan coronavirus” and “SARS-CoV-2” and “2019-nCoV” as used herein are interchangeable, and refer to a coronavirus that has a wild-type sequence, natural isolate sequence, or mutant forms of the wild-type sequence or natural isolate sequence that causes COVID-19. Mutant forms arise naturally through the virus' replication cycles, or through genetic engineering.

“SARS-CoV-2 variant” as used herein refers to a mutant form of SARS-CoV-2 that has developed naturally through the virus' replication cycles as it replicates in and/or transmits between hosts such as humans. Examples of SARS-CoV-2 variants include but are not limited to U.K. variant (also known as 201/501Y.V1, VOC 202012/01, or B.1.1.7), South African variant (also known as 20H/501Y.V2 or B.1.351), and Brazil variant (also known as P.1).

“Natural isolate” as used herein with reference to SARS-CoV-2 refers to a virus such as SARS-CoV-2 that has been isolated from a host (e.g., human, bat, feline, pig, or any other host) or natural reservoir. The sequence of the natural isolate can be identical or have mutations that arose naturally through the virus' replication cycles as it replicates in and/or transmits between hosts, for example, humans.

“Wuhan coronavirus isolate” as used herein refers to a wild-type isolate of SARS-CoV-2 that has Accession ID: EPI_ISL_402119, submitted Jan. 10, 2020, and also referred to as BetaCoV/Wuhan/IVDC-HB-01/2019, SEQ ID NO: 1, which is herein incorporated by reference as though fully set forth in its entirety.

“Washington coronavirus isolate” as used herein refers to a wild-type isolate of SARS-CoV-2 that has GenBank accession no. MN985325.1 as of Jul. 5, 2020, which is herein incorporated by reference as though fully set forth in its entirety.

“Frequently used codons” or “codon usage bias” as used herein refer to differences in the frequency of occurrence of synonymous codons in coding DNA for a particular species, for example, human, coronavirus, or SARS-CoV-2.

“Codon pair bias” as used herein refers to synonymous codon pairs that are used more or less frequently than statistically predicted in a particular species, for example, human, coronavirus, or SARS-CoV-2.

A “subject” as used herein means any animal or artificially modified animal. Animals include, but are not limited to, humans, non-human primates, cows, horses, sheep, pigs, dogs, cats, rabbits, ferrets, rodents such as mice, rats and guinea pigs, bats, snakes, and birds. Artificially modified animals include, but are not limited to, SCID mice with human immune systems. In a preferred embodiment, the subject is a human.

A “viral host” means any animal or artificially modified animal that a virus can infect. Animals include, but are not limited to, humans, non-human primates, cows, horses, sheep, pigs, dogs, cats, rabbits, ferrets, rodents such as mice, rats and guinea pigs, and birds. Artificially modified animals include, but are not limited to, SCID mice with human immune systems. In various embodiments, the viral host is a mammal. In various embodiments, the viral host is a primate. In various embodiments, the viral host is human. Embodiments of birds are domesticated poultry species, including, but not limited to, chickens, turkeys, ducks, and geese.

A “prophylactically effective dose” is any amount of a vaccine or virus composition that, when administered to a subject prone to viral infection or prone to affliction with a virus-associated disorder, induces in the subject an immune response that protects the subject from becoming infected by the virus or afflicted with the disorder. “Protecting” the subject means either reducing the likelihood of the subject's becoming infected with the virus, or lessening the likelihood of the disorder's onset in the subject, by at least two-fold, preferably at least ten-fold, 25-fold, 50-fold, or 100 fold. For example, if a subject has a 1% chance of becoming infected with a virus, a two-fold reduction in the likelihood of the subject becoming infected with the virus would result in the subject having a 0.5% chance of becoming infected with the virus.

As used herein, a “therapeutically effective dose” is any amount of a vaccine or virus composition that, when administered to a subject afflicted with a disorder against which the vaccine is effective, induces in the subject an immune response that causes the subject to experience a reduction, remission or regression of the disorder and/or its symptoms. In preferred embodiments, recurrence of the disorder and/or its symptoms is prevented. In other preferred embodiments, the subject is cured of the disorder and/or its symptoms.

Certain embodiments of any of the instant immunization and therapeutic methods further comprise administering to the subject at least one adjuvant. An “adjuvant” shall mean any agent suitable for enhancing the immunogenicity of an antigen and boosting an immune response in a subject. Numerous adjuvants, including particulate adjuvants, suitable for use with both protein- and nucleic acid-based vaccines, and methods of combining adjuvants with antigens, are well known to those skilled in the art. Suitable adjuvants for nucleic acid based vaccines include, but are not limited to, Quil A, imiquimod, resiquimod, and interleukin-12 delivered in purified protein or nucleic acid form. Adjuvants suitable for use with protein immunization include, but are not limited to, alum, Freund's incomplete adjuvant (FIA), saponin, Quil A, and QS-21.

Described herein are SARS-CoV-2 viruses wherein its genes have been recoded, for example, codon deoptimized or codon pair bias deoptimized. In various embodiments, the viral proteins of SARS-CoV-2 viruses of the present invention have the same amino acid sequences as its parent SARS-CoV-2 virus; however, the nucleotide sequences have been recoded. Recoding of the nucleotide sequence in accordance with the present invention results in reduced protein expression, attenuation or both. These recoded SARS-CoV-2 viruses are useful as vaccines, and particularly, for use as live-attenuated vaccines.

We generated a synthetic highly attenuated live vaccine candidate, COVI-VAC (also referred to as CDX-005; e.g., SEQ ID NO:4)) from wt SARS-CoV-2. While not wishing to be bound by any particular theory, we believe that the most likely mechanism for the attenuation is slowed translation, through errors in translation leading to misfolded proteins, changes in RNA secondary structure, or altered regulatory signals may all contribute to reduced protein production. Whatever the mechanism, the attenuated COVI-VAC virus presents every viral antigen in its wt form, providing the potential for a broad immune response and making it likely to retain efficacy even if there is genetic drift in the target strain. COVI-VAC is expected to be highly resistant to reversion to pathogenicity since hundreds of silent (synonymous) mutations contribute to the phenotype. Our tests of reversion indicate that the vaccine is stable as assessed by bulk sequencing of late passage virus and evaluation of potential changes in the furin cleavage site.

Our hamster studies demonstrate that COVI-VAC is safe in these animals. It is highly attenuated, inducing lower total viral loads in the lungs and olfactory bulb and completely abrogating it in the brain and inducing lower live viral loads in the lung of animals inoculated with COVI-VAC than those with wt WA1. Unlike wt virus, COVI-VAC did not induce weight loss or significant lung pathology in inoculated hamsters.

The hamster studies also suggest that COVI-VAC effectively protect against SARS CoV-2. Assessment of Abs titers demonstrate that it is as effective as wt virus in inducing serum IgG and neutralizing Abs. It is protective against wt challenge; inoculation with COVI-VAC leads to lower lung viral titers and complete protection against virus in the brain. Hamsters inoculated with COVI-VAC also do not exhibit the weight loss observed in vehicle inoculated animals. Moreover, there is no evidence of disease enhancement.

Together our data indicates that COVI-VAC is a part of an important new class of live attenuated vaccines currently being developed for use in animals and humans. It presents all viral antigens similar to their native amino acid sequence, can be administered intranasally, is safe and effective in small animal models with a single dose, is resistant to reversion, and can be grown to high titers at a permissive temperature. Clinical trials are currently underway to test its safety and efficacy in humans.

To construct the deoptimized CDX-005 (e.g., SEQ ID NO:4) and CDX-007 (e.g., SEQ ID NO:7) live attenuated vaccine candidates, first the genome of the wild-type WA1 donor virus was parsed in silico into 19 overlapping fragments. Each fragment shares approximately 200 bp of sequence overlap with each adjacent fragment. F1-F19 were generated from cDNA of wild-type WA1 virus RNA by RT-PCR. The fragments were sequence confirmed by Sanger sequencing. We then exchanged Fragment 16 of the WT WA1 virus for fragment 16 that had the deoptimized spike gene sequence to generate the cDNA genome of CDX-005. Similarly, we exchanged Fragment 14 of the WT WA1 virus for fragment 14 that had the deoptimized spike gene sequence to generate the cDNA genome of CDX-007.

In various embodiments, the molecular parsing of a target Parent virus into small fragments each with about 50 to 300 bp overlaps via RT-PCR and the exchange of any of these fragments is a process that can be used to construct the cDNA genome or genome fragment of any codon-, or codon-pair-deoptimized virus. This cDNA genome with the deoptimized cassette can then be used to recover a deoptimized virus via reverse genetics.

For CDX-005 and CDX-007, w identified one notable difference in the sequence of our WA1 donor virus (Vero cell passage 6) compared to the published WA1 sequence (Vero cell passage 4). During the two additional WA1 virus passages on Vero E6 cells at Codagenix of the WA1 virus received from BEI Resources, a 36 nt deletion occurred in the Spike gene (genome position 23594-23629). The deletion encompasses the 12 amino acids TNSPRRARSVAS (SEQ ID NO:8) that include the polybasic furin cleavage site. The furin cleavage site in SARS-CoV2 Spike has been proposed as a potential driver of the highly pathogenic phenotype of SARS-CoV2 in the human host. While not wishing to be bound by any particular theory, we believe that absence of the furin cleavage is beneficial to the SARS-CoV-2 virus growth in vitro in Vero cells, and that the deletion evolved during passaging in Vero cell culture. We further believe that the absence of the furin cleavage site may contribute to attenuation in the human host of a SARS-CoV-2 virus carrying such mutation. We therefore decided to incorporate the furin cleavage site deletion that was derived into our vaccine candidates CDX-005, and CDX-007. The furin cleavage site deletion is located in assembly fragment F15.

The present invention is based, at least in part, on the foregoing and on the further information as described herein.

In various embodiments, the viral proteins of SARS-CoV-2 viruses of the present invention have the same amino acid sequences as its parent SARS-CoV-2 virus but with up to about 20 amino acid deletion(s), substitution(s), or addition(s). However, the nucleotide sequences have been recoded, which results in reduced protein expression, attenuation or both. In various embodiments, the viral proteins of SARS-CoV-2 viruses of the present invention have the same amino acid sequences as its parent SARS-CoV-2 virus but with up to 10 amino acid deletions, substitutions, or additions; however, the nucleotide sequences have been recoded, which results in reduced protein expression, attenuation or both. In various embodiments, the viral proteins of SARS-CoV-2 viruses of the present invention have the same amino acid sequences as its parent SARS-CoV-2 virus but between 1-5 amino acid deletion, substitution, or addition. In various embodiments, the viral proteins of SARS-CoV-2 viruses of the present invention have the same amino acid sequences as its parent SARS-CoV-2 virus but between 6-10 amino acid deletion, substitution, or addition. In various embodiments, the viral proteins of SARS-CoV-2 viruses of the present invention have the same amino acid sequences as its parent SARS-CoV-2 virus but between 11-15 amino acid deletion, substitution, or addition. In various embodiments, the viral proteins of SARS-CoV-2 viruses of the present invention have the same amino acid sequences as its parent SARS-CoV-2 virus but between 16-20 amino acid deletion, substitution, or addition. Again, however, the nucleotide sequences have been recoded, which results in reduced protein expression, attenuation or both. In various embodiments, the viral proteins of SARS-CoV-2 viruses of the present invention have the same amino acid sequences as its parent SARS-CoV-2 virus but 12 amino acid deletions, substitutions, or additions; however, the nucleotide sequences have been recoded, which results in reduced protein expression, attenuation or both. In various embodiments, the amino acid deletion, substitution, or addition results from nucleic acid deletion(s), substitution(s) or addition(s) before the polyA tail of the nucleic acid sequence of the parent SARS-CoV-2 virus sequence.

In various embodiments, the viral proteins of SARS-CoV-2 viruses of the present invention have the same amino acid sequences as its parent SARS-CoV-2 virus but with a 12 amino acid deletion. In various embodiments, the viral proteins of SARS-CoV-2 viruses of the present invention have the same amino acid sequences as its parent SARS-CoV-2 virus but with a 1-5 amino acid deletion, or a 6-10 amino acid deletion, or a 11-15 amino acid deletion, or a 16-20 amino acid deletion. In various embodiments, the amino acid deletion is in the Spike protein that eliminates the furin cleavage site. In various particular embodiments, the viral proteins of SARS-CoV-2 viruses of the present invention have the same amino acid sequences as its parent SARS-CoV-2 virus but with a 12 amino acid deletion that results in the elimination of the furin cleavage site on the Spike protein. In various embodiments, the amino acid deletion, substitution, or addition results from nucleic acid deletion(s), substitution(s) or addition(s) before the polyA tail of the nucleic acid sequence of the parent SARS-CoV-2 virus sequence.

In various embodiments, the nucleic acid encoding the RNA-dependent RNA polymerase (RdRP) protein of the SARS-CoV-2 virus is recoded. In other embodiments, the nucleic acid encoding the spike protein (also known as S gene) of the SARS-CoV-2 virus is recoded. In still other embodiments, both the RdRP and the spike proteins of the SARS-CoV-2 virus are recoded. In various embodiments, the recoded spike protein comprises a deletion of nucleotides that eliminates the furin cleavage site; for example, a 36 nucleotide sequence having SEQ ID NO:5.

The recoding of RdRP and/or spike protein encoding sequences of the attenuated viruses of the invention have been made or can be made by one of skill in the art in light of disclosure discussed herein. According to various embodiments of the invention, nucleotide substitutions are engineered in multiple locations in the RdRP and/or spike protein coding sequence, wherein the substitutions introduce a plurality of synonymous codons into the genome. In certain embodiments, the synonymous codon substitutions alter codon bias, codon pair bias, the density of infrequent codons or infrequently occurring codon pairs, RNA secondary structure, CG and/or TA (or UA) dinucleotide content, C+G content, translation frameshift sites, translation pause sites, the presence or absence of microRNA recognition sequences or any combination thereof, in the genome. The codon substitutions may be engineered in multiple locations distributed throughout the RdRP and/or spike protein coding sequence, or in the multiple locations restricted to a portion of the RdRP and/or spike protein coding sequence. Because of the large number of defects (i.e., nucleotide substitutions) involved, the invention allows for production of stably attenuated viruses and live vaccines.

As discussed further below, in some embodiments, a virus coding sequence is recoded by substituting one or more codon with synonymous codons used less frequently in the SARS-CoV-2 coronavirus host (e.g., humans, snakes, bats). In some embodiments, a virus coding sequence is recoded by substituting one or more codons with synonymous codons used less frequently in a coronavirus; for example, the SARS-CoV-2 coronavirus. In certain embodiments, the number of codons substituted with synonymous codons is at least 5. In some embodiments, at least 10, 20, 30, 40, 50, 60, 70, 80, 90, 100, 200, 250, 300, 350, 400, 450, or 500 codons are substituted with synonymous codons less frequently used in the host. In certain embodiments, the modified sequence comprises at least 20 codons substituted with synonymous codons less frequently used. In certain embodiments, the modified sequence comprises at least 50 codons substituted with synonymous codons less frequently used. In certain embodiments, the modified sequence comprises at least 100 codons substituted with synonymous codons less frequently used. In certain embodiments, the modified sequence comprises at least 250 codons substituted with synonymous codons less frequently used. In certain embodiments, the modified sequence comprises at least 500 codons substituted with synonymous codons less frequently used.

For example, for the recoded spike protein, the number of codons substituted with synonymous codons less frequently used in the host is at least 5, 10, 20, 30, 40, 50, 60, 70, 80, 90, 100, 150, 200, 250, 300, 350, 400, 450, or 500 codons.

For example, for the recoded RdRP protein, the number of codons substituted with synonymous codons less frequently used in the host is at least 5, 10, 20, 30, 40, 50, 60, 70, 80, 90, 100, 150, 200, 250, or 300 codons.

In some embodiments, the substitution of synonymous codons is with those that are less frequent in the viral host; for example, human. Other examples of viral hosts include but are not limited to those noted above. In some embodiments, the substitution of synonymous codons is with those that are less frequent in the virus itself, for example, the SARS-CoV-2 coronavirus.

In embodiments wherein the modified sequence comprises an increased number of CpG or UpA di-nucleotides compared to a corresponding sequence on the parent virus, the increase is of about 15-55 CpG or UpA di-nucleotides compared the corresponding sequence. In various embodiments, increase is of about 15, 20, 25, 30, 35, 40, 45, or 55 CpG or UpA di-nucleotides compared the corresponding sequence. In some embodiments, the increased number of CpG or UpA di-nucleotides compared to a corresponding sequence is about 10-75, 15-25, 25-50, or 50-75 CpG or UpA di-nucleotides compared the corresponding sequence.

In some embodiments, virus codon pairs are recoded to reduce (i.e., lower the value of) codon-pair bias. In certain embodiments, codon-pair bias is reduced by identifying a codon pair in an RdRP and/or spike coding sequence having a codon-pair score that can be reduced and reducing the codon-pair bias by substituting the codon pair with a codon pair that has a lower codon-pair score. In some embodiments, this substitution of codon pairs takes the form of rearranging existing codons of a sequence. In some such embodiments, a subset of codon pairs is substituted by rearranging a subset of synonymous codons. In other embodiments, codon pairs are substituted by maximizing the number of rearranged synonymous codons. It is noted that while rearrangement of codons leads to codon-pair bias that is reduced (made more negative) for the virus coding sequence overall, and the rearrangement results in a decreased CPS at many locations, there may be accompanying CPS increases at other locations, but on average, the codon pair scores, and thus the CPB of the modified sequence, is reduced. In some embodiments, recoding of codons or codon-pairs can take into account altering the G+C content of the RdRP and/or spike coding sequence. In some embodiments, recoding of codons or codon-pairs can take into account altering the frequency of CG and/or TA dinucleotides in the RdRP and/or spike coding sequence.

In certain embodiments, the recoded RdRP and/or spike protein-encoding sequence has a codon pair bias less than −0.1, or less than −0.2, or less than −0.3, or less than −0.4. In some embodiments, the recoded RdRP and/or spike protein-encoding sequence has a codon pair bias less than −0.05, or less than −0.06, or less than −0.07, or less than −0.08, or less than −0.09, or less than −0.1, or less than −0.11, or less than −0.12, or less than −0.13, or less than −0.14, or less than −0.15, or less than −0.16, or less than −0.17, or less than −0.18, or less than −0.19, or less than −0.2, or less than −0.25, or less than −0.3, or less than −0.35, or less than −0.4, or less than −0.45, or less than −0.5.

In certain embodiments, the codon pair bias of the recoded RdRP and/or spike protein encoding sequence is reduced by at least 0.1, or at least 0.2, or at least 0.3, or at least 0.4, compared to the parent RdRP and/or spike protein encoding sequence from which it is derived (e.g., the parent sequence RdRP and/or spike protein encoding sequence, the wild-type sequence RdRP and/or spike protein encoding sequence). In certain embodiments, rearrangement of synonymous codons of the RdRP and/or spike protein-encoding sequence provides a codon-pair bias reduction of at least 0.1, or at least 0.2, or at least 0.3, or at least 0.4, compared to the parent RdRP and/or spike protein encoding sequence from which it is derived. In certain embodiments, the codon pair bias of the recoded the RdRP and/or spike protein-encoding sequence is reduced by at least 0.05, or at least 0.06, or at least 0.07, or at least 0.08, or at least 0.09, or at least 0.1, or at least 0.11, or at least 0.12, or at least 0.13, or at least 0.14, or at least 0.15, or at least 0.16, or at least 0.17, or at least 0.18, or at least 0.19, or at least 0.2, or at least 0.25, or at least 0.3, or at least 0.35, or at least 0.4, or at least 0.45, or at least 0.5, compared to the corresponding sequence on the parent virus. In certain embodiments, it is in comparison corresponding sequence from which the calculation is to be made; for example, the corresponding sequence of a wild-type virus (e.g., RdRP and/or spike protein-encoding sequence on wild-type virus).

Usually, these substitutions and alterations are made and reduce expression of the encoded virus proteins without altering the amino acid sequence of the encoded protein. In certain embodiments, the invention also includes alterations in the RdRP and/or spike coding sequence that result in substitution of non-synonymous codons and amino acid substitutions in the encoded protein, which may or may not be conservative. In some embodiments, these substitutions and alterations further include substitutions or alterations that results in amino acid deletions, additions, substitutions. For example, the spike protein can be recoded with a 36 nucleotide deletion that results in the elimination of the furin cleavage site.

Most amino acids are encoded by more than one codon. See the genetic code in Table 1. For instance, alanine is encoded by GCU, GCC, GCA, and GCG. Three amino acids (Leu, Ser, and Arg) are encoded by six different codons, while only Trp and Met have unique codons. “Synonymous” codons are codons that encode the same amino acid. Thus, for example, CUU, CUC, CUA, CUG, UUA, and UUG are synonymous codons that code for Leu. Synonymous codons are not used with equal frequency. In general, the most frequently used codons in a particular organism are those for which the cognate tRNA is abundant, and the use of these codons enhances the rate and/or accuracy of protein translation. Conversely, tRNAs for the rarely used codons are found at relatively low levels, and the use of rare codons is thought to reduce translation rate and/or accuracy.

TABLE 1
Genetic Code

| | U | C | A | G | |
|---|---|---|---|---|---|
| U | Phe | Ser | Tyr | Cys | U |
| | Phe | Ser | Tyr | Cys | C |
| | Leu | Ser | STOP | STOP | A |
| | Leu | Ser | STOP | Trp | G |
| C | Leu | Pro | His | Arg | U |
| | Leu | Pro | His | Arg | C |
| | Leu | Pro | Gln | Arg | A |
| | Leu | Pro | Gln | Arg | G |
| A | Ile | Thr | Asn | Ser | U |
| | Ile | Thr | Asn | Ser | C |
| | Ile | Thr | Lys | Arg | A |
| | Met | Thr | Lys | Arg | G |
| G | Val | Ala | Asp | Gly | U |
| | Val | Ala | Asp | Gly | C |
| | Val | Ala | Glu | Gly | A |
| | Val | Ala | Glu | Gly | G |

^{a} The first nucleotide in each codon encoding a particular amino acid is shown in the left-most column; the sec ond nucleotide is shown in the top row; and the third nucleotide is shown in the right-most column.

### Codon Bias

As used herein, a “rare” codon is one of at least two synonymous codons encoding a particular amino acid that is present in an mRNA at a significantly lower frequency than the most frequently used codon for that amino acid. Thus, the rare codon may be present at about a 2-fold lower frequency than the most frequently used codon. Preferably, the rare codon is present at least a 3-fold, more preferably at least a 5-fold, lower frequency than the most frequently used codon for the amino acid. Conversely, a “frequent” codon is one of at least two synonymous codons encoding a particular amino acid that is present in an mRNA at a significantly higher frequency than the least frequently used codon for that amino acid. The frequent codon may be present at about a 2-fold, preferably at least a 3-fold, more preferably at least a 5-fold, higher frequency than the least frequently used codon for the amino acid. For example, human genes use the leucine codon CTG 40% of the time, but use the synonymous CTA only 7% of the time (see Table 2). Thus, CTG is a frequent codon, whereas CTA is a rare codon. Roughly consistent with these frequencies of usage, there are 6 copies in the genome for the gene for the tRNA recognizing CTG, whereas there are only 2 copies of the gene for the tRNA recognizing CTA. Similarly, human genes use the frequent codons TCT and TCC for serine 18% and 22% of the time, respectively, but the rare codon TCG only 5% of the time. TCT and TCC are read, via wobble, by the same tRNA, which has 10 copies of its gene in the genome, while TCG is read by a tRNA with only 4 copies. It is well known that those mRNAs that are very actively translated are strongly biased to use only the most frequent codons. This includes genes for ribosomal proteins and glycolytic enzymes. On the other hand, mRNAs for relatively non-abundant proteins may use the rare codons.

TABLE 2
Codon usage in Homo sapiens (source: www.kazusa.or.jp/codon/″ www.kazusa.or.jp/codon/)

| Amino | | | | | Amino | | | | |
|---|---|---|---|---|---|---|---|---|---|
| Acid | Codon | Number | /1000 | Fraction | Acid | Codon | Number | /1000 | Fraction |
| Gly | GGG | 636457.00 | 16.45 | 0.25 | Trp | TGG | 510256.00 | 13.19 | 1.00 |
| Gly | GGA | 637120.00 | 16.47 | 0.25 | End | TGA | 59528.00 | 1.54 | 0.47 |
| Gly | GGT | 416131.00 | 10.76 | 0.16 | Cys | TGT | 407020.00 | 10.52 | 0.45 |
| Gly | GGC | 862557.00 | 22.29 | 0.34 | Cys | TGC | 487907.00 | 12.61 | 0.55 |
| Glu | GAG | 1532589.00 | 39.61 | 0.58 | End | TAG | 30104.00 | 0.78 | 0.24 |
| Glu | GAA | 1116000.00 | 28.84 | 0.42 | End | TAA | 38222.00 | 0.99 | 0.30 |
| Asp | GAT | 842504.00 | 21.78 | 0.46 | Tyr | TAT | 470083.00 | 12.15 | 0.44 |
| Asp | GAC | 973377.00 | 25.16 | 0.54 | Tyr | TAC | 592163.00 | 15.30 | 0.56 |
| Val | GTG | 1091853.00 | 28.22 | 0.46 | Leu | TTG | 498920.00 | 12.89 | 0.13 |
| Val | GTA | 273515.00 | 7.07 | 0.12 | Leu | TTA | 294684.00 | 7.62 | 0.08 |
| Val | GTT | 426252.00 | 11.02 | 0.18 | Phe | TTT | 676381.00 | 17.48 | 0.46 |
| Val | GTC | 562086.00 | 14.53 | 0.24 | Phe | TTC | 789374.00 | 20.40 | 0.54 |
| Ala | GCG | 286975.00 | 7.42 | 0.11 | Ser | TCG | 171428.00 | 4.43 | 0.05 |
| Ala | GCA | 614754.00 | 15.89 | 0.23 | Ser | TCA | 471469.00 | 12.19 | 0.15 |
| Ala | GCT | 715079.00 | 18.48 | 0.27 | Ser | TCT | 585967.00 | 15.14 | 0.19 |
| Ala | GCC | 1079491.00 | 27.90 | 0.40 | Ser | TCC | 684663.00 | 17.70 | 0.22 |
| Arg | AGG | 461676.00 | 11.93 | 0.21 | Arg | CGG | 443753.00 | 11.47 | 0.20 |
| Arg | AGA | 466435.00 | 12.06 | 0.21 | Arg | CGA | 239573.00 | 6.19 | 0.11 |
| Ser | AGT | 469641.00 | 12.14 | 0.15 | Arg | CGT | 176691.00 | 4.57 | 0.08 |
| Ser | AGC | 753597.00 | 19.48 | 0.24 | Arg | CGC | 405748.00 | 10.49 | 0.18 |
| Lys | AAG | 1236148.00 | 31.95 | 0.57 | Gln | CAG | 1323614.00 | 34.21 | 0.74 |
| Lys | AAA | 940312.00 | 24.30 | 0.43 | Gln | CAA | 473648.00 | 12.24 | 0.26 |
| Asn | AAT | 653566.00 | 16.89 | 0.47 | His | CAT | 419726.00 | 10.85 | 0.42 |
| Asn | AAC | 739007.00 | 19.10 | 0.53 | His | CAC | 583620.00 | 15.08 | 0.58 |
| Met | ATG | 853648.00 | 22.06 | 1.00 | Leu | CTG | 1539118.00 | 39.78 | 0.40 |
| Ile | ATA | 288118.00 | 7.45 | 0.17 | Leu | CTA | 276799.00 | 7.15 | 0.07 |
| Ile | ATT | 615699.00 | 15.91 | 0.36 | Leu | CTT | 508151.00 | 13.13 | 0.13 |
| Ile | ATC | 808306.00 | 20.89 | 0.47 | Leu | CTC | 759527.00 | 19.63 | 0.20 |
| Thr | ACG | 234532.00 | 6.06 | 0.11 | Pro | CCG | 268884.00 | 6.95 | 0.11 |
| Thr | ACA | 580580.00 | 15.01 | 0.28 | Pro | CCA | 653281.00 | 16.88 | 0.28 |
| Thr | ACT | 506277.00 | 13.09 | 0.25 | Pro | CCT | 676401.00 | 17.48 | 0.29 |
| Thr | ACC | 732313.00 | 18.93 | 0.36 | Pro | CCC | 767793.00 | 19.84 | 0.32 |

The propensity for highly expressed genes to use frequent codons is called “codon bias.” A gene for a ribosomal protein might use only the 20 to 25 most frequent of the 61 codons, and have a high codon bias (a codon bias close to 1), while a poorly expressed gene might use all 61 codons, and have little or no codon bias (a codon bias close to 0). It is thought that the frequently used codons are codons where larger amounts of the cognate tRNA are expressed, and that use of these codons allows translation to proceed more rapidly, or more accurately, or both.

### Codon Pair Bias

In addition, a given organism has a preference for the nearest codon neighbor of a given codon A, referred to a bias in codon pair utilization. A change of codon pair bias, without changing the existing codons, can influence the rate of protein synthesis and production of a protein.

Codon pair bias may be illustrated by considering the amino acid pair Ala-Glu, which can be encoded by 8 different codon pairs. If no factors other than the frequency of each individual codon (as shown in Table 2) are responsible for the frequency of the codon pair, the expected frequency of each of the 8 encodings can be calculated by multiplying the frequencies of the two relevant codons. For example, by this calculation the codon pair GCA-GAA would be expected to occur at a frequency of 0.097 out of all Ala-Glu coding pairs (0.23×0.42; based on the frequencies in Table 2). In order to relate the expected (hypothetical) frequency of each codon pair to the actually observed frequency in the human genome the Consensus CDS (CCDS) database of consistently annotated human coding regions, containing a total of 14,795 human genes, was used. This set of genes is the most comprehensive representation of human coding sequences. Using this set of genes, the frequencies of codon usage were re-calculated by dividing the number of occurrences of a codon by the number of all synonymous codons coding for the same amino acid. As expected the frequencies correlated closely with previously published ones such as the ones given in Table 2. Slight frequency variations are possibly due to an oversampling effect in the data provided by the codon usage database at Kazusa DNA Research Institute (www.kazusa.or.jp/codon/codon.html) where 84949 human coding sequences were included in the calculation (far more than the actual number of human genes). The codon frequencies thus calculated were then used to calculate the expected codon-pair frequencies by first multiplying the frequencies of the two relevant codons with each other (see Table 3 expected frequency), and then multiplying this result with the observed frequency (in the entire CCDS data set) with which the amino acid pair encoded by the codon pair in question occurs. In the example of codon pair GCA-GAA, this second calculation gives an expected frequency of 0.098 (compared to 0.097 in the first calculation using the Kazusa dataset). Finally, the actual codon pair frequencies as observed in a set of 14,795 human genes was determined by counting the total number of occurrences of each codon pair in the set and dividing it by the number of all synonymous coding pairs in the set coding for the same amino acid pair (Table 3; observed frequency). Frequency and observed/expected values for the complete set of 3721 (61²) codon pairs, based on the set of 14,795 human genes, are provided herewith as Table 3.

TABLE 3
Codon Pair Scores Exemplified by the Amino Pair
Ala-Glu

| amino | | | | |
|---|---|---|---|---|
| acid | codon | expected | observed | obs/exp |
| pair | pair | frequency | frequency | ratio |
| AE | GCAGAA | 0.098 | 0.163 | 1.65 |
| AE | GCAGAG | 0.132 | 0.198 | 1.51 |
| AE | GCCGAA | 0.171 | 0.031 | 0.18 |
| AE | GCCGAG | 0.229 | 0.142 | 0.62 |
| AE | GCGGAA | 0.046 | 0.027 | 0.57 |
| AE | GCGGAG | 0.062 | 0.089 | 1.44 |
| AE | GCTGAA | 0.112 | 0.145 | 1.29 |
| AE | GCTGAG | 0.150 | 0.206 | 1.37 |
| Total | | 1.000 | 1.000 | |

If the ratio of observed frequency/expected frequency of the codon pair is greater than one the codon pair is said to be overrepresented. If the ratio is smaller than one, it is said to be underrepresented. In the example, the codon pair GCA-GAA is overrepresented 1.65 fold while the coding pair GCC-GAA is more than 5-fold underrepresented.

Many other codon pairs show very strong bias; some pairs are under-represented, while other pairs are over-represented. For instance, the codon pairs GCCGAA (AlaGlu) and GATCTG (AspLeu) are three- to six-fold under-represented (the preferred pairs being GCAGAG and GACCTG, respectively), while the codon pairs GCCAAG (AlaLys) and AATGAA (AsnGlu) are about two-fold over-represented. It is noteworthy that codon pair bias has nothing to do with the frequency of pairs of amino acids, nor with the frequency of individual codons. For instance, the under-represented pair GATCTG (AspLeu) happens to use the most frequent Leu codon, (CTG).

As discussed more fully below, codon pair bias takes into account the score for each codon pair in a coding sequence averaged over the entire length of the coding sequence. According to the invention, codon pair bias is determined by

$$CPB = {\sum }_{i = 1}^{k} \frac{CPSi}{K - 1}$$

Accordingly, similar codon pair bias for a coding sequence can be obtained, for example, by minimized codon pair scores over a subsequence or moderately diminished codon pair scores over the full length of the coding sequence.

### Calculation of Codon Pair Bias

Every individual codon pair of the possible 3721 non-“STOP” containing codon pairs (e.g., GTT-GCT) carries an assigned “codon pair score,” or “CPS” that is specific for a given “training set” of genes. The CPS of a given codon pair is defined as the log ratio of the observed number of occurrences over the number that would have been expected in this set of genes (in this example the human genome). Determining the actual number of occurrences of a particular codon pair (or in other words the likelihood of a particular amino acid pair being encoded by a particular codon pair) is simply a matter of counting the actual number of occurrences of a codon pair in a particular set of coding sequences. Determining the expected number, however, requires additional calculations. The expected number is calculated so as to be independent of both amino acid frequency and codon bias similarly to Gutman and Hatfield. That is, the expected frequency is calculated based on the relative proportion of the number of times an amino acid is encoded by a specific codon. A positive CPS value signifies that the given codon pair is statistically over-represented, and a negative CPS indicates the pair is statistically under-represented in the human genome.

To perform these calculations within the human context, the most recent Consensus CDS (CCDS) database of consistently annotated human coding regions, containing a total of 14,795 genes, was used. This data set provided codon and codon pair, and thus amino acid and amino-acid pair frequencies on a genomic scale.

The paradigm of Federov et al. (2002), was used to further enhanced the approach of Gutman and Hatfield (1989). This allowed calculation of the expected frequency of a given codon pair independent of codon frequency and non-random associations of neighboring codons encoding a particular amino acid pair. The detailed equations used to calculate CPB are disclosed in WO 2008/121992 and WO 2011/044561, which are incorporated by reference.

$$S ( {P}_{ij} ) = ln ( \frac{{N}_{O} ( {P}_{ij} )}{{N}_{E} ( {P}_{ij} )} ) = ln ( \frac{{N}_{O} ( {P}_{ij} )}{F ( {C}_{i} ) F ( {C}_{j} ) {N}_{O} ( {X}_{ij} )} )$$

In the calculation, Pᵢⱼ is a codon pair occurring with a frequency of N_{O}(Pᵢⱼ) in its synonymous group. Cᵢ and Cⱼ are the two codons comprising Pᵢⱼ, occurring with frequencies F(Cᵢ) and F(Cⱼ) in their synonymous groups respectively. More explicitly, F(Cᵢ) is the frequency that corresponding amino acid Xᵢ is coded by codon Cᵢ throughout all coding regions and F(Cᵢ)═N_{O}(Cⱼ)/N_{O}(Xᵢ), where N_{O}(Cᵢ) and N_{O}(Xᵢ) are the observed number of occurrences of codon Cᵢ and amino acid Xᵢ respectively. F(Cⱼ) is calculated accordingly. Further, N_{O}(Xᵢⱼ) is the number of occurrences of amino acid pair Xᵢⱼ throughout all coding regions. The codon pair bias score S(Pᵢⱼ) of Pᵢⱼ was calculated as the log-odds ratio of the observed frequency N_{O}(Pᵢⱼ) over the expected number of occurrences of Nₑ(Pᵢⱼ).

Using the formula above, it was then determined whether individual codon pairs in individual coding sequences are over- or under-represented when compared to the corresponding genomic Nₑ(Pᵢⱼ) values that were calculated by using the entire human CCDS data set. This calculation resulted in positive S(Pᵢⱼ) score values for over-represented and negative values for under-represented codon pairs in the human coding regions.

The “combined” codon pair bias of an individual coding sequence was calculated by averaging all codon pair scores according to the following formula:

$$S ( {P}_{ij} ) = {\sum }_{l = 1}^{k} \frac{S ( {P}_{ij} ) l}{k - 1}$$

The codon pair bias of an entire coding region is thus calculated by adding all of the individual codon pair scores comprising the region and dividing this sum by the length of the coding sequence.

### Calculation of Codon Pair Bias, Implementation of Algorithm to Alter Codon-Pair Bias.

An algorithm was developed to quantify codon pair bias. Every possible individual codon pair was given a “codon pair score”, or “CPS”. CPS is defined as the natural log of the ratio of the observed over the expected number of occurrences of each codon pair over all human coding regions, where humans represent the host species of the instant vaccine virus to be recoded.

$$CPS = ln ( \frac{{F ( AB )}_{O}}{\frac{F ( A ) \times F ( B )}{F ( X ) \times F ( Y )} \times F ( XY )} )$$

Although the calculation of the observed occurrences of a particular codon pair is straightforward (the actual count within the gene set), the expected number of occurrences of a codon pair requires additional calculation. We calculate this expected number to be independent both of amino acid frequency and of codon bias, similar to Gutman and Hatfield. That is, the expected frequency is calculated based on the relative proportion of the number of times an amino acid is encoded by a specific codon. A positive CPS value signifies that the given codon pair is statistically over-represented, and a negative CPS indicates the pair is statistically under-represented in the human genome.

Using these calculated CPSs, any coding region can then be rated as using over- or under-represented codon pairs by taking the average of the codon pair scores, thus giving a Codon Pair Bias (CPB) for the entire gene.

$$CPB = {\sum }_{i = 1}^{k} \frac{CPSi}{k - 1}$$

The CPB has been calculated for all annotated human genes using the equations shown and plotted. Each point in the graph corresponds to the CPB of a single human gene. The peak of the distribution has a positive codon pair bias of 0.07, which is the mean score for all annotated human genes. Also, there are very few genes with a negative codon pair bias. Equations established to define and calculate CPB were then used to manipulate this bias.

### Algorithm for Reducing Codon-Pair Bias.

Recoding of protein-encoding sequences may be performed with or without the aid of a computer, using, for example, a gradient descent, or simulated annealing, or other minimization routine. An example of the procedure that rearranges codons present in a starting sequence can be represented by the following steps:
1) Obtain wild-type viral genome sequence.
2) Select protein coding sequences to target for attenuated design.
3) Lock down known or conjectured DNA segments with non-coding functions.
4) Select desired codon distribution for remaining amino acids in redesigned proteins.
5) Perform random shuffle of at least two synonymous unlocked codon positions and calculate codon-pair score.
6) Further reduce (or increase) codon-pair score optionally employing a simulated annealing procedure.
7) Inspect resulting design for excessive secondary structure and unwanted restriction site:
   if yes->go to step (5) or correct the design by replacing problematic regions with wild-type sequences and go to step (8).
8) Synthesize DNA sequence corresponding to virus design.
9) Create viral construct and assess viral phenotype:
   if too attenuated, prepare subclone construct and go to 9;
   if insufficiently attenuated, go to 2.

Attenuation of viruses by reducing codon pair bias is disclosed in WO 2008/121992 and WO 2011/044561, which are incorporated by reference as though fully set forth.

Methods of obtaining full-length SARS-CoV-2 genome sequence or codon pair deoptimized sequences embedded in a wild-type SARS-CoV-2 genome sequence (or its mutant forms of the wild-type sequence that causes COVID-19) can include for example, constructing an infectious cDNA clone, using BAC vector, using an overlap extension PCR strategy, or long PCR-based fusion strategy.

### Recoded Polynucleotides

Various embodiments of the present invention provide for a polynucleotide encoding one or more viral proteins or one or more fragments thereof of a parent SARS-CoV-2 coronavirus, wherein the polynucleotide is recoded compared to its parent SARS-CoV-2 coronavirus polynucleotide, and wherein the amino acid sequence of the one or more viral proteins, or one or more fragments thereof of the parent SARS-CoV-2 coronavirus encoded by the polynucleotide remains the same. In various embodiments, the amino acid sequence of the one or more viral proteins, or one or more fragments thereof of the parent SARS-CoV-2 coronavirus encoded by the polynucleotide remains the same before the polyA tail of the nucleic acid sequence of the parent SARS-CoV-2 virus sequence.

Various embodiments of the present invention provide for a polynucleotide encoding one or more viral proteins or one or more fragments thereof of a parent SARS-CoV-2 coronavirus, wherein the polynucleotide is recoded compared to its parent SARS-CoV-2 coronavirus polynucleotide, and wherein the amino acid sequence of the one or more viral proteins or one or more fragments thereof of the parent SARS-CoV-2 coronavirus encoded by the polynucleotide comprises up to 20 amino acid substitutions, additions, or deletions. In various embodiments, the amino acid sequence of the one or more viral proteins or one or more fragments thereof of the parent SARS-CoV-2 coronavirus encoded by the polynucleotide comprises up to 20 amino acid substitutions, additions, or deletions is before the polyA tail of the nucleic acid sequence of the parent SARS-CoV-2 virus sequence.

Various embodiments of the present invention provide for a polynucleotide encoding one or more viral proteins or one or more fragments thereof of a parent SARS-CoV-2 coronavirus, wherein the polynucleotide is recoded compared to its parent SARS-CoV-2 coronavirus polynucleotide, and wherein the amino acid sequence of the one or more viral proteins or one or more fragments thereof of the parent SARS-CoV-2 coronavirus encoded by the polynucleotide comprises up to 10 amino acid substitutions, additions, or deletions. In various embodiments, the amino acid sequence of the one or more viral proteins or one or more fragments thereof of the parent SARS-CoV-2 coronavirus encoded by the polynucleotide comprises up to 10 amino acid substitutions, additions, or deletions is before the polyA tail of the nucleic acid sequence of the parent SARS-CoV-2 virus sequence.

Various embodiments of the present invention provide for a polynucleotide encoding one or more viral proteins or one or more fragments thereof of a parent SARS-CoV-2 coronavirus, wherein the polynucleotide is recoded compared to its parent SARS-CoV-2 coronavirus polynucleotide, and wherein the amino acid sequence of the one or more viral proteins or one or more fragments thereof of the parent SARS-CoV-2 coronavirus encoded by the polynucleotide comprises up to 12 amino acid substitutions, additions, or deletions. In various embodiments, the amino acid sequence of the one or more viral proteins or one or more fragments thereof of the parent SARS-CoV-2 coronavirus encoded by the polynucleotide comprises up to 12 amino acid substitutions, additions, or deletions is before the polyA tail of the nucleic acid sequence of the parent SARS-CoV-2 virus sequence.

In various embodiments, the amino acid sequence comprises up to 2, 3, 4, 5, 6, 7, 8, 9, 10, 11, 12, 13, 14, 15, 16, 17, 18, 19 or 20 amino acid substitutions, additions, or deletions. In various embodiments, the amino acid sequence comprises 1-5, 6-10, 11-15, or 16-20 amino acid substitutions, additions, or deletions. In various embodiments, the amino acid deletion, substitution, or addition results from nucleic acid deletion(s), substitution(s) or addition(s) before the polyA tail of the nucleic acid sequence of the parent SARS-CoV-2 virus sequence.

In various embodiments, the amino acid sequence comprises 12 amino acid deletions. In various embodiments, the amino acid sequence comprises 1-5, 6-10, 11-15, or 16-20 amino acid deletions. In various embodiments, the amino acid substitutions, additions, or deletions can be due to one or more point mutations in the recoded sequence. In various embodiments, the amino acid deletion, substitution, or addition results from nucleic acid deletion(s), substitution(s) or addition(s) before the polyA tail of the nucleic acid sequence of the parent SARS-CoV-2 virus sequence.

Thus, in various embodiments for these recoded polynucleotides (with or without the nucleic acid deletion(s), substitution(s) or addition(s)), the recoded polynucleotide can have a different length for the polyA tail; for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49, 50, 51, 52, 53, or 54 consecutive adenines on the 3′ end; or for example, 1-6, 7-12, 13-18, 19-24, 25-30, 31-36, 37-42, 43-48, or 49-54 consecutive adenines on the 3′ end; or for example, 9-37, 12-34, 15-33, 18-30, or 21-27 consecutive adenines on the 3′ end; or for example, 19-25 consecutive adenines on the 3′ end.

In various embodiments, the polynucleotide is recoded by reducing codon-pair bias (CPB) or reducing codon usage bias compared to its parent SARS-CoV-2 coronavirus polynucleotide.

In various embodiments, the polynucleotide is recoded by increasing the number of CpG or UpA di-nucleotides compared to its parent SARS-CoV-2 coronavirus polynucleotide.

In various embodiments, each of the recoded one or more viral proteins, or each of the recoded one or more fragments thereof has a codon pair bias less than, −0.05, less than −0.1, less than −0.2, less than −0.3, or less than −0.4.

In certain embodiments, the recoded viral protein is RdRP and/or spike protein and each of the recoded viral protein or fragment thereof has a codon pair bias less than −0.05, or less than −0.06, or less than −0.07, or less than −0.08, or less than −0.09, or less than −0.1, or less than −0.11, or less than −0.12, or less than −0.13, or less than −0.14, or less than −0.15, or less than −0.16, or less than −0.17, or less than −0.18, or less than −0.19, or less than −0.2, or less than −0.25, or less than −0.3, or less than −0.35, or less than −0.4, or less than −0.45, or less than −0.5.

In certain embodiments, the recoded viral protein is RdRP and/or spike protein and each of the recoded viral protein or fragment thereof is reduced by at least 0.05, or at least 0.06, or at least 0.07, or at least 0.08, or at least 0.09, or at least 0.1, or at least 0.11, or at least 0.12, or at least 0.13, or at least 0.14, or at least 0.15, or at least 0.16, or at least 0.17, or at least 0.18, or at least 0.19, or at least 0.2, or at least 0.25, or at least 0.3, or at least 0.35, or at least 0.4, or at least 0.45, or at least 0.5, compared to the corresponding sequence on the parent sequence. In certain embodiments, it is in comparison corresponding sequence on the parent sequence from which the calculation is to be made; for example, the corresponding sequence of a wild-type virus.

In various embodiments, the parent SARS-CoV-2 coronavirus is wild-type SARS-CoV-2 coronavirus. In various embodiments, the parent SARS-CoV-2 coronavirus is a natural isolate SARS-CoV-2 coronavirus.

In various embodiments, the parent SARS-CoV-2 coronavirus is wild-type BetaCoV/Wuhan/IVDC-HB-01/2019 isolate of SARS-CoV-2 coronavirus. In various embodiments, the parent SARS-CoV-2 coronavirus is wild-type Washington isolate of SARS-CoV-2 coronavirus (GenBank: MN985325.1), herein by reference as though fully set forth in its entirety.

In various embodiments, the parent SARS-CoV-2 coronavirus is a mutant form of the wild-type SARS-CoV-2 coronavirus sequence.

In various embodiments, the parent SARS-CoV-2 coronavirus is a SARS-CoV-2 variant. In various embodiments, SARS-CoV-2 variant is U.K. variant, South Africa variant, or Brazil variant.

Examples of the U.K. variant include but are not limited to GenBank Accession Nos. MW462650 (SARS-CoV-2/human/USA/MN-MDH-2252/2020), MW463056 (SARS-CoV-2/human/USA/FL-BPHL-2270/2020), and MW440433 (SARS-CoV-2/human/USA/NY-Wadsworth-291673-01/2020), all as of Jan. 19, 2021, all incorporated herein by reference as though fully set forth in their entirety. Additional examples of the U.K. variant include but are not limited to GISAID ID Nos. EPI_ISL_778842 (hCoV-19/USA/TX-CDC-9KXP-8438/2020; 2020-12-28), EPI_ISL_802609 (hCoV-19/USA/CA-CDC-STM-050/2020; 2020-12-28), EPI_ISL_802647 (hCoV-19/USA/FL-CDC-STM-043/2020; 2020-12-26), EPI_ISL_832014 (hCoV-19/USA/UT-UPHL-2101178518/2020; 2020-12-31), EPI_ISL_850618 (hCoV-19/USA/IN-CDC-STM-183/2020; 2020-12-31), and EPI_ISL_850960 (hCoV-19/USA/FL-CDC-STM-A100002/2021; 2021-01-04), all as of Jan. 20, 2021, and all incorporated herein by reference as though fully set forth in their entirety.

Examples of the South Africa variant include but are not limited to GISAID ID Nos. EPI_ISL_766709 (hCoV-19/Sweden/20-13194/2020; 2020-12-24), EPI_ISL_768828 (hCoV-19/France/PAC-NRC2933/2020; 2020-12-22), EPI_ISL_770441 (hCoV-19/England/205280030/2020; 2020-12-24), and EPI_ISL_819798 (hCoV-19/England/OXON-F440A7/2020; 2020-12-18), all as of Jan. 20, 2021, and all incorporated herein by reference as though fully set forth in their entirety.

Examples of the Brazil variant include but are not limited to GISAID ID Nos. EPI_ISL_677212 (hCoV-19/USA/VA-DCLS-2187/2020; 2020-11-12), EPI_ISL_723494 (hCoV-19/USA/VA-DCLS-2191/2020; 2020-11-12), EPI_ISL_845768 (hCoV-19/USA/GA-EHC-458R/2021; 2021-01-05), EPI_ISL_848196 (hCoV-19/Canada/LTRI-1192/2020; 2020-12-24), and EPI_ISL_848197 (hCoV-19/Canada/LTRI-1258/2020; 2020-12-24), all as of Jan. 20, 2021, and all incorporated herein by reference as though fully set forth in their entirety.

In various embodiments, the parent SARS-CoV-2 coronavirus is a previously modified viral nucleic acid, or a previously attenuated viral nucleic acid.

In various embodiments, the polynucleotide is CPB deoptimized compared to its parent SARS-CoV-2 coronavirus polynucleotide. In various embodiments, the polynucleotide is codon deoptimized compared to its parent SARS-CoV-2 coronavirus polynucleotide.

In various embodiments, the codon-deoptimized or CPB deoptimized is based on frequently used codons or CPB in humans. In various embodiments, the codon-deoptimized or CPB deoptimized is based on frequently used codons or CPB in a coronavirus. In various embodiments, the codon-deoptimized or CPB deoptimized is based on frequently used codons or CPB in a SARS-CoV-2 coronavirus. In various embodiments, the codon-deoptimized or CPB deoptimized is based on frequently used codons or CPB in a wild-type SARS-CoV-2 coronavirus.

In various embodiments, the polynucleotide comprises a recoded nucleotide sequence selected from RNA-dependent RNA polymerase (RdRP), a fragment of RdRP, a spike protein, a fragment of spike protein, and combinations thereof. In various embodiments, polynucleotide comprises a deletion of nucleotides that results in a deletion of amino acids in the spike protein that eliminates the furin cleavage site. While not wishing to be bound by any particular theory, the inventors believe that eliminating the furin cleavage site will be one of the drivers of safety of the vaccine and/or immune composition.

In various embodiments, the polynucleotide comprises at least one CPB deoptimized region selected from bp 11294-12709, bp 14641-15903, bp 21656-22306, bp 22505-23905, and bp 24110-25381 of SEQ ID NO:1 or SEQ ID NO:2.

In various embodiments, the polynucleotide comprises SEQ ID NO:3 (Wuhan-CoV_101K). In various embodiments, the polynucleotide comprises nucleotides 1-29,877 of SEQ ID NO:3 (e.g., without the polyA tail). In various embodiments, the polynucleotide comprises nucleotides 1-29,877 of SEQ ID NO:3 and 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49, 50, 51, 52, 53, or 54 consecutive adenines on the 3′ end. In various embodiments, the polynucleotide comprises nucleotides 1-29,877 of SEQ ID NO:3 and 1-6, 7-12, 13-18, 19-24, 25-30, 31-36, 37-42, 43-48, or 49-54 consecutive adenines on the 3′ end. In various embodiments, the polynucleotide comprises nucleotides 1-29,877 of SEQ ID NO:3 and 9-37, 12-34, 15-33, 18-30, or 21-27 consecutive adenines on the 3′ end. In various embodiments, the polynucleotide comprises nucleotides 1-29,877 of SEQ ID NO:3 and 19-25 consecutive adenines on the 3′ end.

In various embodiment, the polynucleotide comprises SEQ ID NO:4. SEQ ID NO:4 is the deoptimized sequence in comparison to the wild-type WA-1 sequence (GenBank: MN985325.1 herein incorporated by reference as though fully set forth) (e.g., CDX-005). In various embodiments, the polynucleotide comprises nucleotides 1-29,834 of SEQ ID NO:4 (e.g., without the polyA tail). In various embodiments, the polynucleotide comprises nucleotides 1-29,834 of SEQ ID NO:4 and 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49, 50, 51, 52, 53, or 54 consecutive adenines on the 3′ end. In various embodiments, the polynucleotide comprises 1-29,834 of SEQ ID NO:4 and 1-6, 7-12, 13-18, 19-24, 25-30, 31-36, 37-42, 43-48, or 49-54 consecutive adenines on the 3′ end. In various embodiments, the polynucleotide comprises nucleotides 1-29,834 of SEQ ID NO:4 and 9-37, 12-34, 15-33, 18-30, or 21-27 consecutive adenines on the 3′ end. In various embodiments, the polynucleotide comprises nucleotides 1-29,834 of SEQ ID NO:4 and 19-25 consecutive adenines on the 3′ end.

In various embodiments, the polynucleotide encodes SEQ ID NO:6 (recoded spike protein).

### Vectors, Cells, Polypeptides

Various embodiments provide for a bacterial artificial chromosome (BAC) comprising a polynucleotide of the present invention. The polynucleotides of the present invention are the recoded polypeptides as discussed herein.

Various embodiments provide for a vector comprising a polynucleotide of the present invention. The polynucleotides of the present invention are the recoded polypeptides as discussed herein.

A cell comprising a vector of the present invention. The vectors are those as discussed herein.

In various embodiments, the cell is a Vero cell, HeLa Cell, baby hamster kidney (BHK) cell, MA104 cell, 293T Cell, BSR-T7 Cell, MRC-5 cell, CHO cell, or PER.C6 cell. In particular embodiments, the cell is Vero cell or baby hamster kidney (BHK) cell.

Various embodiments provide for a polypeptide encoded by a polynucleotide of the present invention. The polynucleotides of the present invention are the recoded polypeptides as discussed herein. The polypeptide exhibits properties that are different than a polypeptide encoded by the wild-type SARS-CoV-2 virus, or a polypeptide encoded by a SARS-CoV-2 variant. For example, the polypeptide encoded by recoded polynucleotides and deoptimized polynucleotides as discussed herein can exert attenuating properties to the virus.

### Modified Viruses

Various embodiments of the present invention provide for a modified SARS-CoV-2 coronavirus comprising a polypeptide encoded by a polynucleotide of the present invention. The polynucleotides of the present invention are the recoded polypeptides as discussed herein.

Various embodiments of the present invention provide for a modified SARS-CoV-2 coronavirus comprising a polynucleotide of the present invention. The polynucleotides of the present invention are any one of the recoded polypeptides discussed herein.

In various embodiments, the expression of one or more of its viral proteins is reduced compared to its parent SARS-CoV-2 coronavirus.

In various embodiments, the parent SARS-CoV-2 coronavirus is wild-type SARS-CoV-2 coronavirus. In various embodiments, the parent SARS-CoV-2 coronavirus is a natural isolate SARS-CoV-2 coronavirus.

In various embodiments, the parent SARS-CoV-2 coronavirus is wild-type BetaCoV/Wuhan/IVDC-HB-01/2019 isolate of SARS-CoV-2 coronavirus. In various embodiments, the parent SARS-CoV-2 coronavirus is wild-type Washington isolate of SARS-CoV-2 coronavirus (GenBank: MN985325.1), herein by reference as though fully set forth in its entirety.

In various embodiments, the parent SARS-CoV-2 coronavirus is a mutant form of the wild-type SARS-CoV-2 coronavirus sequence.

In various embodiments, the parent SARS-CoV-2 coronavirus is a SARS-CoV-2 variant. In various embodiments, SARS-CoV-2 variant is U.K. variant, South Africa variant, or Brazil variant.

Examples of the U.K. variant include but are not limited to GenBank Accession Nos. MW462650 (SARS-CoV-2/human/USA/MN-MDH-2252/2020), MW463056 (SARS-CoV-2/human/USA/FL-BPHL-2270/2020), and MW440433 (SARS-CoV-2/human/USA/NY-Wadsworth-291673-01/2020), all as of Jan. 19, 2021, all incorporated herein by reference as though fully set forth in their entirety. Additional examples of the U.K. variant include but are not limited to GISAID ID Nos. EPI_ISL_778842 (hCoV-19/USA/TX-CDC-9KXP-8438/2020; 2020-12-28), EPI_ISL_802609 (hCoV-19/USA/CA-CDC-STM-050/2020; 2020-12-28), EPI_ISL_802647 (hCoV-19/USA/FL-CDC-STM-043/2020; 2020-12-26), EPI_ISL_832014 (hCoV-19/USA/UT-UPHL-2101178518/2020; 2020-12-31), EPI_ISL_850618 (hCoV-19/USA/IN-CDC-STM-183/2020; 2020-12-31), and EPI_ISL_850960 (hCoV-19/USA/FL-CDC-STM-A100002/2021; 2021-01-04), all as of Jan. 20, 2021, and all incorporated herein by reference as though fully set forth in their entirety.

Examples of the South Africa variant include but are not limited to GISAID ID Nos. EPI_ISL_766709 (hCoV-19/Sweden/20-13194/2020; 2020-12-24), EPI_ISL_768828 (hCoV-19/France/PAC-NRC2933/2020; 2020-12-22), EPI_ISL_770441 (hCoV-19/England/205280030/2020; 2020-12-24), and EPI_ISL_819798 (hCoV-19/England/OXON-F440A7/2020; 2020-12-18), all as of Jan. 20, 2021, and all incorporated herein by reference as though fully set forth in their entirety.

Examples of the Brazil variant include but are not limited to GISAID ID Nos. EPI_ISL_677212 (hCoV-19/USA/VA-DCLS-2187/2020; 2020-11-12), EPI_ISL_723494 (hCoV-19/USA/VA-DCLS-2191/2020; 2020-11-12), EPI_ISL_845768 (hCoV-19/USA/GA-EHC-458R/2021; 2021-01-05), EPI_ISL_848196 (hCoV-19/Canada/LTRI-1192/2020; 2020-12-24), and EPI_ISL_848197 (hCoV-19/Canada/LTRI-1258/2020; 2020-12-24), all as of Jan. 20, 2021, and all incorporated herein by reference as though fully set forth in their entirety.

In various embodiments, the parent SARS-CoV-2 coronavirus is a previously modified viral nucleic acid, or a previously attenuated viral nucleic acid.

In various embodiments the reduction in the expression of one or more of its viral proteins is reduced as the result of recoding a region selected RdRP protein, spike protein, and combinations thereof.

In various embodiments, the polynucleotide encodes one or more viral proteins or one or more fragments thereof of a parent SARS-CoV-2 coronavirus, wherein the polynucleotide is recoded compared to its parent SARS-CoV-2 coronavirus polynucleotide, and wherein the amino acid sequence of the one or more viral proteins, or one or more fragments thereof of the parent SARS-CoV-2 coronavirus encoded by the polynucleotide remains the same.

In various embodiments, the polynucleotide encodes one or more viral proteins or one or more fragments thereof of a parent SARS-CoV-2 coronavirus, wherein the polynucleotide is recoded compared to its parent SARS-CoV-2 coronavirus polynucleotide, and wherein the amino acid sequence of the one or more viral proteins or one or more fragments thereof of the parent SARS-CoV-2 coronavirus encoded by the polynucleotide comprises up to 15 amino acid substitutions, additions, or deletions. In various embodiments, the amino acid sequence comprises up to 2, 3, 4, 5, 6, 7, 8, 9, 10, 11, 12, 13, 14, or 15 amino acid substitutions, additions, or deletions. In various embodiments, the amino acid sequence comprises 12 amino acid deletions. In various embodiments, the amino acid sequence comprises 1-3, 4-6, 7-9, 10-12, or 13-15 amino acid deletions. The amino acid substitutions, additions, or deletions can be due to one or more point mutations in the recoded sequence. In various embodiments, the amino acid deletion, substitution, or addition results from nucleic acid deletion(s), substitution(s) or addition(s) before the polyA tail of the nucleic acid sequence of the parent SARS-CoV-2 virus sequence.

In various embodiments, the polynucleotide is recoded by reducing codon-pair bias (CPB) or reducing codon usage bias compared to its parent SARS-CoV-2 coronavirus polynucleotide.

In various embodiments, the polynucleotide is recoded by increasing the number of CpG or UpA di-nucleotides compared to its parent SARS-CoV-2 coronavirus polynucleotide.

In various embodiments, each of the recoded one or more viral proteins, or each of the recoded one or more fragments thereof has a codon pair bias less than, −0.05, less than −0.1, less than −0.2, less than −0.3, or less than −0.4.

In various embodiments, the parent SARS-CoV-2 coronavirus is wild-type SARS-CoV-2 coronavirus. In various embodiments, the parent SARS-CoV-2 coronavirus is a natural isolate SARS-CoV-2 coronavirus.

In various embodiments, the parent SARS-CoV-2 coronavirus is wild-type BetaCoV/Wuhan/IVDC-HB-01/2019 isolate of SARS-CoV-2 coronavirus. In various embodiments, the parent SARS-CoV-2 coronavirus is wild-type Washington isolate of SARS-CoV-2 coronavirus (GenBank: MN985325.1), herein by reference as though fully set forth in its entirety.

In various embodiments, the parent SARS-CoV-2 coronavirus is a mutant form of the wild-type SARS-CoV-2 coronavirus sequence.

In various embodiments, the parent SARS-CoV-2 coronavirus is a SARS-CoV-2 variant. In various embodiments, SARS-CoV-2 variant is U.K. variant, South Africa variant, or Brazil variant.

Examples of the U.K. variant include but are not limited to GenBank Accession Nos. MW462650 (SARS-CoV-2/human/USA/MN-MDH-2252/2020), MW463056 (SARS-CoV-2/human/USA/FL-BPHL-2270/2020), and MW440433 (SARS-CoV-2/human/USA/NY-Wadsworth-291673-01/2020), all as of Jan. 19, 2021, all incorporated herein by reference as though fully set forth in their entirety. Additional examples of the U.K. variant include but are not limited to GISAID ID Nos. EPI_ISL_778842 (hCoV-19/USA/TX-CDC-9KXP-8438/2020; 2020-12-28), EPI_ISL_802609 (hCoV-19/USA/CA-CDC-STM-050/2020; 2020-12-28), EPI_ISL_802647 (hCoV-19/USA/FL-CDC-STM-043/2020; 2020-12-26), EPI_ISL_832014 (hCoV-19/USA/UT-UPHL-2101178518/2020; 2020-12-31), EPI_ISL_850618 (hCoV-19/USA/IN-CDC-STM-183/2020; 2020-12-31), and EPI_ISL_850960 (hCoV-19/USA/FL-CDC-STM-A100002/2021; 2021-01-04), all as of Jan. 20, 2021, and all incorporated herein by reference as though fully set forth in their entirety.

Examples of the South Africa variant include but are not limited to GISAID ID Nos. EPI_ISL_766709 (hCoV-19/Sweden/20-13194/2020; 2020-12-24), EPI_ISL_768828 (hCoV-19/France/PAC-NRC2933/2020; 2020-12-22), EPI_ISL_770441 (hCoV-19/England/205280030/2020; 2020-12-24), and EPI_ISL_819798 (hCoV-19/England/OXON-F440A7/2020; 2020-12-18), all as of Jan. 20, 2021, and all incorporated herein by reference as though fully set forth in their entirety.

Examples of the Brazil variant include but are not limited to GISAID ID Nos. EPI_ISL_677212 (hCoV-19/USA/VA-DCLS-2187/2020; 2020-11-12), EPI_ISL_723494 (hCoV-19/USA/VA-DCLS-2191/2020; 2020-11-12), EPI_ISL_845768 (hCoV-19/USA/GA-EHC-458R/2021; 2021-01-05), EPI_ISL_848196 (hCoV-19/Canada/LTRI-1192/2020; 2020-12-24), and EPI_ISL_848197 (hCoV-19/Canada/LTRI-1258/2020; 2020-12-24), all as of Jan. 20, 2021, and all incorporated herein by reference as though fully set forth in their entirety.

In various embodiments, the parent SARS-CoV-2 coronavirus is a previously modified viral nucleic acid, or a previously attenuated viral nucleic acid.

In various embodiments, the polynucleotide is CPB deoptimized compared to its parent SARS-CoV-2 coronavirus polynucleotide. In various embodiments, the polynucleotide is codon deoptimized compared to its parent SARS-CoV-2 coronavirus polynucleotide.

In various embodiments, the codon-deoptimized or CPB deoptimized is based on frequently used codons or CPB in humans. In various embodiments, the codon-deoptimized or CPB deoptimized is based on frequently used codons or CPB in a coronavirus. In various embodiments, the codon-deoptimized or CPB deoptimized is based on frequently used codons or CPB in a SARS-CoV-2 coronavirus. In various embodiments, the codon-deoptimized or CPB deoptimized is based on frequently used codons or CPB in a wild-type SARS-CoV-2 coronavirus.

In various embodiments, the polynucleotide comprises a recoded nucleotide sequence selected from RNA-dependent RNA polymerase (RdRP), a fragment of RdRP, a spike protein, a fragment of spike protein, and combinations thereof. In various embodiments, polynucleotide comprises a deletion of nucleotides that results in a deletion of amino acids in the spike protein that eliminates the furin cleavage site. While not wishing to be bound by any particular theory, the inventors believe that eliminating the furin cleavage site will be one of the drivers of safety of the vaccine and/or immune composition.

In various embodiments, the polynucleotide comprises at least one CPB deoptimized region selected from bp 11294-12709, bp 14641-15903, bp 21656-22306, bp 22505-23905, and bp 24110-25381 of SEQ ID NO:1 or SEQ ID NO:2.

In various embodiments, the polynucleotide comprises SEQ ID NO:3 (Wuhan-CoV_101K). In various embodiments, the polynucleotide comprises nucleotides 1-29,877 of SEQ ID NO:3 (e.g., without the polyA tail). In various embodiments, the polynucleotide comprises nucleotides 1-29,877 of SEQ ID NO:3 and 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49, 50, 51, 52, 53, or 54 consecutive adenines on the 3′ end. In various embodiments, the polynucleotide comprises nucleotides 1-29,877 of SEQ ID NO:3 and 1-6, 7-12, 13-18, 19-24, 25-30, 31-36, 37-42, 43-48, or 49-54 consecutive adenines on the 3′ end. In various embodiments, the polynucleotide comprises nucleotides 1-29,877 of SEQ ID NO:3 and 9-37, 12-34, 15-33, 18-30, or 21-27 consecutive adenines on the 3′ end. In various embodiments, the polynucleotide comprises nucleotides 1-29,877 of SEQ ID NO:3 and 19-25 consecutive adenines on the 3′ end.

In various embodiment, the polynucleotide comprises SEQ ID NO:4. In various embodiments, the polynucleotide comprises nucleotides 1-29,834 of SEQ ID NO:4 (e.g., without the polyA tail). In various embodiments, the polynucleotide comprises nucleotides 1-29,834 of SEQ ID NO:4 and 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49, 50, 51, 52, 53, or 54 consecutive adenines on the 3′ end. In various embodiments, the polynucleotide comprises 1-29,834 of SEQ ID NO:4 and 1-6, 7-12, 13-18, 19-24, 25-30, 31-36, 37-42, 43-48, or 49-54 consecutive adenines on the 3′ end. In various embodiments, the polynucleotide comprises nucleotides 1-29,834 of SEQ ID NO:4 and 9-37, 12-34, 15-33, 18-30, or 21-27 consecutive adenines on the 3′ end. In various embodiments, the polynucleotide comprises nucleotides 1-29,834 of SEQ ID NO:4 and 19-25 consecutive adenines on the 3′ end.

In various embodiment, the polynucleotide comprises SEQ ID NO:7. In various embodiments, the polynucleotide comprises nucleotides 1-29,834 of SEQ ID NO:7 (e.g., without the polyA tail). In various embodiments, the polynucleotide comprises nucleotides 1-29,834 of SEQ ID NO:7 and 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49, 50, 51, 52, 53, or 54 consecutive adenines on the 3′ end. In various embodiments, the polynucleotide comprises 1-29,834 of SEQ ID NO:7 and 1-6, 7-12, 13-18, 19-24, 25-30, 31-36, 37-42, 43-48, or 49-54 consecutive adenines on the 3′ end. In various embodiments, the polynucleotide comprises nucleotides 1-29,834 of SEQ ID NO:7 and 9-37, 12-34, 15-33, 18-30, or 21-27 consecutive adenines on the 3′ end. In various embodiments, the polynucleotide comprises nucleotides 1-29,834 of SEQ ID NO:7 and 19-25 consecutive adenines on the 3′ end.

In various embodiments, the polynucleotide encodes SEQ ID NO:6 (recoded spike protein).

### Immune and or Vaccines Compositions

Various embodiments provide for an immune composition for inducing an immune response in a subject, comprising: a modified SARS-CoV-2 coronavirus of the present invention. The modified SARS-CoV-2 coronavirus is any one of the modified SARS-CoV-2 coronavirus discussed herein. In various embodiments, the modified SARS-CoV-2 coronavirus of the present invention is a live-attenuated virus. In some embodiments the immune composition further comprises an acceptable excipient or carrier as described herein. In some embodiments, the immune composition further comprises a stabilizer as described herein. In some embodiments, the immune composition further comprise an adjuvant as described herein. In some embodiments, the immune composition further comprises sucrose, glycine or both. In various embodiments, the immune composition further comprises about sucrose (5%) and about glycine (5%). In various embodiments, the acceptable carrier or excipient is selected from the group consisting of a sugar, amino acid, surfactant and combinations thereof. In various embodiments, the amino acid is at a concentration of about 5% w/v. Nonlimiting examples of suitable amino acids include arginine and histidine. Nonlimiting examples of suitable carriers include gelatin and human serum albumin. Nonlimiting examples of suitable surfactants include nonionic surfactants such as Polysorbate 80 at very low concentration of 0.01-0.05%.

In various embodiments, the immune composition is provided at dosages of about 10³-10⁷ PFU. In various embodiments, the immune composition is provided at dosages of about 10⁴-10⁶ PFU. In various embodiments, the immune composition is provided at a dosage of about 10³ PFU. In various embodiments, the immune composition is provided at a dosage of about 10⁴ PFU. In various embodiments, the immune composition is provided at a dosage of about 10⁵ PFU. In various embodiments, the immune composition is provided at a dosage of about 10⁶ PFU. In various embodiments, the immune composition is provided at a dosage of about 10⁷ PFU.

In various embodiments, the immune composition is provided at a dosage of about 5×10³ PFU. In various embodiments, the immune composition is provided at a dosage of about 5×10⁴ PFU. In various embodiments, the immune composition is provided at a dosage of about 5×10⁵ PFU. In various embodiments, the immune composition is provided at a dosage of about 5×10⁶ PFU. In various embodiments, the immune composition is provided at a dosage of about 5×10⁷ PFU.

Various embodiments provide for a vaccine composition for inducing an immune response in a subject, comprising: a modified SARS-CoV-2 coronavirus of the present invention. The modified SARS-CoV-2 coronavirus is any one of the modified SARS-CoV-2 coronavirus discussed herein. In various embodiments, the modified SARS-CoV-2 coronavirus of the present invention is a live-attenuated virus. In some embodiments the vaccine composition further comprises an acceptable carrier or excipient as described herein. In some embodiments, the immune composition further comprises a stabilizer as described herein. In some embodiments, the vaccine composition further comprise an adjuvant as described herein. In some embodiments, the vaccine composition further comprises sucrose, glycine or both. In various embodiments, the vaccine composition further comprises sucrose (5%) and glycine (5%). In various embodiments, the acceptable carrier or excipient is selected from the group consisting of a sugar, amino acid, surfactant and combinations thereof. In various embodiments, the amino acid is at a concentration of about 5% w/v. Nonlimiting examples of suitable amino acids include arginine and histidine. Nonlimiting examples of suitable carriers include gelatin and human serum albumin. Nonlimiting examples of suitable surfactants include nonionic surfactants such as Polysorbate 80 at very low concentration of 0.01-0.05%.

In various embodiments, the vaccine composition is provided at dosages of about 10³-10⁷ PFU. In various embodiments, the vaccine composition is provided at dosages of about 10⁴-10⁶ PFU. In various embodiments, the vaccine composition is provided at a dosage of about 10³ PFU. In various embodiments, the vaccine composition is provided at a dosage of about 10⁴ PFU. In various embodiments, the vaccine composition is provided at a dosage of about 10⁵ PFU. In various embodiments, the vaccine composition is provided at a dosage of about 10⁶ PFU. In various embodiments, the vaccine composition is provided at a dosage of about 10⁷ PFU.

In various embodiments, the immune composition is provided at a dosage of about 5×10³ PFU. In various embodiments, the immune composition is provided at a dosage of about 5×10⁴ PFU. In various embodiments, the immune composition is provided at a dosage of about 5×10⁵ PFU. In various embodiments, the immune composition is provided at a dosage of about 5×10⁶ PFU. In various embodiments, the immune composition is provided at a dosage of about 5×10⁷ PFU.

Various embodiments provide for a vaccine composition for inducing a protective immune response in a subject, comprising: a modified SARS-CoV-2 coronavirus of the present invention. The modified SARS-CoV-2 coronavirus is any one of the modified SARS-CoV-2 coronavirus discussed herein. In various embodiments, the modified SARS-CoV-2 coronavirus of the present invention is a live-attenuated virus. In some embodiments the vaccine composition further comprises an acceptable carrier or excipient as described herein. In some embodiments, the vaccine composition further comprise an adjuvant as described herein. In some embodiments, the vaccine composition further comprises sucrose, glycine or both. In various embodiments, the vaccine composition further comprises sucrose (5%) and glycine (5%). In various embodiments, the acceptable carrier or excipient is selected from the group consisting of a sugar, amino acid, surfactant and combinations thereof. In various embodiments, the amino acid is at a concentration of about 5% w/v. Nonlimiting examples of suitable amino acids include arginine and histidine. Nonlimiting examples of suitable carriers include gelatin and human serum albumin. Nonlimiting examples of suitable surfactants include nonionic surfactants such as Polysorbate 80 at very low concentration of 0.01-0.05%.

In various embodiments, the vaccine composition is provided at dosages of about 10³-10⁷ PFU. In various embodiments, the vaccine composition is provided at dosages of about 10⁴-10⁶ PFU. In various embodiments, the vaccine composition is provided at a dosage of about 10³ PFU. In various embodiments, the vaccine composition is provided at a dosage of about 10⁴ PFU. In various embodiments, the vaccine composition is provided at a dosage of about 10⁵ PFU. In various embodiments, the vaccine composition is provided at a dosage of about 10⁶ PFU. In various embodiments, the vaccine composition is provided at a dosage of about 10⁷ PFU.

In various embodiments, the immune composition is provided at a dosage of about 5×10³ PFU. In various embodiments, the immune composition is provided at a dosage of about 5×10⁴ PFU. In various embodiments, the immune composition is provided at a dosage of about 5×10⁵ PFU. In various embodiments, the immune composition is provided at a dosage of about 5×10⁶ PFU. In various embodiments, the immune composition is provided at a dosage of about 5×10⁷ PFU.

It should be understood that an attenuated virus of the invention, where used to elicit an immune response in a subject (or protective immune response) or to prevent a subject from or reduce the likelihood of becoming afflicted with a virus-associated disease, can be administered to the subject in the form of a composition additionally comprising a pharmaceutically acceptable carrier or excipient. Pharmaceutically acceptable carriers and excipients are known to those skilled in the art and include, but are not limited to, one or more of 0.01-0.1M and preferably 0.05M phosphate buffer, phosphate-buffered saline (PBS), DMEM, L-15, a 10-25% sucrose solution in PBS, a 10-25% sucrose solution in DMEM, or 0.9% saline. Such carriers also include aqueous or non-aqueous solutions, suspensions, and emulsions. Aqueous carriers include water, alcoholic/aqueous solutions, emulsions or suspensions, saline and buffered media. Examples of non-aqueous solvents are propylene glycol, polyethylene glycol, vegetable oils such as olive oil, and injectable organic esters such as ethyl oleate. Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Solid compositions may comprise nontoxic solid carriers such as, for example, glucose, sucrose, mannitol, sorbitol, lactose, starch, magnesium stearate, cellulose or cellulose derivatives, sodium carbonate, gelatin, recombinant human serum albumin, human serum albumin, and/or magnesium carbonate. For administration in an aerosol, such as for pulmonary and/or intranasal delivery, an agent or composition is preferably formulated with a nontoxic surfactant, for example, esters or partial esters of C6 to C22 fatty acids or natural glycerides, and a propellant. Additional carriers such as lecithin may be included to facilitate intranasal delivery. Pharmaceutically acceptable carriers or excipients can further comprise minor amounts of auxiliary substances such as wetting or emulsifying agents, preservatives and other additives, such as, for example, antimicrobials, antioxidants and chelating agents, which enhance the shelf life and/or effectiveness of the active ingredients. The instant compositions can, as is well known in the art, be formulated so as to provide quick, sustained or delayed release of the active ingredient after administration to a subject.

In various embodiments, the vaccine composition or immune composition is formulated for delivery intravenously, or intrathecally, subcutaneously, intramuscularly, intradermally or intranasally. In various embodiments, the vaccine composition or immune composition is formulated for delivery intranasally. In various embodiments, the vaccine composition or immune composition is formulated for delivery via a nasal drop or nasal spray.

### Methods of Using the Compositions of the Present Invention.

Various embodiments provide for a method of eliciting an immune response in a subject, comprising: administering to the subject a dose of an immune composition the present invention. The immune composition is any one of the immune composition discussed herein. In various embodiments, the dose is a prophylactically effective or therapeutically effective dose.

In various embodiments, the immune composition is administered intravenously, or intrathecally, subcutaneously, intramuscularly, intradermally or intranasally. In various embodiments, the immune composition is administered intranasally. In various embodiments, the immune composition is administered via a nasal drop or nasal spray.

Various embodiments provide for a method of eliciting an immune response in a subject, comprising: administering to the subject a dose of a vaccine composition the present invention. The vaccine composition is any one of the vaccine composition discussed herein. In various embodiments, the immune response is a protective immune response. In various embodiments, the dose is a prophylactically effective or therapeutically effective dose.

In various embodiments, the vaccine composition is administered intravenously, or intrathecally, subcutaneously, intramuscularly, intradermally or intranasally. In various embodiments, the vaccine composition is administered intranasally. In various embodiments, the vaccine composition is administered via a nasal drop or nasal spray.

Various embodiments provide for a method of eliciting an immune response in a subject, comprising: administering to the subject a dose of a modified SARS-CoV-2 coronavirus of the present invention. The modified SARS-CoV-2 coronavirus is any one of the modified SARS-CoV-2 coronavirus discussed herein. In various embodiments, the immune response is a protective immune response. In various embodiments, the dose is a prophylactically effective or therapeutically effective dose.

In various embodiments, the dose is about 10³-10⁷ PFU. In various embodiments, the dose is about 10⁴-10⁶ PFU. In various embodiments, the dose is about 10³ PFU. In various embodiments, the dose is about 10⁴ PFU. In various embodiments, the dose is about 10⁵ PFU. In various embodiments, the dose is about 10⁶ PFU. In various embodiments, the dose is about 10⁷ PFU.

In various embodiments, the dose is about 5×10³ PFU. In various embodiments, the dose is about 5×10⁴ PFU. In various embodiments, the dose is about 5×10⁵ PFU. In various embodiments, the dose is about 5×10⁶ PFU. In various embodiments, the dose is about 5×10⁷ PFU.

In various embodiments, the modified SARS-CoV-2 coronavirus is administered intravenously, or intrathecally, subcutaneously, intramuscularly, intradermally or intranasally. In various embodiments, the modified SARS-CoV-2 coronavirus is administered intranasally. In various embodiments, the modified SARS-CoV-2 coronavirus is administered via a nasal drop or nasal spray.

Various embodiments provide for a method of eliciting an immune response in a subject, comprising: administering to the subject a prime dose of a modified SARS-CoV-2 coronavirus of the present invention; and administering to the subject one or more boost doses of a modified SARS-CoV-2 coronavirus of the present invention. The modified SARS-CoV-2 coronavirus is any one of the modified SARS-CoV-2 coronavirus discussed herein. In various embodiments, the dose is a prophylactically effective or therapeutically effective dose.

In various embodiments, the prime dose and/or the one or more boost doses of the modified SARS-CoV-2 coronavirus is administered intravenously, or intrathecally, subcutaneously, intramuscularly, intradermally or intranasally. In various embodiments, the prime dose and/or the one or more boost doses of the modified SARS-CoV-2 coronavirus is administered intranasally. In various embodiments, the prime dose and/or the one or more boost doses of the modified SARS-CoV-2 coronavirus is administered via a nasal drop or nasal spray.

Various embodiments provide for a method of eliciting an immune response in a subject, comprising: administering to the subject a prime dose of an immune composition of the present invention; and administering to the subject one or more boost doses of an immune composition of the present invention. The immune composition is any one of the immune composition discussed herein. In various embodiments, the dose is a prophylactically effective or therapeutically effective dose.

In various embodiments, the prime dose and/or the one or more boost doses of the immune composition is administered intravenously, or intrathecally, subcutaneously, intramuscularly, intradermally or intranasally. In various embodiments, the prime dose and/or the one or more boost doses of the immune composition is administered intranasally. In various embodiments, the prime dose and/or the one or more boost doses of the immune composition is administered via a nasal drop or nasal spray.

Various embodiments provide for a method of eliciting an immune response in a subject, comprising: administering to the subject a prime dose of a vaccine composition of the present invention; and administering to the subject one or more boost doses of a vaccine composition of the present invention. The vaccine composition is any one of the vaccine composition discussed herein. In various embodiments, the dose is a prophylactically effective or therapeutically effective dose.

In various embodiments, the prime dose and/or the one or more boost doses of the vaccine composition is administered intravenously, or intrathecally, subcutaneously, intramuscularly, intradermally or intranasally. In various embodiments, the prime dose and/or the one or more boost doses of the vaccine composition is administered intranasally. In various embodiments, the prime dose and/or the one or more boost doses of the vaccine composition is administered via a nasal drop or nasal spray.

The timing between the prime and boost dosages can vary, for example, depending on the stage of infection or disease (e.g., non-infected, infected, number of days post infection), and the patient's health. In various embodiments, the one or more boost dose is administered about 2 weeks after the prime dose. That is, the prime dose is administered and about two weeks thereafter, a boost dose is administered. In various embodiments, the one or more boost dose is administered about 4 weeks after the prime dose. In various embodiments, the one or more boost dose is administered about 6 weeks after the prime dose. In various embodiments, the one or more boost dose is administered about 8 weeks after the prime dose. In various embodiments, the one or more boost dose is administered about 12 weeks after the prime dose. In various embodiments, the one or more boost dose is administered about 1-12 weeks after the prime dose.

In various embodiments, the one or more boost doses can be given as one boost dose. In other embodiments, the one or more boost doses can be given as a boost dose periodically. For example, it can be given quarterly, every 4 months, every 6 months, yearly, every 2 years, every 3 years, every 4 years, every 5 years, every 6 years, every 7 years, every 8 years, every 9 years, or every 10 years.

In various embodiments, the prime dose and boost does are each about 10³-10⁷ PFU. In various embodiments, the prime dose and boost does are each about 10⁴-10⁶ PFU. In various embodiments, the prime dose and boost does are each about 10³ PFU. In various embodiments, the prime dose and boost does are each about 10⁴ PFU. In various embodiments, the prime dose and boost does are each about 10⁵ PFU. In various embodiments, the prime dose and boost does are each about 10⁶ PFU. In various embodiments, the dose is about 10⁷ PFU.

In various embodiments, the prime dose and boost does are each about 5×10³ PFU. In various embodiments, the prime dose and boost does are each about 5×10⁴ PFU. In various embodiments, the prime dose and boost does are each about 5×10⁵ PFU. In various embodiments, the prime dose and boost does are each about 5×10⁶ PFU. In various embodiments, the prime dose and boost does are each about 5×10⁷ PFU.

In various embodiments, the dosage for the prime dose and the boost dose is the same.

In various embodiments, the dosage amount can vary between the prime and boost dosages. As a non-limiting example, the prime dose can contain fewer copies of the virus compared to the boost dose. For example, the prime dose is about 10³ PFU and the boost dose is about 10⁴-10⁶ PFU, or, the prime dose is about 10⁴ and the boost dose is about 10⁵-10⁷ PFU.

In various embodiments, wherein the boost dose is administered periodically, the subsequent boost doses can be less than the first boost dose.

As another non-limiting example, the prime dose can contain more copies of the virus compared to the boost dose.

In various embodiments, the immune response is a protective immune response.

In various embodiments, the dose is a prophylactically effective or therapeutically effective dose.

In various embodiments, intranasal administration of a modified SARS-CoV-2 coronavirus of the present invention, the immune composition of the present invention or the vaccine composition of the present invention comprises: instructing the subject blow the nose and tilt the head back; optionally, instructing the subject reposition the head to avoid having composition dripping outside of the nose or down the throat; administering about 0.25 mL comprising the dosage into each nostril; instructing the subject to sniff gently; and instructing the subject to not blow the nose for a period of time; for example, about 60 minutes.

In some embodiments, the subject is not taking any immunosuppressive medications. In various embodiments, the subject is not taking any immunosuppressive medications about 180 days, 150 days, 120 days, 90 days, 75 days, 60 days, 45 days, 30 days, 15 days or 7 days before the administration of the modified SARS-CoV-2 coronavirus of the present invention, the immune composition of the present invention or the vaccine composition of the present invention. In various embodiments, the subject does not take any immunosuppressive medications for about 1 day, 7 days, 14 days, 30 days, 45 days, 60 days, 75 days, 90 days, 120 days, 150 days, 180 days, 9 months, 12 months, 15 months, 18 months, 21 months, or 24 months after the administration of the modified SARS-CoV-2 coronavirus of the present invention, the immune composition of the present invention or the vaccine composition of the present invention.

Immunosuppressive medications (including, but not limited to, the following: Corticosteroids (e.g., prednisone (Deltasone, Orasone), budesonide (Entocort EC), prednisolone (Millipred)), Calcineurin inhibitors (e.g., cyclosporine (Neoral, Sandimmune, SangCya), tacrolimus (Astagraf XL, Envarsus XR, Prograf), Mechanistic target of rapamycin (mTOR) inhibitors (e.g., sirolimus (Rapamune), everolimus (Afinitor, Zortress)), Inosine monophosphate dehydrogenase (IMDH) inhibitors, (e.g., azathioprine (Azasan, Imuran), leflunomide (Arava), mycophenolate (CellCept, Myfortic)), Biologics (e.g., abatacept (Orencia), adalimumab (Humira), anakinra (Kineret), certolizumab (Cimzia), etanercept (Enbrel), golimumab (Simponi), infliximab (Remicade), ixekizumab (Taltz), natalizumab (Tysabri), rituximab (Rituxan), secukinumab (Cosentyx), tocilizumab (Actemra), ustekinumab (Stelara), vedolizumab (Entyvio)), Monoclonal antibodies (e.g., basiliximab (Simulect), daclizumab (Zinbryta), muromonab (Orthoclone OKT3)).

### Medical Uses

Various embodiments of the present invention provide for a modified SARS-CoV-2 coronavirus of the present invention, a vaccine composition of the present invention, or an immune composition of the present invention for use in eliciting an immune response, or for therapeutic or prophylactic treatment of COVID-19.

Various embodiments of the present invention provide for a modified SARS-CoV-2 coronavirus of the present invention, a vaccine composition of the present invention, or an immune composition of the present invention for use in eliciting an immune response, or for therapeutic or prophylactic treatment of COVID-19, wherein the use comprises a prime dose of the modified SARS-CoV-2 coronavirus of the present invention, or the vaccine composition of the present invention, or the immune composition of the present invention, and one or more boost doses of the modified SARS-CoV-2 coronavirus of the present invention, or the vaccine composition of the present invention, or the immune composition of the present invention.

Various embodiments of the present invention provide for a use of modified SARS-CoV-2 coronavirus of the present invention, a vaccine composition of the present invention, or an immune composition of the present invention in the manufacture of a medicament for eliciting an immune response, or for therapeutic or prophylactic treatment of COVID-19.

Various embodiments of the present invention provide for a use of modified SARS-CoV-2 coronavirus of the present invention, a vaccine composition of the present invention, or an immune composition of the present invention in the manufacture of a medicament for use in eliciting an immune response, or for therapeutic or prophylactic treatment of COVID-19, wherein the medicament comprises a prime dose of the modified SARS-CoV-2 coronavirus of the present invention, or the vaccine composition of the present invention, or the immune composition of the present invention, and one or more boost doses of the modified SARS-CoV-2 coronavirus of the present invention, or the vaccine composition of the present invention, or the immune composition of the present invention.

The modified SARS-CoV-2 coronavirus of the present invention is any one of the modified SARS-CoV-2 coronavirus discussed herein. The vaccine composition of the present invention is any one of the vaccine compositions discussed herein. The immune composition of the present invention is any one of the immune compositions discussed herein.

In various embodiments, the immune response is a protective immune response.

### Methods of Making

Various embodiments provide for a method of making a modified SARS-CoV-2 coronavirus, comprising: obtaining a nucleotide sequence encoding one or more proteins of a parent SARS-CoV-2 coronavirus or one or more fragments thereof, recoding the nucleotide sequence to reduce protein expression of the one or more proteins, or the one or more fragments thereof, and substituting a nucleic acid having the recoded nucleotide sequence into the parent SARS-CoV-2 coronavirus genome to make the modified SARS-CoV-2 coronavirus genome, wherein expression of the recoded nucleotide sequence is reduced compared to the parent virus.

In various embodiments, the parent SARS-CoV-2 coronavirus is a wild-type (wt) viral nucleic acid. In various embodiments, the parent SARS-CoV-2 coronavirus is a natural isolate viral nucleic acid. In various embodiments, the parent SARS-CoV-2 coronavirus is a previously modified viral nucleic acid, or a previously attenuated viral nucleic acid. In various embodiments, the parent SARS-CoV-2 coronavirus is a SARS-CoV-2 variant.

In various embodiment, making the modified SARS-CoV-2 coronavirus genome comprises using a cloning host.

In various embodiment, making the modified SARS-CoV-2 coronavirus genome comprises constructing an infectious cDNA clone, using BAC vector, using an overlap extension PCR strategy, or long PCR-based fusion strategy.

In various embodiment, the modified SARS-CoV-2 coronavirus genome further comprises one or more mutations, including deletion, substitutions and additions. One or more can be 1-5, 6-10, 11-15, 16-20, 21-25, 26-30, 31-35, 36-40, 41-45, 46-50, 51-60, 61-70, 71-80, 81-90, or 91-100 mutations.

In various embodiments, recoding the nucleotide sequence to reduce protein expression of the one or more proteins, or the one or more fragments thereof is by way of reducing codon-pair bias (CPB) compared to its parent SARS-CoV-2 coronavirus polynucleotide, reducing codon usage bias compared to its parent SARS-CoV-2 coronavirus polynucleotide, or increasing the number of CpG or UpA di-nucleotides compared to its parent SARS-CoV-2 coronavirus polynucleotide, as discuss herein.

### Attenuated Virus Generation

Various embodiments of the present invention provide for a method of generating an attenuated, comprising: transfection a population of cells with a vector comprising the viral genome; passaging the population of cells in a cell culture at least one time; collecting supernatant from cell culture.

In various embodiments, the method further comprises concentrating the supernatant.

In various embodiments, the method comprises passaging the population of cells 2 to 15 times; and collecting supernatant from the cell culture of the population of cells. In various embodiments, the method comprises passaging the population of cells 2 to 10 times; and collecting supernatant from the cell culture of the population of cells. In various embodiments, the method comprises passaging the population of cells 2 to 7 times; and collecting supernatant from the cell culture of the population of cells. In various embodiments, the method comprises passaging the population of cells 2 to 5 times; and collecting supernatant from the cell culture of the population of cells. In various embodiments, the method comprises passaging the population of cells 2, 3, 4, 5, 6, 7, 8, or 10 times; and collecting supernatant from the cell culture of the population of cells. In various embodiments, collecting supernatant from the cell culture is done during each passage of the population of cells. In other embodiments, collecting supernatant from the cell culture is done during one or more passages of the population of cells. For example, it can be done every other passage; every two passage, every three passage, etc.

### Kits

The present invention is also directed to a kit to vaccinate a subject, to elicit an immune response or to elicit a protective immune response in a subject. The kit is useful for practicing the inventive method of elicit an immune response or to elicit a protective immune response. The kit is an assemblage of materials or components, including at least one of the inventive compositions. Thus, in some embodiments the kit contains a composition including any one of the modified SARS-CoV-2 virus discussed herein, any one of the immune compositions discussed herein, or any one of the vaccine compositions discussed herein of the present invention. Thus, in some embodiments the kit contains unitized single dosages of the composition including the modified SARS-CoV-2 virus, the immune compositions, or the vaccine compositions of the present invention as described herein; for example, each vial contains enough for a dose of about 10³-10⁷ PFU of the modified SARS-CoV-2 virus, or more particularly, 10⁴-10⁶ PFU of the modified SARS-CoV-2 virus, 10⁴ PFU of the modified SARS-CoV-2 virus, 10⁵ PFU of the modified SARS-CoV-2 virus, or 10⁶ PFU of the modified SARS-CoV-2 virus; or more particularly, 5×10⁴-5×10⁶ PFU of the modified SARS-CoV-2 virus, 5×10⁴ PFU of the modified SARS-CoV-2 virus, 5×10⁵ PFU of the modified SARS-CoV-2 virus, or 5×10⁶ PFU of the modified SARS-CoV-2 virus. In various embodiments, the kit contains multiple dosages of the composition including the modified SARS-CoV-2 virus, the immune compositions, or the vaccine compositions of the present invention as described herein; for example, if the kit contains 10 dosages per vial, each vial contains about 10×10³-10⁷ PFU of the modified SARS-CoV-2 virus, or more particularly, 10×10⁴-10⁶ PFU of the modified SARS-CoV-2 virus, 10×10⁴ PFU of the modified SARS-CoV-2 virus, 10×10⁵ PFU of the modified SARS-CoV-2 virus, or 10×10⁶ PFU of the modified SARS-CoV-2 virus, or more particularly, 50×10⁴-50×10⁶ PFU of the modified SARS-CoV-2 virus, 50×10⁴ PFU of the modified SARS-CoV-2 virus, 50×10⁵ PFU of the modified SARS-CoV-2 virus, or 50×10⁶ PFU of the modified SARS-CoV-2 virus.

The exact nature of the components configured in the inventive kit depends on its intended purpose. For example, some embodiments are configured for the purpose of vaccinating a subject, for eliciting an immune response or for eliciting a protective immune response in a subject. In one embodiment, the kit is configured particularly for the purpose of prophylactically treating mammalian subjects. In another embodiment, the kit is configured particularly for the purpose of prophylactically treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to vaccinate a subject, to elicit an immune response or to elicit a protective immune response in a subject. For example, for nasal administration, instructions for use can include but are not limited to instructions for the subject to blow the nose and tilt the head back, instructions for the subject reposition the head to avoid having composition dripping outside of the nose or down the throat, instructions for administering about 0.25 mL comprising the dosage into each nostril; instructions for the subject to sniff gently, and/or instructions for the subject to not blow the nose for a period of time; for example, about 60 minutes. Further instructions can include instruction for the subject to not take any immunosuppressive medications

Optionally, the kit also contains other useful components, such as, diluents, buffers, pharmaceutically acceptable carriers, syringes, droppers, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by known methods, preferably to provide a sterile, contaminant-free environment. The packaging materials employed in the kit are those customarily utilized in vaccines. As used herein, the term “package” refers to a suitable solid matrix or material such as glass, plastic, paper, foil, and the like, capable of holding the individual kit components. Thus, for example, a package can be a glass vial used to contain suitable quantities of an inventive composition containing modified SARS-CoV-2 virus, the immune compositions, or the vaccine compositions of the present invention as described herein. The packaging material generally has an external label which indicates the contents and/or purpose of the kit and/or its components.

Sequences

SEQ ID NO: 2-restriction site-modified reference sequence, modified to knock
out BsaI and BsmBI sites; knocked out existing BsaI: al7973g (Arg AGA to
AGG), c24106 t (Asp GAC to GAT); knocked out existing BsmBI sites: c2197t
(Asp GAC to GAT), a9754g (Arg AGA to AGG), gl7331a (Glu GAG to GAA)
ATTAAAGGTTTATACCTTCCCAGGTAACAAACCAACCAACTTTCGATCTCTTG
TAGATCTGTTCTCTAAACGAACTTTAAAATCTGTGTGGCTGTCACTCGGCTGCATGCTTAG
TGCACTCACGCAGTATAATTAATAACTAATTACTGTCGTTGACAGGACACGAGTAACTCG
TCTATCTTCTGCAGGCTGCTTACGGTTTCGTCCGTGTTGCAGCCGATCATCAGCACATCTA
GGTTTCGTCCGGGTGTGACCGAAAGGTAAGATGGAGAGCCTTGTCCCTGGTTTCAACGAG
AAAACACACGTCCAACTCAGTTTGCCTGTTTTACAGGTTCGCGACGTGCTCGTACGTGGC
TTTGGAGACTCCGTGGAGGAGGTCTTATCAGAGGCACGTCAACATCTTAAAGATGGCACT
TGTGGCTTAGTAGAAGTTGAAAAAGGCGTTTTGCCTCAACTTGAACAGCCCTATGTGTTC
ATCAAACGTTCGGATGCTCGAACTGCACCTCATGGTCATGTTATGGTTGAGCTGGTAGCA
GAACTCGAAGGCATTCAGTACGGTCGTAGTGGTGAGACACTTGGTGTCCTTGTCCCTCAT
GTGGGCGAAATACCAGTGGCTTACCGCAAGGTTCTTCTTCGTAAGAACGGTAATAAAGG
AGCTGGTGGCCATAGTTACGGCGCCGATCTAAAGTCATTTGACTTAGGCGACGAGCTTGG
CACTGATCCTTATGAAGATTTTCAAGAAAACTGGAACACTAAACATAGCAGTGGTGTTAC
CCGTGAACTCATGCGTGAGCTTAACGGAGGGGCATACACTCGCTATGTCGATAACAACTT
CTGTGGCCCTGATGGCTACCCTCTTGAGTGCATTAAAGACCTTCTAGCACGTGCTGGTAA
AGCTTCATGCACTTTGTCCGAACAACTGGACTTTATTGACACTAAGAGGGGTGTATACTG
CTGCCGTGAACATGAGCATGAAATTGCTTGGTACACGGAACGTTCTGAAAAGAGCTATG
AATTGCAGACACCTTTTGAAATTAAATTGGCAAAGAAATTTGACACCTTCAATGGGGAAT
GTCCAAATTTTGTATTTCCCTTAAATTCCATAATCAAGACTATTCAACCAAGGGTTGAAA
AGAAAAAGCTTGATGGCTTTATGGGTAGAATTCGATCTGTCTATCCAGTTGCGTCACCAA
ATGAATGCAACCAAATGTGCCTTTCAACTCTCATGAAGTGTGATCATTGTGGTGAAACTT
CATGGCAGACGGGCGATTTTGTTAAAGCCACTTGCGAATTTTGTGGCACTGAGAATTTGA
CTAAAGAAGGTGCCACTACTTGTGGTTACTTACCCCAAAATGCTGTTGTTAAAATTTATT
GTCCAGCATGTCACAATTCAGAAGTAGGACCTGAGCATAGTCTTGCCGAATACCATAATG
AATCTGGCTTGAAAACCATTCTTCGTAAGGGTGGTCGCACTATTGCCTTTGGAGGCTGTG
TGTTCTCTTATGTTGGTTGCCATAACAAGTGTGCCTATTGGGTTCCACGTGCTAGCGCTAA
CATAGGTTGTAACCATACAGGTGTTGTTGGAGAAGGTTCCGAAGGTCTTAATGACAACCT
TCTTGAAATACTCCAAAAAGAGAAAGTCAACATCAATATTGTTGGTGACTTTAAACTTAA
TGAAGAGATCGCCATTATTTTGGCATCTTTTTCTGCTTCCACAAGTGCTTTTGTGGAAACT
GTGAAAGGTTTGGATTATAAAGCATTCAAACAAATTGTTGAATCCTGTGGTAATTTTAAA
GTTACAAAAGGAAAAGCTAAAAAAGGTGCCTGGAATATTGGTGAACAGAAATCAATACT
GAGTCCTCTTTATGCATTTGCATCAGAGGCTGCTCGTGTTGTACGATCAATTTTCTCCCGC
ACTCTTGAAACTGCTCAAAATTCTGTGCGTGTTTTACAGAAGGCCGCTATAACAATACTA
GATGGAATTTCACAGTATTCACTGAGACTCATTGATGCTATGATGTTCACATCTGATTTG
GCTACTAACAATCTAGTTGTAATGGCCTACATTACAGGTGGTGTTGTTCAGTTGACTTCG
CAGTGGCTAACTAACATCTTTGGCACTGTTTATGAAAAACTCAAACCCGTCCTTGATTGG
CTTGAAGAGAAGTTTAAGGAAGGTGTAGAGTTTCTTAGAGAtGGTTGGGAAATTGTTAAA
TTTATCTCAACCTGTGCTTGTGAAATTGTCGGTGGACAAATTGTCACCTGTGCAAAGGAA
ATTAAGGAGAGTGTTCAGACATTCTTTAAGCTTGTAAATAAATTTTTGGCTTTGTGTGCTG
ACTCTATCATTATTGGTGGAGCTAAACTTAAAGCCTTGAATTTAGGTGAAACATTTGTCA
CGCACTCAAAGGGATTGTACAGAAAGTGTGTTAAATCCAGAGAAGAAACTGGCCTACTC
ATGCCTCTAAAAGCCCCAAAAGAAATTATCTTCTTAGAGGGAGAAACACTTCCCACAGA
AGTGTTAACAGAGGAAGTTGTCTTGAAAACTGGTGATTTACAACCATTAGAACAACCTAC
TAGTGAAGCTGTTGAAGCTCCATTGGTTGGTACACCAGTTTGTATTAACGGGCTTATGTT
GCTCGAAATCAAAGACACAGAAAAGTACTGTGCCCTTGCACCTAATATGATGGTAACAA
ACAATACCTTCACACTCAAAGGCGGTGCACCAACAAAGGTTACTTTTGGTGATGACACTG
TGATAGAAGTGCAAGGTTACAAGAGTGTGAATATCACTTTTGAACTTGATGAAAGGATT
GATAAAGTACTTAATGAGAAGTGCTCTGCCTATACAGTTGAACTCGGTACAGAAGTAAA
TGAGTTCGCCTGTGTTGTGGCAGATGCTGTCATAAAAACTTTGCAACCAGTATCTGAATT
ACTTACACCACTGGGCATTGATTTAGATGAGTGGAGTATGGCTACATACTACTTATTTGA
TGAGTCTGGTGAGTTTAAATTGGCTTCACATATGTATTGTTCTTTCTACCCTCCAGATGAG
GATGAAGAAGAAGGTGATTGTGAAGAAGAAGAGTTTGAGCCATCAACTCAATATGAGTA
TGGTACTGAAGATGATTACCAAGGTAAACCTTTGGAATTTGGTGCCACTTCTGCTGCTCT
TCAACCTGAAGAAGAGCAAGAAGAAGATTGGTTAGATGATGATAGTCAACAAACTGTTG
GTCAACAAGACGGCAGTGAGGACAATCAGACAACTACTATTCAAACAATTGTTGAGGTT
CAACCTCAATTAGAGATGGAACTTACACCAGTTGTTCAGACTATTGAAGTGAATAGTTTT
AGTGGTTATTTAAAACTTACTGACAATGTATACATTAAAAATGCAGACATTGTGGAAGAA
GCTAAAAAGGTAAAACCAACAGTGGTTGTTAATGCAGCCAATGTTTACCTTAAACATGG
AGGAGGTGTTGCAGGAGCCTTAAATAAGGCTACTAACAATGCCATGCAAGTTGAATCTG
ATGATTACATAGCTACTAATGGACCACTTAAAGTGGGTGGTAGTTGTGTTTTAAGCGGAC
ACAATCTTGCTAAACACTGTCTTCATGTTGTCGGCCCAAATGTTAACAAAGGTGAAGACA
TTCAACTTCTTAAGAGTGCTTATGAAAATTTTAATCAGCACGAAGTTCTACTTGCACCATT
ATTATCAGCTGGTATTTTTGGTGCTGACCCTATACATTCTTTAAGAGTTTGTGTAGATACT
GTTCGCACAAATGTCTACTTAGCTGTCTTTGATAAAAATCTCTATGACAAACTTGTTTCAA
GCTTTTTGGAAATGAAGAGTGAAAAGCAAGTTGAACAAAAGATCGCTGAGATTCCTAAA
GAGGAAGTTAAGCCATTTATAACTGAAAGTAAACCTTCAGTTGAACAGAGAAAACAAGA
TGATAAGAAAATCAAAGCTTGTGTTGAAGAAGTTACAACAACTCTGGAAGAAACTAAGT
TCCTCACAGAAAACTTGTTACTTTATATTGACATTAATGGCAATCTTCATCCAGATTCTGC
CACTCTTGTTAGTGACATTGACATCACTTTCTTAAAGAAAGATGCTCCATATATAGTGGG
TGATGTTGTTCAAGAGGGTGTTTTAACTGCTGTGGTTATACCTACTAAAAAGGCTGGTGG
CACTACTGAAATGCTAGCGAAAGCTTTGAGAAAAGTGCCAACAGACAATTATATAACCA
CTTACCCGGGTCAGGGTTTAAATGGTTACACTGTAGAGGAGGCAAAGACAGTGCTTAAA
AAGTGTAAAAGTGCCTTTTACATTCTACCATCTATTATCTCTAATGAGAAGCAAGAAATT
CTTGGAACTGTTTCTTGGAATTTGCGAGAAATGCTTGCACATGCAGAAGAAACACGCAA
ATTAATGCCTGTCTGTGTGGAAACTAAAGCCATAGTTTCAACTATACAGCGTAAATATAA
GGGTATTAAAATACAAGAGGGTGTGGTTGATTATGGTGCTAGATTTTACTTTTACACCAG
TAAAACAACTGTAGCGTCACTTATCAACACACTTAACGATCTAAATGAAACTCTTGTTAC
AATGCCACTTGGCTATGTAACACATGGCTTAAATTTGGAAGAAGCTGCTCGGTATATGAG
ATCTCTCAAAGTGCCAGCTACAGTTTCTGTTTCTTCACCTGATGCTGTTACAGCGTATAAT
GGTTATCTTACTTCTTCTTCTAAAACACCTGAAGAACATTTTATTGAAACCATCTCACTTG
CTGGTTCCTATAAAGATTGGTCCTATTCTGGACAATCTACACAACTAGGTATAGAATTTC
TTAAGAGAGGTGATAAAAGTGTATATTACACTAGTAATCCTACCACATTCCACCTAGATG
GTGAAGTTATCACCTTTGACAATCTTAAGACACTTCTTTCTTTGAGAGAAGTGAGGACTA
TTAAGGTGTTTACAACAGTAGACAACATTAACCTCCACACGCAAGTTGTGGACATGTCAA
TGACATATGGACAACAGTTTGGTCCAACTTATTTGGATGGAGCTGATGTTACTAAAATAA
AACCTCATAATTCACATGAAGGTAAAACATTTTATGTTTTACCTAATGATGACACTCTAC
GTGTTGAGGCTTTTGAGTACTACCACACAACTGATCCTAGTTTTCTGGGTAGGTACATGT
CAGCATTAAATCACACTAAAAAGTGGAAATACCCACAAGTTAATGGTTTAACTTCTATTA
AATGGGCAGATAACAACTGTTATCTTGCCACTGCATTGTTAACACTCCAACAAATAGAGT
TGAAGTTTAATCCACCTGCTCTACAAGATGCTTATTACAGAGCAAGGGCTGGTGAAGCTG
CTAACTTTTGTGCACTTATCTTAGCCTACTGTAATAAGACAGTAGGTGAGTTAGGTGATG
TTAGAGAAACAATGAGTTACTTGTTTCAACATGCCAATTTAGATTCTTGCAAAAGAGTCT
TGAACGTGGTGTGTAAAACTTGTGGACAACAGCAGACAACCCTTAAGGGTGTAGAAGCT
GTTATGTACATGGGCACACTTTCTTATGAACAATTTAAGAAAGGTGTTCAGATACCTTGT
ACGTGTGGTAAACAAGCTACAAAATATCTAGTACAACAGGAGTCACCTTTTGTTATGATG
TCAGCACCACCTGCTCAGTATGAACTTAAGCATGGTACATTTACTTGTGCTAGTGAGTAC
ACTGGTAATTACCAGTGTGGTCACTATAAACATATAACTTCTAAAGAAACTTTGTATTGC
ATAGACGGTGCTTTACTTACAAAGTCCTCAGAATACAAAGGTCCTATTACGGATGTTTTC
TACAAAGAAAACAGTTACACAACAACCATAAAACCAGTTACTTATAAATTGGATGGTGT
TGTTTGTACAGAAATTGACCCTAAGTTGGACAATTATTATAAGAAAGACAATTCTTATTT
CACAGAGCAACCAATTGATCTTGTACCAAACCAACCATATCCAAACGCAAGCTTCGATA
ATTTTAAGTTTGTATGTGATAATATCAAATTTGCTGATGATTTAAACCAGTTAACTGGTTA
TAAGAAACCTGCTTCAAGAGAGCTTAAAGTTACATTTTTCCCTGACTTAAATGGTGATGT
GGTGGCTATTGATTATAAACACTACACACCCTCTTTTAAGAAAGGAGCTAAATTGTTACA
TAAACCTATTGTTTGGCATGTTAACAATGCAACTAATAAAGCCACGTATAAACCAAATAC
CTGGTGTATACGTTGTCTTTGGAGCACAAAACCAGTTGAAACATCAAATTCGTTTGATGT
ACTGAAGTCAGAGGACGCGCAGGGAATGGATAATCTTGCCTGCGAAGATCTAAAACCAG
TCTCTGAAGAAGTAGTGGAAAATCCTACCATACAGAAAGACGTTCTTGAGTGTAATGTG
AAAACTACCGAAGTTGTAGGAGACATTATACTTAAACCAGCAAATAATAGTTTAAAAAT
TACAGAAGAGGTTGGCCACACAGATCTAATGGCTGCTTATGTAGACAATTCTAGTCTTAC
TATTAAGAAACCTAATGAATTATCTAGAGTATTAGGTTTGAAAACCCTTGCTACTCATGG
TTTAGCTGCTGTTAATAGTGTCCCTTGGGATACTATAGCTAATTATGCTAAGCCTTTTCTT
AACAAAGTTGTTAGTACAACTACTAACATAGTTACACGGTGTTTAAACCGTGTTTGTACT
AATTATATGCCTTATTTCTTTACTTTATTGCTACAATTGTGTACTTTTACTAGAAGTACAA
ATTCTAGAATTAAAGCATCTATGCCGACTACTATAGCAAAGAATACTGTTAAGAGTGTCG
GTAAATTTTGTCTAGAGGCTTCATTTAATTATTTGAAGTCACCTAATTTTTCTAAACTGAT
AAATATTATAATTTGGTTTTTACTATTAAGTGTTTGCCTAGGTTCTTTAATCTACTCAACC
GCTGCTTTAGGTGTTTTAATGTCTAATTTAGGCATGCCTTCTTACTGTACTGGTTACAGAG
AAGGCTATTTGAACTCTACTAATGTCACTATTGCAACCTACTGTACTGGTTCTATACCTTG
TAGTGTTTGTCTTAGTGGTTTAGATTCTTTAGACACCTATCCTTCTTTAGAAACTATACAA
ATTACCATTTCATCTTTTAAATGGGATTTAACTGCTTTTGGCTTAGTTGCAGAGTGGTTTT
TGGCATATATTCTTTTCACTAGGTTTTTCTATGTACTTGGATTGGCTGCAATCATGCAATT
GTTTTTCAGCTATTTTGCAGTACATTTTATTAGTAATTCTTGGCTTATGTGGTTAATAATTA
ATCTTGTACAAATGGCCCCGATTTCAGCTATGGTTAGAATGTACATCTTCTTTGCATCATT
TTATTATGTATGGAAAAGTTATGTGCATGTTGTAGACGGTTGTAATTCATCAACTTGTATG
ATGTGTTACAAACGTAATAGAGCAACAAGAGTCGAATGTACAACTATTGTTAATGGTGTT
AGAAGGTCCTTTTATGTCTATGCTAATGGAGGTAAAGGCTTTTGCAAACTACACAATTGG
AATTGTGTTAATTGTGATACATTCTGTGCTGGTAGTACATTTATTAGTGATGAAGTTGCGA
GAGACTTGTCACTACAGTTTAAAAGACCAATAAATCCTACTGACCAGTCTTCTTACATCG
TTGATAGTGTTACAGTGAAGAATGGTTCCATCCATCTTTACTTTGATAAAGCTGGTCAAA
AGACTTATGAAAGACATTCTCTCTCTCATTTTGTTAACTTAGACAACCTGAGAGCTAATA
ACACTAAAGGTTCATTGCCTATTAATGTTATAGTTTTTGATGGTAAATCAAAATGTGAAG
AATCATCTGCAAAATCAGCGTCTGTTTACTACAGTCAGCTTATGTGTCAACCTATACTGTT
ACTAGATCAGGCATTAGTGTCTGATGTTGGTGATAGTGCGGAAGTTGCAGTTAAAATGTT
TGATGCTTACGTTAATACGTTTTCATCAACTTTTAACGTACCAATGGAAAAACTCAAAAC
ACTAGTTGCAACTGCAGAAGCTGAACTTGCAAAGAATGTGTCCTTAGACAATGTCTTATC
TACTTTTATTTCAGCAGCTCGGCAAGGGTTTGTTGATTCAGATGTAGAAACTAAAGATGT
TGTTGAATGTCTTAAATTGTCACATCAATCTGACATAGAAGTTACTGGCGATAGTTGTAA
TAACTATATGCTCACCTATAACAAAGTTGAAAACATGACACCCCGTGACCTTGGTGCTTG
TATTGACTGTAGTGCGCGTCATATTAATGCGCAGGTAGCAAAAAGTCACAACATTGCTTT
GATATGGAACGTTAAAGATTTCATGTCATTGTCTGAACAACTACGAAAACAAATACGTA
GTGCTGCTAAAAAGAATAACTTACCTTTTAAGTTGACATGTGCAACTACTAGACAAGTTG
TTAATGTTGTAACAACAAAGATAGCACTTAAGGGTGGTAAAATTGTTAATAATTGGTTGA
AGCAGTTAATTAAAGTTACACTTGTGTTCCTTTTTGTTGCTGCTATTTTCTATTTAATAAC
ACCTGTTCATGTCATGTCTAAACATACTGACTTTTCAAGTGAAATCATAGGATACAAGGC
TATTGATGGTGGTGTCACTCGTGACATAGCATCTACAGATACTTGTTTTGCTAACAAACA
TGCTGATTTTGACACATGGTTTAGCCAGCGTGGTGGTAGTTATACTAATGACAAAGCTTG
CCCATTGATTGCTGCAGTCATAACAAGAGAAGTGGGTTTTGTCGTGCCTGGTTTGCCTGG
CACGATATTACGCACAACTAATGGTGACTTTTTGCATTTCTTACCTAGAGTTTTTAGTGCA
GTTGGTAACATCTGTTACACACCATCAAAACTTATAGAGTACACTGACTTTGCAACATCA
GCTTGTGTTTTGGCTGCTGAATGTACAATTTTTAAAGATGCTTCTGGTAAGCCAGTACCAT
ATTGTTATGATACCAATGTACTAGAAGGTTCTGTTGCTTATGAAAGTTTACGCCCTGACA
CACGTTATGTGCTCATGGATGGCTCTATTATTCAATTTCCTAACACCTACCTTGAAGGTTC
TGTTAGAGTGGTAACAACTTTTGATTCTGAGTACTGTAGGCACGGCACTTGTGAAAGATC
AGAAGCTGGTGTTTGTGTATCTACTAGTGGTAGATGGGTACTTAACAATGATTATTACAG
ATCTTTACCAGGAGTTTTCTGTGGTGTAGATGCTGTAAATTTACTTACTAATATGTTTACA
CCACTAATTCAACCTATTGGTGCTTTGGACATATCAGCATCTATAGTAGCTGGTGGTATT
GTAGCTATCGTAGTAACATGCCTTGCCTACTATTTTATGAGGTTTAGAAGAGCTTTTGGTG
AATACAGTCATGTAGTTGCCTTTAATACTTTACTATTCCTTATGTCATTCACTGTACTCTG
TTTAACACCAGTTTACTCATTCTTACCTGGTGTTTATTCTGTTATTTACTTGTACTTGACAT
TTTATCTTACTAATGATGTTTCTTTTTTAGCACATATTCAGTGGATGGTTATGTTCACACCT
TTAGTACCTTTCTGGATAACAATTGCTTATATCATTTGTATTTCCACAAAGCATTTCTATT
GGTTCTTTAGTAATTACCTAAAGAGgCGTGTAGTCTTTAATGGTGTTTCCTTTAGTACTTTT
GAAGAAGCTGCGCTGTGCACCTTTTTGTTAAATAAAGAAATGTATCTAAAGTTGCGTAGT
GATGTGCTATTACCTCTTACGCAATATAATAGATACTTAGCTCTTTATAATAAGTACAAG
TATTTTAGTGGAGCAATGGATACAACTAGCTACAGAGAAGCTGCTTGTTGTCATCTCGCA
AAGGCTCTCAATGACTTCAGTAACTCAGGTTCTGATGTTCTTTACCAACCACCACAAACC
TCTATCACCTCAGCTGTTTTGCAGAGTGGTTTTAGAAAAATGGCATTCCCATCTGGTAAA
GTTGAGGGTTGTATGGTACAAGTAACTTGTGGTACAACTACACTTAACGGTCTTTGGCTT
GATGACGTAGTTTACTGTCCAAGACATGTGATCTGCACCTCTGAAGACATGCTTAACCCT
AATTATGAAGATTTACTCATTCGTAAGTCTAATCATAATTTCTTGGTACAGGCTGGTAAT
GTTCAACTCAGGGTTATTGGACATTCTATGCAAAATTGTGTACTTAAGCTTAAGGTTGAT
ACAGCCAATCCTAAGACACCTAAGTATAAGTTTGTTCGCATTCAACCAGGACAGACTTTT
TCAGTGTTAGCTTGTTACAATGGTTCACCATCTGGTGTTTACCAATGTGCTATGAGGCCCA
ATTTCACTATTAAGGGTTCATTCCTTAATGGTTCATGTGGTAGTGTTGGTTTTAACATAGA
TTATGACTGTGTCTCTTTTTGTTACATGCACCATATGGAATTACCAACTGGAGTTCATGCT
GGCACAGACTTAGAAGGTAACTTTTATGGACCTTTTGTTGACAGGCAAACAGCACAAGC
AGCTGGTACGGACACAACTATTACAGTTAATGTTTTAGCTTGGTTGTACGCTGCTGTTAT
AAATGGAGACAGGTGGTTTCTCAATCGATTTACCACAACTCTTAATGACTTTAACCTTGT
GGCTATGAAGTACAATTATGAACCTCTAACACAAGACCATGTTGACATACTAGGACCTCT
TTCTGCTCAAACTGGAATTGCCGTTTTAGATATGTGTGCTTCATTAAAAGAATTACTGCA
AAATGGTATGAATGGACGTACCATATTGGGTAGTGCTTTATTAGAAGATGAATTTACACC
TTTTGATGTTGTTAGACAATGCTCAGGTGTTACTTTCCAAAGTGCAGTGAAAAGAACAAT
CAAGGGTACACACCACTGGTTGTTACTCACAATTTTGACTTCACTTTTAGTTTTAGTCCAG
AGTACTCAATGGTCTTTGTTCTTTTTTTTGTATGAAAATGCCTTTTTACCTTTTGCTATGGG
TATTATTGCTATGTCTGCTTTTGCAATGATGTTTGTCAAACATAAGCATGCATTTCTCTGT
TTGTTTTTGTTACCTTCTCTTGCCACTGTAGCTTATTTTAATATGGTCTATATGCCTGCTAG
TTGGGTGATGCGTATTATGACATGGTTGGATATGGTTGATACTAGTTTGTCTGGTTTTAAG
CTAAAAGACTGTGTTATGTATGCATCAGCTGTAGTGTTACTAATCCTTATGACAGCAAGA
ACTGTGTATGATGATGGTGCTAGGAGAGTGTGGACACTTATGAATGTCTTGACACTCGTT
TATAAAGTTTATTATGGTAATGCTTTAGATCAAGCCATTTCCATGTGGGCTCTTATAATCT
CTGTTACTTCTAACTACTCAGGTGTAGTTACAACTGTCATGTTTTTGGCCAGAGGTATTGT
TTTTATGTGTGTTGAGTATTGCCCTATTTTCTTCATAACTGGTAATACACTTCAGTGTATA
ATGCTAGTTTATTGTTTCTTAGGCTATTTTTGTACTTGTTACTTTGGCCTCTTTTGTTTACT
CAACCGCTACTTTAGACTGACTCTTGGTGTTTATGATTACTTAGTTTCTACACAGGAGTTT
AGATATATGAATTCACAGGGACTACTCCCACCCAAGAATAGCATAGATGCCTTCAAACTC
AACATTAAATTGTTGGGTGTTGGTGGCAAACCTTGTATCAAAGTAGCCACTGTACAGTCT
AAAATGTCAGATGTAAAGTGCACATCAGTAGTCTTACTCTCAGTTTTGCAACAACTCAGA
GTAGAATCATCATCTAAATTGTGGGCTCAATGTGTCCAGTTACACAATGACATTCTCTTA
GCTAAAGATACTACTGAAGCCTTTGAAAAAATGGTTTCACTACTTTCTGTTTTGCTTTCCA
TGCAGGGTGCTGTAGACATAAACAAGCTTTGTGAAGAAATGCTGGACAACAGGGCAACC
TTACAAGCTATAGCCTCAGAGTTTAGTTCCCTTCCATCATATGCAGCTTTTGCTACTGCTC
AAGAAGCTTATGAGCAGGCTGTTGCTAATGGTGATTCTGAAGTTGTTCTTAAAAAGTTGA
AGAAGTCTTTGAATGTGGCTAAATCTGAATTTGACCGTGATGCAGCCATGCAACGTAAGT
TGGAAAAGATGGCTGATCAAGCTATGACCCAAATGTATAAACAGGCTAGATCTGAGGAC
AAGAGGGCAAAAGTTACTAGTGCTATGCAGACAATGCTTTTCACTATGCTTAGAAAGTTG
GATAATGATGCACTCAACAACATTATCAACAATGCAAGAGATGGTTGTGTTCCCTTGAAC
ATAATACCTCTTACAACAGCAGCCAAACTAATGGTTGTCATACCAGACTATAACACATAT
AAAAATACGTGTGATGGTACAACATTTACTTATGCATCAGCATTGTGGGAAATCCAACAG
GTTGTAGATGCAGATAGTAAAATTGTTCAACTTAGTGAAATTAGTATGGACAATTCACCT
AATTTAGCATGGCCTCTTATTGTAACAGCTTTAAGGGCCAATTCTGCTGTCAAATTACAG
AATAATGAGCTTAGTCCTGTTGCACTACGACAGATGTCTTGTGCTGCCGGTACTACACAA
ACTGCTTGCACTGATGACAATGCGTTAGCTTACTACAACACAACAAAGGGAGGTAGGTTT
GTACTTGCACTGTTATCCGATTTACAGGATTTGAAATGGGCTAGATTCCCTAAGAGTGAT
GGAACTGGTACTATCTATACAGAACTGGAACCACCTTGTAGGTTTGTTACAGACACACCT
AAAGGTCCTAAAGTGAAGTATTTATACTTTATTAAAGGATTAAACAACCTAAATAGAGGT
ATGGTACTTGGTAGTTTAGCTGCCACAGTACGTCTACAAGCTGGTAATGCAACAGAAGTG
CCTGCCAATTCAACTGTATTATCTTTCTGTGCTTTTGCTGTAGATGCTGCTAAAGCTTACA
AAGATTATCTAGCTAGTGGGGGACAACCAATCACTAATTGTGTTAAGATGTTGTGTACAC
ACACTGGTACTGGTCAGGCAATAACAGTTACACCGGAAGCCAATATGGATCAAGAATCC
TTTGGTGGTGCATCGTGTTGTCTGTACTGCCGTTGCCACATAGATCATCCAAATCCTAAA
GGATTTTGTGACTTAAAAGGTAAGTATGTACAAATACCTACAACTTGTGCTAATGACCCT
GTGGGTTTTACACTTAAAAACACAGTCTGTACCGTCTGCGGTATGTGGAAAGGTTATGGC
TGTAGTTGTGATCAACTCCGCGAACCCATGCTTCAGTCAGCTGATGCACAATCGTTTTTA
AACGGGTTTGCGGTGTAAGTGCAGCCCGTCTTACACCGTGCGGCACAGGCACTAGTACTG
ATGTCGTATACAGGGCTTTTGACATCTACAATGATAAAGTAGCTGGTTTTGCTAAATTCC
TAAAAACTAATTGTTGTCGCTTCCAAGAAAAGGACGAAGATGACAATTTAATTGATTCTT
ACTTTGTAGTTAAGAGACACACTTTCTCTAACTACCAACATGAAGAAACAATTTATAATT
TACTTAAGGATTGTCCAGCTGTTGCTAAACATGACTTCTTTAAGTTTAGAATAGACGGTG
ACATGGTACCACATATATCACGTCAACGTCTTACTAAATACACAATGGCAGACCTCGTCT
ATGCTTTAAGGCATTTTGATGAAGGTAATTGTGACACATTAAAAGAAATACTTGTCACAT
ACAATTGTTGTGATGATGATTATTTCAATAAAAAGGACTGGTATGATTTTGTAGAAAACC
CAGATATATTACGCGTATACGCCAACTTAGGTGAACGTGTACGCCAAGCTTTGTTAAAAA
CAGTACAATTCTGTGATGCCATGCGAAATGCTGGTATTGTTGGTGTACTGACATTAGATA
ATCAAGATCTCAATGGTAACTGGTATGATTTCGGTGATTTCATACAAACCACGCCAGGTA
GTGGAGTTCCTGTTGTAGATTCTTATTATTCATTGTTAATGCCTATATTAACCTTGACCAG
GGCTTTAACTGCAGAGTCACATGTTGACACTGACTTAACAAAGCCTTACATTAAGTGGGA
TTTGTTAAAATATGACTTCACGGAAGAGAGGTTAAAACTCTTTGACCGTTATTTTAAATA
TTGGGATCAGACATACCACCCAAATTGTGTTAACTGTTTGGATGACAGATGCATTCTGCA
TTGTGCAAACTTTAATGTTTTATTCTCTACAGTGTTCCCACCTACAAGTTTTGGACCACTA
GTGAGAAAAATATTTGTTGATGGTGTTCCATTTGTAGTTTCAACTGGATACCACTTCAGA
GAGCTAGGTGTTGTACATAATCAGGATGTAAACTTACATAGCTCTAGACTTAGTTTTAAG
GAATTACTTGTGTATGCTGCTGACCCTGCTATGCACGCTGCTTCTGGTAATCTATTACTAG
ATAAACGCACTACGTGCTTTTCAGTAGCTGCACTTACTAACAATGTTGCTTTTCAAACTGT
CAAACCCGGTAATTTTAACAAAGACTTCTATGACTTTGCTGTGTCTAAGGGTTTCTTTAAG
GAAGGAAGTTCTGTTGAATTAAAACACTTCTTCTTTGCTCAGGATGGTAATGCTGCTATC
AGCGATTATGACTACTATCGTTATAATCTACCAACAATGTGTGATATCAGACAACTACTA
TTTGTAGTTGAAGTTGTTGATAAGTACTTTGATTGTTACGATGGTGGCTGTATTAATGCTA
ACCAAGTCATCGTCAACAACCTAGACAAATCAGCTGGTTTTCCATTTAATAAATGGGGTA
AGGCTAGACTTTATTATGATTCAATGAGTTATGAGGATCAAGATGCACTTTTCGCATATA
CAAAACGTAATGTCATCCCTACTATAACTCAAATGAATCTTAAGTATGCCATTAGTGCAA
AGAATAGAGCTCGCACCGTAGCTGGTGTCTCTATCTGTAGTACTATGACCAATAGACAGT
TTCATCAAAAATTATTGAAATCAATAGCCGCCACTAGAGGAGCTACTGTAGTAATTGGAA
CAAGCAAATTCTATGGTGGTTGGCACAACATGTTAAAAACTGTTTATAGTGATGTAGAAA
ACCCTCACCTTATGGGTTGGGATTATCCTAAATGTGATAGAGCCATGCCTAACATGCTTA
GAATTATGGCCTCACTTGTTCTTGCTCGCAAACATACAACGTGTTGTAGCTTGTCACACC
GTTTCTATAGATTAGCTAATGAGTGTGCTCAAGTATTGAGTGAAATGGTCATGTGTGGCG
GTTCACTATATGTTAAACCAGGTGGAACCTCATCAGGAGATGCCACAACTGCTTATGCTA
ATAGTGTTTTTAACATTTGTCAAGCTGTCACGGCCAATGTTAATGCACTTTTATCTACTGA
TGGTAACAAAATTGCCGATAAGTATGTCCGCAATTTACAACACAGACTTTATGAGTGTCT
CTATAGAAATAGAGATGTTGACACAGACTTTGTGAATGAGTTTTACGCATATTTGCGTAA
ACATTTCTCAATGATGATACTCTCTGACGATGCTGTTGTGTGTTTCAATAGCACTTATGCA
TCTCAAGGTCTAGTGGCTAGCATAAAGAACTTTAAGTCAGTTCTTTATTATCAAAACAAT
GTTTTTATGTCTGAAGCAAAATGTTGGACTGAGACTGACCTTACTAAAGGACCTCATGAA
TTTTGCTCTCAACATACAATGCTAGTTAAACAGGGTGATGATTATGTGTACCTTCCTTACC
CAGATCCATCAAGAATCCTAGGGGCCGGCTGTTTTGTAGATGATATCGTAAAAACAGAT
GGTACACTTATGATTGAACGGTTCGTGTCTTTAGCTATAGATGCTTACCCACTTACTAAAC
ATCCTAATCAGGAGTATGCTGATGTCTTTCATTTGTACTTACAATACATAAGAAAGCTAC
ATGATGAGTTAACAGGACACATGTTAGACATGTATTCTGTTATGCTTACTAATGATAACA
CTTCAAGGTATTGGGAACCTGAGTTTTATGAGGCTATGTACACACCGCATACAGTCTTAC
AGGCTGTTGGGGCTTGTGTTCTTTGCAATTCACAGACTTCATTAAGATGTGGTGCTTGCAT
ACGTAGACCATTCTTATGTTGTAAATGCTGTTACGACCATGTCATATCAACATCACATAA
ATTAGTCTTGTCTGTTAATCCGTATGTTTGCAATGCTCCAGGTTGTGATGTCACAGATGTG
ACTCAACTTTACTTAGGAGGTATGAGCTATTATTGTAAATCACATAAACCACCCATTAGT
TTTCCATTGTGTGCTAATGGACAAGTTTTTGGTTTATATAAAAATACATGTGTTGGTAGCG
ATAATGTTACTGACTTTAATGCAATTGCAACATGTGACTGGACAAATGCTGGTGATTACA
TTTTAGCTAACACCTGTACTGAAAGACTCAAGCTTTTTGCAGCAGAAACGCTCAAAGCTA
CTGAGGAGACATTTAAACTGTCTTATGGTATTGCTACTGTACGTGAAGTGCTGTCTGACA
GAGAATTACATCTTTCATGGGAAGTTGGTAAACCTAGACCACCACTTAACCGAAATTATG
TCTTTACTGGTTATCGTGTAACTAAAAACAGTAAAGTACAAATAGGAGAGTACACCTTTG
AAAAAGGTGACTATGGTGATGCTGTTGTTTACCGAGGTACAACAACTTACAAATTAAATG
TTGGTGATTATTTTGTGCTGACATCACATACAGTAATGCCATTAAGTGCACCTACACTAG
TGCCACAAGAGCACTATGTTAGAATTACTGGCTTATACCCAACACTCAATATCTCAGATG
AGTTTTCTAGCAATGTTGCAAATTATCAAAAGGTTGGTATGCAAAAGTATTCTACACTCC
AGGGACCACCTGGTACTGGTAAGAGTCATTTTGCTATTGGCCTAGCTCTCTACTACCCTTC
TGCTCGCATAGTGTATACAGCTTGCTCTCATGCCGCTGTTGATGCACTATGTGAGAAGGC
ATTAAAATATTTGCCTATAGATAAATGTAGTAGAATTATACCTGCACGTGCTCGTGTAGA
GTGTTTTGATAAATTCAAAGTGAATTCAACATTAGAACAGTATGTCTTTTGTACTGTAAA
TGCATTGCCTGAaACGACAGCAGATATAGTTGTCTTTGATGAAATTTCAATGGCCACAAA
TTATGATTTGAGTGTTGTCAATGCCAGATTACGTGCTAAGCACTATGTGTACATTGGCGA
CCCTGCTCAATTACCTGCACCACGCACATTGCTAACTAAGGGCACACTAGAACCAGAATA
TTTCAATTCAGTGTGTAGACTTATGAAAACTATAGGTCCAGACATGTTCCTCGGAACTTG
TCGGCGTTGTCCTGCTGAAATTGTTGACACTGTGAGTGCTTTGGTTTATGATAATAAGCTT
AAAGCACATAAAGACAAATCAGCTCAATGCTTTAAAATGTTTTATAAGGGTGTTATCACG
CATGATGTTTCATCTGCAATTAACAGGCCACAAATAGGCGTGGTAAGAGAATTCCTTACA
CGTAACCCTGCTTGGAGAAAAGCTGTCTTTATTTCACCTTATAATTCACAGAATGCTGTA
GCCTCAAAGATTTTGGGACTACCAACTCAAACTGTTGATTCATCACAGGGCTCAGAATAT
GACTATGTCATATTCACTCAAACCACTGAAACAGCTCACTCTTGTAATGTAAACAGATTT
AATGTTGCTATTACCAGAGCAAAAGTAGGCATACTTTGCATAATGTCTGATAGgGACCTT
TATGACAAGTTGCAATTTACAAGTCTTGAAATTCCACGTAGGAATGTGGCAACTTTACAA
GCTGAAAATGTAACAGGACTCTTTAAAGATTGTAGTAAGGTAATCACTGGGTTACATCCT
ACACAGGCACCTACACACCTCAGTGTTGACACTAAATTCAAAACTGAAGGTTTATGTGTT
GACATACCTGGCATACCTAAGGACATGACCTATAGAAGACTCATCTCTATGATGGGTTTT
AAAATGAATTATCAAGTTAATGGTTACCCTAACATGTTTATCACCCGCGAAGAAGCTATA
AGACATGTACGTGCATGGATTGGCTTCGATGTCGAGGGGTGTCATGCTACTAGAGAAGCT
GTTGGTACCAATTTACCTTTACAGCTAGGTTTTTCTACAGGTGTTAACCTAGTTGCTGTAC
CTACAGGTTATGTTGATACACCTAATAATACAGATTTTTCCAGAGTTAGTGCTAAACCAC
CGCCTGGAGATCAATTTAAACACCTCATACCACTTATGTACAAAGGACTTCCTTGGAATG
TAGTGCGTATAAAGATTGTACAAATGTTAAGTGACACACTTAAAAATCTCTCTGACAGAG
TCGTATTTGTCTTATGGGCACATGGCTTTGAGTTGACATCTATGAAGTATTTTGTGAAAAT
AGGACCTGAGCGCACCTGTTGTCTATGTGATAGACGTGCCACATGCTTTTCCACTGCTTC
AGACACTTATGCCTGTTGGCATCATTCTATTGGATTTGATTACGTCTATAATCCGTTTATG
ATTGATGTTCAACAATGGGGTTTTACAGGTAACCTACAAAGCAACCATGATCTGTATTGT
CAAGTCCATGGTAATGCACATGTAGCTAGTTGTGATGCAATCATGACTAGGTGTCTAGCT
GTCCACGAGTGCTTTGTTAAGCGTGTTGACTGGACTATTGAATATCCTATAATTGGTGAT
GAACTGAAGATTAATGCGGCTTGTAGAAAGGTTCAACACATGGTTGTTAAAGCTGCATTA
TTAGCAGACAAATTCCCAGTTCTTCACGACATTGGTAACCCTAAAGCTATTAAGTGTGTA
CCTCAAGCTGATGTAGAATGGAAGTTCTATGATGCACAGCCTTGTAGTGACAAAGCTTAT
AAAATAGAAGAATTATTCTATTCTTATGCCACACATTCTGACAAATTCACAGATGGTGTA
TGCCTATTTTGGAATTGCAATGTCGATAGATATCCTGCTAATTCCATTGTTTGTAGATTTG
ACACTAGAGTGCTATCTAACCTTAACTTGCCTGGTTGTGATGGTGGCAGTTTGTATGTAA
ATAAACATGCATTCCACACACCAGCTTTTGATAAAAGTGCTTTTGTTAATTTAAAACAAT
TACCATTTTTCTATTACTCTGACAGTCCATGTGAGTCTCATGGAAAACAAGTAGTGTCAG
ATATAGATTATGTACCACTAAAGTCTGCTACGTGTATAACACGTTGCAATTTAGGTGGTG
CTGTCTGTAGACATCATGCTAATGAGTACAGATTGTATCTCGATGCTTATAACATGATGA
TCTCAGCTGGCTTTAGCTTGTGGGTTTACAAACAATTTGATACTTATAACCTCTGGAACAC
TTTTACAAGACTTCAGAGTTTAGAAAATGTGGCTTTTAATGTTGTAAATAAGGGACACTT
TGATGGACAACAGGGTGAAGTACCAGTTTCTATCATTAATAACACTGTTTACACAAAAGT
TGATGGTGTTGATGTAGAATTGTTTGAAAATAAAACAACATTACCTGTTAATGTAGCATT
TGAGCTTTGGGCTAAGCGCAACATTAAACCAGTACCAGAGGTGAAAATACTCAATAATT
TGGGTGTGGACATTGCTGCTAATACTGTGATCTGGGACTACAAAAGAGATGCTCCAGCAC
ATATATCTACTATTGGTGTTTGTTCTATGACTGACATAGCCAAGAAACCAACTGAAACGA
TTTGTGCACCACTCACTGTCTTTTTTGATGGTAGAGTTGATGGTCAAGTAGACTTATTTAG
AAATGCCCGTAATGGTGTTCTTATTACAGAAGGTAGTGTTAAAGGTTTACAACCATCTGT
AGGTCCCAAACAAGCTAGTCTTAATGGAGTCACATTAATTGGAGAAGCCGTAAAAACAC
AGTTCAATTATTATAAGAAAGTTGATGGTGTTGTCCAACAATTACCTGAAACTTACTTTA
CTCAGAGTAGAAATTTACAAGAATTTAAACCCAGGAGTCAAATGGAAATTGATTTCTTAG
AATTAGCTATGGATGAATTCATTGAACGGTATAAATTAGAAGGCTATGCCTTCGAACATA
TCGTTTATGGAGATTTTAGTCATAGTCAGTTAGGTGGTTTACATCTACTGATTGGACTAGC
TAAACGTTTTAAGGAATCACCTTTTGAATTAGAAGATTTTATTCCTATGGACAGTACAGT
TAAAAACTATTTCATAACAGATGCGCAAACAGGTTCATCTAAGTGTGTGTGTTCTGTTAT
TGATTTATTACTTGATGATTTTGTTGAAATAATAAAATCCCAAGATTTATCTGTAGTTTCT
AAGGTTGTCAAAGTGACTATTGACTATACAGAAATTTCATTTATGCTTTGGTGTAAAGAT
GGCCATGTAGAAACATTTTACCCAAAATTACAATCTAGTCAAGCGTGGCAACCGGGTGTT
GCTATGCCTAATCTTTACAAAATGCAAAGAATGCTATTAGAAAAGTGTGACCTTCAAAAT
TATGGTGATAGTGCAACATTACCTAAAGGCATAATGATGAATGTCGCAAAATATACTCA
ACTGTGTCAATATTTAAACACATTAACATTAGCTGTACCCTATAATATGAGAGTTATACA
TTTTGGTGCTGGTTCTGATAAAGGAGTTGCACCAGGTACAGCTGTTTTAAGACAGTGGTT
GCCTACGGGTACGCTGCTTGTCGATTCAGATCTTAATGACTTTGTCTCTGATGCAGATTCA
ACTTTGATTGGTGATTGTGCAACTGTACATACAGCTAATAAATGGGATCTCATTATTAGT
GATATGTACGACCCTAAGACTAAAAATGTTACAAAAGAAAATGACTCTAAAGAGGGTTT
TTTCACTTACATTTGTGGGTTTATACAACAAAAGCTAGCTCTTGGAGGTTCCGTGGCTATA
AAGATAACAGAACATTCTTGGAATGCTGATCTTTATAAGCTCATGGGACACTTCGCATGG
TGGACAGCCTTTGTTACTAATGTGAATGCGTCATCATCTGAAGCATTTTTAATTGGATGTA
ATTATCTTGGCAAACCACGCGAACAAATAGATGGTTATGTCATGCATGCAAATTACATAT
TTTGGAGGAATACAAATCCAATTCAGTTGTCTTCCTATTCTTTATTTGACATGAGTAAATT
TCCCCTTAAATTAAGGGGTACTGCTGTTATGTCTTTAAAAGAAGGTCAAATCAATGATAT
GATTTTATCTCTTCTTAGTAAAGGTAGACTTATAATTAGAGAAAACAACAGAGTTGTTAT
TTCTAGTGATGTTCTTGTTAACAACTAAACGAACAATGTTTGTTTTTCTTGTTTTATTGCC
ACTAGTCTCTAGTCAGTGTGTTAATCTTACAACCAGAACTCAATTACCCCCTGCATACAC
TAATTCTTTCACACGTGGTGTTTATTACCCTGACAAAGTTTTCAGATCCTCAGTTTTACAT
TCAACTCAGGACTTGTTCTTACCTTTCTTTTCCAATGTTACTTGGTTCCATGCTATACATGT
CTCTGGGACCAATGGTACTAAGAGGTTTGATAACCCTGTCCTACCATTTAATGATGGTGT
TTATTTTGCTTCCACTGAGAAGTCTAACATAATAAGAGGCTGGATTTTTGGTACTACTTTA
GATTCGAAGACCCAGTCCCTACTTATTGTTAATAACGCTACTAATGTTGTTATTAAAGTCT
GTGAATTTCAATTTTGTAATGATCCATTTTTGGGTGTTTATTACCACAAAAACAACAAAA
GTTGGATGGAAAGTGAGTTCAGAGTTTATTCTAGTGCGAATAATTGCACTTTTGAATATG
TCTCTCAGCCTTTTCTTATGGACCTTGAAGGAAAACAGGGTAATTTCAAAAATCTTAGGG
AATTTGTGTTTAAGAATATTGATGGTTATTTTAAAATATATTCTAAGCACACGCCTATTAA
TTTAGTGCGTGATCTCCCTCAGGGTTTTTCGGCTTTAGAACCATTGGTAGATTTGCCAATA
GGTATTAACATCACTAGGTTTCAAACTTTACTTGCTTTACATAGAAGTTATTTGACTCCTG
GTGATTCTTCTTCAGGTTGGACAGCTGGTGCTGCAGCTTATTATGTGGGTTATCTTCAACC
TAGGACTTTTCTATTAAAATATAATGAAAATGGAACCATTACAGATGCTGTAGACTGTGC
ACTTGACCCTCTCTCAGAAACAAAGTGTACGTTGAAATCCTTCACTGTAGAAAAAGGAAT
CTATCAAACTTCTAACTTTAGAGTCCAACCAACAGAATCTATTGTTAGATTTCCTAATATT
ACAAACTTGTGCCCTTTTGGTGAAGTTTTTAACGCCACCAGATTTGCATCTGTTTATGCTT
GGAACAGGAAGAGAATCAGCAACTGTGTTGCTGATTATTCTGTCCTATATAATTCCGCAT
CATTTTCCACTTTTAAGTGTTATGGAGTGTCTCCTACTAAATTAAATGATCTCTGCTTTAC
TAATGTCTATGCAGATTCATTTGTAATTAGAGGTGATGAAGTCAGACAAATCGCTCCAGG
GCAAACTGGAAAGATTGCTGATTATAATTATAAATTACCAGATGATTTTACAGGCTGCGT
TATAGCTTGGAATTCTAACAATCTTGATTCTAAGGTTGGTGGTAATTATAATTACCTGTAT
AGATTGTTTAGGAAGTCTAATCTCAAACCTTTTGAGAGAGATATTTCAACTGAAATCTAT
CAGGCCGGTAGCACACCTTGTAATGGTGTTGAAGGTTTTAATTGTTACTTTCCTTTACAAT
CATATGGTTTCCAACCCACTAATGGTGTTGGTTACCAACCATACAGAGTAGTAGTACTTT
CTTTTGAACTTCTACATGCACCAGCAACTGTTTGTGGACCTAAAAAGTCTACTAATTTGGT
TAAAAACAAATGTGTCAATTTCAACTTCAATGGTTTAACAGGCACAGGTGTTCTTACTGA
GTCTAACAAAAAGTTTCTGCCTTTCCAACAATTTGGCAGAGACATTGCTGACACTACTGA
TGCTGTCCGTGATCCACAGACACTTGAGATTCTTGACATTACACCATGTTCTTTTGGTGGT
GTCAGTGTTATAACACCAGGAACAAATACTTCTAACCAGGTTGCTGTTCTTTATCAGGAT
GTTAACTGCACAGAAGTCCCTGTTGCTATTCATGCAGATCAACTTACTCCTACTTGGCGT
GTTTATTCTACAGGTTCTAATGTTTTTCAAACACGTGCAGGCTGTTTAATAGGGGCTGAA
CATGTCAACAACTCATATGAGTGTGACATACCCATTGGTGCAGGTATATGCGCTAGTTAT
CAGACTCAGACTAATTCTCCTCGGCGGGCACGTAGTGTAGCTAGTCAATCCATCATTGCC
TACACTATGTCACTTGGTGCAGAAAATTCAGTTGCTTACTCTAATAACTCTATTGCCATAC
CCACAAATTTTACTATTAGTGTTACCACAGAAATTCTACCAGTGTCTATGACCAAGACAT
CAGTAGATTGTACAATGTACATTTGTGGTGATTCAACTGAATGCAGCAATCTTTTGTTGC
AATATGGCAGTTTTTGTACACAATTAAACCGTGCTTTAACTGGAATAGCTGTTGAACAAG
ACAAAAACACCCAAGAAGTTTTTGCACAAGTCAAACAAATTTACAAAACACCACCAATT
AAAGATTTTGGTGGTTTTAATTTTTCACAAATATTACCAGATCCATCAAAACCAAGCAAG
AGGTCATTTATTGAAGATCTACTTTTCAACAAAGTGACACTTGCAGATGCTGGCTTCATC
AAACAATATGGTGATTGCCTTGGTGATATTGCTGCTAGAGAtCTCATTTGTGCACAAAAGT
TTAACGGCCTTACTGTTTTGCCACCTTTGCTCACAGATGAAATGATTGCTCAATACACTTC
TGCACTGTTAGCGGGTACAATCACTTCTGGTTGGACCTTTGGTGCAGGTGCTGCATTACA
AATACCATTTGCTATGCAAATGGCTTATAGGTTTAATGGTATTGGAGTTACACAGAATGT
TCTCTATGAGAACCAAAAATTGATTGCCAACCAATTTAATAGTGCTATTGGCAAAATTCA
AGACTCACTTTCTTCCACAGCAAGTGCACTTGGAAAACTTCAAGATGTGGTCAACCAAAA
TGCACAAGCTTTAAACACGCTTGTTAAACAACTTAGCTCCAATTTTGGTGCAATTTCAAG
TGTTTTAAATGATATCCTTTCACGTCTTGACAAAGTTGAGGCTGAAGTGCAAATTGATAG
GTTGATCACAGGCAGACTTCAAAGTTTGCAGACATATGTGACTCAACAATTAATTAGAGC
TGCAGAAATCAGAGCTTCTGCTAATCTTGCTGCTACTAAAATGTCAGAGTGTGTACTTGG
ACAATCAAAAAGAGTTGATTTTTGTGGAAAGGGCTATCATCTTATGTCCTTCCCTCAGTC
AGCACCTCATGGTGTAGTCTTCTTGCATGTGACTTATGTCCCTGCACAAGAAAAGAACTT
CACAACTGCTCCTGCCATTTGTCATGATGGAAAAGCACACTTTCCTCGTGAAGGTGTCTT
TGTTTCAAATGGCACACACTGGTTTGTAACACAAAGGAATTTTTATGAACCACAAATCAT
TACTACAGACAACACATTTGTGTCTGGTAACTGTGATGTTGTAATAGGAATTGTCAACAA
CACAGTTTATGATCCTTTGCAACCTGAATTAGACTCATTCAAGGAGGAGTTAGATAAATA
TTTTAAGAATCATACATCACCAGATGTTGATTTAGGTGACATCTCTGGCATTAATGCTTCA
GTTGTAAACATTCAAAAAGAAATTGACCGCCTCAATGAGGTTGCCAAGAATTTAAATGA
ATCTCTCATCGATCTCCAAGAACTTGGAAAGTATGAGCAGTATATAAAATGGCCATGGTA
CATTTGGCTAGGTTTTATAGCTGGCTTGATTGCCATAGTAATGGTGACAATTATGCTTTGC
TGTATGACCAGTTGCTGTAGTTGTCTCAAGGGCTGTTGTTCTTGTGGATCCTGCTGCAAAT
TTGATGAAGACGACTCTGAGCCAGTGCTCAAAGGAGTCAAATTACATTACACATAAACG
AACTTATGGATTTGTTTATGAGAATCTTCACAATTGGAACTGTAACTTTGAAGCAAGGTG
AAATCAAGGATGCTACTCCTTCAGATTTTGTTCGCGCTACTGCAACGATACCGATACAAG
CCTCACTCCCTTTCGGATGGCTTATTGTTGGCGTTGCACTTCTTGCTGTTTTTCAGAGCGC
TTCCAAAATCATAACCCTCAAAAAGAGATGGCAACTAGCACTCTCCAAGGGTGTTCACTT
TGTTTGCAACTTGCTGTTGTTGTTTGTAACAGTTTACTCACACCTTTTGCTCGTTGCTGCTG
GCCTTGAAGCCCCTTTTCTCTATCTTTATGCTTTAGTCTACTTCTTGCAGAGTATAAACTTT
GTAAGAATAATAATGAGGCTTTGGCTTTGCTGGAAATGCCGTTCCAAAAACCCATTACTT
TATGATGCCAACTATTTTCTTTGCTGGCATACTAATTGTTACGACTATTGTATACCTTACA
ATAGTGTAACTTCTTCAATTGTCATTACTTCAGGTGATGGCACAACAAGTCCTATTTCTGA
ACATGACTACCAGATTGGTGGTTATACTGAAAAATGGGAATCTGGAGTAAAAGACTGTG
TTGTATTACACAGTTACTTCACTTCAGACTATTACCAGCTGTACTCAACTCAATTGAGTAC
AGACACTGGTGTTGAACATGTTACCTTCTTCATCTACAATAAAATTGTTGATGAGCCTGA
AGAACATGTCCAAATTCACACAATCGACGGTTCATCCGGAGTTGTTAATCCAGTAATGGA
ACCAATTTATGATGAACCGACGACGACTACTAGCGTGCCTTTGTAAGCACAAGCTGATGA
GTACGAACTTATGTACTCATTCGTTTCGGAAGAGACAGGTACGTTAATAGTTAATAGCGT
ACTTCTTTTTCTTGCTTTCGTGGTATTCTTGCTAGTTACACTAGCCATCCTTACTGCGCTTC
GATTGTGTGCGTACTGCTGCAATATTGTTAACGTGAGTCTTGTAAAACCTTCTTTTTACGT
TTACTCTCGTGTTAAAAATCTGAATTCTTCTAGAGTTCCTGATCTTCTGGTCTAAACGAAC
TAAATATTATATTAGTTTTTCTGTTTGGAACTTTAATTTTAGCCATGGCAGATTCCAACGG
TACTATTACCGTTGAAGAGCTTAAAAAGCTCCTTGAACAATGGAACCTAGTAATAGGTTT
CCTATTCCTTACATGGATTTGTCTTCTACAATTTGCCTATGCCAACAGGAATAGGTTTTTG
TATATAATTAAGTTAATTTTCCTCTGGCTGTTATGGCCAGTAACTTTAGCTTGTTTTGTGC
TTGCTGCTGTTTACAGAATAAATTGGATCACCGGTGGAATTGCTATCGCAATGGCTTGTC
TTGTAGGCTTGATGTGGCTCAGCTACTTCATTGCTTCTTTCAGACTGTTTGCGCGTACGCG
TTCCATGTGGTCATTCAATCCAGAAACTAACATTCTTCTCAACGTGCCACTCCATGGCACT
ATTCTGACCAGACCGCTTCTAGAAAGTGAACTCGTAATCGGAGCTGTGATCCTTCGTGGA
CATCTTCGTATTGCTGGACACCATCTAGGACGCTGTGACATCAAGGACCTGCCTAAAGAA
ATCACTGTTGCTACATCACGAACGCTTTCTTATTACAAATTGGGAGCTTCGCAGCGTGTA
GCAGGTGACTCAGGTTTTGCTGCATACAGTCGCTACAGGATTGGCAACTATAAATTAAAC
ACAGACCATTCCAGTAGCAGTGACAATATTGCTTTGCTTGTACAGTAAGTGACAACAGAT
GTTTCATCTCGTTGACTTTCAGGTTACTATAGCAGAGATATTACTAATTATTATGAGGACT
TTTAAAGTTTCCATTTGGAATCTTGATTACATCATAAACCTCATAATTAAAAATTTATCTA
AGTCACTAACTGAGAATAAATATTCTCAATTAGATGAAGAGCAACCAATGGAGATTGAT
TAAACGAACATGAAAATTATTCTTTTCTTGGCACTGATAACACTCGCTACTTGTGAGCTTT
ATCACTACCAAGAGTGTGTTAGAGGTACAACAGTACTTTTAAAAGAACCTTGCTCTTCTG
GAACATACGAGGGCAATTCACCATTTCATCCTCTAGCTGATAACAAATTTGCACTGACTT
GCTTTAGCACTCAATTTGCTTTTGCTTGTCCTGACGGCGTAAAACACGTCTATCAGTTACG
TGCCAGATCAGTTTCACCTAAACTGTTCATCAGACAAGAGGAAGTTCAAGAACTTTACTC
TCCAATTTTTCTTATTGTTGCGGCAATAGTGTTTATAACACTTTGCTTCACACTCAAAAGA
AAGACAGAATGATTGAACTTTCATTAATTGACTTCTATTTGTGCTTTTTAGCCTTTCTGCT
ATTCCTTGTTTTAATTATGCTTATTATCTTTTGGTTCTCACTTGAACTGCAAGATCATAATG
AAACTTGTCACGCCTAAACGAACATGAAATTTCTTGTTTTCTTAGGAATCATCACAACTG
TAGCTGCATTTCACCAAGAATGTAGTTTACAGTCATGTACTCAACATCAACCATATGTAG
TTGATGACCCGTGTCCTATTCACTTCTATTCTAAATGGTATATTAGAGTAGGAGCTAGAA
AATCAGCACCTTTAATTGAATTGTGCGTGGATGAGGCTGGTTCTAAATCACCCATTCAGT
ACATCGATATCGGTAATTATACAGTTTCCTGTTTACCTTTTACAATTAATTGCCAGGAACC
TAAATTGGGTAGTCTTGTAGTGCGTTGTTCGTTCTATGAAGACTTTTTAGAGTATCATGAC
GTTCGTGTTGTTTTAGATTTCATCTAAACGAACAAACTAAAATGTCTGATAATGGACCCC
AAAATCAGCGAAATGCACCCCGCATTACGTTTGGTGGACCCTCAGATTCAACTGGCAGTA
ACCAGAATGGAGAACGCAGTGGGGCGCGATCAAAACAACGTCGGCCCCAAGGTTTACCC
AATAATACTGCGTCTTGGTTCACCGCTCTCACTCAACATGGCAAGGAAGACCTTAAATTC
CCTCGAGGACAAGGCGTTCCAATTAACACCAATAGCAGTCCAGATGACCAAATTGGCTA
CTACCGAAGAGCTACCAGACGAATTCGTGGTGGTGACGGTAAAATGAAAGATCTCAGTC
CAAGATGGTATTTCTACTACCTAGGAACTGGGCCAGAAGCTGGACTTCCCTATGGTGCTA
ACAAAGACGGCATCATATGGGTTGCAACTGAGGGAGCCTTGAATACACCAAAAGATCAC
ATTGGCACCCGCAATCCTGCTAACAATGCTGCAATCGTGCTACAACTTCCTCAAGGAACA
ACATTGCCAAAAGGCTTCTACGCAGAAGGGAGCAGAGGCGGCAGTCAAGCCTCTTCTCG
TTCCTCATCACGTAGTCGCAACAGTTCAAGAAATTCAACTCCAGGCAGCAGTAGGGGAA
CTTCTCCTGCTAGAATGGCTGGCAATGGCGGTGATGCTGCTCTTGCTTTGCTGCTGCTTGA
CAGATTGAACCAGCTTGAGAGCAAAATGTCTGGTAAAGGCCAACAACAACAAGGCCAAA
CTGTCACTAAGAAATCTGCTGCTGAGGCTTCTAAGAAGCCTCGGCAAAAACGTACTGCCA
CTAAAGCATACAATGTAACACAAGCTTTCGGCAGACGTGGTCCAGAACAAACCCAAGGA
AATTTTGGGGACCAGGAACTAATCAGACAAGGAACTGATTACAAACATTGGCCGCAAAT
TGCACAATTTGCCCCCAGCGCTTCAGCGTTCTTCGGAATGTCGCGCATTGGCATGGAAGT
CACACCTTCGGGAACGTGGTTGACCTACACAGGTGCCATCAAATTGGATGACAAAGATC
CAAATTTCAAAGATCAAGTCATTTTGCTGAATAAGCATATTGACGCATACAAAACATTCC
CACCAACAGAGCCTAAAAAGGACAAAAAGAAGAAGGCTGATGAAACTCAAGCCTTACC
GCAGAGACAGAAGAAACAGCAAACTGTGACTCTTCTTCCTGCTGCAGATTTGGATGATTT
CTCCAAACAATTGCAACAATCCATGAGCAGTGCTGACTCAACTCAGGCCTAAACTCATGC
AGACCACACAAGGCAGATGGGCTATATAAACGTTTTCGCTTTTCCGTTTACGATATATAG
TCTACTCTTGTGCAGAATGAATTCTCGTAACTACATAGCACAAGTAGATGTAGTTAACTT
TAATCTCACATAGCAATCTTTAATCAGTGTGTAACATTAGGGAGGACTTGAAAGAGCCAC
CACATTTTCACCGAGGCCACGCGGAGTACGATCGAGTGTACAGTGAACAATGCTAGGGA
GAGCTGCCTATATGGAAGAGCCCTAATGTGTAAAATTAATTTTAGTAGTGCTATCCCCAT
GTGATTTTAATAGCTTCTTAGGAGAATGACAAAAAAAAAAAAAAAAAAAAA
SEQ ID NO: 3-Deoptimized SARS-CoV-2 coronavirus (Wuhan-CoV_101K)
(deoptimized in reference to BetaCoV/Wuhan/IVDC-HB-01/2019). regions
deoptimized: 11294-12709, 14641-15903 (nsp12 (e.g., RNA-dependent RNA
polymerase “RdRP”) domain), 21656-22306 (Spike beginning), 22505-23905
(Spike middle), 24110-25381 (Spike end).
ATTAAAGGTTTATACCTTCCCAGGTAACAAACCAACCAACTTTCGATCTCTTG
TAGATCTGTTCTCTAAACGAACTTTAAAATCTGTGTGGCTGTCACTCGGCTGCATGCTTAG
TGCACTCACGCAGTATAATTAATAACTAATTACTGTCGTTGACAGGACACGAGTAACTCG
TCTATCTTCTGCAGGCTGCTTACGGTTTCGTCCGTGTTGCAGCCGATCATCAGCACATCTA
GGTTTCGTCCGGGTGTGACCGAAAGGTAAGATGGAGAGCCTTGTCCCTGGTTTCAACGAG
AAAACACACGTCCAACTCAGTTTGCCTGTTTTACAGGTTCGCGACGTGCTCGTACGTGGC
TTTGGAGACTCCGTGGAGGAGGTCTTATCAGAGGCACGTCAACATCTTAAAGATGGCACT
TGTGGCTTAGTAGAAGTTGAAAAAGGCGTTTTGCCTCAACTTGAACAGCCCTATGTGTTC
ATCAAACGTTCGGATGCTCGAACTGCACCTCATGGTCATGTTATGGTTGAGCTGGTAGCA
GAACTCGAAGGCATTCAGTACGGTCGTAGTGGTGAGACACTTGGTGTCCTTGTCCCTCAT
GTGGGCGAAATACCAGTGGCTTACCGCAAGGTTCTTCTTCGTAAGAACGGTAATAAAGG
AGCTGGTGGCCATAGTTACGGCGCCGATCTAAAGTCATTTGACTTAGGCGACGAGCTTGG
CACTGATCCTTATGAAGATTTTCAAGAAAACTGGAACACTAAACATAGCAGTGGTGTTAC
CCGTGAACTCATGCGTGAGCTTAACGGAGGGGCATACACTCGCTATGTCGATAACAACTT
CTGTGGCCCTGATGGCTACCCTCTTGAGTGCATTAAAGACCTTCTAGCACGTGCTGGTAA
AGCTTCATGCACTTTGTCCGAACAACTGGACTTTATTGACACTAAGAGGGGTGTATACTG
CTGCCGTGAACATGAGCATGAAATTGCTTGGTACACGGAACGTTCTGAAAAGAGCTATG
AATTGCAGACACCTTTTGAAATTAAATTGGCAAAGAAATTTGACACCTTCAATGGGGAAT
GTCCAAATTTTGTATTTCCCTTAAATTCCATAATCAAGACTATTCAACCAAGGGTTGAAA
AGAAAAAGCTTGATGGCTTTATGGGTAGAATTCGATCTGTCTATCCAGTTGCGTCACCAA
ATGAATGCAACCAAATGTGCCTTTCAACTCTCATGAAGTGTGATCATTGTGGTGAAACTT
CATGGCAGACGGGCGATTTTGTTAAAGCCACTTGCGAATTTTGTGGCACTGAGAATTTGA
CTAAAGAAGGTGCCACTACTTGTGGTTACTTACCCCAAAATGCTGTTGTTAAAATTTATT
GTCCAGCATGTCACAATTCAGAAGTAGGACCTGAGCATAGTCTTGCCGAATACCATAATG
AATCTGGCTTGAAAACCATTCTTCGTAAGGGTGGTCGCACTATTGCCTTTGGAGGCTGTG
TGTTCTCTTATGTTGGTTGCCATAACAAGTGTGCCTATTGGGTTCCACGTGCTAGCGCTAA
CATAGGTTGTAACCATACAGGTGTTGTTGGAGAAGGTTCCGAAGGTCTTAATGACAACCT
TCTTGAAATACTCCAAAAAGAGAAAGTCAACATCAATATTGTTGGTGACTTTAAACTTAA
TGAAGAGATCGCCATTATTTTGGCATCTTTTTCTGCTTCCACAAGTGCTTTTGTGGAAACT
GTGAAAGGTTTGGATTATAAAGCATTCAAACAAATTGTTGAATCCTGTGGTAATTTTAAA
GTTACAAAAGGAAAAGCTAAAAAAGGTGCCTGGAATATTGGTGAACAGAAATCAATACT
GAGTCCTCTTTATGCATTTGCATCAGAGGCTGCTCGTGTTGTACGATCAATTTTCTCCCGC
ACTCTTGAAACTGCTCAAAATTCTGTGCGTGTTTTACAGAAGGCCGCTATAACAATACTA
GATGGAATTTCACAGTATTCACTGAGACTCATTGATGCTATGATGTTCACATCTGATTTG
GCTACTAACAATCTAGTTGTAATGGCCTACATTACAGGTGGTGTTGTTCAGTTGACTTCG
CAGTGGCTAACTAACATCTTTGGCACTGTTTATGAAAAACTCAAACCCGTCCTTGATTGG
CTTGAAGAGAAGTTTAAGGAAGGTGTAGAGTTTCTTAGAGATGGTTGGGAAATTGTTAA
ATTTATCTCAACCTGTGCTTGTGAAATTGTCGGTGGACAAATTGTCACCTGTGCAAAGGA
AATTAAGGAGAGTGTTCAGACATTCTTTAAGCTTGTAAATAAATTTTTGGCTTTGTGTGCT
GACTCTATCATTATTGGTGGAGCTAAACTTAAAGCCTTGAATTTAGGTGAAACATTTGTC
ACGCACTCAAAGGGATTGTACAGAAAGTGTGTTAAATCCAGAGAAGAAACTGGCCTACT
CATGCCTCTAAAAGCCCCAAAAGAAATTATCTTCTTAGAGGGAGAAACACTTCCCACAG
AAGTGTTAACAGAGGAAGTTGTCTTGAAAACTGGTGATTTACAACCATTAGAACAACCT
ACTAGTGAAGCTGTTGAAGCTCCATTGGTTGGTACACCAGTTTGTATTAACGGGCTTATG
TTGCTCGAAATCAAAGACACAGAAAAGTACTGTGCCCTTGCACCTAATATGATGGTAAC
AAACAATACCTTCACACTCAAAGGCGGTGCACCAACAAAGGTTACTTTTGGTGATGACA
CTGTGATAGAAGTGCAAGGTTACAAGAGTGTGAATATCACTTTTGAACTTGATGAAAGG
ATTGATAAAGTACTTAATGAGAAGTGCTCTGCCTATACAGTTGAACTCGGTACAGAAGTA
AATGAGTTCGCCTGTGTTGTGGCAGATGCTGTCATAAAAACTTTGCAACCAGTATCTGAA
TTACTTACACCACTGGGCATTGATTTAGATGAGTGGAGTATGGCTACATACTACTTATTT
GATGAGTCTGGTGAGTTTAAATTGGCTTCACATATGTATTGTTCTTTCTACCCTCCAGATG
AGGATGAAGAAGAAGGTGATTGTGAAGAAGAAGAGTTTGAGCCATCAACTCAATATGAG
TATGGTACTGAAGATGATTACCAAGGTAAACCTTTGGAATTTGGTGCCACTTCTGCTGCT
CTTCAACCTGAAGAAGAGCAAGAAGAAGATTGGTTAGATGATGATAGTCAACAAACTGT
TGGTCAACAAGACGGCAGTGAGGACAATCAGACAACTACTATTCAAACAATTGTTGAGG
TTCAACCTCAATTAGAGATGGAACTTACACCAGTTGTTCAGACTATTGAAGTGAATAGTT
TTAGTGGTTATTTAAAACTTACTGACAATGTATACATTAAAAATGCAGACATTGTGGAAG
AAGCTAAAAAGGTAAAACCAACAGTGGTTGTTAATGCAGCCAATGTTTACCTTAAACAT
GGAGGAGGTGTTGCAGGAGCCTTAAATAAGGCTACTAACAATGCCATGCAAGTTGAATC
TGATGATTACATAGCTACTAATGGACCACTTAAAGTGGGTGGTAGTTGTGTTTTAAGCGG
ACACAATCTTGCTAAACACTGTCTTCATGTTGTCGGCCCAAATGTTAACAAAGGTGAAGA
CATTCAACTTCTTAAGAGTGCTTATGAAAATTTTAATCAGCACGAAGTTCTACTTGCACC
ATTATTATCAGCTGGTATTTTTGGTGCTGACCCTATACATTCTTTAAGAGTTTGTGTAGAT
ACTGTTCGCACAAATGTCTACTTAGCTGTCTTTGATAAAAATCTCTATGACAAACTTGTTT
CAAGCTTTTTGGAAATGAAGAGTGAAAAGCAAGTTGAACAAAAGATCGCTGAGATTCCT
AAAGAGGAAGTTAAGCCATTTATAACTGAAAGTAAACCTTCAGTTGAACAGAGAAAACA
AGATGATAAGAAAATCAAAGCTTGTGTTGAAGAAGTTACAACAACTCTGGAAGAAACTA
AGTTCCTCACAGAAAACTTGTTACTTTATATTGACATTAATGGCAATCTTCATCCAGATTC
TGCCACTCTTGTTAGTGACATTGACATCACTTTCTTAAAGAAAGATGCTCCATATATAGT
GGGTGATGTTGTTCAAGAGGGTGTTTTAACTGCTGTGGTTATACCTACTAAAAAGGCTGG
TGGCACTACTGAAATGCTAGCGAAAGCTTTGAGAAAAGTGCCAACAGACAATTATATAA
CCACTTACCCGGGTCAGGGTTTAAATGGTTACACTGTAGAGGAGGCAAAGACAGTGCTT
AAAAAGTGTAAAAGTGCCTTTTACATTCTACCATCTATTATCTCTAATGAGAAGCAAGAA
ATTCTTGGAACTGTTTCTTGGAATTTGCGAGAAATGCTTGCACATGCAGAAGAAACACGC
AAATTAATGCCTGTCTGTGTGGAAACTAAAGCCATAGTTTCAACTATACAGCGTAAATAT
AAGGGTATTAAAATACAAGAGGGTGTGGTTGATTATGGTGCTAGATTTTACTTTTACACC
AGTAAAACAACTGTAGCGTCACTTATCAACACACTTAACGATCTAAATGAAACTCTTGTT
ACAATGCCACTTGGCTATGTAACACATGGCTTAAATTTGGAAGAAGCTGCTCGGTATATG
AGATCTCTCAAAGTGCCAGCTACAGTTTCTGTTTCTTCACCTGATGCTGTTACAGCGTATA
ATGGTTATCTTACTTCTTCTTCTAAAACACCTGAAGAACATTTTATTGAAACCATCTCACT
TGCTGGTTCCTATAAAGATTGGTCCTATTCTGGACAATCTACACAACTAGGTATAGAATT
TCTTAAGAGAGGTGATAAAAGTGTATATTACACTAGTAATCCTACCACATTCCACCTAGA
TGGTGAAGTTATCACCTTTGACAATCTTAAGACACTTCTTTCTTTGAGAGAAGTGAGGAC
TATTAAGGTGTTTACAACAGTAGACAACATTAACCTCCACACGCAAGTTGTGGACATGTC
AATGACATATGGACAACAGTTTGGTCCAACTTATTTGGATGGAGCTGATGTTACTAAAAT
AAAACCTCATAATTCACATGAAGGTAAAACATTTTATGTTTTACCTAATGATGACACTCT
ACGTGTTGAGGCTTTTGAGTACTACCACACAACTGATCCTAGTTTTCTGGGTAGGTACAT
GTCAGCATTAAATCACACTAAAAAGTGGAAATACCCACAAGTTAATGGTTTAACTTCTAT
TAAATGGGCAGATAACAACTGTTATCTTGCCACTGCATTGTTAACACTCCAACAAATAGA
GTTGAAGTTTAATCCACCTGCTCTACAAGATGCTTATTACAGAGCAAGGGCTGGTGAAGC
TGCTAACTTTTGTGCACTTATCTTAGCCTACTGTAATAAGACAGTAGGTGAGTTAGGTGA
TGTTAGAGAAACAATGAGTTACTTGTTTCAACATGCCAATTTAGATTCTTGCAAAAGAGT
CTTGAACGTGGTGTGTAAAACTTGTGGACAACAGCAGACAACCCTTAAGGGTGTAGAAG
CTGTTATGTACATGGGCACACTTTCTTATGAACAATTTAAGAAAGGTGTTCAGATACCTT
GTACGTGTGGTAAACAAGCTACAAAATATCTAGTACAACAGGAGTCACCTTTTGTTATGA
TGTCAGCACCACCTGCTCAGTATGAACTTAAGCATGGTACATTTACTTGTGCTAGTGAGT
ACACTGGTAATTACCAGTGTGGTCACTATAAACATATAACTTCTAAAGAAACTTTGTATT
GCATAGACGGTGCTTTACTTACAAAGTCCTCAGAATACAAAGGTCCTATTACGGATGTTT
TCTACAAAGAAAACAGTTACACAACAACCATAAAACCAGTTACTTATAAATTGGATGGT
GTTGTTTGTACAGAAATTGACCCTAAGTTGGACAATTATTATAAGAAAGACAATTCTTAT
TTCACAGAGCAACCAATTGATCTTGTACCAAACCAACCATATCCAAACGCAAGCTTCGAT
AATTTTAAGTTTGTATGTGATAATATCAAATTTGCTGATGATTTAAACCAGTTAACTGGTT
ATAAGAAACCTGCTTCAAGAGAGCTTAAAGTTACATTTTTCCCTGACTTAAATGGTGATG
TGGTGGCTATTGATTATAAACACTACACACCCTCTTTTAAGAAAGGAGCTAAATTGTTAC
ATAAACCTATTGTTTGGCATGTTAACAATGCAACTAATAAAGCCACGTATAAACCAAATA
CCTGGTGTATACGTTGTCTTTGGAGCACAAAACCAGTTGAAACATCAAATTCGTTTGATG
TACTGAAGTCAGAGGACGCGCAGGGAATGGATAATCTTGCCTGCGAAGATCTAAAACCA
GTCTCTGAAGAAGTAGTGGAAAATCCTACCATACAGAAAGACGTTCTTGAGTGTAATGT
GAAAACTACCGAAGTTGTAGGAGACATTATACTTAAACCAGCAAATAATAGTTTAAAAA
TTACAGAAGAGGTTGGCCACACAGATCTAATGGCTGCTTATGTAGACAATTCTAGTCTTA
CTATTAAGAAACCTAATGAATTATCTAGAGTATTAGGTTTGAAAACCCTTGCTACTCATG
GTTTAGCTGCTGTTAATAGTGTCCCTTGGGATACTATAGCTAATTATGCTAAGCCTTTTCT
TAACAAAGTTGTTAGTACAACTACTAACATAGTTACACGGTGTTTAAACCGTGTTTGTAC
TAATTATATGCCTTATTTCTTTACTTTATTGCTACAATTGTGTACTTTTACTAGAAGTACA
AATTCTAGAATTAAAGCATCTATGCCGACTACTATAGCAAAGAATACTGTTAAGAGTGTC
GGTAAATTTTGTCTAGAGGCTTCATTTAATTATTTGAAGTCACCTAATTTTTCTAAACTGA
TAAATATTATAATTTGGTTTTTACTATTAAGTGTTTGCCTAGGTTCTTTAATCTACTCAAC
CGCTGCTTTAGGTGTTTTAATGTCTAATTTAGGCATGCCTTCTTACTGTACTGGTTACAGA
GAAGGCTATTTGAACTCTACTAATGTCACTATTGCAACCTACTGTACTGGTTCTATACCTT
GTAGTGTTTGTCTTAGTGGTTTAGATTCTTTAGACACCTATCCTTCTTTAGAAACTATACA
AATTACCATTTCATCTTTTAAATGGGATTTAACTGCTTTTGGCTTAGTTGCAGAGTGGTTT
TTGGCATATATTCTTTTCACTAGGTTTTTCTATGTACTTGGATTGGCTGCAATCATGCAAT
TGTTTTTCAGCTATTTTGCAGTACATTTTATTAGTAATTCTTGGCTTATGTGGTTAATAATT
AATCTTGTACAAATGGCCCCGATTTCAGCTATGGTTAGAATGTACATCTTCTTTGCATCAT
TTTATTATGTATGGAAAAGTTATGTGCATGTTGTAGACGGTTGTAATTCATCAACTTGTAT
GATGTGTTACAAACGTAATAGAGCAACAAGAGTCGAATGTACAACTATTGTTAATGGTG
TTAGAAGGTCCTTTTATGTCTATGCTAATGGAGGTAAAGGCTTTTGCAAACTACACAATT
GGAATTGTGTTAATTGTGATACATTCTGTGCTGGTAGTACATTTATTAGTGATGAAGTTGC
GAGAGACTTGTCACTACAGTTTAAAAGACCAATAAATCCTACTGACCAGTCTTCTTACAT
CGTTGATAGTGTTACAGTGAAGAATGGTTCCATCCATCTTTACTTTGATAAAGCTGGTCA
AAAGACTTATGAAAGACATTCTCTCTCTCATTTTGTTAACTTAGACAACCTGAGAGCTAA
TAACACTAAAGGTTCATTGCCTATTAATGTTATAGTTTTTGATGGTAAATCAAAATGTGA
AGAATCATCTGCAAAATCAGCGTCTGTTTACTACAGTCAGCTTATGTGTCAACCTATACT
GTTACTAGATCAGGCATTAGTGTCTGATGTTGGTGATAGTGCGGAAGTTGCAGTTAAAAT
GTTTGATGCTTACGTTAATACGTTTTCATCAACTTTTAACGTACCAATGGAAAAACTCAA
AACACTAGTTGCAACTGCAGAAGCTGAACTTGCAAAGAATGTGTCCTTAGACAATGTCTT
ATCTACTTTTATTTCAGCAGCTCGGCAAGGGTTTGTTGATTCAGATGTAGAAACTAAAGA
TGTTGTTGAATGTCTTAAATTGTCACATCAATCTGACATAGAAGTTACTGGCGATAGTTG
TAATAACTATATGCTCACCTATAACAAAGTTGAAAACATGACACCCCGTGACCTTGGTGC
TTGTATTGACTGTAGTGCGCGTCATATTAATGCGCAGGTAGCAAAAAGTCACAACATTGC
TTTGATATGGAACGTTAAAGATTTCATGTCATTGTCTGAACAACTACGAAAACAAATACG
TAGTGCTGCTAAAAAGAATAACTTACCTTTTAAGTTGACATGTGCAACTACTAGACAAGT
TGTTAATGTTGTAACAACAAAGATAGCACTTAAGGGTGGTAAAATTGTTAATAATTGGTT
GAAGCAGTTAATTAAAGTTACACTTGTGTTCCTTTTTGTTGCTGCTATTTTCTATTTAATA
ACACCTGTTCATGTCATGTCTAAACATACTGACTTTTCAAGTGAAATCATAGGATACAAG
GCTATTGATGGTGGTGTCACTCGTGACATAGCATCTACAGATACTTGTTTTGCTAACAAA
CATGCTGATTTTGACACATGGTTTAGCCAGCGTGGTGGTAGTTATACTAATGACAAAGCT
TGCCCATTGATTGCTGCAGTCATAACAAGAGAAGTGGGTTTTGTCGTGCCTGGTTTGCCT
GGCACGATATTACGCACAACTAATGGTGACTTTTTGCATTTCTTACCTAGAGTTTTTAGTG
CAGTTGGTAACATCTGTTACACACCATCAAAACTTATAGAGTACACTGACTTTGCAACAT
CAGCTTGTGTTTTGGCTGCTGAATGTACAATTTTTAAAGATGCTTCTGGTAAGCCAGTACC
ATATTGTTATGATACCAATGTACTAGAAGGTTCTGTTGCTTATGAAAGTTTACGCCCTGA
CACACGTTATGTGCTCATGGATGGCTCTATTATTCAATTTCCTAACACCTACCTTGAAGGT
TCTGTTAGAGTGGTAACAACTTTTGATTCTGAGTACTGTAGGCACGGCACTTGTGAAAGA
TCAGAAGCTGGTGTTTGTGTATCTACTAGTGGTAGATGGGTACTTAACAATGATTATTAC
AGATCTTTACCAGGAGTTTTCTGTGGTGTAGATGCTGTAAATTTACTTACTAATATGTTTA
CACCACTAATTCAACCTATTGGTGCTTTGGACATATCAGCATCTATAGTAGCTGGTGGTA
TTGTAGCTATCGTAGTAACATGCCTTGCCTACTATTTTATGAGGTTTAGAAGAGCTTTTGG
TGAATACAGTCATGTAGTTGCCTTTAATACTTTACTATTCCTTATGTCATTCACTGTACTC
TGTTTAACACCAGTTTACTCATTCTTACCTGGTGTTTATTCTGTTATTTACTTGTACTTGAC
ATTTTATCTTACTAATGATGTTTCTTTTTTAGCACATATTCAGTGGATGGTTATGTTCACA
CCTTTAGTACCTTTCTGGATAACAATTGCTTATATCATTTGTATTTCCACAAAGCATTTCT
ATTGGTTCTTTAGTAATTACCTAAAGAGGCGTGTAGTCTTTAATGGTGTTTCCTTTAGTAC
TTTTGAAGAAGCTGCGCTGTGCACCTTTTTGTTAAATAAAGAAATGTATCTAAAGTTGCG
TAGTGATGTGCTATTACCTCTTACGCAATATAATAGATACTTAGCTCTTTATAATAAGTAC
AAGTATTTTAGTGGAGCAATGGATACAACTAGCTACAGAGAAGCTGCTTGTTGTCATCTC
GCAAAGGCTCTCAATGACTTCAGTAACTCAGGTTCTGATGTTCTTTACCAACCACCACAA
ACCTCTATCACCTCAGCTGTTTTGCAGAGTGGTTTTAGAAAAATGGCATTCCCATCTGGT
AAAGTTGAGGGTTGTATGGTACAAGTAACTTGTGGTACAACTACACTTAACGGTCTTTGG
CTTGATGACGTAGTTTACTGTCCAAGACATGTGATCTGCACCTCTGAAGACATGCTTAAC
CCTAATTATGAAGATTTACTCATTCGTAAGTCTAATCATAATTTCTTGGTACAGGCTGGTA
ATGTTCAACTCAGGGTTATTGGACATTCTATGCAAAATTGTGTACTTAAGCTTAAGGTTG
ATACAGCCAATCCTAAGACACCTAAGTATAAGTTTGTTCGCATTCAACCAGGACAGACTT
TTTCAGTGTTAGCTTGTTACAATGGTTCACCATCTGGTGTTTACCAATGTGCTATGAGGCC
CAATTTCACTATTAAGGGTTCATTCCTTAATGGTTCATGTGGTAGTGTTGGTTTTAACATA
GATTATGACTGTGTCTCTTTTTGTTACATGCACCATATGGAATTACCAACTGGAGTTCATG
CTGGCACAGACTTAGAAGGTAACTTTTATGGACCTTTTGTTGACAGGCAAACAGCACAAG
CAGCTGGTACGGACACAACTATTACAGTTAATGTTTTAGCTTGGTTGTACGCTGCTGTTAT
AAATGGAGACAGGTGGTTTCTCAATCGATTTACCACAACTCTTAATGACTTTAACCTTGT
GGCTATGAAGTACAATTATGAACCTCTAACACAAGACCATGTTGACATACTAGGACCTCT
TTCTGCTCAAACTGGAATTGCCGTTTTAGATATGTGTGCTTCATTAAAAGAATTACTGCA
AAATGGTATGAATGGACGTACCATATTGGGTAGTGCTTTATTAGAAGATGAATTTACACC
TTTTGATGTTGTTAGACAATGCTCAGGTGTTACTTTCCAAAGTGCAGTGAAAAGAACAAT
CAAGGGTACACACCACTGGTTGTTACTCACAATTTTGACTTCACTTTTAGTTTTAGTCCAG
AGTACTCAATGGTCTTTGTTCTTTTTTTTGTATGAAAATGCCTTTTTACCTTTTGCTATGGG
TATTATTGCTATGTCTGCTTTTGCAATGATGTTTGTCAAACATAAGCATGCATTTCTCTGT
TTGTTTTTGTTACCTTCTCTTGCCACTGTAGCTTATTTTAATATGGTCTATATGCCTGCTAG
TTGGGTGATGCGTATTATGACATGGTTGGATATGGTTGATACTAGTTTGTCTGGTTTTAAA
CTTAAGGATTGTGTTATGTACGCTAGTGCCGTTGTGTTATTGATTCTTATGACTGCTAGAA
CAGTTTATGACGACGGAGCTAGACGCGTTTGGACACTTATGAACGTACTTACACTCGTAT
ATAAGGTATATTATGGTAACGCACTCGATCAGGCTATATCTATGTGGGCATTGATTATAT
CCGTTACGTCTAATTATTCAGGCGTAGTTACTACAGTTATGTTTCTCGCTAGAGGTATCGT
TTTTATGTGTGTTGAGTATTGTCCTATATTTTTTATAACCGGTAATACATTGCAATGTATT
ATGTTAGTTTATTGTTTTCTCGGTTATTTTTGTACATGTTATTTCGGTCTATTTTGTTTGCTT
AATAGGTATTTTAGATTGACATTAGGCGTATACGATTATCTCGTTAGTACACAGGAATTT
AGATATATGAATTCACAGGGTCTATTGCCTCCTAAAAACTCAATCGACGCATTCAAACTT
AATATTAAGCTATTAGGCGTAGGTGGTAAGCCTTGTATTAAAGTCGCTACAGTGCAATCT
AAAATGTCTGACGTTAAGTGTACATCAGTCGTTCTGTTATCAGTGTTACAGCAATTGAGA
GTCGAATCTAGTTCTAAGTTATGGGCTCAATGCGTTCAATTGCACAACGATATACTGTTA
GCTAAGGATACTACTGAGGCATTCGAAAAAATGGTTAGTCTATTATCAGTATTGCTATCT
ATGCAGGGTGCAGTCGATATTAATAAGTTATGCGAAGAGATGCTCGATAATAGGGCTAC
ATTGCAGGCAATCGCTAGCGAATTTTCATCACTACCATCTTATGCCGCATTCGCAACTGC
TCAGGAAGCATACGAACAAGCCGTAGCTAACGGTGATTCAGAGGTTGTGCTTAAAAAAC
TTAAAAAATCACTTAACGTTGCTAAATCCGAATTCGATAGGGACGCCGCTATGCAACGTA
AACTCGAAAAAATGGCCGATCAGGCTATGACACAAATGTATAAACAGGCTAGATCCGAA
GATAAGAGAGCTAAGGTTACTAGTGCAATGCAAACTATGCTTTTTACTATGTTGCGTAAA
CTCGATAACGACGCACTTAATAATATTATTAATAACGCTAGGGACGGTTGTGTACCACTT
AACATTATACCACTTACTACTGCCGCTAAACTTATGGTCGTTATACCCGATTATAATACTT
ATAAAAATACATGTGACGGTACTACTTTTACATACGCTAGTGCATTATGGGAGATACAAC
AGGTAGTCGATGCCGATTCTAAAATCGTTCAATTGTCAGAGATATCTATGGATAATTCAC
CTAATCTCGCATGGCCTCTTATCGTTACCGCACTTAGAGCTAATTCTGCCGTTAAGTTACA
GAATAACGAATTGTCACCCGTTGCCCTACGACAGATGTCTTGTGCTGCCGGTACTACACA
AACTGCTTGCACTGATGACAATGCGTTAGCTTACTACAACACAACAAAGGGAGGTAGGT
TTGTACTTGCACTGTTATCCGATTTACAGGATTTGAAATGGGCTAGATTCCCTAAGAGTG
ATGGAACTGGTACTATCTATACAGAACTGGAACCACCTTGTAGGTTTGTTACAGACACAC
CTAAAGGTCCTAAAGTGAAGTATTTATACTTTATTAAAGGATTAAACAACCTAAATAGAG
GTATGGTACTTGGTAGTTTAGCTGCCACAGTACGTCTACAAGCTGGTAATGCAACAGAAG
TGCCTGCCAATTCAACTGTATTATCTTTCTGTGCTTTTGCTGTAGATGCTGCTAAAGCTTA
CAAAGATTATCTAGCTAGTGGGGGACAACCAATCACTAATTGTGTTAAGATGTTGTGTAC
ACACACTGGTACTGGTCAGGCAATAACAGTTACACCGGAAGCCAATATGGATCAAGAAT
CCTTTGGTGGTGCATCGTGTTGTCTGTACTGCCGTTGCCACATAGATCATCCAAATCCTAA
AGGATTTTGTGACTTAAAAGGTAAGTATGTACAAATACCTACAACTTGTGCTAATGACCC
TGTGGGTTTTACACTTAAAAACACAGTCTGTACCGTCTGCGGTATGTGGAAAGGTTATGG
CTGTAGTTGTGATCAACTCCGCGAACCCATGCTTCAGTCAGCTGATGCACAATCGTTTTT
AAACGGGTTTGCGGTGTAAGTGCAGCCCGTCTTACACCGTGCGGCACAGGCACTAGTACT
GATGTCGTATACAGGGCTTTTGACATCTACAATGATAAAGTAGCTGGTTTTGCTAAATTC
CTAAAAACTAATTGTTGTCGCTTCCAAGAAAAGGACGAAGATGACAATTTAATTGATTCT
TACTTTGTAGTTAAGAGACACACTTTCTCTAACTACCAACATGAAGAAACAATTTATAAT
TTACTTAAGGATTGTCCAGCTGTTGCTAAACATGACTTCTTTAAGTTTAGAATAGACGGT
GACATGGTACCACATATATCACGTCAACGTCTTACTAAATACACAATGGCAGACCTCGTC
TATGCTTTAAGGCATTTTGATGAAGGTAATTGTGACACATTAAAAGAAATACTTGTCACA
TACAATTGTTGTGATGATGATTATTTCAATAAAAAGGACTGGTATGATTTTGTAGAAAAC
CCAGATATATTACGCGTATACGCCAACTTAGGTGAACGTGTACGCCAAGCTTTGTTAAAA
ACAGTACAATTCTGTGATGCCATGCGAAATGCTGGTATTGTTGGTGTACTGACATTAGAT
AATCAAGATCTCAATGGTAACTGGTATGATTTCGGTGATTTCATACAAACCACGCCAGGT
AGTGGAGTTCCTGTTGTAGATTCTTATTATTCATTGTTAATGCCTATATTAACCTTGACCA
GGGCTTTAACTGCAGAGTCACATGTTGACACTGACTTAACAAAGCCTTACATTAAGTGGG
ATTTGTTAAAATATGACTTCACGGAAGAGAGGTTAAAACTCTTTGACCGTTATTTTAAAT
ATTGGGATCAGACATACCACCCAAATTGTGTTAACTGTTTGGATGACAGATGCATTCTGC
ATTGTGCAAACTTTAATGTTTTATTCTCTACAGTGTTCCCACCTACAAGTTTTGGACCACT
AGTGAGAAAAATATTTGTTGATGGTGTTCCATTTGTAGTTTCAACTGGATACCACTTCAG
AGAGCTAGGTGTTGTACATAATCAGGATGTAAACTTACATAGCTCTAGACTTAGTTTTAA
GGAATTACTTGTGTATGCTGCTGACCCTGCTATGCACGCTGCTTCTGGTAATCTATTACTA
GATAAACGCACTACGTGCTTTTCAGTAGCTGCACTTACTAATAACGTCGCATTTCAAACC
GTTAAACCAGGTAATTTTAATAAGGATTTTTACGATTTCGCCGTTAGTAAGGGATTTTTTA
AAGAGGGTAGTTCAGTCGAACTTAAACACTTCTTTTTCGCACAAGACGGTAACGCCGCTA
TTAGCGATTACGATTATTATAGATATAATCTACCTACTATGTGTGATATTAGACAATTGTT
ATTCGTAGTCGAGGTAGTCGATAAGTATTTCGATTGTTATGACGGAGGTTGTATTAACGC
TAATCAGGTAATCGTTAATAACCTTGATAAGTCTGCCGGTTTTCCTTTTAATAAATGGGGT
AAGGCTAGATTGTATTATGATTCTATGTCATACGAAGACCAAGACGCTCTATTCGCATAT
ACTAAACGTAACGTTATACCTACAATTACGCAAATGAATCTTAAGTATGCTATTAGCGCT
AAAAATCGCGCTAGAACAGTCGCCGGAGTGTCAATCTGTAGTACTATGACTAATCGTCA
ATTTCACCAAAAATTGCTTAAATCAATCGCCGCTACTAGAGGCGCTACAGTCGTTATCGG
TACATCTAAATTTTATGGCGGATGGCATAATATGCTTAAAACGGTTTATTCTGACGTTGA
GAATCCACACCTTATGGGTTGGGATTATCCTAAATGCGATCGCGCTATGCCTAATATGCT
TAGAATTATGGCTAGTCTCGTACTCGCACGTAAACATACAACATGTTGTAGTCTATCACA
TAGATTTTATAGATTAGCTAACGAATGTGCACAAGTGCTTAGCGAAATGGTTATGTGTGG
TGGATCACTATATGTTAAACCCGGTGGTACATCTAGCGGTGACGCTACTACCGCATACGC
TAATAGCGTATTCAATATATGTCAAGCCGTTACCGCTAACGTTAACGCTCTACTATCTACT
GACGGTAATAAGATAGCCGATAAGTATGTTAGAAACTTACAACATAGATTGTATGAGTG
TCTTTATCGTAATCGCGATGTCGATACTGATTTCGTTAACGAATTCTATGCTTATCTTAGA
AAACACTTTAGTATGATGATACTATCTGACGATGCCGTAGTGTGTTTTAACTCTACTTATG
CTAGTCAGGGATTAGTCGCATCAATCAAAAATTTTAAATCAGTGTTATATTATCAAAATA
ACGTTTTTATGTCAGAAGCTAAATGTTGGACTGAGACTGATCTTACTAAGGGTCCACACG
AATTTTGTTCACAACATACTATGTTAGTTAAACAGGGTGATGATTATGTGTACCTTCCTTA
CCCAGATCCATCAAGAATCCTAGGGGCCGGCTGTTTTGTAGATGATATCGTAAAAACAG
ATGGTACACTTATGATTGAACGGTTCGTGTCTTTAGCTATAGATGCTTACCCACTTACTAA
ACATCCTAATCAGGAGTATGCTGATGTCTTTCATTTGTACTTACAATACATAAGAAAGCT
ACATGATGAGTTAACAGGACACATGTTAGACATGTATTCTGTTATGCTTACTAATGATAA
CACTTCAAGGTATTGGGAACCTGAGTTTTATGAGGCTATGTACACACCGCATACAGTCTT
ACAGGCTGTTGGGGCTTGTGTTCTTTGCAATTCACAGACTTCATTAAGATGTGGTGCTTGC
ATACGTAGACCATTCTTATGTTGTAAATGCTGTTACGACCATGTCATATCAACATCACAT
AAATTAGTCTTGTCTGTTAATCCGTATGTTTGCAATGCTCCAGGTTGTGATGTCACAGATG
TGACTCAACTTTACTTAGGAGGTATGAGCTATTATTGTAAATCACATAAACCACCCATTA
GTTTTCCATTGTGTGCTAATGGACAAGTTTTTGGTTTATATAAAAATACATGTGTTGGTAG
CGATAATGTTACTGACTTTAATGCAATTGCAACATGTGACTGGACAAATGCTGGTGATTA
CATTTTAGCTAACACCTGTACTGAAAGACTCAAGCTTTTTGCAGCAGAAACGCTCAAAGC
TACTGAGGAGACATTTAAACTGTCTTATGGTATTGCTACTGTACGTGAAGTGCTGTCTGA
CAGAGAATTACATCTTTCATGGGAAGTTGGTAAACCTAGACCACCACTTAACCGAAATTA
TGTCTTTACTGGTTATCGTGTAACTAAAAACAGTAAAGTACAAATAGGAGAGTACACCTT
TGAAAAAGGTGACTATGGTGATGCTGTTGTTTACCGAGGTACAACAACTTACAAATTAAA
TGTTGGTGATTATTTTGTGCTGACATCACATACAGTAATGCCATTAAGTGCACCTACACT
AGTGCCACAAGAGCACTATGTTAGAATTACTGGCTTATACCCAACACTCAATATCTCAGA
TGAGTTTTCTAGCAATGTTGCAAATTATCAAAAGGTTGGTATGCAAAAGTATTCTACACT
CCAGGGACCACCTGGTACTGGTAAGAGTCATTTTGCTATTGGCCTAGCTCTCTACTACCC
TTCTGCTCGCATAGTGTATACAGCTTGCTCTCATGCCGCTGTTGATGCACTATGTGAGAA
GGCATTAAAATATTTGCCTATAGATAAATGTAGTAGAATTATACCTGCACGTGCTCGTGT
AGAGTGTTTTGATAAATTCAAAGTGAATTCAACATTAGAACAGTATGTCTTTTGTACTGT
AAATGCATTGCCTGAAACGACAGCAGATATAGTTGTCTTTGATGAAATTTCAATGGCCAC
AAATTATGATTTGAGTGTTGTCAATGCCAGATTACGTGCTAAGCACTATGTGTACATTGG
CGACCCTGCTCAATTACCTGCACCACGCACATTGCTAACTAAGGGCACACTAGAACCAG
AATATTTCAATTCAGTGTGTAGACTTATGAAAACTATAGGTCCAGACATGTTCCTCGGAA
CTTGTCGGCGTTGTCCTGCTGAAATTGTTGACACTGTGAGTGCTTTGGTTTATGATAATAA
GCTTAAAGCACATAAAGACAAATCAGCTCAATGCTTTAAAATGTTTTATAAGGGTGTTAT
CACGCATGATGTTTCATCTGCAATTAACAGGCCACAAATAGGCGTGGTAAGAGAATTCCT
TACACGTAACCCTGCTTGGAGAAAAGCTGTCTTTATTTCACCTTATAATTCACAGAATGC
TGTAGCCTCAAAGATTTTGGGACTACCAACTCAAACTGTTGATTCATCACAGGGCTCAGA
ATATGACTATGTCATATTCACTCAAACCACTGAAACAGCTCACTCTTGTAATGTAAACAG
ATTTAATGTTGCTATTACCAGAGCAAAAGTAGGCATACTTTGCATAATGTCTGATAGGGA
CCTTTATGACAAGTTGCAATTTACAAGTCTTGAAATTCCACGTAGGAATGTGGCAACTTT
ACAAGCTGAAAATGTAACAGGACTCTTTAAAGATTGTAGTAAGGTAATCACTGGGTTAC
ATCCTACACAGGCACCTACACACCTCAGTGTTGACACTAAATTCAAAACTGAAGGTTTAT
GTGTTGACATACCTGGCATACCTAAGGACATGACCTATAGAAGACTCATCTCTATGATGG
GTTTTAAAATGAATTATCAAGTTAATGGTTACCCTAACATGTTTATCACCCGCGAAGAAG
CTATAAGACATGTACGTGCATGGATTGGCTTCGATGTCGAGGGGTGTCATGCTACTAGAG
AAGCTGTTGGTACCAATTTACCTTTACAGCTAGGTTTTTCTACAGGTGTTAACCTAGTTGC
TGTACCTACAGGTTATGTTGATACACCTAATAATACAGATTTTTCCAGAGTTAGTGCTAA
ACCACCGCCTGGAGATCAATTTAAACACCTCATACCACTTATGTACAAAGGACTTCCTTG
GAATGTAGTGCGTATAAAGATTGTACAAATGTTAAGTGACACACTTAAAAATCTCTCTGA
CAGAGTCGTATTTGTCTTATGGGCACATGGCTTTGAGTTGACATCTATGAAGTATTTTGTG
AAAATAGGACCTGAGCGCACCTGTTGTCTATGTGATAGACGTGCCACATGCTTTTCCACT
GCTTCAGACACTTATGCCTGTTGGCATCATTCTATTGGATTTGATTACGTCTATAATCCGT
TTATGATTGATGTTCAACAATGGGGTTTTACAGGTAACCTACAAAGCAACCATGATCTGT
ATTGTCAAGTCCATGGTAATGCACATGTAGCTAGTTGTGATGCAATCATGACTAGGTGTC
TAGCTGTCCACGAGTGCTTTGTTAAGCGTGTTGACTGGACTATTGAATATCCTATAATTG
GTGATGAACTGAAGATTAATGCGGCTTGTAGAAAGGTTCAACACATGGTTGTTAAAGCT
GCATTATTAGCAGACAAATTCCCAGTTCTTCACGACATTGGTAACCCTAAAGCTATTAAG
TGTGTACCTCAAGCTGATGTAGAATGGAAGTTCTATGATGCACAGCCTTGTAGTGACAAA
GCTTATAAAATAGAAGAATTATTCTATTCTTATGCCACACATTCTGACAAATTCACAGAT
GGTGTATGCCTATTTTGGAATTGCAATGTCGATAGATATCCTGCTAATTCCATTGTTTGTA
GATTTGACACTAGAGTGCTATCTAACCTTAACTTGCCTGGTTGTGATGGTGGCAGTTTGT
ATGTAAATAAACATGCATTCCACACACCAGCTTTTGATAAAAGTGCTTTTGTTAATTTAA
AACAATTACCATTTTTCTATTACTCTGACAGTCCATGTGAGTCTCATGGAAAACAAGTAG
TGTCAGATATAGATTATGTACCACTAAAGTCTGCTACGTGTATAACACGTTGCAATTTAG
GTGGTGCTGTCTGTAGACATCATGCTAATGAGTACAGATTGTATCTCGATGCTTATAACA
TGATGATCTCAGCTGGCTTTAGCTTGTGGGTTTACAAACAATTTGATACTTATAACCTCTG
GAACACTTTTACAAGACTTCAGAGTTTAGAAAATGTGGCTTTTAATGTTGTAAATAAGGG
ACACTTTGATGGACAACAGGGTGAAGTACCAGTTTCTATCATTAATAACACTGTTTACAC
AAAAGTTGATGGTGTTGATGTAGAATTGTTTGAAAATAAAACAACATTACCTGTTAATGT
AGCATTTGAGCTTTGGGCTAAGCGCAACATTAAACCAGTACCAGAGGTGAAAATACTCA
ATAATTTGGGTGTGGACATTGCTGCTAATACTGTGATCTGGGACTACAAAAGAGATGCTC
CAGCACATATATCTACTATTGGTGTTTGTTCTATGACTGACATAGCCAAGAAACCAACTG
AAACGATTTGTGCACCACTCACTGTCTTTTTTGATGGTAGAGTTGATGGTCAAGTAGACT
TATTTAGAAATGCCCGTAATGGTGTTCTTATTACAGAAGGTAGTGTTAAAGGTTTACAAC
CATCTGTAGGTCCCAAACAAGCTAGTCTTAATGGAGTCACATTAATTGGAGAAGCCGTAA
AAACACAGTTCAATTATTATAAGAAAGTTGATGGTGTTGTCCAACAATTACCTGAAACTT
ACTTTACTCAGAGTAGAAATTTACAAGAATTTAAACCCAGGAGTCAAATGGAAATTGATT
TCTTAGAATTAGCTATGGATGAATTCATTGAACGGTATAAATTAGAAGGCTATGCCTTCG
AACATATCGTTTATGGAGATTTTAGTCATAGTCAGTTAGGTGGTTTACATCTACTGATTGG
ACTAGCTAAACGTTTTAAGGAATCACCTTTTGAATTAGAAGATTTTATTCCTATGGACAG
TACAGTTAAAAACTATTTCATAACAGATGCGCAAACAGGTTCATCTAAGTGTGTGTGTTC
TGTTATTGATTTATTACTTGATGATTTTGTTGAAATAATAAAATCCCAAGATTTATCTGTA
GTTTCTAAGGTTGTCAAAGTGACTATTGACTATACAGAAATTTCATTTATGCTTTGGTGTA
AAGATGGCCATGTAGAAACATTTTACCCAAAATTACAATCTAGTCAAGCGTGGCAACCG
GGTGTTGCTATGCCTAATCTTTACAAAATGCAAAGAATGCTATTAGAAAAGTGTGACCTT
CAAAATTATGGTGATAGTGCAACATTACCTAAAGGCATAATGATGAATGTCGCAAAATA
TACTCAACTGTGTCAATATTTAAACACATTAACATTAGCTGTACCCTATAATATGAGAGT
TATACATTTTGGTGCTGGTTCTGATAAAGGAGTTGCACCAGGTACAGCTGTTTTAAGACA
GTGGTTGCCTACGGGTACGCTGCTTGTCGATTCAGATCTTAATGACTTTGTCTCTGATGCA
GATTCAACTTTGATTGGTGATTGTGCAACTGTACATACAGCTAATAAATGGGATCTCATT
ATTAGTGATATGTACGACCCTAAGACTAAAAATGTTACAAAAGAAAATGACTCTAAAGA
GGGTTTTTTCACTTACATTTGTGGGTTTATACAACAAAAGCTAGCTCTTGGAGGTTCCGTG
GCTATAAAGATAACAGAACATTCTTGGAATGCTGATCTTTATAAGCTCATGGGACACTTC
GCATGGTGGACAGCCTTTGTTACTAATGTGAATGCGTCATCATCTGAAGCATTTTTAATT
GGATGTAATTATCTTGGCAAACCACGCGAACAAATAGATGGTTATGTCATGCATGCAAAT
TACATATTTTGGAGGAATACAAATCCAATTCAGTTGTCTTCCTATTCTTTATTTGACATGA
GTAAATTTCCCCTTAAATTAAGGGGTACTGCTGTTATGTCTTTAAAAGAAGGTCAAATCA
ATGATATGATTTTATCTCTTCTTAGTAAAGGTAGACTTATAATTAGAGAAAACAACAGAG
TTGTTATTTCTAGTGATGTTCTTGTTAACAACTAAACGAACAATGTTTGTTTTTCTTGTTTT
ATTGCCACTAGTCTCTAGTCAGTGTGTTAATCTTACAACCAGAACTCAATTACCCCCTGC
ATACACTAATTCTTTTACTAGGGGGGTTTATTATCCTGATAAGGTTTTTCGTAGTTCAGTC
TTGCATAGTACTCAGGATCTATTTTTACCTTTCTTTTCGAACGTTACTTGGTTTCACGCTAT
ACACGTTTCCGGTACTAACGGTACTAAAAGATTCGATAATCCTGTCTTACCATTTAACGA
CGGAGTGTATTTCGCTAGTACCGAAAAATCTAATATTATTAGGGGTTGGATATTCGGTAC
TACTCTTGACTCTAAAACGCAATCATTGTTAATCGTTAATAACGCTACTAACGTCGTTATT
AAGGTATGCGAATTTCAGTTTTGTAACGATCCTTTCTTAGGCGTTTATTATCATAAAAATA
ATAAGTCTTGGATGGAATCCGAATTTAGAGTGTATAGTTCCGCTAATAATTGTACATTCG
AATACGTTTCGCAACCATTTCTTATGGATCTTGAGGGTAAGCAGGGTAATTTTAAAAATT
TGCGTGAATTCGTTTTTAAAAATATAGACGGTTATTTTAAGATATACTCTAAACATACCC
CTATTAATCTCGTTAGGGATCTACCTCAGGGTTTTTCCGCTCTTGAGCCATTAGTCGATTT
GCCAATAGGTATTAATATTACTAGATTTCAGACTCTTTTAGCGTTACATAGATCTTATTTG
ACTCCTGGTGATTCTTCTTCAGGTTGGACAGCTGGTGCTGCAGCTTATTATGTGGGTTATC
TTCAACCTAGGACTTTTCTATTAAAATATAATGAAAATGGAACCATTACAGATGCTGTAG
ACTGTGCACTTGACCCTCTCTCAGAAACAAAGTGTACGTTGAAATCCTTCACTGTAGAAA
AAGGAATCTATCAAACATCTAATTTTAGAGTGCAACCTACCGAATCAATCGTTAGATTTC
CTAATATTACTAACTTATGTCCATTTGGGGAAGTTTTTAACGCTACTAGATTCGCTAGCGT
TTACGCTTGGAATCGTAAACGTATATCTAATTGCGTTGCCGATTATTCCGTACTTTATAAT
TCCGCTAGTTTTTCTACTTTTAAGTGTTACGGAGTTAGTCCAACTAAGCTTAACGATCTAT
GTTTTACTAACGTATACGCTGATTCATTCGTTATTAGAGGTGACGAAGTTAGACAGATTG
CACCAGGTCAAACAGGTAAGATTGCCGATTATAATTATAAGTTGCCTGACGATTTTACAG
GTTGCGTTATCGCTTGGAATTCTAATAATCTCGATTCTAAAGTCGGAGGTAATTATAATT
ATCTATATAGATTGTTTCGTAAGTCTAACCTTAAACCATTCGAACGGGATATTAGTACTG
AGATTTATCAGGCAGGTAGTACACCTTGTAACGGTGTCGAAGGTTTTAATTGTTATTTTCC
ACTTCAATCTTACGGTTTTCAACCTACTAACGGAGTCGGATATCAACCATATAGAGTAGT
CGTTCTATCATTCGAACTGTTACATGCACCCGCTACAGTTTGTGGTCCTAAAAAATCTACT
AACTTAGTTAAGAATAAATGCGTTAATTTTAACTTTAACGGTCTTACAGGTACAGGCGTA
CTTACCGAATCTAACAAAAAATTTTTACCATTTCAACAATTTGGTAGGGATATAGCCGAT
ACTACTGACGCAGTTAGGGATCCACAAACACTTGAGATACTCGATATTACACCTTGTTCT
TTTGGCGGGGTTAGCGTAATTACACCCGGTACTAATACATCTAATCAGGTCGCAGTACTT
TATCAAGACGTTAATTGTACTGAAGTGCCAGTCGCTATACATGCTGATCAATTGACACCT
ACATGGCGGGTTTATTCTACCGGTTCTAACGTTTTTCAGACTAGAGCCGGTTGTCTTATAG
GCGCTGAACATGTTAATAACTCATACGAATGCGATATTCCTATAGGCGCAGGTATTTGCG
CATCATATCAAACACAGACTAACTCACCTAGACGTGCTAGATCCGTTGCTAGTCAATCTA
TTATCGCTTATACTATGTCTTTAGGCGCTGAAAACTCAGTCGCATACTCTAATAACTCAAT
CGCTATACCTACTAATTTTACAATTAGCGTTACTACTGAGATTTTACCCGTTAGTATGACT
AAGACTTCAGTCGATTGTACTATGTATATTTGCGGTGACTCTACCGAATGTTCTAATCTGT
TATTGCAATACGGATCTTTTTGTACACAATTGAATCGTGCACTTACAGGTATTGCAGTCG
AACAGGATAAGAATACACAAGAGGTTTTTGCACAAGTCAAACAAATTTACAAAACACCA
CCAATTAAAGATTTTGGTGGTTTTAATTTTTCACAAATATTACCAGATCCATCAAAACCA
AGCAAGAGGTCATTTATTGAAGATCTACTTTTCAACAAAGTGACACTTGCAGATGCTGGC
TTCATCAAACAATATGGTGATTGCCTTGGTGATATTGCTGCTAGAGATCTCATTTGCGCTC
AAAAATTTAACGGACTTACAGTTTTACCACCTTTACTTACTGACGAAATGATTGCGCAAT
ATACATCCGCATTGTTAGCCGGAACTATTACATCCGGATGGACTTTTGGCGCAGGCGCAG
CATTACAGATTCCATTCGCTATGCAAATGGCTTATAGGTTTAACGGTATAGGCGTTACGC
AAAACGTACTTTATGAGAATCAAAAACTTATCGCTAACCAATTTAATTCCGCTATCGGTA
AGATTCAGGATTCATTGTCTAGTACTGCTAGTGCACTCGGTAAGTTGCAAGACGTAGTGA
ATCAAAACGCTCAAGCACTTAATACACTCGTTAAACAGCTTAGTTCTAATTTTGGCGCAA
TTTCTAGTGTGCTTAACGATATACTATCTAGACTCGATAAAGTCGAAGCCGAAGTGCAAA
TCGATAGATTGATTACCGGTAGGTTGCAATCATTGCAAACATACGTTACACAGCAATTGA
TTAGGGCCGCAGAGATACGCGCTAGCGCTAATCTCGCAGCTACTAAAATGTCTGAATGC
GTACTCGGACAATCTAAACGTGTCGATTTTTGCGGTAAGGGATATCATCTTATGTCTTTTC
CACAATCTGCACCTCACGGAGTCGTGTTTTTACACGTTACTTATGTGCCAGCTCAAGAGA
AAAATTTTACAACCGCTCCTGCTATTTGTCATGACGGTAAGGCACATTTTCCTAGAGAGG
GCGTATTCGTTTCTAACGGTACACATTGGTTCGTTACACAACGTAATTTTTACGAACCTCA
AATTATTACTACTGATAATACATTCGTATCAGGTAATTGTGACGTAGTGATAGGTATCGT
TAATAATACAGTTTACGATCCACTTCAACCTGAACTCGATAGTTTTAAAGAGGAACTCGA
TAAGTATTTTAAAAATCATACATCACCTGACGTCGACTTAGGCGATATTTCAGGTATTAA
CGCTAGTGTCGTTAACATTCAAAAAGAGATTGATAGACTTAACGAAGTCGCTAAAAATCT
TAACGAATCACTTATCGATCTGCAAGAGTTAGGTAAGTATGAGCAATATATTAAATGGCC
TTGGTATATTTGGTTAGGCTTTATAGCCGGATTGATCGCAATCGTTATGGTTACAATTATG
TTATGTTGTATGACATCATGTTGTTCATGTCTTAAGGGATGTTGTTCATGCGGATCATGTT
GTAAATTTGACGAAGACGATTCCGAACCAGTGCTTAAAGGCGTTAAGTTACATTATACAT
AAACGAACTTATGGATTTGTTTATGAGAATCTTCACAATTGGAACTGTAACTTTGAAGCA
AGGTGAAATCAAGGATGCTACTCCTTCAGATTTTGTTCGCGCTACTGCAACGATACCGAT
ACAAGCCTCACTCCCTTTCGGATGGCTTATTGTTGGCGTTGCACTTCTTGCTGTTTTTCAG
AGCGCTTCCAAAATCATAACCCTCAAAAAGAGATGGCAACTAGCACTCTCCAAGGGTGT
TCACTTTGTTTGCAACTTGCTGTTGTTGTTTGTAACAGTTTACTCACACCTTTTGCTCGTTG
CTGCTGGCCTTGAAGCCCCTTTTCTCTATCTTTATGCTTTAGTCTACTTCTTGCAGAGTATA
AACTTTGTAAGAATAATAATGAGGCTTTGGCTTTGCTGGAAATGCCGTTCCAAAAACCCA
TTACTTTATGATGCCAACTATTTTCTTTGCTGGCATACTAATTGTTACGACTATTGTATAC
CTTACAATAGTGTAACTTCTTCAATTGTCATTACTTCAGGTGATGGCACAACAAGTCCTAT
TTCTGAACATGACTACCAGATTGGTGGTTATACTGAAAAATGGGAATCTGGAGTAAAAG
ACTGTGTTGTATTACACAGTTACTTCACTTCAGACTATTACCAGCTGTACTCAACTCAATT
GAGTACAGACACTGGTGTTGAACATGTTACCTTCTTCATCTACAATAAAATTGTTGATGA
GCCTGAAGAACATGTCCAAATTCACACAATCGACGGTTCATCCGGAGTTGTTAATCCAGT
AATGGAACCAATTTATGATGAACCGACGACGACTACTAGCGTGCCTTTGTAAGCACAAG
CTGATGAGTACGAACTTATGTACTCATTCGTTTCGGAAGAGACAGGTACGTTAATAGTTA
ATAGCGTACTTCTTTTTCTTGCTTTCGTGGTATTCTTGCTAGTTACACTAGCCATCCTTACT
GCGCTTCGATTGTGTGCGTACTGCTGCAATATTGTTAACGTGAGTCTTGTAAAACCTTCTT
TTTACGTTTACTCTCGTGTTAAAAATCTGAATTCTTCTAGAGTTCCTGATCTTCTGGTCTA
AACGAACTAAATATTATATTAGTTTTTCTGTTTGGAACTTTAATTTTAGCCATGGCAGATT
CCAACGGTACTATTACCGTTGAAGAGCTTAAAAAGCTCCTTGAACAATGGAACCTAGTA
ATAGGTTTCCTATTCCTTACATGGATTTGTCTTCTACAATTTGCCTATGCCAACAGGAATA
GGTTTTTGTATATAATTAAGTTAATTTTCCTCTGGCTGTTATGGCCAGTAACTTTAGCTTG
TTTTGTGCTTGCTGCTGTTTACAGAATAAATTGGATCACCGGTGGAATTGCTATCGCAAT
GGCTTGTCTTGTAGGCTTGATGTGGCTCAGCTACTTCATTGCTTCTTTCAGACTGTTTGCG
CGTACGCGTTCCATGTGGTCATTCAATCCAGAAACTAACATTCTTCTCAACGTGCCACTC
CATGGCACTATTCTGACCAGACCGCTTCTAGAAAGTGAACTCGTAATCGGAGCTGTGATC
CTTCGTGGACATCTTCGTATTGCTGGACACCATCTAGGACGCTGTGACATCAAGGACCTG
CCTAAAGAAATCACTGTTGCTACATCACGAACGCTTTCTTATTACAAATTGGGAGCTTCG
CAGCGTGTAGCAGGTGACTCAGGTTTTGCTGCATACAGTCGCTACAGGATTGGCAACTAT
AAATTAAACACAGACCATTCCAGTAGCAGTGACAATATTGCTTTGCTTGTACAGTAAGTG
ACAACAGATGTTTCATCTCGTTGACTTTCAGGTTACTATAGCAGAGATATTACTAATTATT
ATGAGGACTTTTAAAGTTTCCATTTGGAATCTTGATTACATCATAAACCTCATAATTAAA
AATTTATCTAAGTCACTAACTGAGAATAAATATTCTCAATTAGATGAAGAGCAACCAATG
GAGATTGATTAAACGAACATGAAAATTATTCTTTTCTTGGCACTGATAACACTCGCTACT
TGTGAGCTTTATCACTACCAAGAGTGTGTTAGAGGTACAACAGTACTTTTAAAAGAACCT
TGCTCTTCTGGAACATACGAGGGCAATTCACCATTTCATCCTCTAGCTGATAACAAATTT
GCACTGACTTGCTTTAGCACTCAATTTGCTTTTGCTTGTCCTGACGGCGTAAAACACGTCT
ATCAGTTACGTGCCAGATCAGTTTCACCTAAACTGTTCATCAGACAAGAGGAAGTTCAAG
AACTTTACTCTCCAATTTTTCTTATTGTTGCGGCAATAGTGTTTATAACACTTTGCTTCAC
ACTCAAAAGAAAGACAGAATGATTGAACTTTCATTAATTGACTTCTATTTGTGCTTTTTA
GCCTTTCTGCTATTCCTTGTTTTAATTATGCTTATTATCTTTTGGTTCTCACTTGAACTGCA
AGATCATAATGAAACTTGTCACGCCTAAACGAACATGAAATTTCTTGTTTTCTTAGGAAT
CATCACAACTGTAGCTGCATTTCACCAAGAATGTAGTTTACAGTCATGTACTCAACATCA
ACCATATGTAGTTGATGACCCGTGTCCTATTCACTTCTATTCTAAATGGTATATTAGAGTA
GGAGCTAGAAAATCAGCACCTTTAATTGAATTGTGCGTGGATGAGGCTGGTTCTAAATCA
CCCATTCAGTACATCGATATCGGTAATTATACAGTTTCCTGTTTACCTTTTACAATTAATT
GCCAGGAACCTAAATTGGGTAGTCTTGTAGTGCGTTGTTCGTTCTATGAAGACTTTTTAG
AGTATCATGACGTTCGTGTTGTTTTAGATTTCATCTAAACGAACAAACTAAAATGTCTGA
TAATGGACCCCAAAATCAGCGAAATGCACCCCGCATTACGTTTGGTGGACCCTCAGATTC
AACTGGCAGTAACCAGAATGGAGAACGCAGTGGGGCGCGATCAAAACAACGTCGGCCC
CAAGGTTTACCCAATAATACTGCGTCTTGGTTCACCGCTCTCACTCAACATGGCAAGGAA
GACCTTAAATTCCCTCGAGGACAAGGCGTTCCAATTAACACCAATAGCAGTCCAGATGA
CCAAATTGGCTACTACCGAAGAGCTACCAGACGAATTCGTGGTGGTGACGGTAAAATGA
AAGATCTCAGTCCAAGATGGTATTTCTACTACCTAGGAACTGGGCCAGAAGCTGGACTTC
CCTATGGTGCTAACAAAGACGGCATCATATGGGTTGCAACTGAGGGAGCCTTGAATACA
CCAAAAGATCACATTGGCACCCGCAATCCTGCTAACAATGCTGCAATCGTGCTACAACTT
CCTCAAGGAACAACATTGCCAAAAGGCTTCTACGCAGAAGGGAGCAGAGGCGGCAGTCA
AGCCTCTTCTCGTTCCTCATCACGTAGTCGCAACAGTTCAAGAAATTCAACTCCAGGCAG
CAGTAGGGGAACTTCTCCTGCTAGAATGGCTGGCAATGGCGGTGATGCTGCTCTTGCTTT
GCTGCTGCTTGACAGATTGAACCAGCTTGAGAGCAAAATGTCTGGTAAAGGCCAACAAC
AACAAGGCCAAACTGTCACTAAGAAATCTGCTGCTGAGGCTTCTAAGAAGCCTCGGCAA
AAACGTACTGCCACTAAAGCATACAATGTAACACAAGCTTTCGGCAGACGTGGTCCAGA
ACAAACCCAAGGAAATTTTGGGGACCAGGAACTAATCAGACAAGGAACTGATTACAAAC
ATTGGCCGCAAATTGCACAATTTGCCCCCAGCGCTTCAGCGTTCTTCGGAATGTCGCGCA
TTGGCATGGAAGTCACACCTTCGGGAACGTGGTTGACCTACACAGGTGCCATCAAATTGG
ATGACAAAGATCCAAATTTCAAAGATCAAGTCATTTTGCTGAATAAGCATATTGACGCAT
ACAAAACATTCCCACCAACAGAGCCTAAAAAGGACAAAAAGAAGAAGGCTGATGAAAC
TCAAGCCTTACCGCAGAGACAGAAGAAACAGCAAACTGTGACTCTTCTTCCTGCTGCAG
ATTTGGATGATTTCTCCAAACAATTGCAACAATCCATGAGCAGTGCTGACTCAACTCAGG
CCTAAACTCATGCAGACCACACAAGGCAGATGGGCTATATAAACGTTTTCGCTTTTCCGT
TTACGATATATAGTCTACTCTTGTGCAGAATGAATTCTCGTAACTACATAGCACAAGTAG
ATGTAGTTAACTTTAATCTCACATAGCAATCTTTAATCAGTGTGTAACATTAGGGAGGAC
TTGAAAGAGCCACCACATTTTCACCGAGGCCACGCGGAGTACGATCGAGTGTACAGTGA
ACAATGCTAGGGAGAGCTGCCTATATGGAAGAGCCCTAATGTGTAAAATTAATTTTAGTA
GTGCTATCCCCATGTGATTTTAATAGCTTCTTAGGAGAATGACAAAAAAAAAAAAAAAA
AAAAA
SEQ ID NO: 4 (deoptimized in reference to Washington isolate (GenBank:
MN985325.1), with a 36 nucleotide deletion in the spike protein).
attaaaggtttataccttcccaggtaacaaaccaaccaactttcgatctcttgtagatctgttctctaaacgaactttaaaatctgtgtggctgtcactcgg
ctgcatgcttagtgcactcacgcagtataattaataactaattactgtcgttgacaggacacgagtaactcgtctatcttctgcaggctgcttacggtttc
gtccgtgttgcagccgatcatcagcacatctaggtttcgtccgggtgtgaccgaaaggtaagatggagagccttgtccctggtttcaacgagaaaac
acacgtccaactcagtttgcctgttttacaggttcgcgacgtgctcgtacgtggctttggagactccgtggaggaggtcttatcagaggcacgtcaac
atcttaaagatggcacttgtggcttagtagaagttgaaaaaggcgttttgcctcaacttgaacagccctatgtgttcatcaaacgttcggatgctcgaac
tgcacctcatggtcatgttatggttgagctggtagcagaactcgaaggcattcagtacggtcgtagtggtgagacacttggtgtccttgtccctcatgt
gggcgaaataccagtggcttaccgcaaggttcttcttcgtaagaacggtaataaaggagctggtggccatagttacggcgccgatctaaagtcattt
gacttaggcgacgagcttggcactgatccttatgaagattttcaagaaaactggaacactaaacatagcagtggtgttacccgtgaactcatgcgtga
gcttaacggaggggcatacactcgctatgtcgataacaacttctgtggccctgatggctaccctcttgagtgcattaaagaccttctagcacgtgctg
gtaaagcttcatgcactttgtccgaacaactggactttattgacactaagaggggtgtatactgctgccgtgaacatgagcatgaaattgcttggtaca
cggaacgttctgaaaagagctatgaattgcagacaccttttgaaattaaattggcaaagaaatttgacaccttcaatggggaatgtccaaattttgtattt
cccttaaattccataatcaagactattcaaccaagggttgaaaagaaaaagcttgatggctttatgggtagaattcgatctgtctatccagttgcgtcac
caaatgaatgcaaccaaatgtgcctttcaactctcatgaagtgtgatcattgtggtgaaacttcatggcagacgggcgattttgttaaagccacttgcg
aattttgtggcactgagaatttgactaaagaaggtgccactacttgtggttacttaccccaaaatgctgttgttaaaatttattgtccagcatgtcacaattc
agaagtaggacctgagcatagtcttgccgaataccataatgaatctggcttgaaaaccattcttcgtaagggtggtcgcactattgcctttggaggctg
tgtgttctcttatgttggttgccataacaagtgtgcctattgggttccacgtgctagcgctaacataggttgtaaccatacaggtgttgttggagaaggtt
ccgaaggtcttaatgacaaccttcttgaaatactccaaaaagagaaagtcaacatcaatattgttggtgactttaaacttaatgaagagatcgccattatt
ttggcatctttttctgcttccacaagtgcttttgtggaaactgtgaaaggtttggattataaagcattcaaacaaattgttgaatcctgtggtaattttaaagtt
acaaaaggaaaagctaaaaaaggtgcctggaatattggtgaacagaaatcaatactgagtcctctttatgcatttgcatcagaggctgctcgtgttgta
cgatcaattttctcccgcactcttgaaactgctcaaaattctgtgcgtgttttacagaaggccgctataacaatactagatggaatttcacagtattcactg
agactcattgatgctatgatgttcacatctgatttggctactaacaatctagttgtaatggcctacattacaggtggtgttgttcagttgacttcgcagtgg
ctaactaacatctttggcactgtttatgaaaaactcaaacccgtccttgattggcttgaagagaagtttaaggaaggtgtagagtttcttagagacggtt
gggaaattgttaaatttatctcaacctgtgcttgtgaaattgtcggtggacaaattgtcacctgtgcaaaggaaattaaggagagtgttcagacattcttt
aagcttgtaaataaatttttggctttgtgtgctgactctatcattattggtggagctaaacttaaagccttgaatttaggtgaaacatttgtcacgcactcaa
agggattgtacagaaagtgtgttaaatccagagaagaaactggcctactcatgcctctaaaagccccaaaagaaattatcttcttagagggagaaac
acttcccacagaagtgttaacagaggaagttgtcttgaaaactggtgatttacaaccattagaacaacctactagtgaagctgttgaagctccattggtt
ggtacaccagtttgtattaacgggcttatgttgctcgaaatcaaagacacagaaaagtactgtgcccttgcacctaatatgatggtaacaaacaatacc
ttcacactcaaaggcggtgcaccaacaaaggttacttttggtgatgacactgtgatagaagtgcaaggttacaagagtgtgaatatcacttttgaactt
gatgaaaggattgataaagtacttaatgagaagtgctctgcctatacagttgaactcggtacagaagtaaatgagttcgcctgtgttgtggcagatgct
gtcataaaaactttgcaaccagtatctgaattacttacaccactgggcattgatttagatgagtggagtatggctacatactacttatttgatgagtctggt
gagtttaTattggcttcacatatgtattgttctttctaccctccagatgaggatgaagaagaaggtgattgtgaagaagaagagtttgagccatcaactc
aatatgagtatggtactgaagatgattaccaaggtaaacctttggaatttggtgccacttctgctgctcttcaacctgaagaagagcaagaagaagatt
ggttagatgatgatagtcaacaaactgttggtcaacaagacggcagtgaggacaatcagacaactactattcaaacaattgttgaggttcaacctcaa
ttagagatggaacttacaccagttgttcagactattgaagtgaatagttttagtggttatttaaaacttactgacaatgtatacattaaaaatgcagacattg
tggaagaagctaaaaaggtaaaaccaacagtggttgttaatgcagccaatgtttaccttaaacatggaggaggtgttgcaggagccttaaataaggc
tactaacaatgccatgcaagttgaatctgatgattacatagctactaatggaccacttaaagtgggtggtagttgtgttttaagcggacacaatcttgcta
aacactgtcttcatgttgtcggcccaaatgttaacaaaggtgaagacattcaacttcttaagagtgcttatgaaaattttaatcagcacgaagttctacttg
caccattattatcagctggtatttttggtgctgaccctatacattctttaagagtttgtgtagatactgttcgcacaaatgtctacttagctgtctttgataaaa
atctctatgacaaacttgtttcaagctttttggaaatgaagagtgaaaagcaagttgaacaaaagatcgctgagattcctaaagaggaagttaagccat
ttataactgaaagtaaaccttcagttgaacagagaaaacaagatgataagaaaatcaaagcttgtgttgaagaagttacaacaactctggaagaaact
aagttcctcacagaaaacttgttactttatattgacattaatggcaatcttcatccagattctgccactcttgttagtgacattgacatcactttcttaaagaa
agatgctccatatatagtgggtgatgttgttcaagagggtgttttaactgctgtggttatacctactaaaaaggctggtggcactactgaaatgctagcg
aaagctttgagaaaagtgccaacagacaattatataaccacttacccgggtcagggtttaaatggttacactgtagaggaggcaaagacagtgctta
aaaagtgtaaaagtgccttttacattctaccatctattatctctaatgagaagcaagaaattcttggaactgtttcttggaatttgcgagaaatgcttgcac
atgcagaagaaacacgcaaattaatgcctgtctgtgtggaaactaaagccatagtttcaactatacagcgtaaatataagggtattaaaatacaagag
ggtgtggttgattatggtgctagattttacttttacaccagtaaaacaactgtagcgtcacttatcaacacacttaacgatctaaatgaaactcttgttacaa
tgccacttggctatgtaacacatggcttaaatttggaagaagctgctcggtatatgagatctctcaaagtgccagctacagtttctgtttcttcacctgat
gctgttacagcgtataatggttatcttacttcttcttctaaaacacctgaagaacattttattgaaaccatctcacttgctggttcctataaagattggtcctat
tctggacaatctacacaactaggtatagaatttcttaagagaggtgataaaagtgtatattacactagtaatcctaccacattccacctagatggtgaagt
tatcacctttgacaatcttaagacacttctttctttgagagaagtgaggactattaaggtgtttacaacagtagacaacattaacctccacacgcaagttg
tggacatgtcaatgacatatggacaacagtttggtccaacttatttggatggagctgatgttactaaaataaaacctcataattcacatgaaggtaaaac
attttatgttttacctaatgatgacactctacgtgttgaggcttttgagtactaccacacaactgatcctagttttctgggtaggtacatgtcagcattaaatc
acactaaaaagtggaaatacccacaagttaatggtttaacttctattaaatgggcagataacaactgttatcttgccactgcattgttaacactccaacaa
atagagttgaagtttaatccacctgctctacaagatgcttattacagagcaagggctggtgaagctgctaacttttgtgcacttatcttagcctactgtaat
aagacagtaggtgagttaggtgatgttagagaaacaatgagttacttgtttcaacatgccaatttagattcttgcaaaagagtcttgaacgtggtgtgta
aaacttgtggacaacagcagacaacccttaagggtgtagaagctgttatgtacatgggcacactttcttatgaacaatttaagaaaggtgttcagatac
cttgtacgtgtggtaaacaagctacaaaatatctagtacaacaggagtcaccttttgttatgatgtcagcaccacctgctcagtatgaacttaagcatgg
tacatttacttgtgctagtgagtacactggtaattaccagtgtggtcactataaacatataacttctaaagaaactttgtattgcatagacggtgctttactt
acaaagtcctcagaatacaaaggtcctattacggatgttttctacaaagaaaacagttacacaacaaccataaaaccagttacttataaattggatggt
gttgtttgtacagaaattgaccctaagttggacaattattataagaaagacaattcttatttcacagagcaaccaattgatcttgtaccaaaccaaccatat
ccaaacgcaagcttcgataattttaagtttgtatgtgataatatcaaatttgctgatgatttaaaccagttaactggttataagaaacctgcttcaagagag
cttaaagttacatttttccctgacttaaatggtgatgtggtggctattgattataaacactacacaccctcttttaagaaaggagctaaattgttacataaac
ctattgtttggcatgttaacaatgcaactaataaagccacgtataaaccaaatacctggtgtatacgttgtctttggagcacaaaaccagttgaaacatc
aaattcgtttgatgtactgaagtcagaggacgcgcagggaatggataatcttgcctgcgaagatctaaaaccagtctctgaagaagtagtggaaaat
cctaccatacagaaagacgttcttgagtgtaatgtgaaaactaccgaagttgtaggagacattatacttaaaccagcaaataatagtttaaaaattacag
aagaggttggccacacagatctaatggctgcttatgtagacaattctagtcttactattaagaaacctaatgaattatctagagtattaggtttgaaaacc
cttgctactcatggtttagctgctgttaatagtgtcccttgggatactatagctaattatgctaagccttttcttaacaaagttgttagtacaactactaacat
agttacacggtgtttaaaccgtgtttgtactaattatatgccttatttctttactttattgctacaattgtgtacttttactagaagtacaaattctagaatta
aagcatctatgccgactactatagcaaagaatactgttaagagtgtcggtaaattttgtctagaggcttcatttaattatttgaagtcacctaatttttctaaa
ctgataaatattataatttggtttttactattaagtgtttgcctaggttctttaatctactcaaccgctgctttaggtgttttaatgtctaatttaggcatgcc
ttcttactgtactggttacagagaaggctatttgaactctactaatgtcactattgcaacctactgtactggttctataccttgtagtgtttgtcttagtggtt
tagattctttagacacctatccttctttagaaactatacaaattaccatttcatcttttaaatgggatttaactgcttttggcttagttgcagagtggtttttg
gcatatattcttttcactaggtttttctatgtacttggattggctgcaatcatgcaattgtttttcagctattttgcagtacattttattagtaattcttggctt
atgtggttaataattaatcttgtacaaatggccccgatttcagctatggttagaatgtacatcttctttgcatcattttattatgtatggaaaagttatgtgcat
gttgtagacggttgtaattcatcaacttgtatgatgtgttacaaacgtaatagagcaacaagagtcgaatgtacaactattgttaatggtgttagaaggtcctt
ttatgtctatgctaatggaggtaaaggcttttgcaaactacacaattggaattgtgttaattgtgatacattctgtgctggtagtacatttattagtgatgaag
ttgcgagagacttgtcactacagtttaaaagaccaataaatcctactgaccagtcttcttacatcgttgatagtgttacagtgaagaatggttccatccatctt
tactttgataaagctggtcaaaagacttatgaaagacattctctctctcattttgttaacttagacaacctgagagctaataacactaaaggttcattgcctat
taatgttatagtttttgatggtaaatcaaaatgtgaagaatcatctgcaaaatcagcgtctgtttactacagtcagcttatgtgtcaacctatactgttactag
atcaggcattagtgtctgatgttggtgatagtgcggaagttgcagttaaaatgtttgatgcttacgttaatacgttttcatcaacttttaacgtaccaatggaa
aaactcaaaacactagttgcaactgcagaagctgaacttgcaaagaatgtgtccttagacaatgtcttatctacttttatttcagcagctcggcaagggtttgt
tgattcagatgtagaaactaaagatgttgttgaatgtcttaaattgtcacatcaatctgacatagaagttactggcgatagttgtaataactatatgctcacct
ataacaaagttgaaaacatgacaccccgtgaccttggtgcttgtattgactgtagtgcgcgtcatattaatgcgcaggtagcaaaaagtcacaacattgct
ttgatatggaacgttaaagatttcatgtcattgtctgaacaactacgaaaacaaatacgtagtgctgctaaaaagaataacttaccttttaagttgacatgt
gcaactactagacaagttgttaatgttgtaacaacaaagatagcacttaagggtggtaaaattgttaataattggttgaagcagttaattaaagttacact
tgtgttcctttttgttgctgctattttctatttaataacacctgttcatgtcatgtctaaacatactgacttttcaagtgaaatcataggatacaaggctattg
atggtggtgtcactcgtgacatagcatctacagatacttgttttgctaacaaacatgctgattttgacacatggtttagtcagcgtggtggtagttatactaat
gacaaagcttgcccattgattgctgcagtcataacaagagaagtgggttttgtcgtgcctggtttgcctggcacgatattacgcacaactaatggtgac
tttttgcatttcttacctagagtttttagtgcagttggtaacatctgttacacaccatcaaaacttatagagtacactgactttgcaacatcagcttgtgttttg
gctgctgaatgtacaatttttaaagatgcttctggtaagccagtaccatattgttatgataccaatgtactagaaggttctgttgcttatgaaagtttacgcc
ctgacacacgttatgtgctcatggatggctctattattcaatttcctaacacctaccttgaaggttctgttagagtggtaacaacCtttgattctgagtact
gtaggcacggcacttgtgaaagatcagaagctggtgtttgtgtatctactagtggtagatgggtacttaacaatgattattacagatctttaccaggagt
tttctgtggtgtagatgctgtaaatttacttactaatatgtttacaccactaattcaacctattggtgctttggacatatcagcatctatagtagctggtggtat
tgtagctatcgtagtaacatgccttgcctactattttatgaggtttagaagagcttttggtgaatacagtcatgtagttgcctttaatactttactattccttat
gtcattcactgtactctgtttaacaccagtttactcattcttacctggtgtttattctgttatttacttgtacttgacattttatcttactaatgatgtttcttt
tttagcacatattcagtggatggttatgttcacacctttagtacctttctggataacaattgcttatatcatttgtatttccacaaagcatttctattggttctt
tagtaattacctaaagagacgtgtagtctttaatggtgtttcctttagtacttttgaagaagctgcgctgtgcacctttttgttaaataaagaaatgtatctaaa
gttgcgtagtgatgtgctattacctcttacgcaatataatagatacttagctctttataataagtacaagtattttagtggagcaatggatacaactagctacag
agaagctgcttgttgtcatctcgcaaaggctctcaatgacttcagtaactcaggttctgatgttctttaccaaccaccacaaacctctatcacctcagctgtttt
gcagagtggttttagaaaaatggcattcccatctggtaaagttgagggttgtatggtacaagtaacttgtggtacaactacacttaacggtctttggctt
gatgacgtagtttactgtccaagacatgtgatctgcacctctgaagacatgcttaaccctaattatgaagatttactcattcgtaagtctaatcataatttct
tggtacaggctggtaatgttcaactcagggttattggacattctatgcaaaattgtgtacttaagcttaaggttgatacagccaatcctaagacacctaa
gtataagtttgttcgcattcaaccaggacagactttttcagtgttagcttgttacaatggttcaccatctggtgtttaccaatgtgctatgaggcccaatttc
actattaagggttcattccttaatggttcatgtggtagtgttggttttaacatagattatgactgtgtctctttttgttacatgcaccatatggaattaccaact
ggagttcatgctggcacagacttagaaggtaacttttatggaccttttgttgacaggcaaacagcacaagcagctggtacggacacaactattacagt
taatgttttagcttggttgtacgctgctgttataaatggagacaggtggtttctcaatcgatttaccacaactcttaatgactttaaccttgtggctatgaagt
acaattatgaacctctaacacaagaccatgttgacatactaggacctctttctgctcaaactggaattgccgttttagatatgtgtgcttcattaaaagaat
tactgcaaaatggtatgaatggacgtaccatattgggtagtgctttattagaagatgaatttacaccttttgatgttgttagacaatgctcaggtgttacttt
ccaaagtgcagtgaaaagaacaatcaagggtacacaccactggttgttactcacaattttgacttcacttttagttttagtccagagtactcaatggtcttt
gttcttttttttgtatgaaaatgcctttttaccttttgctatgggtattattgctatgtctgcttttgcaatgatgtttgtcaaacataagcatgcatttctctg
tttgtttttgttaccttctcttgccactgtagcttattttaatatggtctatatgcctgctagttgggtgatgcgtattatgacatggttggatatggttgatac
tagtttgtctggttttaagctaaaagactgtgttatgtatgcatcagctgtagtgttactaatccttatgacagcaagaactgtgtatgatgatggtgctaggag
agtgtggacacttatgaatgtcttgacactcgtttataaagtttattatggtaatgctttagatcaagccatttccatgtgggctcttataatctctgttacttc
taactactcaggtgtagttacaactgtcatgttcttggccagaggtattgtttttatgtgtgttgagtattgccctattttcttcataactggtaatacacttca
gtgtataatgctagtttattgtttcttaggctatttttgtacttgttactttggcctcttttgtttactcaaccgctactttagactgactcttggtgtttatga
ttacttagtttctacacaggagtttagatatatgaattcacagggactactcccacccaagaatagcatagatgccttcaaactcaacattaaattgttgggtgt
tggtggcaaaccttgtatcaaagtagccactgtacagtctaaaatgtcagatgtaaagtgcacatcagtagtcttactctcagttttgcaacaactcagagtag
aatcatcatctaaattgtgggctcaatgtgtccagttacacaatgacattctcttagctaaagatactactgaagcctttgaaaaaatggtttcactactttc
tgttttgctttccatgcagggtgctgtagacataaacaagctttgtgaagaaatgctggacaacagggcaaccttacaagctatagcctcagagtttag
ttcccttccatcatatgcagcttttgctactgctcaagaagcttatgagcaggctgttgctaatggtgattctgaagttgttcttaaaaagttgaagaagtct
ttgaatgtggctaaatctgaatttgaccgtgatgcagccatgcaacgtaagttggaaaagatggctgatcaagctatgacccaaatgtataaacaggc
tagatctgaggacaagagggcaaaagttactagtgctatgcagacaatgcttttcactatgcttagaaagttggataatgatgcactcaacaacattat
caacaatgcaagagatggttgtgttcccttgaacataatacctcttacaacagcagccaaactaatggttgtcataccagactataacacatataaaaat
acgtgtgatggtacaacatttacttatgcatcagcattgtgggaaatccaacaggttgtagatgcagatagtaaaattgttcaacttagtgaaattagtat
ggacaattcacctaatttagcatggcctcttattgtaacagctttaagggccaattctgctgtcaaattacagaataatgagcttagtcctgttgcactacg
acagatgtcttgtgctgccggtactacacaaactgcttgcactgatgacaatgcgttagcttactacaacacaacaaagggaggtaggtttgtacttgc
actgttatccgatttacaggatttgaaatgggctagattccctaagagtgatggaactggtactatctatacagaactggaaccaccttgtaggtttgtta
cagacacacctaaaggtcctaaagtgaagtatttatactttattaaaggattaaacaacctaaatagaggtatggtacttggtagtttagctgccacagt
acgtctacaagctggtaatgcaacagaagtgcctgccaattcaactgtattatctttctgtgcttttgctgtagatgctgctaaagcttacaaagattatct
agctagtgggggacaaccaatcactaattgtgttaagatgttgtgtacacacactggtactggtcaggcaataacagttacaccggaagccaatatg
gatcaagaatcctttggtggtgcatcgtgttgtctgtactgccgttgccacatagatcatccaaatcctaaaggattttgtgacttaaaaggtaagtatgt
acaaatacctacaacttgtgctaatgaccctgtgggttttacacttaaaaacacagtctgtaccgtctgcggtatgtggaaaggttatggctgtagttgt
gatcaactccgcgaacccatgcttcagtcagctgatgcacaatcgtttttaaacgggtttgcggtgtaagtgcagcccgtcttacaccgtgcggcaca
ggcactagtactgatgtcgtatacagggcttttgacatctacaatgataaagtagctggttttgctaaattcctaaaaactaattgttgtcgcttccaagaa
aaggacgaagatgacaatttaattgattcttactttgtagttaagagacacactttctctaactaccaacatgaagaaacaatttataatttacttaaggatt
gtccagctgttgctaaacatgacttctttaagtttagaatagacggtgacatggtaccacatatatcacgtcaacgtcttactaaatacacaatggcaga
cctcgtctatgctttaaggcattttgatgaaggtaattgtgacacattaaaagaaatacttgtcacatacaattgttgtgatgatgattatttcaataaaaag
gactggtatgattttgtagaaaacccagatatattacgcgtatacgccaacttaggtgaacgtgtacgccaagctttgttaaaaacagtacaattctgtg
atgccatgcgaaatgctggtattgttggtgtactgacattagataatcaagatctcaatggtaactggtatgatttcggtgatttcatacaaaccacgcca
ggtagtggagttcctgttgtagattcttattattcattgttaatgcctatattaaccttgaccagggctttaactgcagagtcacatgttgacactgacttaa
caaagccttacattaagtgggatttgttaaaatatgacttcacggaagagaggttaaaactctttgaccgttattttaaatattgggatcagacataccac
ccaaattgtgttaactgtttggatgacagatgcattctgcattgtgcaaactttaatgttttattctctacagtgttcccacctacaagttttggaccactagt
gagaaaaatatttgttgatggtgttccatttgtagtttcaactggataccacttcagagagctaggtgttgtacataatcaggatgtaaacttacatagctc
tagacttagttttaaggaattacttgtgtatgctgctgaccctgctatgcacgctgcttctggtaatctattactagataaacgcactacgtgcttttcagta
gctgcacttactaacaatgttgcttttcaaactgtcaaacccggtaattttaacaaagacttctatgactttgctgtgtctaagggtttctttaaggaaggaa
gttctgttgaattaaaacacttcttctttgctcaggatggtaatgctgctatcagcgattatgactactatcgttataatctaccaacaatgtgtgatatcag
acaactactatttgtagttgaagttgttgataagtactttgattgttacgatggtggctgtattaatgctaaccaagtcatcgtcaacaacctagacaaatc
agctggttttccatttaataaatggggtaaggctagactttattatgattcaatgagttatgaggatcaagatgcacttttcgcatatacaaaacgtaatgt
catccctactataactcaaatgaatcttaagtatgccattagtgcaaagaatagagctcgcaccgtagctggtgtctctatctgtagtactatgaccaata
gacagtttcatcaaaaattattgaaatcaatagccgccactagaggagctactgtagtaattggaacaagcaaattctatggtggttggcacaacatgt
taaaaactgtttatagtgatgtagaaaaccctcaccttatgggttgggattatcctaaatgtgatagagccatgcctaacatgcttagaattatggcctca
cttgttcttgctcgcaaacatacaacgtgttgtagcttgtcacaccgtttctatagattagctaatgagtgtgctcaagtattgagtgaaatggtcatgtgt
ggcggttcactatatgttaaaccaggtggaacctcatcaggagatgccacaactgcttatgctaatagtgtttttaacatttgtcaagctgtcacggcca
atgttaatgcacttttatctactgatggtaacaaaattgccgataagtatgtccgcaatttacaacacagactttatgagtgtctctatagaaatagagatg
ttgacacagactttgtgaatgagttttacgcatatttgcgtaaacatttctcaatgatgatactctctgacgatgctgttgtgtgtttcaatagcacttatgca
tctcaaggtctagtggctagcataaagaactttaagtcagttctttattatcaaaacaatgtttttatgtctgaagcaaaatgttggactgagactgacctta
ctaaaggacctcatgaattttgctctcaacatacaatgctagttaaacagggtgatgattatgtgtaccttccttacccagatccatcaagaatcctaggg
gccggctgttttgtagatgatatcgtaaaaacagatggtacacttatgattgaacggttcgtgtctttagctatagatgcttacccacttactaaacatcct
aatcaggagtatgctgatgtctttcatttgtacttacaatacataagaaagctacatgatgagttaacaggacacatgttagacatgtattctgttatgctta
ctaatgataacacttcaaggtattgggaacctgagttttatgaggctatgtacacaccgcatacagtcttacaggctgttggggcttgtgttctttgcaatt
cacagacttcattaagatgtggtgcttgcatacgtagaccattcttatgttgtaaatgctgttacgaccatgtcatatcaacatcacataaattagtcttgtc
tgttaatccgtatgtttgcaGtgctccaggttgtgatgtcacagatgtgactcaactttacttaggaggtatgagctattattgtaaatcacataaaccac
ccattagttttccattgtgtgctaatggacaagtttttggtttatataaaaatacatgtgttggtagcgataatgttactgactttaatgcaattgcaacatgtg
actggacaaatgctggtgattacattttagctaacacctgtactgaaagactcaagctttttgcagcagaaacgctcaaagctactgaggagacattta
aactgtcttatggtattgctactgtacgtgaagtgctgtctgacagagaattacatctttcatgggaagttggtaaacctagaccaccacttaaccgaaa
ttatgtctttactggttatcgtgtaactaaaaacagtaaagtacaaataggagagtacacctttgaaaaaggtgactatggtgatgctgttgtttaccgag
gtacaacaacttacaaattaaatgttggtgattattttgtgctgacatcacatacagtaatgccattaagtgcacctacactagtgccacaagagcactat
gttagaattactggcttatacccaacactcaatatctcagatgagttttctagcaatgttgcaaattatcaaaaggttggtatgcaaaagtattctacactc
cagggaccacctggtactggtaagagtcattttgctattggcctagctctctactacccttctgctcgcatagtgtatacagcttgctctcatgccgctgt
tgatgcactatgtgagaaggcattaaaatatttgcctatagataaatgtagtagaattatacctgcacgtgctcgtgtagagtgttttgataaattcaaagt
gaattcaacattagaacagtatgtcttttgtactgtaaatgcattgcctgagacgacagcagatatagttgtctttgatgaaatttcaatggccacaaatta
tgatttgagtgttgtcaatgccagattacgtgctaagcactatgtgtacattggcgaccctgctcaattacctgcaccacgcacattgctaactaagggc
acactagaaccagaatatttcaattcagtgtgtagacttatgaaaactataggtccagacatgttcctcggaacttgtcggcgttgtcctgctgaaattgt
tgacactgtgagtgctttggtttatgataataagcttaaagcacataaagacaaatcagctcaatgctttaaaatgttttataagggtgttatcacgcatga
tgtttcatctgcaattaacaggccacaaataggcgtggtaagagaattccttacacgtaaccctgcttggagaaaagctgtctttatttcaccttataattc
acagaatgctgtagcctcaaagattttgggactaccaactcaaactgttgattcatcacagggctcagaatatgactatgtcatattcactcaaaccact
gaaacagctcactcttgtaatgtaaacagatttaatgttgctattaccagagcaaaagtaggcatactttgcataatgtctgatagagacctttatgacaa
gttgcaatttacaagtcttgaaattccacgtaggaatgtggcaactttacaagctgaaaatgtaacaggactttttaaagattgtagtaaggtaatcactg
ggttacatcctacacaggcacctacacacctcagtgttgacactaaattcaaaactgaaggtttatgtgttgacatacctggcatacctaaggacatga
cctatagaagactcatctctatgatgggttttaaaatgaattatcaagttaatggttaccctaacatgtttatcacccgcgaagaagctataagacatgta
cgtgcatggattggcttcgatgtcgaggggtgtcatgctactagagaagctgttggtaccaatttacctttacagctaggtttttctacaggtgttaacct
agttgctgtacctacaggttatgttgatacacctaataatacagatttttccagagttagtgctaaaccaccgcctggagatcaatttaaacacctcatac
cacttatgtacaaaggacttccttggaatgtagtgcgtataaagattgtacaaatgttaagtgacacacttaaaaatctctctgacagagtcgtatttgtct
tatgggcacatggctttgagttgacatctatgaagtattttgtgaaaataggacctgagcgcacctgttgtctatgtgatagacgtgccacatgcttttcc
actgcttcagacacttatgcctgttggcatcattctattggatttgattacgtctataatccgtttatgattgatgttcaacaatggggttttacaggtaacct
acaaagcaaccatgatctgtattgtcaagtccatggtaatgcacatgtagctagttgtgatgcaatcatgactaggtgtctagctgtccacgagtgcttt
gttaagcgtgttgactggactattgaatatcctataattggtgatgaactgaagattaatgcggcttgtagaaaggttcaacacatggttgttaaagctgc
attattagcagacaaattcccagttcttcacgacattggtaaccctaaagctattaagtgtgtacctcaagctgatgtagaatggaagttctatgatgcac
agccttgtagtgacaaagcttataaaatagaagaattattctattcttatgccacacattctgacaaattcacagatggtgtatgcctattttggaattgcaa
tgtcgatagatatcctgctaattccattgtttgtagatttgacactagagtgctatctaaccttaacttgcctggttgtgatggtggcagtttgtatgtaaata
aacatgcattccacacaccagcttttgataaaagtgcttttgttaatttaaaacaattaccatttttctattactctgacagtccatgtgagtctcatggaaaa
caagtagtgtcagatatagattatgtaccactaaagtctgctacgtgtataacacgttgcaatttaggtggtgctgtctgtagacatcatgctaatgagta
cagattgtatctcgatgcttataacatgatgatctcagctggctttagcttgtgggtttacaaacaatttgatacttataacctctggaacacttttacaaga
cttcagagtttagaaaatgtggcttttaatgttgtaaataagggacactttgatggacaacagggtgaagtaccagtttctatcattaataacactgtttac
acaaaagttgatggtgttgatgtagaattgtttgaaaataaaacaacattacctgttaatgtagcatttgagctttgggctaagcgcaacattaaaccagt
accagaggtgaaaatactcaataatttgggtgtggacattgctgctaatactgtgatctgggactacaaaagagatgctccagcacatatatctactatt
ggtgtttgttctatgactgacatagccaagaaaccaactgaaacgatttgtgcaccactcactgtcttttttgatggtagagttgatggtcaagtagactt
atttagaaatgcccgtaatggtgttcttattacagaaggtagtgttaaaggtttacaaccatctgtaggtcccaaacaagctagtcttaatggagtcacat
taattggagaagccgtaaaaacacagttcaattattataagaaagttgatggtgttgtccaacaattacctgaaacttactttactcagagtagaaattta
caagaatttaaacccaggagtcaaatggaaattgatttcttagaattagctatggatgaattcattgaacggtataaattagaaggctatgccttcgaac
atatcgtttatggagattttagtcatagtcagttaggtggtttacatctactgattggactagctaaacgttttaaggaatcaccttttgaattagaagatttta
ttcctatggacagtacagttaaaaactatttcataacagatgcgcaaacaggttcatctaagtgtgtgtgttctgttattgatttattacttgatgattttgttg
aaataataaaatcccaagatttatctgtagtttctaaggttgtcaaagtgactattgactatacagaaatttcatttatgctttggtgtaaagatggccatgt
agaaacattttacccaaaattacaatctagtcaagcgtggcaaccgggtgttgctatgcctaatctttacaaaatgcaaagaatgctattagaaaagtgt
gaccttcaaaattatggtgatagtgcaacattacctaaaggcataatgatgaatgtcgcaaaatatactcaactgtgtcaatatttaaacacattaacatt
agctgtaccctataatatgagagttatacattttggtgctggttctgataaaggagttgcaccaggtacagctgttttaagacagtggttgcctacgggt
acgctgcttgtcgattcagatcttaatgactttgtctctgatgcagattcaactttgattggtgattgtgcaactgtacatacagctaataaatgggatctca
ttattagtgatatgtacgaccctaagactaaaaatgttacaaaagaaaatgactctaaagagggttttttcacttacatttgtgggtttatacaacaaaagc
tagctcttggaggttccgtggctataaagataacagaacattcttggaatgctgatctttataagctcatgggacacttcgcatggtggacagcctttgtt
actaatgtgaatgcgtcatcatctgaagcatttttaattggatgtaattatcttggcaaaccacgcgaacaaatagatggttatgtcatgcatgcaaattac
atattttggaggaatacaaatccaattcagttgtcttcctattctttatttgacatgagtaaatttccccttaaattaaggggtactgctgttatgtctttaaaa
gaaggtcaaatcaatgatatgattttatctcttcttagtaaaggtagacttataattagagaaaacaacagagttgttatttctagtgatgttcttgttaacaa
ctaaacgaacaatgtttgtttttcttgttttattgccactagtctctagtcagtgtgttaatcttacaaccagaactcaattaccccctgcatacactaattctt
tcacacgtggtgtttattaccctgacaaagttttcagatcctcagttttacattcaactcaggacttgttcttacctttcttttccaatgttacttggttccatg
ctatacatgtctctgggaccaatggtactaagaggtttgataaccctgtcctaccatttaatgatggtgtttattttgcttccaTtgagaagtctaacataata
agaggctggatttttggtactactttagattcgaagacccagtccctacttattgttaataacgctactaatgttgttattaaagtctgtgaatttcaattttgt
aatgatccatttttgggtgtttattaccacaaaaacaacaaaagttggatggaaagtgagttcagagtttattctagtgcgaataattgcacttttgaatat
gtctctcagccttttcttatggaccttgaaggaaaacagggtaatttcaaaaatcttagggaatttgtgtttaagaatattgatggttattttaaaatatattct
aagcacacgcctattaatttagtgcgtgatctccctcagggtttttcggctttagaaccattggtagatttgccaataggtattaacatcactaggtttcaa
actttacttgctttacGtagaagttatttgactcctggtgattcttcttcaggttggacagctggtgctgcagcttattatgtgggttatcttcaacctagga
cttttctattaaaatataatgaaaatggaaccattacagatgctgtagactgtgcacttgaccctctctcagaaacaaagtgtacgttgaaatccttcactg
tagaaaaaggaatctatcaaacttctaactttagagtccaaccaacagaatctattgttagatttcctaatattacaaacttgtgcccttttggtgaagttttt
aacgccaccagatttgcatctgtttatgcttggaacaggaagagaatcagcaactgtgttgctgattattctgtcctatataattccgcatcattttccactt
ttaagtgttatggagtgtctcctactaaattaaatgatctctgctttactaatgtctatgcagattcatttgtaattagaggtgatgaagtcagacaaatcgct
ccagggcaaactggaaagattgctgattataattataaattaccagatgattttacaggctgcgttatagcttggaattctaacaatcttgattctaaggtt
ggtggtaattataattacctgtatagattgtttaggaagtctaatctcaaaccttttgagagagatatttcaactgaaatctatcaggccggtagcacacct
tgtaatggtgttgaaggttttaattgttactttcctttacaatcatatggtttccaacccactaatggtgttggttaccaaccatacagagtagtagtactttct
tttgaacttctacatgcaccagcaactgtttgtggacctaaaaagtctactaatttggttaaaaacaaatgtgtcaatttcaacttcaatggtttaacaggc
acaggtgttcttactgagtctaacaaaaagtttctgcctttccaacaatttggcagagacattgctgacactactgatgctgtccgtgatccacagacac
ttgagattcttgacattacaccatgttcttttggtggtgtcagtgttataacaccaggaacaaatacttctaaccaggttgctgttctttatcaggatgttaac
tgcacagaagtccctgttgctattcatgcagatcaacttactcctacttggcgtgtttattctacaggttctaatgtttttcaaacacgtgcaggctgtttaat
aggggctgaacatgtcaacaactcatatgagtgtgacatacccattggtgcaggtatatgcgctagttatcagactcagcaatccatcattgcctacac
tatgtcacttggtgcagaaaattcagttgcttactctaataactctattgccatacccacaaattttactattagtgttaccacagaaattctaccagtgtcta
tgaccaagacatcagtagattgtacaatgtacatttgtggtgattcaactgaatgcagcaatcttttgttgcaatatggcagtttttgtacacaattaaacc
gtgctttaactggaatagctgttgaacaagacaaaaacacccaagaagtttttgcacaagtcaaacaaatttacaaaacaccaccaattaaagattttg
gtggttttaatttttcacaaatattaccagatccatcaaaaccaagcaagaggtcatttattgaagatctacttttcaacaaagtgacacttgcagatgctg
gcttcatcaaacaatatggtgattgccttggtgatattgctgctagagatctcatttgcgctcaaaaatttaacggacttacagttttaccacctttacttact
gacgaaatgattgcgcaatatacatccgcattgttagccggaactattacatccggatggacttttggcgcaggcgTagcattacagattccattcgc
tatgcaaatggcttataggtttaacggtataggcgttacgcaaaacgtactttatgagaatcaaaaacttatcgctaaccaatttaattccgctatcggta
agattcaggattcattgtctagtactgctagtgcactcggtaagttgcaagacgtagtgaatcaaaacgctcaagcacttaatacactcgttaaacagct
tagttctaattttggcgcaatttctagtgtgcttaacgatatactatctagactcgataaagtcgaagccgaagtgcaaatcgatagattgattaccggta
ggttgcaatcattgcaaacatacgttacacagcaattgattagggccgcagagatacgcgctagcgctaatctcgcagctactaaaatgtctgaatgc
gtactcggacaatctaaacgtgtcgatttttgcggtaagggatatcatcttatgtcttttccacaatctgcacctcacggagtcgtgtttttacacgttactt
atgtgccagctcaagagaaaaattttacaaccgctcctgctatttgtcatgacggtaaggcacattttcctagagagggcgtattcgtttctaacggtac
acattggttcgttacacaacgtaatttttacgaacctcaaattattactactgataatacattcgtatcaggtaattgtgacgtagtgataggtatcgttaata
atacagtttacgatccacttcaacctgaactcgatagttttaaagaggaactcgataagtattttaaaaatcatacatcacctgacgtcgacttaggcgat
atttcaggtattaacgctagtgtcgttaacattcaaaaagagattgatagacttaacgaagtcgctaaaaatcttaacgaatcacttatcgatctgcaaga
gttaggtaagtatgagcaatatattaaatggccttggtatatttggttaggctttatagccggattgatcgcaatcgttatggttacaattatgttatgttgta
tgacatcatgttgttcatgtcttaagggatgttgttcatgcggatcatgttgtaaatttgacgaagacgattccgaaccagtgcttaaaggcgttaagtta
cattatacataaacgaacttatggatttgtttatgagaatcttcacaattggaactgtaactttgaagcaaggtgaaatcaaggatgctactccttcagatt
ttgttcgcgctactgcaacgataccgatacaagcctcactccctttcggatggcttattgttggcgttgcacttcttgctgtttttcagagcgcttccaaaa
tcataaccctcaaaaagagatggcaactagcactctccaagggtgttcactttgtttgcaacttgctgttgttgtttgtaacagtttactcacaccttttgct
cgttgctgctggccttgaagccccttttctctatctttatgctttagtctacttcttgcagagtataaactttgtaagaataataatgaggctttggctttgctg
gaaatgccgttccaaaaacccattactttatgatgccaactattttctttgctggcatactaattgttacgactattgtataccttacaatagtgtaacttcttc
aattgtcattacttcaggtgatggcacaacaagtcctatttctgaacatgactaccagattggtggttatactgaaaaatgggaatctggagtaaaagac
tgtgttgtattacacagttacttcacttcagactattaccagctgtactcaactcaattgagtacagacactggtgttgaacatgttaccttcttcatctacaa
taaaattgttgatgagcctgaagaacatgtccaaattcacacaatcgacggttcatccggagttgttaatccagtaatggaaccaatttatgatgaaccg
acgacgactactagcgtgcctttgtaagcacaagctgatgagtacgaacttatgtactcattcgtttcggaagagacaggtacgttaatagttaatagc
gtacttctttttcttgctttcgtggtattcttgctagttacactagccatccttactgcgcttcgattgtgtgcgtactgctgcaatattgttaacgtgagtctt
gtaaaaccttctttttacgtttactctcgtgttaaaaatctgaattcttctagagttcctgatcttctggtctaaacgaactaaatattatattagtttttctgt
ttggaactttaattttagccatggcagattccaacggtactattaccgttgaagagcttaaaaagctccttgaacaatggaacctagtaataggtttcctattcc
ttacatggatttgtcttctacaatttgcctatgccaacaggaataggtttttgtatataattaagttaattttcctctggctgttatggccagtaactttagctt
gttttgtgcttgctgctgtttacagaataaattggatcaccggtggaattgctatcgcaatggcttgtcttgtaggcttgatgtggctcagctacttcattgctt
ctttcagactgtttgcgcgtacgcgttccatgtggtcattcaatccagaaactaacattcttctcaacgtgccactccatggcactattctgaccagaccg
cttctagaaagtgaactcgtaatcggagctgtgatccttcgtggacatcttcgtattgctggacaccatctaggacgctgtgacatcaaggacctgcct
aaagaaatcactgttgctacatcacgaacgctttcttattacaaattgggagcttcgcagcgtgtagcaggtgactcaggttttgctgcatacagtcgct
acaggattggcaactataaattaaacacagaccattccagtagcagtgacaatattgctttgcttgtacagtaagtgacaacagatgtttcatctcgttg
actttcaggttactatagcagagatattactaattattatgaggacttttaaagtttccatttggaatcttgattacatcataaacctcataattaaaaatttat
ctaagtcactaactgagaataaatattctcaattagatgaagagcaaccaatggagattgattaaacgaacatgaaaattattcttttcttggcactgataa
cactcgctacttgtgagctttatcactaccaagagtgtgttagaggtacaacagtacttttaaaagaaccttgctcttctggaacatacgagggcaattc
accatttcatcctctagctgataacaaatttgcactgacttgctttagcactcaatttgcttttgcttgtcctgacggcgtaaaacacgtctatcagttacgt
gccagatcagtttcacctaaactgttcatcagacaagaggaagttcaagaactttactctccaatttttcttattgttgcggcaatagtgtttataacacttt
gcttcacactcaaaagaaagacagaatgattgaactttcattaattgacttctatttgtgctttttagcctttctgctattccttgttttaattatgcttattat
cttttggttctcacttgaactgcaagatcataatgaaacttgtcacgcctaaacgaacatgaaatttcttgttttcttaggaatcatcacaactgtagctgcatt
tcaccaagaatgtagtttacagtcatgtactcaacatcaaccatatgtagttgatgacccgtgtcctattcacttctattctaaatggtatattagagtaggag
ctagaaaatcagcacctttaattgaattgtgcgtggatgaggctggttctaaatcacccattcagtacatcgatatcggtaattatacagtttcctgttcac
cttttacaattaattgccaggaacctaaattgggtagtcttgtagtgcgttgttcgttctatgaagactttttagagtatcatgacgttcgtgttgttttagatt
tcatctaaacgaacaaactaaaatgtctgataatggaccccaaaatcagcgaaatgcaccccgcattacgtttggtggaccctcagattcaactggca
gtaaccagaatggagaacgcagtggggcgcgatcaaaacaacgtcggccccaaggtttacccaataatactgcgtcttggttcaccgctctcactc
aacatggcaaggaagaccttaaattccctcgaggacaaggcgttccaattaacaccaatagcagtccagatgaccaaattggctactaccgaagag
ctaccagacgaattcgtggtggtgacggtaaaatgaaagatctcagtccaagatggtatttctactacctaggaactgggccagaagctggacttcc
ctatggtgctaacaaagacggcatcatatgggttgcaactgagggagccttgaatacaccaaaagatcacattggcacccgcaatcctgctaacaat
gctgcaatcgtgctacaacttcctcaaggaacaacattgccaaaaggcttctacgcagaagggagcagaggcggcagtcaagcctcttctcgttcct
catcacgtagtcgcaacagttcaagaaattcaactccaggcagcagtaggggaacttctcctgctagaatggctggcaatggcggtgatgctgctct
tgctttgctgctgcttgacagattgaaccagcttgagagcaaaatgtctggtaaaggccaacaacaacaaggccaaactgtcactaagaaatctgct
gctgaggcttctaagaagcctcggcaaaaacgtactgccactaaagcatacaatgtaacacaagctttcggcagacgtggtccagaacaaaccca
aggaaattttggggaccaggaactaatcagacaaggaactgattacaaacattggccgcaaattgcacaatttgcccccagcgcttcagcgttcttc
ggaatgtcgcgcattggcatggaagtcacaccttcgggaacgtggttgacctacacaggtgccatcaaattggatgacaaagatccaaatttcaaag
atcaagtcattttgctgaataagcatattgacgcatacaaaacattcccaccaacagagcctaaaaaggacaaaaagaagaaggctgatgaaactca
agccttaccgcagagacagaagaaacagcaaactgtgactcttcttcctgctgcagatttggatgatttctccaaacaattgcaacaatccatgagca
gtgctgactcaactcaggcctaaactcatgcagaccacacaaggcagatgggctatataaacgttttcgcttttccgtttacgatatatagtctactcttg
tgcagaatgaattctcgtaactacatagcacaagtagatgtagttaactttaatctcacatagcaatctttaatcagtgtgtaacattagggaggacttga
aagagccaccacattttcaccgaggccacgcggagtacgatcgagtgtacagtgaacaatgctagggagagctgcctatatggaagagccctaat
gtgtaaaattaattttagtagtgctatccccatgtgattttaatagcttcttaggagaatgacAAAAAAAAAAAAAAAAAAAAA
n various embodiments, in reference to SEQ ID NO: 4, a mutation occurs at 3009 T to A
(Ile to Lys): [3009:3009]; 9199 C to T silent: [9199:9199]; 11524 C to T silent: [11524:11524];
16388 G to A (Ser to Asn): [16388:16388]; 21846 t to C (Ile to Thr): [21846:21846]; 22296 g to A
(Arg to His): [22296:22296]; 24201 T to C (Val to Ala): [24237:24237]; and/or 36 nt addition of
actaattctcctcggcgggcacgtagtgtagctagt (SEQ ID NO: 5): [23594],
SEQ ID NO: 6 (recoded spike protein in comparison to USA/WA1/2020 wild-type spike).
MFVFLVLLPLVSSQCVNLTTRTQLPPAYTNSFTRGVYYPDKVFRSSVLHSTQDLFLPFFSNVT
WFHAIHVSGTNGTKRFDNPVLPFNDGVYFASIEKSNIIRGWIFGTTLDSKTQSLLIVNNATNVV
IKVCEFQFCNDPFLGVYYHKNNKSWMESEFRVYSSANNCTFEYVSQPFLMDLEGKQGNFKN
LREFVFKNIDGYFKIYSKHTPINLVRDLPQGFSALEPLVDLPIGINITRFQTLLALRRSYLTPGDS
SSGWTAGAAAYYVGYLQPRTFLLKYNENGTITDAVDCALDPLSETKCTLKSFTVEKGIYQTS
NFRVQPTESIVRFPNITNLCPFGEVFNATRFASVYAWNRKRISNCVADYSVLYNSASFSTFKC
YGVSPTKLNDLCFTNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYFPLQSYGFQPTNGVG
YQPYRVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQDVNCTEVPVAIHADQL
TPTWRVYSTGSNVFQTRAGCLIGAEHVNNSYECDIPIGAGICASYQTQQSIIAYTMSLGAENS
VAYSNNSIAIPTNFTISVTTEILPVSMTKTSVDCTMYICGDSTECSNLLLQYGSFCTQLNRALT
GIAVEQDKNTQEVFAQVKQIYKTPPIKDFGGFNFSQILPDPSKPSKRSFIEDLLFNKVTLADAG
FIKQYGDCLGDIAARDLICAQKFNGLTVLPPLLTDEMIAQYTSALLAGTITSGWTFGAGVALQ
IPFAMQMAYRFNGIGVTQNVLYENQKLIANQFNSAIGKIQDSLSSTASALGKLQDVVNQNAQ
ALNTLVKQLSSNFGAISSVLNDILSRLDKVEAEVQIDRLITGRLQSLQTYVTQQLIRAAEIRAS
ANLAATKMSECVLGQSKRVDFCGKGYHLMSFPQSAPHGVVFLHVTYVPAQEKNFTTAPAIC
HDGKAHFPREGVFVSNGTHWFVTQRNFYEPQIITTDNTFVSGNCDVVIGIVNNTVYDPLQPEL
DSFKEELDKYFKNHTSPDVDLGDISGINASVVNIQKEIDRLNEVAKNLNESLIDLQELGKYEQ
YIKWPWYIWLGFIAGLIAIVMVTIMLCCMTSCCSCLKGCCSCGSCCKFDEDDSEPVLKGVKL
HYT
SEQ ID NO: 7-(Deoptimized SARS-CoV-2); 29853 bp, furin cleavage site removed
attaaaggtttataccttcccaggtaacaaaccaaccaactttcgatctcttgtagatctgttctctaaacgaactttaaaatctgtgtggctgtcactcgg
ctgcatgcttagtgcactcacgcagtataattaataactaattactgtcgttgacaggacacgagtaactcgtctatcttctgcaggctgcttacggtttc
gtccgtgttgcagccgatcatcagcacatctaggtttcgtccgggtgtgaccgaaaggtaagatggagagccttgtccctggtttcaacgagaaaac
acacgtccaactcagtttgcctgttttacaggttcgcgacgtgctcgtacgtggctttggagactccgtggaggaggtcttatcagaggcacgtcaac
atcttaaagatggcacttgtggcttagtagaagttgaaaaaggcgttttgcctcaacttgaacagccctatgtgttcatcaaacgttcggatgctcgaac
tgcacctcatggtcatgttatggttgagctggtagcagaactcgaaggcattcagtacggtcgtagtggtgagacacttggtgtccttgtccctcatgt
gggcgaaataccagtggcttaccgcaaggttcttcttcgtaagaacggtaataaaggagctggtggccatagttacggcgccgatctaaagtcattt
gacttaggcgacgagcttggcactgatccttatgaagattttcaagaaaactggaacactaaacatagcagtggtgttacccgtgaactcatgcgtga
gcttaacggaggggcatacactcgctatgtcgataacaacttctgtggccctgatggctaccctcttgagtgcattaaagaccttctagcacgtgctg
gtaaagcttcatgcactttgtccgaacaactggactttattgacactaagaggggtgtatactgctgccgtgaacatgagcatgaaattgcttggtaca
cggaacgttctgaaaagagctatgaattgcagacaccttttgaaattaaattggcaaagaaatttgacaccttcaatggggaatgtccaaattttgtattt
cccttaaattccataatcaagactattcaaccaagggttgaaaagaaaaagcttgatggctttatgggtagaattcgatctgtctatccagttgcgtcac
caaatgaatgcaaccaaatgtgcctttcaactctcatgaagtgtgatcattgtggtgaaacttcatggcagacgggcgattttgttaaagccacttgcg
aattttgtggcactgagaatttgactaaagaaggtgccactacttgtggttacttaccccaaaatgctgttgttaaaatttattgtccagcatgtcacaattc
agaagtaggacctgagcatagtcttgccgaataccataatgaatctggcttgaaaaccattcttcgtaagggtggtcgcactattgcctttggaggctg
tgtgttctcttatgttggttgccataacaagtgtgcctattgggttccacgtgctagcgctaacataggttgtaaccatacaggtgttgttggagaaggtt
ccgaaggtcttaatgacaaccttcttgaaatactccaaaaagagaaagtcaacatcaatattgttggtgactttaaacttaatgaagagatcgccattatt
ttggcatctttttctgcttccacaagtgcttttgtggaaactgtgaaaggtttggattataaagcattcaaacaaattgttgaatcctgtggtaattttaaagtt
acaaaaggaaaagctaaaaaaggtgcctggaatattggtgaacagaaatcaatactgagtcctctttatgcatttgcatcagaggctgctcgtgttgta
cgatcaattttctcccgcactcttgaaactgctcaaaattctgtgcgtgttttacagaaggccgctataacaatactagatggaatttcacagtattcactg
agactcattgatgctatgatgttcacatctgatttggctactaacaatctagttgtaatggcctacattacaggtggtgttgttcagttgacttcgcagtgg
ctaactaacatctttggcactgtttatgaaaaactcaaacccgtccttgattggcttgaagagaagtttaaggaaggtgtagagtttcttagagacggtt
gggaaattgttaaatttatctcaacctgtgcttgtgaaattgtcggtggacaaattgtcacctgtgcaaaggaaattaaggagagtgttcagacattcttt
aagcttgtaaataaatttttggctttgtgtgctgactctatcattattggtggagctaaacttaaagccttgaatttaggtgaaacatttgtcacgcactcaa
agggattgtacagaaagtgtgttaaatccagagaagaaactggcctactcatgcctctaaaagccccaaaagaaattatcttcttagagggagaaac
acttcccacagaagtgttaacagaggaagttgtcttgaaaactggtgatttacaaccattagaacaacctactagtgaagctgttgaagctccattggtt
ggtacaccagtttgtattaacgggcttatgttgctcgaaatcaaagacacagaaaagtactgtgcccttgcacctaatatgatggtaacaaacaatacc
ttcacactcaaaggcggtgcaccaacaaaggttacttttggtgatgacactgtgatagaagtgcaaggttacaagagtgtgaatatcacttttgaactt
gatgaaaggattgataaagtacttaatgagaagtgctctgcctatacagttgaactcggtacagaagtaaatgagttcgcctgtgttgtggcagatgct
gtcataaaaactttgcaaccagtatctgaattacttacaccactgggcattgatttagatgagtggagtatggctacatactacttatttgatgagtctggt
gagtttaaattggcttcacatatgtattgttctttctaccctccagatgaggatgaagaagaaggtgattgtgaagaagaagagtttgagccatcaactc
aatatgagtatggtactgaagatgattaccaaggtaaacctttggaatttggtgccacttctgctgctcttcaacctgaagaagagcaagaagaagatt
ggttagatgatgatagtcaacaaactgttggtcaacaagacggcagtgaggacaatcagacaactactattcaaacaattgttgaggttcaacctcaa
ttagagatggaacttacaccagttgttcagactattgaagtgaatagttttagtggttatttaaaacttactgacaatgtatacattaaaaatgcagacattg
tggaagaagctaaaaaggtaaaaccaacagtggttgttaatgcagccaatgtttaccttaaacatggaggaggtgttgcaggagccttaaataaggc
tactaacaatgccatgcaagttgaatctgatgattacatagctactaatggaccacttaaagtgggtggtagttgtgttttaagcggacacaatcttgcta
aacactgtcttcatgttgtcggcccaaatgttaacaaaggtgaagacattcaacttcttaagagtgcttatgaaaattttaatcagcacgaagttctacttg
caccattattatcagctggtatttttggtgctgaccctatacattctttaagagtttgtgtagatactgttcgcacaaatgtctacttagctgtctttgataaaa
atctctatgacaaacttgtttcaagctttttggaaatgaagagtgaaaagcaagttgaacaaaagatcgctgagattcctaaagaggaagttaagccat
ttataactgaaagtaaaccttcagttgaacagagaaaacaagatgataagaaaatcaaagcttgtgttgaagaagttacaacaactctggaagaaact
aagttcctcacagaaaacttgttactttatattgacattaatggcaatcttcatccagattctgccactcttgttagtgacattgacatcactttcttaaagaa
agatgctccatatatagtgggtgatgttgttcaagagggtgttttaactgctgtggttatacctactaaaaaggctggtggcactactgaaatgctagcg
aaagctttgagaaaagtgccaacagacaattatataaccacttacccgggtcagggtttaaatggttacactgtagaggaggcaaagacagtgctta
aaaagtgtaaaagtgccttttacattctaccatctattatctctaatgagaagcaagaaattcttggaactgtttcttggaatttgcgagaaatgcttgcac
atgcagaagaaacacgcaaattaatgcctgtctgtgtggaaactaaagccatagtttcaactatacagcgtaaatataagggtattaaaatacaagag
ggtgtggttgattatggtgctagattttacttttacaccagtaaaacaactgtagcgtcacttatcaacacacttaacgatctaaatgaaactcttgttacaa
tgccacttggctatgtaacacatggcttaaatttggaagaagctgctcggtatatgagatctctcaaagtgccagctacagtttctgtttcttcacctgat
gctgttacagcgtataatggttatcttacttcttcttctaaaacacctgaagaacattttattgaaaccatctcacttgctggttcctataaagattggtcctat
tctggacaatctacacaactaggtatagaatttcttaagagaggtgataaaagtgtatattacactagtaatcctaccacattccacctagatggtgaagt
tatcacctttgacaatcttaagacacttctttctttgagagaagtgaggactattaaggtgtttacaacagtagacaacattaacctccacacgcaagttg
tggacatgtcaatgacatatggacaacagtttggtccaacttatttggatggagctgatgttactaaaataaaacctcataattcacatgaaggtaaaac
attttatgttttacctaatgatgacactctacgtgttgaggcttttgagtactaccacacaactgatcctagttttctgggtaggtacatgtcagcattaaatc
acactaaaaagtggaaatacccacaagttaatggtttaacttctattaaatgggcagataacaactgttatcttgccactgcattgttaacactccaacaa
atagagttgaagtttaatccacctgctctacaagatgcttattacagagcaagggctggtgaagctgctaacttttgtgcacttatcttagcctactgtaat
aagacagtaggtgagttaggtgatgttagagaaacaatgagttacttgtttcaacatgccaatttagattcttgcaaaagagtcttgaacgtggtgtgta
aaacttgtggacaacagcagacaacccttaagggtgtagaagctgttatgtacatgggcacactttcttatgaacaatttaagaaaggtgttcagatac
cttgtacgtgtggtaaacaagctacaaaatatctagtacaacaggagtcaccttttgttatgatgtcagcaccacctgctcagtatgaacttaagcatgg
tacatttacttgtgctagtgagtacactggtaattaccagtgtggtcactataaacatataacttctaaagaaactttgtattgcatagacggtgctttactt
acaaagtcctcagaatacaaaggtcctattacggatgttttctacaaagaaaacagttacacaacaaccataaaaccagttacttataaattggatggt
gttgtttgtacagaaattgaccctaagttggacaattattataagaaagacaattcttatttcacagagcaaccaattgatcttgtaccaaaccaaccatat
ccaaacgcaagcttcgataattttaagtttgtatgtgataatatcaaatttgctgatgatttaaaccagttaactggttataagaaacctgcttcaagagag
cttaaagttacatttttccctgacttaaatggtgatgtggtggctattgattataaacactacacaccctcttttaagaaaggagctaaattgttacataaac
ctattgtttggcatgttaacaatgcaactaataaagccacgtataaaccaaatacctggtgtatacgttgtctttggagcacaaaaccagttgaaacatc
aaattcgtttgatgtactgaagtcagaggacgcgcagggaatggataatcttgcctgcgaagatctaaaaccagtctctgaagaagtagtggaaaat
cctaccatacagaaagacgttcttgagtgtaatgtgaaaactaccgaagttgtaggagacattatacttaaaccagcaaataatagtttaaaaattacag
aagaggttggccacacagatctaatggctgcttatgtagacaattctagtcttactattaagaaacctaatgaattatctagagtattaggtttgaaaacc
cttgctactcatggtttagctgctgttaatagtgtcccttgggatactatagctaattatgctaagccttttcttaacaaagttgttagtacaactactaacat
agttacacggtgtttaaaccgtgtttgtactaattatatgccttatttctttactttattgctacaattgtgtacttttactagaagtacaaattctagaatta
aagcatctatgccgactactatagcaaagaatactgttaagagtgtcggtaaattttgtctagaggcttcatttaattatttgaagtcacctaatttttctaaa
ctgataaatattataatttggtttttactattaagtgtttgcctaggttctttaatctactcaaccgctgctttaggtgttttaatgtctaatttaggcatgcc
ttcttactgtactggttacagagaaggctatttgaactctactaatgtcactattgcaacctactgtactggttctataccttgtagtgtttgtcttagtggtt
tagattctttagacacctatccttctttagaaactatacaaattaccatttcatcttttaaatgggatttaactgcttttggcttagttgcagagtggtttttg
gcatatattcttttcactaggtttttctatgtacttggattggctgcaatcatgcaattgtttttcagctattttgcagtacattttattagtaattcttggct
tatgtggttaataattaatcttgtacaaatggccccgatttcagctatggttagaatgtacatcttctttgcatcattttattatgtatggaaaagttatgtgc
atgttgtagacggttgtaattcatcaacttgtatgatgtgttacaaacgtaatagagcaacaagagtcgaatgtacaactattgttaatggtgttagaaggtcc
ttttatgtctatgctaatggaggtaaaggcttttgcaaactacacaattggaattgtgttaattgtgatacattctgtgctggtagtacatttattagtgatga
agttgcgagagacttgtcactacagtttaaaagaccaataaatcctactgaccagtcttcttacatcgttgatagtgttacagtgaagaatggttccatccatc
tttactttgataaagctggtcaaaagacttatgaaagacattctctctctcattttgttaacttagacaacctgagagctaataacactaaaggttcattgcct
attaatgttatagtttttgatggtaaatcaaaatgtgaagaatcatctgcaaaatcagcgtctgtttactacagtcagcttatgtgtcaacctatactgttact
agatcaggcattagtgtctgatgttggtgatagtgcggaagttgcagttaaaatgtttgatgcttacgttaatacgttttcatcaacttttaacgtaccaatgg
aaaaactcaaaacactagttgcaactgcagaagctgaacttgcaaagaatgtgtccttagacaatgtcttatctacttttatttcagcagctcggcaagggttt
gttgattcagatgtagaaactaaagatgttgttgaatgtcttaaattgtcacatcaatctgacatagaagttactggcgatagttgtaataactatatgctcac
ctataacaaagttgaaaacatgacaccccgtgaccttggtgcttgtattgactgtagtgcgcgtcatattaatgcgcaggtagcaaaaagtcacaacattgct
ttgatatggaacgttaaagatttcatgtcattgtctgaacaactacgaaaacaaatacgtagtgctgctaaaaagaataacttaccttttaagttgacatgt
gcaactactagacaagttgttaatgttgtaacaacaaagatagcacttaagggtggtaaaattgttaataattggttgaagcagttaattaaagttacact
tgtgttcctttttgttgctgctattttctatttaataacacctgttcatgtcatgtctaaacatactgacttttcaagtgaaatcataggatacaaggctattga
tggtggtgtcactcgtgacatagcatctacagatacttgttttgctaacaaacatgctgattttgacacatggtttagtcagcgtggtggtagttatactaat
gacaaagcttgcccattgattgctgcagtcataacaagagaagtgggttttgtcgtgcctggtttgcctggcacgatattacgcacaactaatggtgac
tttttgcatttcttacctagagtttttagtgcagttggtaacatctgttacacaccatcaaaacttatagagtacactgactttgcaacatcagcttgtgttttg
gctgctgaatgtacaatttttaaagatgcttctggtaagccagtaccatattgttatgataccaatgtactagaaggttctgttgcttatgaaagtttacgcc
ctgacacacgttatgtgctcatggatggctctattattcaatttcctaacacctaccttgaaggttctgttagagtggtaacaacttttgattctgagtactgt
aggcacggcacttgtgaaagatcagaagctggtgtttgtgtatctactagtggtagatgggtacttaacaatgattattacagatctttaccaggagtttt
ctgtggtgtagatgctgtaaatttacttactaatatgtttacaccactaattcaacctattggtgctttggacatatcagcatctatagtagctggtggtattg
tagctatcgtagtaacatgccttgcctactattttatgaggtttagaagagcttttggtgaatacagtcatgtagttgcctttaatactttactattccttatgt
cattcactgtactctgtttaacaccagtttactcattcttacctggtgtttattctgttatttacttgtacttgacattttatcttactaatgatgtttcttttt
tagcacatattcagtggatggttatgttcacacctttagtacctttctggataacaattgcttatatcatttgtatttccacaaagcatttctattggttcttta
gtaattacctaaagagacgtgtagtctttaatggtgtttcctttagtacttttgaagaagctgcgctgtgcacctttttgttaaataaagaaatgtatctaaagt
tgcgtagtgatgtgctattacctcttacgcaatataatagatacttagctctttataataagtacaagtattttagtggagcaatggatacaactagctacagag
aagctgcttgttgtcatctcgcaaaggctctcaatgacttcagtaactcaggttctgatgttctttaccaaccaccacaaacctctatcacctcagctgttttgc
agagtggttttagaaaaatggcattcccatctggtaaagttgagggttgtatggtacaagtaacttgtggtacaactacacttaacggtctttggcttgatga
cgtagtttactgtccaagacatgtgatctgcacctctgaagacatgcttaaccctaattatgaagatttactcattcgtaagtctaatcataatttcttggta
caggctggtaatgttcaactcagggttattggacattctatgcaaaattgtgtacttaagcttaaggttgatacagccaatcctaagacacctaagtataa
gtttgttcgcattcaaccaggacagactttttcagtgttagcttgttacaatggttcaccatctggtgtttaccaatgtgctatgaggcccaatttcactatta
agggttcattccttaatggttcatgtggtagtgttggttttaacatagattatgactgtgtctctttttgttacatgcaccatatggaattaccaactggagttc
atgctggcacagacttagaaggtaacttttatggaccttttgttgacaggcaaacagcacaagcagctggtacggacacaactattacagttaatgtttt
agcttggttgtacgctgctgttataaatggagacaggtggtttctcaatcgatttaccacaactcttaatgactttaaccttgtggctatgaagtacaattat
gaacctctaacacaagaccatgttgacatactaggacctctttctgctcaaactggaattgccgttttagatatgtgtgcttcattaaaagaattactgca
aaatggtatgaatggacgtaccatattgggtagtgctttattagaagatgaatttacaccttttgatgttgttagacaatgctcaggtgttactttccaaagt
gcagtgaaaagaacaatcaagggtacacaccactggttgttactcacaattttgacttcacttttagttttagtccagagtactcaatggtctttgttcttttt
tttgtatgaaaatgcctttttaccttttgctatgggtattattgctatgtctgcttttgcaatgatgtttgtcaaacataagcatgcatttctctgtttgttttt
gttaccttctcttgccactgtagcttattttaatatggtctatatgcctgctagttgggtgatgcgtattatgacatggttggatatggttgatactagtttgtc
tggttttaagctaaaagactgtgttatgtatgcatcagctgtagtgttactaatccttatgacagcaagaactgtgtatgatgatggtgctaggagagtgtgga
cacttatgaatgtcttgacactcgtttataaagtttattatggtaatgctttagatcaagccatttccatgtgggctcttataatctctgttacttctaactact
caggtgtagttacaactgtcatgtttttggccagaggtattgtttttatgtgtgttgagtattgccctattttcttcataactggtaatacacttcagtgtataa
tgctagtttattgtttcttaggctatttttgtacttgttactttggcctcttttgtttactcaaccgctactttagactgactcttggtgtttatgattacttag
tttctacacaggagtttagatatatgaattcacagggactactcccacccaagaatagcatagatgccttcaaactcaacattaaattgttgggtgttggtggca
aaccttgtatcaaagtagccactgtacagtctaaaatgtcagatgtaaagtgcacatcagtagtcttactctcagttttgcaacaactcagagtagaatcat
catctaaattgtgggctcaatgtgtccagttacacaatgacattctcttagctaaagatactactgaagcctttgaaaaaatggtttcactactttctgttttg
ctttccatgcagggtgctgtagacataaacaagctttgtgaagaaatgctggacaacagggcaaccttacaagctatagcctcagagtttagttccctt
ccatcatatgcagcttttgctactgctcaagaagcttatgagcaggctgttgctaatggtgattctgaagttgttcttaaaaagttgaagaagtctttgaat
gtggctaaatctgaatttgaccgtgatgcagccatgcaacgtaagttggaaaagatggctgatcaagctatgacccaaatgtataaacaggctagatc
tgaggacaagagggcaaaagttactagtgctatgcagacaatgcttttcactatgcttagaaagttggataatgatgcactcaacaacattatcaacaa
tgcaagagatggttgtgttcccttgaacataatacctcttacaacagcagccaaactaatggttgtcataccagactataacacatataaaaatacgtgt
gatggtacaacatttacttatgcatcagcattgtgggaaatccaacaggttgtagatgcagatagtaaaattgttcaacttagtgaaattagtatggaca
attcacctaatttagcatggcctcttattgtaacagctttaagggccaattctgctgtcaaattacagaataatgagcttagtcctgttgcactacgacaga
tgtcttgtgctgccggtactacacaaactgcttgcactgatgacaatgcgttagcttactacaacacaacaaagggaggtaggtttgtacttgcactgtt
atccgatttacaggatttgaaatgggctagattccctaagagtgatggaactggtactatctatacagaactggaaccaccttgtaggtttgttacagac
acacctaaaggtcctaaagtgaagtatttatactttattaaaggattaaacaacctaaatagaggtatggtacttggtagtttagctgccacagtacgtct
acaagctggtaatgcaacagaagtgcctgccaattcaactgtattatctttctgtgcttttgctgtagatgctgctaaagcttacaaagattatctagctag
tgggggacaaccaatcactaattgtgttaagatgttgtgtacacacactggtactggtcaggcaataacagttacaccggaagccaatatggatcaag
aatcctttggtggtgcatcgtgttgtctgtactgccgttgccacatagatcatccaaatcctaaaggattttgtgacttaaaaggtaagtatgtacaaatac
ctacaacttgtgctaatgaccctgtgggttttacacttaaaaacacagtctgtaccgtctgcggtatgtggaaaggttatggctgtagttgtgatcaactc
cgcgaacccatgcttcagtcagctgatgcacaatcgtttttaaacgggtttgcggtgtaagtgcagcccgtcttacaccgtgcggcacaggcactagt
actgatgtcgtatacagggcttttgacatctacaatgataaagtagctggttttgctaaattcctaaaaactaattgttgtcgcttccaagaaaaggacga
agatgacaatttaattgattcttactttgtagttaagagacacactttctctaactaccaacatgaagaaacaatttataatttacttaaggattgtccagctg
ttgctaaacatgacttctttaagtttagaatagacggtgacatggtaccacatatatcacgtcaacgtcttactaaatacacaatggcagacctcgtctat
gctttaaggcattttgatgaaggtaattgtgacacattaaaagaaatacttgtcacatacaattgttgtgatgatgattatttcaataaaaaggactggtat
gattttgtagaaaacccagatatattacgcgtatacgccaacttaggtgaacgtgtacgccaagctttgttaaaaacagtacaattctgtgatgccatgc
gaaatgctggtattgttggtgtactgacattagataatcaagatctcaatggtaactggtatgatttcggtgatttcatacaaaccacgccaggtagtgg
agttcctgttgtagattcttattattcattgttaatgcctatattaaccttgaccagggctttaactgcagagtcacatgttgacactgacttaacaaagcctt
acattaagtgggatttgttaaaatatgacttcacggaagagaggttaaaactctttgaccgttattttaaatattgggatcagacataccacccaaattgt
gttaactgtttggatgacagatgcattctgcattgtgcaaactttaatgttttattctctacagtgttcccacctacaagttttggaccactagtgagaaaaa
tatttgttgatggtgttccatttgtagtttcaactggataccacttcagagagctaggtgttgtacataatcaggatgtaaacttacatagctctagacttag
ttttaaggaattacttgtgtatgctgctgaccctgctatgcacgctgcttctggtaatctattactagataaacgcactacgtgcttttcagtagctgcactt
actaacaatgttgcttttcaaactgtcaaacccggtaattttaacaaagacttctatgactttgctgtgtctaagggtttctttaaggaaggaagttctgttg
aattaaaacacttcttctttgctcaggatggtaatgctgctatcagcgattatgactactatcgttataatctaccaacaatgtgtgatatcagacaactact
atttgtagttgaagttgttgataagtactttgattgttacgatggtggctgtattaatgctaaccaagtcatcgtcaacaacctagacaaatcagctggtttt
ccatttaataaatggggtaaggctagactttattatgattcaatgagttatgaggatcaagatgcacttttcgcatatacaaaacgtaatgtcatccctact
ataactcaaatgaatcttaagtatgccattagtgcaaagaatagagctcgcaccgtagctggtgtctctatctgtagtactatgaccaatagacagtttc
atcaaaaattattgaaatcaatagccgccactagaggagctactgtagtaattggaacaagcaaattctatggtggttggcacaacatgttaaaaactg
tttatagtgatgtagaaaaccctcaccttatgggttgggattatcctaaatgtgatagagccatgcctaacatgcttagaattatggcctcacttgttcttg
ctcgcaaacatacaacgtgttgtagcttgtcacaccgtttctatagattagctaatgagtgtgctcaagtattgagtgaaatggtcatgtgtggcggttca
ctatatgttaaaccaggtggaacctcatcaggagatgccacaactgcttatgctaatagtgtttttaacatttgtcaagctgtcacggccaatgttaatgc
acttttatctactgatggtaacaaaattgccgataagtatgtccgcaatttacaacacagactttatgagtgtctctatagaaatagagatgttgacacag
actttgtgaatgagttttacgcatatttgcgtaaacatttctcaatgatgatactctctgacgatgctgttgtgtgtttcaatagcacttatgcatctcaaggt
ctagtggctagcataaagaactttaagtcagttctttattatcaaaacaatgtttttatgtctgaagcaaaatgttggactgagactgaccttactaaagga
cctcatgaattttgctctcaacatacaatgctagttaaacagggtgatgattatgtgtaccttccttacccagatccatcaagaatcctaggggccggct
gttttgtagatgatatcgtaaaaacagatggtacacttatgattgaacggttcgtgtctttagctatagatgcttacccacttactaaacatcctaatcagg
agtatgctgatgtctttcatttgtacttacaatacataagaaagctacatgatgagttaacaggacacatgttagacatgtattctgttatgcttactaatga
taacacttcaaggtattgggaacctgagttttatgaggctatgtacacaccgcatacagtcttacaggctgttggggcttgtgttctttgcaattcacaga
cttcattaagatgtggtgcttgcatacgtagaccattcttatgttgtaaatgctgttacgaccatgtcatatcaacatcacataaattagtcttgtctgttaat
ccgtatgtttgcaatgctccaggttgtgatgtcacagatgtgactcaactttacttaggaggtatgagctattattgtaaatcacataaaccacccattagt
tttccattgtgtgctaatggacaagtttttggtttatataaaaatacatgtgttggtagcgataatgttactgactttaatgcaattgcaacatgtgactggac
aaatgctggtgattacattttagctaacacctgtactgaaagactcaagctttttgcagcagaaacgctcaaagctactgaggagacatttaaactgtct
tatggtattgctactgtacgtgaagtgctgtctgacagagaattacatctttcatgggaagttggtaaacctagaccaccacttaaccgaaattatgtcttt
actggttatcgtgtaactaaaaacagtaaagtacaaataggagagtacacctttgaaaaaggtgactatggtgatgctgttgtttaccgaggtacaaca
acttacaaattaaatgttggtgattattttgtgctgacatcacatacagtaatgccattaagtgcacctacactagtgccacaagagcactatgttagaatt
actggcttatacccaacactcaatatctcagatgagttttctagcaatgttgcaaattatcaaaaggttggtatgcaaaagtattctacactccagggacc
acctggtactggtaagagtcattttgctattggcctagctctctactacccttctgctcgcatagtgtatacagcttgctctcatgccgctgttgatgcact
atgtgagaaggcattaaaatatttgcctatagataaatgtagtagaattatacctgcacgtgctcgtgtagagtgttttgataaattcaaagtgaattcaac
attagaacagtatgtcttttgtactgtaaatgcattgcctgagacgacagcagatatagttgtctttgatgaaatttcaatggccacaaattatgatttgagt
gttgtcaatgccagattacgtgctaagcactatgtgtacattggcgaccctgctcaattacctgcaccacgcacattgctaactaagggcacactaga
accagaatatttcaattcagtgtgtagacttatgaaaactataggtccagacatgttcctcggaacttgtcggcgttgtcctgctgaaattgttgacactg
tgagtgctttggtttatgataataagcttaaagcacataaagacaaatcagctcaatgctttaaaatgttttataagggtgttatcacgcatgatgtttcatc
tgcaattaacaggccacaaataggcgtggtaagagaattccttacacgtaaccctgcttggagaaaagctgtctttatttcaccttataattcacagaat
gctgtagcctcaaagattttgggactaccaactcaaactgttgattcatcacagggctcagaatatgactatgtcatattcactcaaaccactgaaacag
ctcactcttgtaatgtaaacagatttaatgttgctattaccagagcaaaagtaggcatactttgcataatgtctgatagagacctttatgacaagttgcaat
ttacaagtcttgaaattccacgtaggaatgtggcaactttacaagctgaaaatgtaacaggactttttaaagattgtagtaaggtaatcactgggttacat
cctacacaggcacctacacacctcagtgttgacactaaattcaaaactgaaggtttatgtgttgacatacctggcatacctaaggacatgacctataga
agactcatctctatgatgggttttaaaatgaattatcaagttaatggttaccctaacatgtttatcacccgcgaagaagctataagacatgtacgtgcatg
gattggcttcgatgtcgaggggtgtcatgctactagagaagctgttggtaccaatttacctttacagctaggtttttctacaggtgttaacctagttgctgt
acctacaggttatgttgatacacctaataatacagatttttccagagttagtgctaaaccaccgcctggagatcaatttaaacacctcataccacttatgt
acaaaggacttccttggaatgtagtgcgtataaagattgtacaaatgttaagtgacacacttaaaaatctctctgacagagtcgtatttgtcttatgggca
catggctttgagttgacatctatgaagtattttgtgaaaataggacctgagcgcacctgttgtctatgtgatagacgtgccacatgcttttccactgcttca
gacacttatgcctgttggcatcattctattggatttgattacgtctataatccgtttatgattgatgttcaacaatggggttttacaggtaacctacaaagca
accatgatctgtattgtcaagtccatggtaatgcacatgtagctagttgtgatgcaatcatgactaggtgtctagctgtccacgagtgctttgttaagcgt
gttgactggactattgaatatcctataattggtgatgaactgaagattaatgcggcttgtagaaaggttcaacacatggttgttaaagctgcattattagc
agacaaattcccagttcttcacgacattggtaaccctaaagctattaagtgtgtacctcaagctgatgtagaatggaagttctatgatgcacagccttgt
agtgacaaagcttataaaatagaagaattattctattcttatgccacacattctgacaaattcacagatggtgtatgcctattttggaattgcaatgtcgata
gatatcctgctaattccattgtttgtagatttgacactagagtgctatctaaccttaacttgcctggttgtgatggtggcagtttgtatgtaaataaacatgc
attccacacaccagcttttgataaaagtgcttttgttaatttaaaacaattaccatttttctattactctgacagtccatgtgagtctcatggaaaacaagtag
tgtcagatatagattatgtaccactaaagtctgctacgtgtataacacgttgcaatttaggtggtgctgtctgtagacatcatgctaatgagtacagattgt
atctcgatgcttataacatgatgatctcagctggctttagcttgtgggtttacaaacaatttgatacttataacctctggaacacttttacaagacttcagag
tttagaaaatgtggcttttaatgttgtaaataagggacactttgatggacaacagggtgaagtaccagtttctatcattaataacactgtttacacaaaagt
tgatggtgttgatgtagaattgtttgaaaataaaacaacattacctgttaatgtagcatttgagctttgggctaagcgcaacattaaaccagtaccagag
gtgaaaatactcaataatttgggtgtggacattgctgctaatactgtgatctgggactacaaaagagatgctccagcacatatatctactattggtgtttg
ttctatgactgacatagccaagaaaccaactgaaacgatttgtgcaccactcactgtcttttttgatggtagagttgatggtcaagtagacttatttagaa
atgcccgtaatggtgttcttattacagaaggtagtgttaaaggtttacaaccatctgtaggtcccaaacaagctagtcttaatggagtcacattaattgga
gaagccgtaaaaacacagttcaattattataagaaagttgatggtgttgtccaacaattacctgaaacttactttactcagagtagaaatttacaagaattt
aaacccaggagtcaaatggaaattgatttcttagaattagctatggatgaattcattgaacggtataaattagaaggctatgccttcgaacatatcgttta
tggagattttagtcatagtcagttaggtggtttacatctactgattggactagctaaacgttttaaggaatcaccttttgaattagaagattttattcctatgg
acagtacagttaaaaactatttcataacagatgcgcaaacaggttcatctaagtgtgtgtgttctgttattgatttattacttgatgattttgttgaaataataa
aatcccaagatttatctgtagtttctaaggttgtcaaagtgactattgactatacagaaatttcatttatgctttggtgtaaagatggccatgtagaaacattt
tacccaaaattacaatctagtcaagcgtggcaaccgggtgttgctatgcctaatctttacaaaatGCAAAGAATGCTATTAGAAA
AGTGTGACCTTCAAAATTATGGTGATAGTGCAACATTACCTAAAGGCATAATGATGAATG
TCGCAAAATATACTCAACTGTGTCAATATTTAAACACATTAACATTAGCTGTACCCTATA
ATATGAGAGTTATACATTTTGGTGCTGGTTCTGATAAAGGAGTTGCACCAGGTACAGCTG
TTTTAAGACAGTGGTTGCCTACGGGTACGCTGCTTGTCGATTCAGATCTTAATGACTTTGT
CTCTGATGCAGATTCAACTTTGATTGGTGATTGTGCAACTGTACATACAGCTAATAAATG
GGATCTCATTATTAGTGATATGTACGACCCTAAGACTAAAAATGTTACAAAAGAAAATG
ACTCTAAAGAGGGTTTTTTCACTTACATTTGTGGGTTTATACAACAAAAGCTAGCTCTTG
GAGGTTCCGTGGCTATAAAGATAACAGAACATTCTTGGAATGCTGATCTTTATAAGCTCA
TGGGACACTTCGCATGGTGGACAGCCTTTGTTACTAATGTGAATGCGTCATCATCTGAAG
CATTTTTAATTGGATGTAATTATCTTGGCAAACCACGCGAACAAATAGATGGTTATGTCA
TGCATGCAAATTACATATTTTGGAGGAATACAAATCCAATTCAGTTGTCTTCCTATTCTTT
ATTTGACATGAGTAAATTTCCCCTTAAATTAAGGGGTACTGCTGTTATGTCTTTAAAAGA
AGGTCAAATCAATGATATGATTTTATCTCTTCTTAGTAAAGGTAGACTTATAATTAGAGA
AAACAACAGAGTTGTTATTTCTAGTGATGTTCTTGTTAACAACTAAACGAACAATGTTTG
TTTTTCTTGTTTTATTGCCACTAGTCTCTAGTCAGTGTGTTAATCTTACAACCAGAACTCA
ATTACCCCCTGCATACACTAATTCTTTTACTAGGGGGGTTTATTATCCTGATAAGGTTTTT
CGTAGTTCAGTCTTGCATAGTACTCAGGATCTATTTTTACCTTTCTTTTCGAACGTTACTT
GGTTTCACGCTATACACGTTTCCGGTACTAACGGTACTAAAAGATTCGATAATCCTGTCT
TACCATTTAACGACGGAGTGTATTTCGCTAGTACCGAAAAATCTAATATTATTAGGGGTT
GGATATTCGGTACTACTCTTGACTCTAAAACGCAATCATTGTTAATCGTTAATAACGCTA
CTAACGTCGTTATTAAGGTATGCGAATTTCAGTTTTGTAACGATCCTTTCTTAGGCGTTTA
TTATCATAAAAATAATAAGTCTTGGATGGAATCCGAATTTAGAGTGTATAGTTCCGCTAA
TAATTGTACATTCGAATACGTTTCGCAACCATTTCTTATGGATCTTGAGGGTAAGCAGGG
TAATTTTAAAAATTTGCGTGAATTCGTTTTTAAAAATATAGACGGTTATTTTAAGATATAC
TCTAAACATACCCCTATTAATCTCGTTAGGGATCTACCTCAGGGTTTTTCCGCTCTTGAGC
CATTAGTCGATTTGCCAATAGGTATTAATATTACTAGATTTCAGACTCTTTTAGCGTTACA
TAGATCTTATTTGACTCCTGGTGATTCTTCTTCAGGTTGGACAGCTGGTGCTGCAGCTTAT
TATGTGGGTTATCTTCAACCTAGGACTTTTCTATTAAAATATAATGAAAATGGAACCATT
ACAGATGCTGTAGACTGTGCACTTGACCCTCTCTCAGAAACAAAGTGTACGTTGAAATCC
TTCACTGTAGAAAAAGGAATCTATCaaacttctaactttagagtccaaccaacagaatctattgttagatttcctaatattacaa
acttgtgcccttttggtgaagtttttaacgccaccagatttgcatctgtttatgcttggaacaggaagagaatcagcaactgtgttgctgattattctgtcct
atataattccgcatcattttccacttttaagtgttatggagtgtctcctactaaattaaatgatctctgctttactaatgtctatgcagattcatttgtaattag
aggtgatgaagtcagacaaatcgctccagggcaaactggaaagattgctgattataattataaattaccagatgattttacaggctgcgttatagcttgg
aattctaacaatcttgattctaaggttggtggtaattataattacctgtatagattgtttaggaagtctaatctcaaaccttttgagagagatatttcaactga
aatctatcaggccggtagcacaccttgtaatggtgttgaaggttttaattgttactttcctttacaatcatatggtttccaacccactaatggtgttggttacc
aaccatacagagtagtagtactttcttttgaacttctacatgcaccagcaactgtttgtggacctaaaaagtctactaatttggttaaaaacaaatgtgtca
atttcaacttcaatggtttaacaggcacaggtgttcttactgagtctaacaaaaagtttctgcctttccaacaatttggcagagacattgctgacactactg
atgctgtccgtgatccacagacacttgagattcttgacattacaccatgttcttttggtggtgtcagtgttataacaccaggaacaaatacttctaaccag
gttgctgttctttatcaggatgttaactgcacagaagtccctgttgctattcatgcagatcaacttactcctacttggcgtgtttattctacaggttctaatgtt
tttcaaacacgtgcaggctgtttaataggggctgaacatgtcaacaactcatatgagtgtgacatacccattggtgcaggtatatgcgctagttatcag
actcagcaatccatcattgcctacactatgtcacttggtgcagaaaattcagttgcttactctaataactctattgccatacccacaaattttactattagtgt
taccacagaaattctaccagtgtctatgaccaagacatcagtagattgtacaatgtacatttgtggtgattcaactgaatgcagcaatcttttgttgcaata
tggcagtttttgtacacaattaaaccgtgctttaactggaatagctgttgaacaagacaaaaacacccaagaagtttttgcacaagtcaaacaaatttac
aaaacaccaccaattaaagattttggtggttttaatttttcacaaatattaccagatccatcaaaaccaagcaagaggtcatttattgaagatctacttttca
acaaagtgacacttgcagatgctggcttcatcaaacaatatggtgattgccttggtgatattgctgctagagacctcatttgtgcacaaaagtttaacgg
ccttactgttttgccacctttgctcacagatgaaatgattgctcaatacacttctgcactgttagcgggtacaatcacttctggttggacctttggtgcagg
tgctgcattacaaataccatttgctatgcaaatggcttataggtttaatggtattggagttacacagaatgttctctatgagaaccaaaaattgattgccaa
ccaatttaatagtgctattggcaaaattcaagactcactttcttccacagcaagtgcacttggaaaacttcaagatgtggtcaaccaaaatgcacaagct
ttaaacacgcttgttaaacaacttagctccaattttggtgcaatttcaagtgttttaaatgatatcctttcacgtcttgacaaagttgaggctgaagtgcaaa
ttgataggttgatcacaggcagacttcaaagtttgcagacatatgtgactcaacaattaattagagctgcagaaatcagagcttctgctaatcttgctgc
tactaaaatgtcagagtgtgtacttggacaatcaaaaagagttgatttttgtggaaagggctatcatcttatgtccttccctcagtcagcacctcatggtg
tagtcttcttgcatgtgacttatgtccctgcacaagaaaagaacttcacaactgctcctgccatttgtcatgatggaaaagcacactttcctcgtgaaggt
gtctttgtttcaaatggcacacactggtttgtaacacaaaggaatttttatgaaccacaaatcattactacagacaacacatttgtgtctggtaactgtgat
gttgtaataggaattgtcaacaacacagtttatgatcctttgcaacctgaattagactcattcaaggaggagttagataaatattttaagaatcatacatca
ccagatgttgatttaggtgacatctctggcattaatgcttcagttgtaaacattcaaaaagaaattgaccgcctcaatgaggttgccaagaatttaaatga
atctctcatcgatctccaagaacttggaaagtatgagcagtatataaaatggccatggtacatttggctaggttttatagctggcttgattgccatagtaa
tggtgacaattatgctttgctgtatgaccagttgctgtagttgtctcaagggctgttgttcttgtggatcctgctgcaaatttgatgaagacgactctgagc
cagtgctcaaaggagtcaaattacattacacataaacgaacttatggatttgtttatgagaatcttcacaattggaactgtaactttgaagcaaggtgaa
atcaaggatgctactccttcagattttgttcgcgctactgcaacgataccgatacaagcctcactccctttcggatggcttattgttggcgttgcacttctt
gctgtttttcagagcgcttccaaaatcataaccctcaaaaagagatggcaactagcactctccaagggtgttcactttgtttgcaacttgctgttgttgttt
gtaacagtttactcacaccttttgctcgttgctgctggccttgaagccccttttctctatctttatgctttagtctacttcttgcagagtataaactttgtaaga
ataataatgaggctttggctttgctggaaatgccgttccaaaaacccattactttatgatgccaactattttctttgctggcatactaattgttacgactattg
tataccttacaatagtgtaacttcttcaattgtcattacttcaggtgatggcacaacaagtcctatttctgaacatgactaccagattggtggttatactgaa
aaatgggaatctggagtaaaagactgtgttgtattacacagttacttcacttcagactattaccagctgtactcaactcaattgagtacagacactggtgt
tgaacatgttaccttcttcatctacaataaaattgttgatgagcctgaagaacatgtccaaattcacacaatcgacggttcatccggagttgttaatccag
taatggaaccaatttatgatgaaccgacgacgactactagcgtgcctttgtaagcacaagctgatgagtacgaacttatgtactcattcgtttcggaag
agacaggtacgttaatagttaatagcgtacttctttttcttgctttcgtggtattcttgctagttacactagccatccttactgcgcttcgattgtgtgcgtact
gctgcaatattgttaacgtgagtcttgtaaaaccttctttttacgtttactctcgtgttaaaaatctgaattcttctagagttcctgatcttctggtctaaacga
actaaatattatattagtttttctgtttggaactttaattttagccatggcagattccaacggtactattaccgttgaagagcttaaaaagctccttgaacaat
ggaacctagtaataggtttcctattccttacatggatttgtcttctacaatttgcctatgccaacaggaataggtttttgtatataattaagttaattttcctct
ggctgttatggccagtaactttagcttgttttgtgcttgctgctgtttacagaataaattggatcaccggtggaattgctatcgcaatggcttgtcttgtaggc
ttgatgtggctcagctacttcattgcttctttcagactgtttgcgcgtacgcgttccatgtggtcattcaatccagaaactaacattcttctcaacgtgccac
tccatggcactattctgaccagaccgcttctagaaagtgaactcgtaatcggagctgtgatccttcgtggacatcttcgtattgctggacaccatctag
gacgctgtgacatcaaggacctgcctaaagaaatcactgttgctacatcacgaacgctttcttattacaaattgggagcttcgcagcgtgtagcaggt
gactcaggttttgctgcatacagtcgctacaggattggcaactataaattaaacacagaccattccagtagcagtgacaatattgctttgcttgtacagt
aagtgacaacagatgtttcatctcgttgactttcaggttactatagcagagatattactaattattatgaggacttttaaagtttccatttggaatcttgattac
atcataaacctcataattaaaaatttatctaagtcactaactgagaataaatattctcaattagatgaagagcaaccaatggagattgattaaacgaacat
gaaaattattcttttcttggcactgataacactcgctacttgtgagctttatcactaccaagagtgtgttagaggtacaacagtacttttaaaagaaccttg
ctcttctggaacatacgagggcaattcaccatttcatcctctagctgataacaaatttgcactgacttgctttagcactcaatttgcttttgcttgtcctgac
ggcgtaaaacacgtctatcagttacgtgccagatcagtttcacctaaactgttcatcagacaagaggaagttcaagaactttactctccaatttttcttatt
gttgcggcaatagtgtttataacactttgcttcacactcaaaagaaagacagaatgattgaactttcattaattgacttctatttgtgctttttagcctttctgc
tattccttgttttaattatgcttattatcttttggttctcacttgaactgcaagatcataatgaaacttgtcacgcctaaacgaacatgaaatttcttgttttct
taggaatcatcacaactgtagctgcatttcaccaagaatgtagtttacagtcatgtactcaacatcaaccatatgtagttgatgacccgtgtcctattcacttc
tattctaaatggtatattagagtaggagctagaaaatcagcacctttaattgaattgtgcgtggatgaggctggttctaaatcacccattcagtacatcga
tatcggtaattatacagtttcctgttcaccttttacaattaattgccaggaacctaaattgggtagtcttgtagtgcgttgttcgttctatgaagactttttaga
gtatcatgacgttcgtgttgttttagatttcatctaaacgaacaaactaaaatgtctgataatggaccccaaaatcagcgaaatgcaccccgcattacgtt
tggtggaccctcagattcaactggcagtaaccagaatggagaacgcagtggggcgcgatcaaaacaacgtcggccccaaggtttacccaataata
ctgcgtcttggttcaccgctctcactcaacatggcaaggaagaccttaaattccctcgaggacaaggcgttccaattaacaccaatagcagtccagat
gaccaaattggctactaccgaagagctaccagacgaattcgtggtggtgacggtaaaatgaaagatctcagtccaagatggtatttctactacctagg
aactgggccagaagctggacttccctatggtgctaacaaagacggcatcatatgggttgcaactgagggagccttgaatacaccaaaagatcacat
tggcacccgcaatcctgctaacaatgctgcaatcgtgctacaacttcctcaaggaacaacattgccaaaaggcttctacgcagaagggagcagagg
cggcagtcaagcctcttctcgttcctcatcacgtagtcgcaacagttcaagaaattcaactccaggcagcagtaggggaacttctcctgctagaatgg
ctggcaatggcggtgatgctgctcttgctttgctgctgcttgacagattgaaccagcttgagagcaaaatgtctggtaaaggccaacaacaacaagg
ccaaactgtcactaagaaatctgctgctgaggcttctaagaagcctcggcaaaaacgtactgccactaaagcatacaatgtaacacaagctttcggc
agacgtggtccagaacaaacccaaggaaattttggggaccaggaactaatcagacaaggaactgattacaaacattggccgcaaattgcacaattt
gcccccagcgcttcagcgttcttcggaatgtcgcgcattggcatggaagtcacaccttcgggaacgtggttgacctacacaggtgccatcaaattgg
atgacaaagatccaaatttcaaagatcaagtcattttgctgaataagcatattgacgcatacaaaacattcccaccaacagagcctaaaaaggacaaa
aagaagaaggctgatgaaactcaagccttaccgcagagacagaagaaacagcaaactgtgactcttcttcctgctgcagatttggatgatttctccaa
acaattgcaacaatccatgagcagtgctgactcaactcaggcctaaactcatgcagaccacacaaggcagatgggctatataaacgttttcgcttttc
cgtttacgatatatagtctactcttgtgcagaatgaattctcgtaactacatagcacaagtagatgtagttaactttaatctcacatagcaatctttaatcagt
gtgtaacattagggaggacttgaaagagccaccacattttcaccgaggccacgcggagtacgatcgagtgtacagtgaacaatgctagggagagc
tgcctatatggaagagccctaatgtgtaaaattaattttagtagtgctatccccatgtgattttaatagcttcttaggagaatgacaaaaaaaaaaaaaaa
aaaa

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Overlapping PCR

Step 1: PCR and Gel Purification. Primers and Templates. To Start from T7 Promoter

| | SEQ ID | | |
|---|---|---|---|
| Primers | NO: | Sequences | Template |
| 1785-COV-1- | 9 | TAATACGACTCACTATTATTAAAGGTTTATAC | Order fragment 1 |
| T7 | | CTTCCCAGGTAAC | (Start from T7 |
| | | | promoter) |
| 1786-COV-2 | 10 | GATGCCAAAATAATGGCGATCTC | |
| 1787-COV-3 | 11 | GTTGGTTGCCATAACAAGTGTG | Order fragment 2 |
| 1788-COV-4 | 12 | CTAATTGAGGTTGAACCTCAACAATTG | |
| 1789-COV-5 | 13 | GAGTATGGTACTGAAGATGATTACCAAG | Order fragment 3 |
| 1790-COV-6 | 14 | CTAGGTGGAATGTGGTAGGATTAC | |
| 1791-COV-7 | 15 | GCTGTTACAGCGTATAATGGTTATCTTAC | Order fragment 4 |
| 1792-COV-8 | 16 | GCTGGTTTAAGTATAATGTCTCCTACAAC | |
| 1793-COV-9 | 17 | GCACAAAACCAGTTGAAACATCAAATTC | Order fragment 5 |
| 1794-COV-10 | 18 | GCAACTAGTGTTTTGAGTTTTTCCATTG | |
| 1795-COV-ll | 19 | GTGAAGAATCATCTGCAAAATCAGC | Order fragment 6 |
| 1796-COV-12 | 20 | CAAATGATATAAGCAATTGTTATCCAGAAAGG | |
| 1797-COV-13 | 21 | GCCTTTAATACTTTACTATTCCTTATGTCATT | Order fragment 7 |
| | | CAC | |
| 1798-COV-14 | 22 | CCAGACAAACTAGTATCAACCATATCC | |
| 1799-COV-15 | 23 | GCTATGGGTATTATTGCTATGTCTG | Order fragment 8 - |
| | | | with WT and Min |
| | | | versions, need to |
| | | | purify individually. |
| 1800-COV-16 | 24 | CCTACAAGGTGGTTCCAGTTC | |
| 1801-COV-17 | 25 | CGACAGATGTCTTGTGCTG | Order fragment 9 |
| 1802-COV-18 | 26 | GGTATCCAGTTGAAACTACAAATGG | |
| 1803-COV-19 | 27 | GATCAGACATACCACCCAAATTG | Order fragment 10 - |
| | | | with WT and Min |
| | | | versions, need to |
| | | | purify individually. |
| 1804-COV-20 | 28 | CTTATGTATTGTAAGTACAAATGAAAGACATC | |
| | | AG | |
| 1805-COV-21 | 29 | GGTGATGATTATGTGTACCTTCCTTAC | Order fragment 11 |
| 1806-COV-22 | 30 | CTGTTAATTGCAGATGAAACATCATGC | |
| 1807-COV-23 | 31 | GTGTGTAGACTTATGAAAACTATAGGTCC | Order fragment 12 |
| 1808-COV-24 | 32 | CATACAAACTGCCACCATCAC | |
| 1809-COV-25 | 33 | CCTTGTAGTGACAAAGCTTATAAAATAGAAG | Order fragment 13 |
| 1810-COV-26 | 34 | CTGGTGCAACTCCTTTATCAG | |
| 1811-COV-27 | 35 | GCAAAGAATGCTATTAGAAAAGTGTGAC | Order fragment 14 - |
| | | | with WT and Min |
| | | | versions, need to |
| | | | purify individually. |
| 1812-COV-28 | 36 | GATAGATTCCTTTTTCTACAGTGAAGGATTTC | |
| 1813-COV-29 | 37 | GACTCCTGGTGATTCTTCTTCAG | Order fragment 15 - |
| | | | with WT and Min |
| | | | versions, need to |
| | | | purify individually. |
| 1814-COV-30 | 38 | CTCTAGCAGCAATATCACCAAGG | |
| 1815-COV-31 | 39 | GCACAAGTCAAACAAATTTACAAAACAC | Order fragment 16 - |
| | | | with WT and Min |
| | | | versions, need to |
| | | | purify individually. |
| 1816-COV-32 | 40 | CAAAAGGTGTGAGTAAACTGTTACAAAC | |
| 1817-COV-33 | 41 | CTCACTCCCTTTCGGATGG | Order fragment 17 |
| 1818-COV-34 | 42 | GAGGTTTATGATGTAATCAAGATTCCAAATGG | |
| 1819-COV-35 | 43 | GCTACAGGATTGGCAACTATAAATTAAAC | Order fragment 18 |
| 1820-COV-36 | 44 | CCATTCTAGCAGGAGAAGTTCC | |
| 1821-COV-37 | 45 | GCAATCCTGCTAACAATGCTG | Order fragment 19 |
| | | | Alternative primer |
| | | | for end of CoV |
| | | | genome, end of |
| | | | ordered fragment 19. |
| 1822-COV-38 | 46 | ttttTTTTTTTTTTTTTTTTTTTTTGTCATTC | |
| | | TCCTAAGAAGCTATTAAAATC | |
| 1865- | 47 | GAAGACTTGGTACACTTGAGACG | |
| CoVFragl9-R | | | |

To include the pCMV in fragment 0

| | SEQ ID | | |
|---|---|---|---|
| | NO: | | |
| 1862-CoVFrag0-F | 48 | aattttGACGTCgatc | With Zra I site |
| | | cCGTTGACATTGATTA | GACGTC, start |
| | | TTGAC | at nt81 in |
| | | | fragment 0 |
| 1863-CoVFrag0-R | 49 | CCACACAGATTTTAAA | |
| | | GTTCGTTTAGAG | |
| 1864-CoVFrag1-F | 50 | GCAAATGGGCGGTAGG | |

Step 2: Overlapping PCR

May need to overlap 4-5 pieces first—purify the products—then overlap to get the full length genome.

Step 3:

Option 1: Use directly for in vitro transcription/store as overlapping PCR product;

Option 2: Clone into pCC1BAC vector

To clone the CoV genome into pCC1BAC vector, use #1883 and 1890 as forward and reverse primers for amplifying fragment0 and fragment 19, as well as in final amplification after overlapping. Assemble with Bsa I carrying pCC1BAC using NEB Golden gate assembly.

| | | |
|---|---|---|
| 1883- | 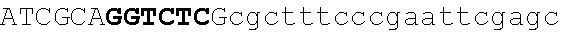 | For pCC1BAC assembly-full CoV, |
| pGGAfrag1-F | (SEQ ID NO: 51) | start at nt50 in fragment 0 |
| 1890- | 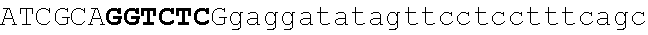 | For pCC1BAC assembly-full CoV |
| pGGAfrag4-R | (SEQ ID NO: 52) | |

Clone into pGGA Vector (from Golden Gate Clone Kit) Through Bsa I Sites, then Clone into pCC1BAC Vector Through Bsa I Site

pCC1BAC obtained from existing pCC1-CysS-CD plasmid and need modification—see “pCC1BAC Modification”

Step 1: PCR and Gel Purification

Primers and Templates—To start from T7 promoter

| | SEQ ID | | |
|---|---|---|---|
| Primers | NO: | Sequences | Templates |
| 1823-CoVfrag1-BsaFl | 53 | 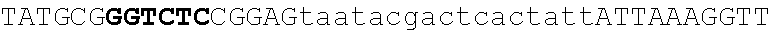 | Ordered fragment 1 |
| | | | For pGGA clone, |
| 1824-CoVfrag1-BsaR | 54 | 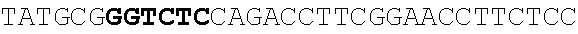 | Ordered fragment 1-4, |
| | | | T7 promoter-first 10nt |
| | | | Viral genome start at |
| | | | T7 promoter |
| 1825-CoVfrag2-BsaF | 55 | 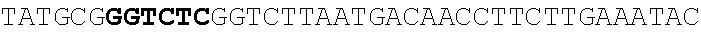 | Ordered fragment 2 |
| 1826-CoVfrag2-BsaR | 56 | 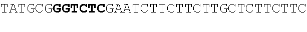 | |
| 1827-CoVfrag3-BsaF | 57 | 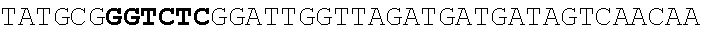 | Ordered fragment 3 |
| 1828-CoVfrag3-BsaR | 58 | 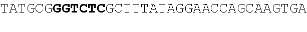 | |
| 1829-CoVfrag4-BsaF | 59 | 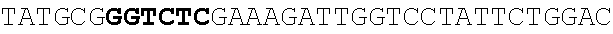 | Ordered fragment 4 |
| 1830-CoVfrag4-BsaR | 60 | 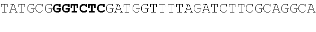 | End at nt6400 |
| 1831-CoVfrag5-BsaF | 61 | 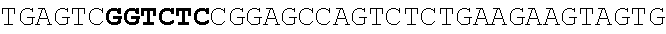 | Ordered fragment 5 |
| 1832-CoVfrag5-BsaR | 62 | 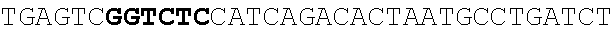 | For pGGA clone, Ordered |
| | | | fragment 5-9 |
| 1833-CoVfrag6-BsaF | 63 | 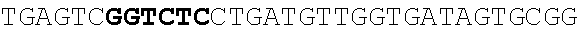 | Ordered fragment 6 |
| 1834-CoVfrag6-BsaR | 64 | 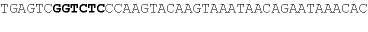 | |
| 1835-CoVfrag7-BsaF | 65 | 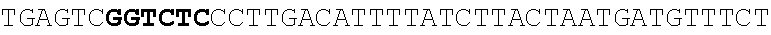 | Ordered fragment 7 |
| 1836-CoVfrag7-BsaR | 66 | 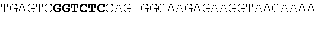 | |
| 1837-CoVfrag8-BsaF | 67 | 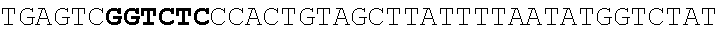 | Ordered fragment 8-with |
| 1838-CoVfrag8-BsaR | 68 | 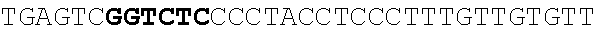 | WT and Min versions, |
| | | | need to purify |
| | | | individually. |
| 1839-CoVfrag9-BsaF | 69 | 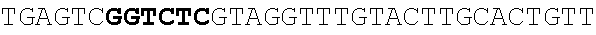 | Ordered fragment 9 |
| 1840-CoVfrag9-BsaR | 70 | 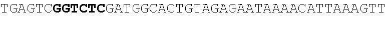 | End at nt 14400 |
| 1841-CoVfrag10- | 71 | 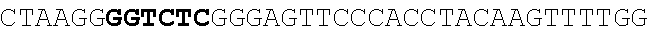 | Ordered fragment 10 |
| BsaF | | | For pGGA clone, Ordered |
| 1842-CoVfrag10- | 72 | 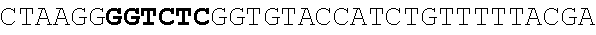 | fragment-with |
| BsaR | | | WT and Min versions, |
| | | | need to purify |
| | | | individually. 10-14 |
| 1843-CoVfrag11- | 73 | 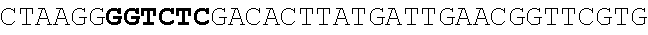 | Ordered fragment 11 |
| BsaF | | | |
| 1844-CoVfrag11-BsaR | 74 | 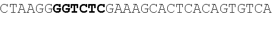 | |
| 1845-CoVfrag12- | 75 | 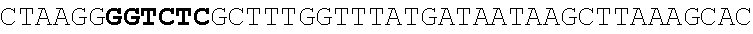 | Ordered fragment 12 |
| BsaF | | | |
| 1846-CoVfrag12- | 76 | 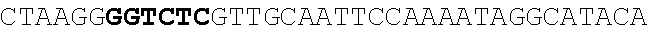 | |
| BsaR | | | |
| 1847-CoVfrag13- | 77 | 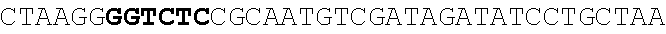 | Ordered fragment 13 |
| BsaF | | | |
| 1848-CoVfrag13-BsaR | 78 | 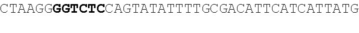 | |
| 1849-CoVfrag14- | 79 | 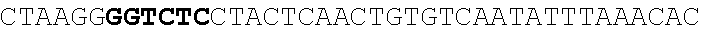 | Ordered fragment 14 |
| BsaF | | | End at nt22400-with |
| 1850-CoVfrag14- | 80 | 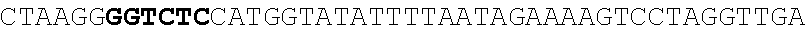 | WT and Min versions, |
| BsaR | | | need to purify |
| | | | individually. |
| 1866-CoVfrag15- | 81 | 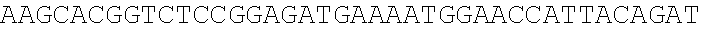 | Ordered fragment 15 |
| BsaF | | | For pGGA clone, Ordered |
| 1867-CoVfrag15- | 82 | 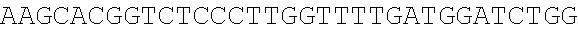 | fragment 15-19 with |
| BsaR | | | polyA signal |
| | | | sequence-with |
| | | | WT and Min versions, |
| | | | need to purify |
| | | | individually. |
| 1868-CoVfrag16- | 83 | 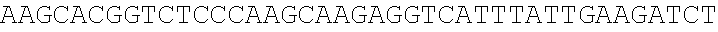 | Ordered fragment 16-with |
| BsaF | | | WT and Min versions, |
| 1869-CoVfrag16- | 84 | 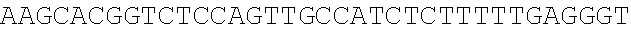 | need to purify |
| BsaR | | | individually. |
| 1870-CoVfrag17- | 85 | 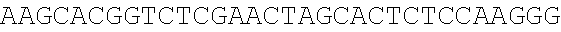 | Ordered fragment 17 |
| BsaF | | | |
| 1871-CoVfrag17-BsaR | 86 | 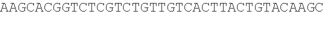 | |
| 1872-CoVfrag18- | 87 | 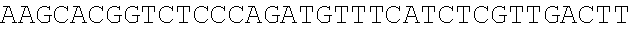 | Ordered fragment 18 |
| BsaF | | | |
| 1873-CoVfrag18-BsaR | 88 | 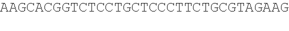 | |
| 1874-CoVfrag19- | 89 | 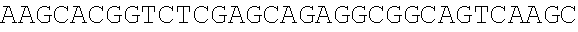 | Ordered fragment 19 |
| BsaF | | | ends before the |
| 1875-CoVfrag19- | 90 | 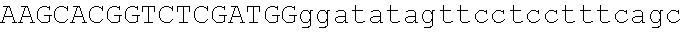 | Internal BsaI in |
| BsaR | | | ordered fragment 19 |

To include the pCMV in ordered fragment 0

| | | |
|---|---|---|
| 1876-CoVFrag0- | 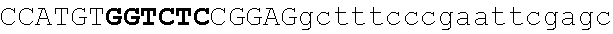 | Ordered fragment 0, nt51 |
| BsaF | (SEQ ID NO: 91) | fOR pGGA clone, Ordered |
| 1877-CoVFrag0- | 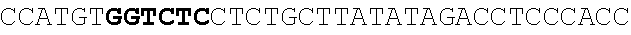 | fragment 0-4, start after the |
| BsaR | (SEQ ID NO: 92) | Internal BsaI within ordered |
| | | fragment 0 |
| 1878-CoVfrag1- | 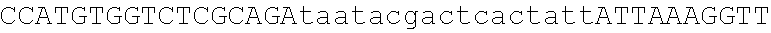 | Ordered fragment 1 |
| BsaF2 | (SEQ ID NO: 93) | |
| 1879-Covfrag1- | 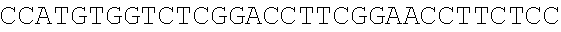 | |
| BsaF2 | (SEQ NO: 94) | |
| 1880-CoVfrag2- | 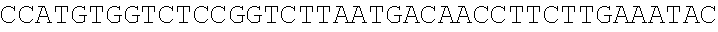 | use #1826-1830 for rest |
| BsaF2 | (SEQ NO. 95) | of the inserts |

Step 2: Assembly viral genome into 4 large fragments into pGGA using NEB Golden gate assembly kit

Fragment 0 (or 1)-4, 5-9, 10-14 and 15-19.

Follow NEB Golden gate assembly kit menu

Step 3 PCR viral fragments from pGGA clones for next step assembly (also introduce new Bsa I sites)

Step 3.1: Re-Introduce Bsa I Through PCR and Gel Purification (Individually)

| | SEQ | | |
|---|---|---|---|
| | ID NO: | | |
| 1881-pCC1-F | 96 | ATCGCAGGTCTCGcctcgaccaattctcatgtttg | pCC1BAC modified. |
| 1882-pCC1-R | 97 | 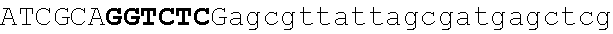 | For pCCIBAC assembly-full |
| | | | CoV |
| 1883- | 51 | 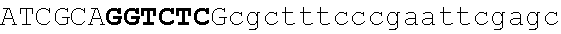 | pGGA-CoV0-4 |
| pGGAfrag1-F | | | |
| 1884- | 98 | 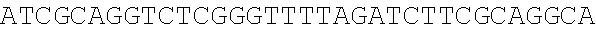 | |
| pGGAfrag1-R | | | |
| 1885- | 99 | 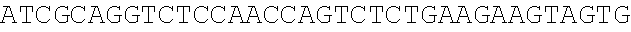 | pGGA-CoV5-9 |
| pGGAfrag2-F | | | |
| 1886- | 100 | 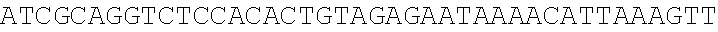 | |
| pGGAfrag2-R | | | |
| 1887- | 101 | 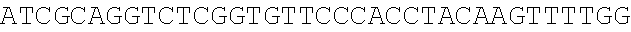 | pGGA-CoV10-14 |
| pGGAfrag3-F | | | |
| 1888- | 102 | 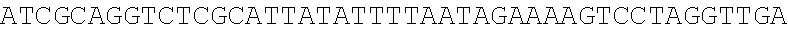 | |
| pGGAfrag3-R | | | |
| 1889- | 103 | 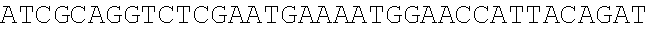 | pGGA-CoV15-19 |
| pGGAfrag4-F | | | CoV |
| 1890- | 52 | 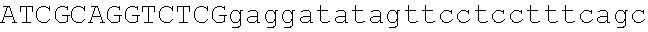 | For pCCIBAC assembly-full |
| pGGAfrag4-R | | | |
| 1891-pCC1- | 104 | 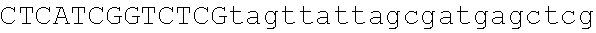 | For pCCIBAC assembly- |
| R2 | | | fCoV-start from T7, |
| | | | other primers same as |
| | | | full assembly |
| 1892- | 105 | 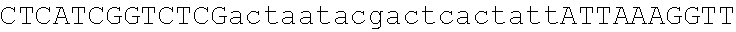 | For pCCIBAC assembly- |
| pGGAfrag1- | | | fCoV-start from T7, |
| F2 | | | other primers same as |
| | | | full assembly |

Step 3.2 Assemble the 4 Large Fragments into pCC1BAC Using NEB Golden Gate Assembly Kit

Assemble all Fragments into pCC1BAC Vector Directly

Step 1: PCR and Gel Purification

| | SEQ | | |
|---|---|---|---|
| | ID NO: | | |
| 1893- | 106 | 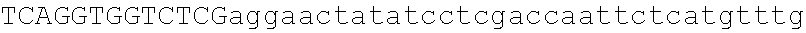 | pCC1BAC modified |
| pCC1FWD | | | For insert all CoVfrag Insto |
| 1894- | 107 | 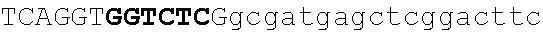 | pCC1 in one reaction |
| pCC1REV | | | pCC1BAC(modified) ends |
| | | | at nt7439 |
| 1895-CoVInst0 | 108 | 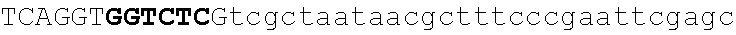 | Ordered fragment 0 |
| FWD | | | |
| 1896-CoVInst0 | 109 | 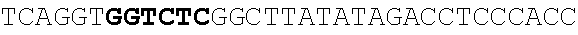 | |
| REV | | | |
| 1897-CoVInst1 | 110 | 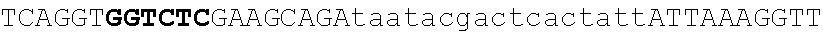 | Ordered fragment 1 |
| FWD | | | |
| 1898-CoVInst1 | 111 | 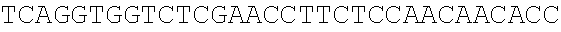 | |
| REV | | | |
| 1899-CoVInst2 | 112 | 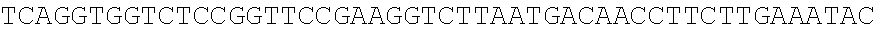 | Ordered fragment 2 |
| FWD | | | |
| 1900-CoVInst2 | 113 | 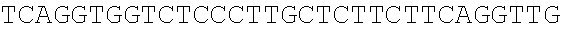 | |
| REV | | | |
| 1901-CoVInst3 | 114 | 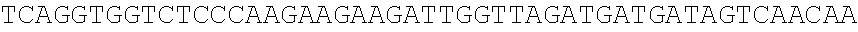 | Ordered fragment 3 |
| FWD | | | |
| 1902-CoVInst3 | 115 | 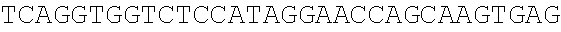 | |
| REV | | | |
| 1903-CoVInst4 | 116 | 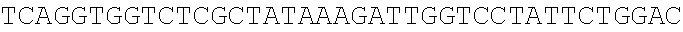 | Ordered fragment 4 |
| FWD | | | |
| 1904-CoVInst4 | 117 | 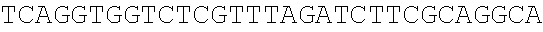 | |
| REV | | | |
| 1905-CoVInst5 | 118 | 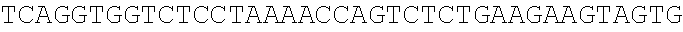 | Ordered fragment 5 |
| FWD | | | |
| 1906-CoVInst5 | 119 | 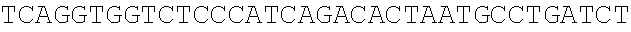 | |
| REV | | | |
| 1907-CoVInst6 | 120 | 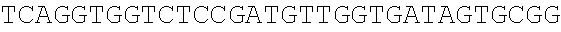 | Ordered fragment 6 |
| FWD | | | |
| 1908-CoVInst6 | 121 | 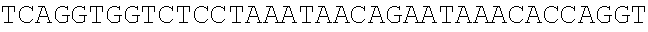 | |
| REV | | | |
| 1909-CoVInst7 | 122 | 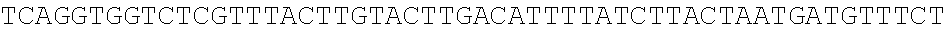 | Ordered fragment 7 |
| FWD | | | |
| 1910-CoVInst7 | 123 | 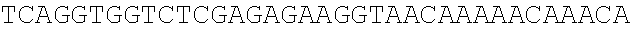 | |
| REV | | | |
| 1911-CoVInst8 | 124 | 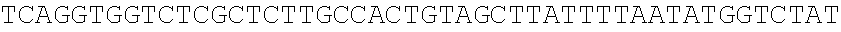 | Ordered fragment 8 |
| FWD | | | - with WT and Min |
| 1912-CoVInst8 | 125 | 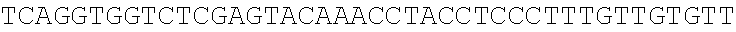 | versions, need to purify |
| REV | | | individually |
| 1913-CoVInst9 | 126 | 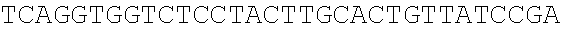 | Ordered fragment 9 |
| FWD | | | |
| 1914-CoVInst9 | 127 | 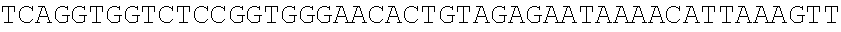 | |
| REV | | | |
| 1915-CoVInst10 | 128 | 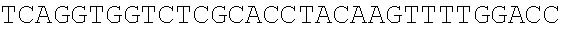 | Ordered fragment 10 |
| FWD | | | - with WT and Min |
| 1916-CoVInst10 | 129 | 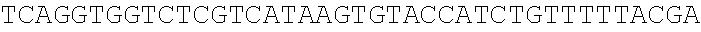 | versions, need to purify |
| REV | | | individually |
| 1917-CoVInst11 | 130 | 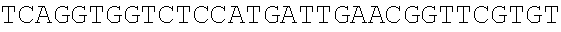 | Ordered fragment 11 |
| FWD | | | |
| 1918-CoVInst11 | 131 | 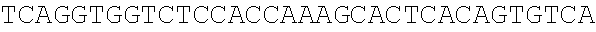 | |
| REV | | | |
| 1919-CoVInst12 | 132 | 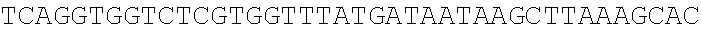 | Ordered fragment 12 |
| FWD | | | |
| 1920-CoVInst12 | 133 | 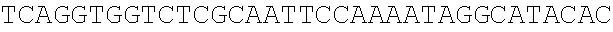 | |
| REV | | | |
| 1921-CoVInst13 | 134 | 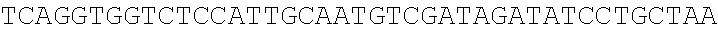 | Ordered fragment 13 |
| FWD | | | |
| 1922-CoVInst13 | 135 | 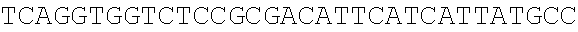 | |
| REV | | | |
| 1923-CoVInst14 | 136 | 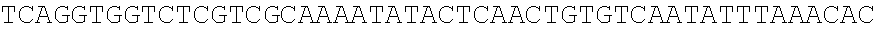 | Ordered fragment 14 |
| FWD | | | - with WT and Min |
| 1924-CoVInst14 | 137 | 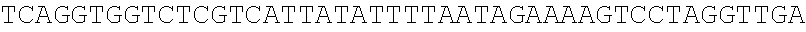 | versions, need to purify |
| REV | | | individually |
| 1925-CoVInst15 | 138 | 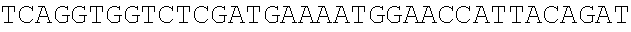 | Ordered fragment 15 |
| FWD | | | - with WT and Min |
| 1926-CoVInst15 | 139 | 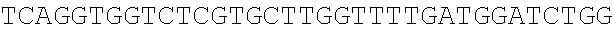 | versions, need to purify |
| REV | | | individually |
| 1927-CoVInst16 | 140 | 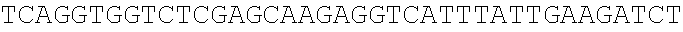 | Ordered fragment 16 |
| FWD | | | - with WT and Min |
| 1928-CoVInst16 | 141 | 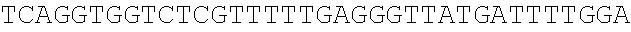 | versions, need to purify |
| REV | | | individually |
| 1929-CoVInst17 | 142 | 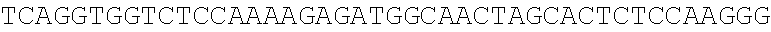 | Ordered fragment 17 |
| FWD | | | |
| 1930-CoVInst17 | 143 | 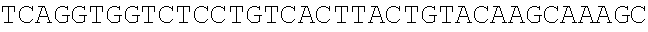 | |
| REV | | | |
| 1931-CoVInst18 | 144 | 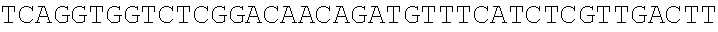 | Ordered fragment 18 |
| FWD | | | |
| 1932-CoVInst18 | 145 | 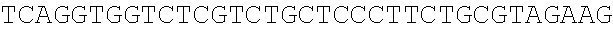 | |
| REV | | | |
| 1933-CoVInst19 | 146 | 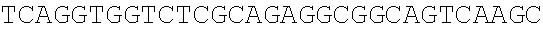 | Ordered fragment 19 |
| FWD | | | |
| 1934-CoVInst19 | 147 | 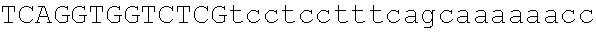 | |
| REV | | | |

Step 2: Assembly Full Viral Genome in pCC1BAC Vector Following NEB Golden Gate Assembly Kit Menu

### NEBuilder HiFi DNA Assembly

Step 1: PCR and gel purification. Same as example 1. For fragment 0 and fragment 19, need to use alternative primer for PCR and for 5′ and 3′ end of pCC1BAC.

For intersection between pCC1BAC and viral genome

### 5′ Intersection

| | | |
|---|---|---|
| 1935- | GTCCGAGCTCATCGCTAATA | Forward primer |
| CoVfrag0- | ACgctttcccgaattcgagc | for fragment 0, |
| F2 | (SEQ ID NO: 148) | for NEBuilder |
| 1936- | gctcgaattcgggaaagcGT | Replace #1861 |
| pCC1-R2 | TATTAGCGATGAGCTCGGAC | when modify |
| | (SEQ ID NO: 149) | pCC1 for |
| | | NEBuilder |

### 3′ Intersection

| | | |
|---|---|---|
| 1937- | CTGTCAAACATGAGAATTGGTC | Last 23nt in |
| CoVfrag19- | GAgaagacttggtacacttgag | Fragment 19, |
| R2 | acg (SEQ ID NO: 47) | first 24 in |
| | | pCC1, for |
| | | NEBuilder |
| 1938-pCC1- | cgtctcaagtgtaccaagtctt | Last 23nt in |
| F2 | cTCGACCAATTCTCATGTTTGA | Fragment 19, |
| | CAG (SEQ ID NO: 150) | first 24 in |
| | | pCC1, for |
| | | NEBuilder |

Step 2: Assembly Follow NEBuilder HiFi Menu

Modification of pCC1BAC vector. The backbone if the pCC1BAC is obtained from pCC1-CysS-CD plasmid. It carries internal Bsa I and Bsm BI sites. This modification is to remove these sites. It also removes the region between the 2 Not I sites, and nt8104-8139(?).

Step 1: PCR and Gel Purification

| | SEQ | | |
|---|---|---|---|
| | ID | | |
| | NO: | | |
| 1851-pCC1-NotI-F | 151 | AAGGAAAAAAGCGGCCGCccgggccg | |
| 1852-pCC1-NotI-F2 | 152 | AAGGAAAAAAGCGGCCGCtcgaccaattctcatgtttgac | pCC1 generated |
| | | agc | new Not I site |
| 1853-BsmBI-mut1-F | 153 | ctcacccagggattggcTGAtACGaaaaacatattctc | pCC1 mutate BsmBI |
| 1854-BsmBI-mutl-R | 154 | gagaatatgtttttCGTaTCAgccaatccctgggtgag | pCC1 mutate BsmBI |
| 1855-BsmBI-mut2-F | 155 | cttacgtgccgatcaaCGaCTCAttttcgccaaaagttgg | pCC1 mutate BsmBI |
| 1856-BsmBI-mut2-R | 156 | ccaacttttggcgaaaaTGAGtCGttgatcggcacgtaag | pCC1 mutate BsmBI |
| 1857-BsmBI-mut3-F | 157 | ggatggctcaggcatCGTgTCTctgaaaatcgactggatc | pCC1 mutate BsmBI |
| 1858-BsmBI-mut3-R | 158 | gatccagtcgattttcagAGAcACGatgcctgagccatcc | pCC1 mutate BsmBI |
| 1859-BsaI-mut-F | 159 | cttaacctggacaGGTCaCgtgttccaactgagtgtatag | pCC1 mutate BsaI |
| 1860-BsaI-mut-R | 160 | ctatacactcagttggaacacGtGACCtgtccaggttaag | pCC1 mutate BsaI |
| 1861-pCC1-ZraI-R | 161 | aattttGACGTCgttattagcgatgagctcggacttcc | |

| Rxn | Primer F/R | Template |
|---|---|---|
| 1 | *1851/1854 OR *1852/1854 | pCC1-CysS-CD, excise Cys-CD |
| 2 | 1853//1856 | with Not I digestion, purify the |
| 3 | 1855/1858 | back bone. |
| 4 | 1857/1860 | |
| 5 | 1859/*1861 | |

*To facilitate the following assembly step, one can use 1893 instead of 1851 or 1852, use
1894 instead of 1861.

Step 2: Overlapping PCR. For this method—NEBuilder, use alternative primers.

### Example 2

### Procedures

### RT-PCR

Coronavirus strain 2019-nCoV/USA-WA1/2020 (“WA1”) (BEI Resources NR-52281, Lot 70034262) was distributed by BEI Resources after 3 passages on Vero (CCL81) at CDC, and one passage on Vero E6 at BEI Resources. The full virus genome sequence after 4 passages was determined by CDC and found to contain no nucleotide differences (Harcourt et al., 2020) compared to the clinical specimen from which it was derived (GenBank Accession MN985325) Upon receipt, WA1 and was amplified by a further two passages on Vero E6 cells in DMEM containing 2% FBS at 37° C.

Passage 6 WA1 virus was used to purify viral genome RNA by extraction with Trizol reagent (Thermo Fisher) according to standard protocols. Briefly, 0.5 ml virus sample with a titer of 1×10\hat{ }7 PFU/ml was extracted with an equal volume of Trizol. The procedure had previously been validated in four separate experiment to completely inactivate SARS-CoV2 virus infectivity. After phase separation by addition of 0.1 ml chloroform, the RNA in aqueous phase was precipitated with an equal volume of isopropanol. The precipitated RNA was washed in 70% ethanol, dried, and resuspended in 20 ul RNAse-free water.

Viral cDNA Generation

Wild-type cDNA were synthesized using SuperScript IV First Strand Synthesis system. In each reaction, a total reaction volume of 13 μl for Tube #1 was set up as follows:
1. 50 μM Oligo d(T)20: 1 ul
2. 50 ng/μl Random Hexamer: 1 μl
3. 10 mM dNTP: 1 μl
4. WT RNA: 2-10 μl
5. H2O: add to 13 μl

The sample was mixed and incubated at 65° C. for 5 minutes, then immediately put on ice for 1 minute. Another tube (Tube #2) was prepared with a total reaction volume of 7 μl:
1. 5× Buffer: 4 μl
2. 100 mM DTT: 1 μl
3. Rnase Inhibitor (40 U/μl): 1 μl
4. SuperScript IV enzyme: 1 μl

We mixed Tube #1 and Tube #2, for a total reaction volume of 20 μl, and incubated at 23° C. for 10 minutes, followed by 50° C. for 50 minutes, and 80° C. for 10 minutes to generate cDNA.

### Overlapping Polymerase Chain Reaction

Q5 High-Fidelity 2× Master Mixture (NEB, Ipswich, Mass.) were used to amplify genome fragments from cDNA.

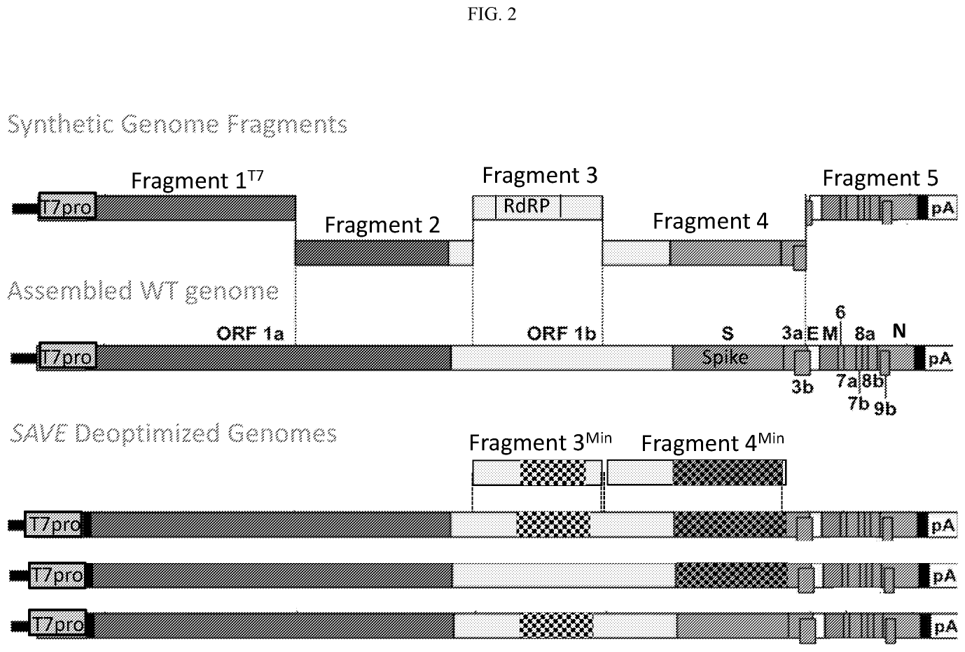

The 20 μl reaction containing 1 μl fresh-made cDNA, 1 μl of forward and reverse primers (detailed in Table 4) at 0.5 μM concentration, 10 μl of the 2×Q5 master mixture and H₂O. Reaction parameters were as follows: 98° C. 30 sec to initiate the reaction, followed by 30 cycles of 98° C. for 10 sec, 60° C. for 30 seconds, and 65° C. for 1 min and a final extension at 65° C. for 5 min. Totally 19 genome fragments, all about 1.8 Kb except fragment 19 (about 1.2 Kb) were obtained, which cover the whole viral genome with 200 bp overlapping region between any two of them using specific primers (Table 4). Amplicons were verified by agarose gel electrophoresis (FIG. 2A) and purified using the QIAquick PCR Purification Kit (Qiagen). Elutions were quantified by Nanodrop.

Q5® High-Fidelity DNA Polymerase (NEB, Ipswich, Mass.) were used to re-construct the whole COVID-19 genome.

First, all 19 genome fragments were used in an overlapping reaction to reconstruct the full genome. Briefly, a mixture with 30-40 ng of each DNA fragment (the molar ratio among all pieces are at 1:1), 10 μl 5× reaction buffer, 1 μl 10 mM dNTP, 0.5 μl Q5 polymerase and H₂O to a final volume of 50 μl was made. The reaction was carried out under following condition: 98° C. for 30 sec, and 72° C. for 16 min 30 sec for 10 cycles.

Next, 2 μl overlapping reaction product were mixed with 4 μl 5× reaction buffer, 1 μl 10 mM dNTP, 1 μl of each flanking primers at 0.5 μM, 0.241 Q5 polymerase and H₂O to a final volume of 20 μl and PCR was carried out as follows: 98° C. 30 sec to initiate the reaction, followed by 15 cycles of 98° C. for 10 sec, 60° C. for 45 sec, and 72° C. for 16 minutes 30 seconds, and a final extension at 65° C. for 5 min. To check the results, 5 μl PCR product was visualized on 0.4% agarose gel (FIG. 2B).

TABLE 4

| Primers for RT-PCR | | | |
|---|---|---|---|
| SEQ | | | |
| ID | | | |
| NO: | No. | Name | oligo sequence 5′-3′ |
| 164 | 2312 | 2312-Frl-T7G-F3 | 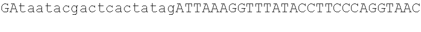 |
| 10 | 1786 | 1786-COV-2 | GATGCCAAAATAATGGCGATCTC |
| 11 | 1787 | 1787-COV-3 | GTTGGTTGCCATAACAAGTGTG |
| 12 | 1788 | 1788-COV-4 | CTAATTGAGGTTGAACCTCAACAATTG |
| 13 | 1789 | 1789-COV-5 | GAGTATGGTACTGAAGATGATTACCAAG |
| 14 | 1790 | 1790-COV-6 | CTAGGTGGAATGTGGTAGGATTAC |
| 15 | 1791 | 1791-COV-7 | GCTGTTACAGCGTATAATGGTTATCTTAC |
| 16 | 1792 | 1792-COV-8 | GCTGGTTTAAGTATAATGTCTCCTACAAC |
| 17 | 1793 | 1793-COV-9 | GCACAAAACCAGTTGAAACATCAAATTC |
| 18 | 1794 | 1794-COV-10 | GCAACTAGTGTTTTGAGTTTTTCCATTG |
| 19 | 1795 | 1795-COV-11 | GTGAAGAATCATCTGCAAAATCAGC |
| 20 | 1796 | 1796-COV-12 | CAAATGATATAAGCAATTGTTATCCAGAAAGG |
| 21 | 1797 | 1797-COV-13 | GCCTTTAATACTTTACTATTCCTTATGTCATTCAC |
| 22 | 1798 | 1798-COV-14 | CCAGACAAACTAGTATCAACCATATCC |
| 23 | 1799 | 1799-COV-15 | GCTATGGGTATTATTGCTATGTCTG |
| 24 | 1800 | 1800-COV-16 | CCTACAAGGTGGTTCCAGTTC |
| 25 | 1801 | 1801-COV-17 | CGACAGATGTCTTGTGCTG |
| 26 | 1802 | 1802-COV-18 | GGTATCCAGTTGAAACTACAAATGG |
| 27 | 1803 | 1803-COV-19 | GATCAGACATACCACCCAAATTG |
| 28 | 1804 | 1804-COV-20 | CTTATGTATTGTAAGTACAAATGAAAGACATCAG |
| 29 | 1805 | 1805-COV-21 | GGTGATGATTATGTGTACCTTCCTTAC |
| 30 | 1806 | 1806-COV-22 | CTGTTAATTGCAGATGAAACATCATGC |
| 31 | 1807 | 1807-COV-23 | GTGTGTAGACTTATGAAAACTATAGGTCC |
| 32 | 1808 | 1808-COV-24 | CATACAAACTGCCACCATCAC |
| 33 | 1809 | 1809-COV-25 | CCTTGTAGTGACAAAGCTTATAAAATAGAAG |
| 34 | 1810 | 1810-COV-26 | CTGGTGCAACTCCTTTATCAG |
| 35 | 1811 | 1811-COV-27 | GCAAAGAATGCTATTAGAAAAGTGTGAC |
| 36 | 1812 | 1812-COV-28 | GATAGATTCCTTTTTCTACAGTGAAGGATTTC |
| 37 | 1813 | 1813-COV-29 | GACTCCTGGTGATTCTTCTTCAG |
| 38 | 1814 | 1814-COV-30 | CTCTAGCAGCAATATCACCAAGG |
| 39 | 1815 | 1815-COV-31 | GCACAAGTCAAACAAATTTACAAAACAC |
| 40 | 1816 | 1816-COV-32 | CAAAAGGTGTGAGTAAACTGTTACAAAC |
| 41 | 1817 | 1817-COV-33 | CTCACTCCCTTTCGGATGG |
| 42 | 1818 | 1818-COV-34 | GAGGTTTATGATGTAATCAAGATTCCAAATGG |
| 43 | 1819 | 1819-COV-35 | GCTACAGGATTGGCAACTATAAATTAAAC |
| 44 | 1820 | 1820-COV-36 | CCATTCTAGCAGGAGAAGTTCC |
| 45 | 1821 | 1821-COV-37 | GCAATCCTGCTAACAATGCTG |
| 46 | 1822 | 1822-COV-38 | ttttTTTTTTTTTTTTTTTTTTTTTGTCATTCTCCTAAGAAGCTATTAAAATC |

### In Vitro Transcription

DNA templates amplified from full-length PCR were purified using conventional phenol/chloroform extraction followed by Ethanol precipitation in the presence of 3M Sodium Acetate prior to RNA work. RNA transcripts was in vitro synthesized using the HiScribe T7 Transcription Kit (New England Biolabs) according to the manufacturer's instruction with some modifications. A 20 μl reaction was set up by adding 500 ng DNA template and 2.4 μl 50 mM GTP (cap analog-to-GTP ratio is 1:1). The reaction was incubated at 37° C. for 3 hr. Then RNA was precipitated and purified by Lithium Chloride precipitation and washed once with 70% Ethanol. The N gene DNA template was also prepared by PCR from cDNA using specific forward primer (2320-N-F: GAAtaatacgactcactataggGACGTTCGTGTTGTTTTAGATTTCATCTAAACG (SEQ ID NO:162), the lowercase sequence represents T7 promoter; the underlined sequence represents the 5′ NTR upstream of the N gene ORF) and reverse primer (2130-N-R, tttt GTCATTCTCCTAAGAAGCTATTAAAATCACATGG (SEQ ID NO:163)).

### Transfection of Vero E6 Cells by RNA Electroporation

Vero E6 cells were obtained from ATTC (CRL-1586) and maintained in DMEM high glucose supplemented with 10% FBS. To transfect viral RNA, 10 μg of purified full length genome RNA transcripts, together with 5 ug of capped WA1-N mRNA, were electroporated into Vero E6 cells using the Maxcyte ATX system according manufacturer's instructions. Briefly, 3-4×10⁶ Vero E6 cells were once washed in Maxcyte electroporation buffer and resuspended in 100 μl of the same. The cell suspension was mixed gently with the RNA sample, and the RNA/cell mixture transferred to Maxcyte OC-100 processing assemblies. Electroporation was performed using the pre-programmed Vero cell electroporation protocol. After 30 minutes recovery of the transfected cells at 37 C/5% CO₂, cells were resuspended in warm DMEM/10% FBS and distributed among three T25 flasks at various seeding densities (½, ⅓, ⅙ of the total cells). Transfected cells were incubated at 37° C./5% CO₂ for 6 days or until CPE appeared. Infection medium was collected on days 2, 4, and 6, with completely media change at day 2 and day 4 (DMEM/5% FBS). The generated viruses were detectable by plaque assay as early as 2 days post transfection, with peak virus generation between days 4-6.

### Passaging of Stock Virus and Plaque Titration of SARS-CoV-2 in Vero E6 Cells

Serial 10-fold dilutions were prepared in DMEM/2% FBS. 0.5 ml of each dilution were added to 12-wells of Vero E6 cells that were 80% confluent. After 1 hour incubation at 37° C., the inoculum was removed, and 2 ml of semisolid overlay was added per well, containing 1×DMEM, 0.3% Gum Tragacanth, 2% FBS and 1× Penicillin/Streptomycin. After 3 or 4 day incubation at 37° C./5% CO₂ the overlay was removed, wells were rinsed gently with PBS, followed by fixation and staining with Crystal Violet.

RNA obtained from In vitro transcription was used to transfect Vero E6 cells with wt WA1 and CDX-005 and recover live virus that was titrated in Vero E6 cells. After incubation for 3 days, plaque assays were stained.

### Multistep Virus Growth Kinetics

Vero cells (WHO 10-87) were grown for 3 days in 12 well plates containing 1 ml DMEM with 5% fetal bovine serum (FBS) until they reached near confluency. Prior to infection, spent cell culture medium was replaced with 0.5 ml fresh DMEM containing 1% FBS and 30 PFU of the indicated viruses (0.0001 MOI). After a 1-hour incubation at 33° C. or 37° C./5% CO2, inoculum was discarded, cell monolayers were washed once with 1 ml Dulbecco's PBS, followed by addition of 1 ml DMEM containing 1% FBS. Infected cells were incubated at 33° C. or 37° C. for 0, 6, 24, 48, or 72 hrs. At the indicated timepoints, cells and supernatants were collected (one well per time point), frozen once at −80 C and thawed. Infectious virus titers in the lysates were determined by plaque assay on Vero E6 at 37° C.

### Results

Generation of individual genome fragments 1-19 and the whole genomic DNA generated by overlapping PCR went well, with clear bands visible on 0.4% agarose gels (FIG. 2A).

In vitro transcription produced RNA used to transfect Vero E6 cells with S-WWW (WT) and S-WWD and recover live virus that was titrated in Vero E6 cells. After incubation for 3 days, the plaque assays were stained and we observed smaller plaques observed in the partially spike-deoptimized S-WWD candidate (FIG. 3) and a 40% reduced final titer.

### Example 3

The CDX-005 pre-master virus seed (preMVS) was developed as follows: RNA of SARS-COV-2 BetaCoV/USA/WA1/2020 (GenBank: MN985325.1) was extracted from infected, characterized Vero E6 cells (ATCC CRL-1586 Lot #70010177) and converted to 19 overlapping DNA fragments by RT-PCR using commercially available reagents and kits. Overlapping PCR was used to stitch together 19 1.8 kb wt genome fragments along with one deoptimized Spike gene cassette. Specifically, 1,272 nucleotides of the Spike ORF were human codon pair deoptimized from genome position 24115-25387 resulting in 283 silent mutations changes relative to parental WA1/2020 virus. The resulting full-length cDNA was transcribed in vitro to make full-length viral RNA. Viral recovery was conducted in a new BSL-3 laboratory at Stony Brook University (NY) that was commissioned for the first time in April 2020, with our project being the only project ever to occur in the lab. This viral RNA was then electroporated in characterized Vero E6 cells (Lot #70010177). This yielded CDX-005 virus (FIG. 3) that was subsequently passaged an additional time on Vero E6 cells to yield passage 1, P1 (Lot #1-060820-9-1). P1 material was used in the hamster study described below.

### Example 4

To produce a seed virus (preMVS) suitable for entrance into GMP, the P1 lot was passaged at Codagenix in the our characterized 10-87 WHO Vero cells (Lot: 563173-MCB1, COA and characterization testing) supplemented with qualified 2% fetal bovine serum (FBS) sourced from New Zealand. Resulting virus was clarified by centrifugation, sterile filtered and filled into 2 ml cryovials to yield preMVS (Lot #1-061720-1) with the titer of 5×10⁵ 2.6×10⁶ pfu/ml. The preMVS is to be sent to BioReliance (Glasgow, UK) for sterility and mycoplasma testing under BSL3. Furthermore, the Vero culture that produced 1-061720-1 was allowed to grow for additional two-days to full cytopathic effect and then vialed to conduct and for comprehensive, molecular-based adventitious virus testing including simian, human, porcine and bovine viruses at Charles River Laboratories, Malvern, Pa., USA (Table 5).

TABLE 5
Adventitious virus testing panel for CDX-005 preMVS.

| Human | Simian | Porcine | Bovine |
|---|---|---|---|
| Adenovirus | Squirrel Monkey Retrovirus | Circovirus | Bovine |
| Adeno-associated virus (AAV) | (SMRV) | Type 1 and 2 | Polyoma virus |
| Cytomegalovirus (CMV) | Simian T-Lymphotropic Virus | | Bovine |
| Epstein-Barr virus (EBV) | (STLV) | | circovirus |
| Hepatitis A, B and C | Simian Immunodeficiency | | |
| Herpes 6, 7 and 8 | Virus (SIV) | | |
| Human Immunodeficiency virus | Simian Foamy Virus (SFV) | | |
| (HIV-1 and HIV-2 | Simian Virus 40 (SV40) | | |
| Human T Lymphotrophic virus | Simian Retrovirus -1, -2 and -3 | | |
| (HTLV-1 and HTLV-2) | (SRV -1, -2 and -3) | | |
| Human Foamy Virus (HFV) | | | |

Master Seed Virus (MSV) is used as Phase I trial material and is cGMP manufactured. CDX-005 will be produced in Vero (ATCC CCL-81) cell line, and is tested using qualified methods and release the product for clinical testing. A formulation for CDX-005 is as currently employed for their intranasal, live-attenuated influenza vaccine in Phase III trials, which was shown to provide stability and safety. A manufacturing process is shown below.

Manufacturing process for CDX-005

### Example 5A

The WHO ad hoc Expert Working Group on COVID-19 modelling concluded that both rhesus macaques and ferrets appear to reproduce mild to moderate human disease, but more recent work (Chan, Sia) suggests that Syrian Golden Hamsters may be a more useful model to replicate more severe pulmonary manifestations of this infection.

To investigate the in vivo properties of CDX-005 we turned to Syrian Golden hamsters. A recent survey of current animal models indicates that these hamsters best recapitulate the characteristics of human COVID-19 disease. SARS-CoV-2 replicates efficiently in hamster lungs causing severe pathological lesions following intranasal infection. Viral antigens are present in nasal mucosa, bronchial epithelium, and areas of lung consolidation on Days 2 and 5 after SARS-CoV-2 inoculation, that is cleared on Day 7. Clinical signs include rapid breathing, weight loss, histopathological changes in the lung/airway, intestinal involvement, spleen and lymphoid atrophy, and cytokine activation within one week of virus challenge. Infected hamsters can infect other hamsters housed in the same cage, and neutralizing antibodies (Abs) are detected on Day 14 post-challenge.

The inventors evaluated attenuation of CDX-005 P1 (Lot #1-060820-9-1) compared to WT BetaCoV/USA/WA1/2020 in the hamster model under BSL-3. Further, our challenge with WT 14 days post vaccination will examine the presence/absence of vaccine enhancement.

Thirty-six male hamsters 5-8 weeks old were dosed by intranasal dropper on Day 0 (12 per group) with 0.05 ml of either nominal doses of 5×10⁴ PFU/ml or 5×10³ PFU/ml of wild-type WA1/2020 SARS-CoV-2 or 5×10⁴ PFU/ml CDX-005 and followed for 2, 4, 14, and 16 days. Endpoints include cage side observations twice daily, weight daily and temperature twice daily through day 5 and then daily through day 14. Viral load will be measured by qPCR and TCID₅₀ in nasal wash, lung tissue, brain and kidney on Days 2, 4 and 6 post-dosing. The right lung, right kidney and right brain hemisphere will be fixed for histopathologic examination at the same timepoints. On Day 14, the remaining 3 animals dosed with CDX-005 will be challenged with 5×10⁴ PFU/ml wild-type WA1/2020 and nasal wash, lung, brain, and kidney viral load will be measured on Day 16 along with histopathology of the same organs.

TABLE 6
Evaluation of CDX-005 in Syrian Gold Hamsters.
Evaluation of CDX-005 in Syrian Gold Hamsters

| | | | | | | Body |
|---|---|---|---|---|---|---|
| | | | | | Organs/NW | Temp/ |
| | | | PFU/ml | Challenge | n = 3 | Weights |
| Group | Virus | N | dose | (Day) | (day) | (Days) |
| 1 | CDX-005 | 12 | 5 × 10⁴ | 14 | 2, 4, 6, 16 | 0-16 |
| | 1-060820-9-1 | | | | | |
| 2 | WA1/2020 | 12 | 5 × 10⁴ | n/a | 2, 4, 6 | 0-14 |
| 3 | WA1/2020 | 12 | 5 × 10³ | n/a | 2, 4, 6 | 0-14 |

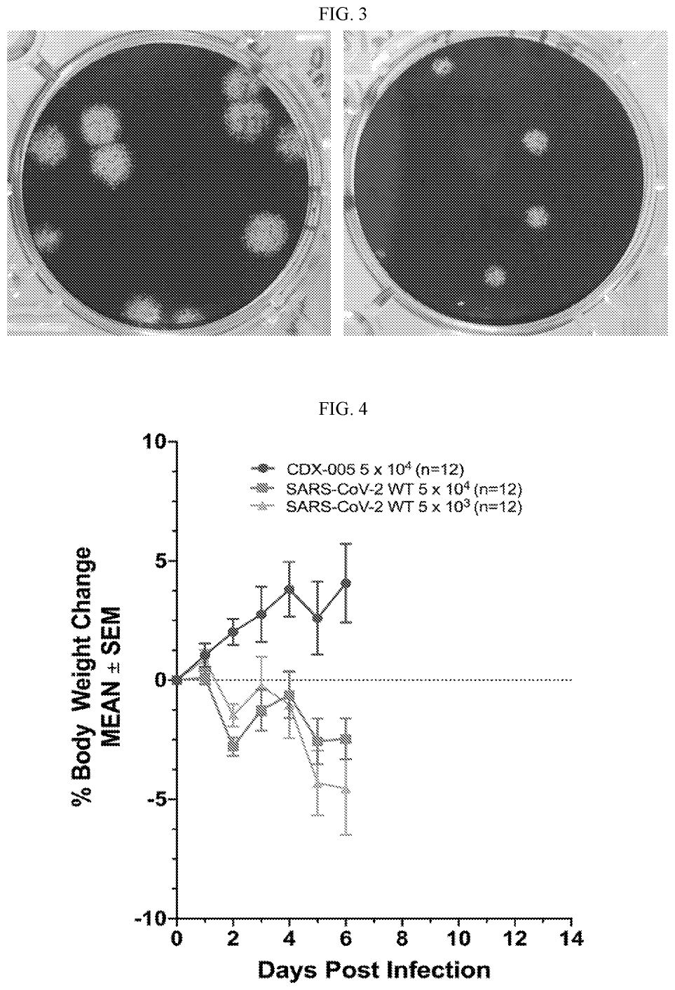

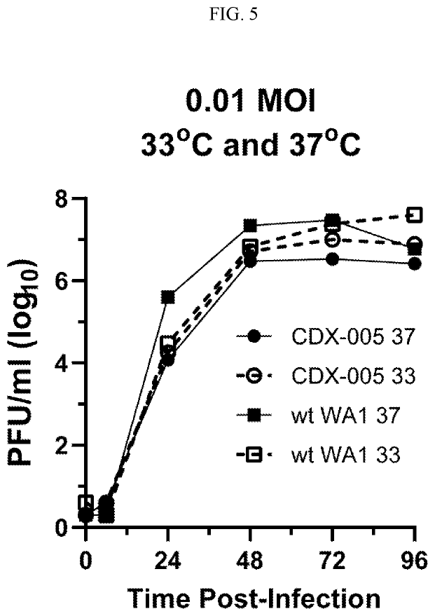

Initial data measuring weight loss for 6-days post-inoculation from this study is shown in FIG. 4. CDX-005 appears to be significantly attenuated compared to wild-type. Hamsters dosed with nominal dose of 5×10⁴ of CDX-005 experienced on average a weight gain of 4.1%, whereas, hamsters dosed with wild-type SARS-Cov-2 at nominal doses of 5×10⁴ and 5×10³ experienced weight losses averages of −2.5%. Back titration of doses on-going to determine exact doses delivered in this study.

Given an approximate hamster weight of 0.1 kg, extrapolation of hamster vaccine dose of 5×10⁴ to a 70 kg human would be equivalent to a dose of 3.5×10⁷, which is higher than likely maximum clinical dose to be tested.

Further, our challenge with WT 14 days post vaccination examines the presence/absence of vaccine enhancement.

Lastly, we appreciate the small sample size; however, the urgent nature of the pandemic combined with BSL-3 space scarcity and this demonstration of marked attenuation should support testing CDX-005 in healthy low-risk adults, who often have asymptomatic infection even with wild-type SARS-CoV-2.

A single 5.0×10⁴ PFU dose of CDX-005 was protective from wild-type challenge 16 days post dose. This pharmacological dose resulted in no distribution to the brain or kidney and limited distribution to the lung with minimal histopathological findings.

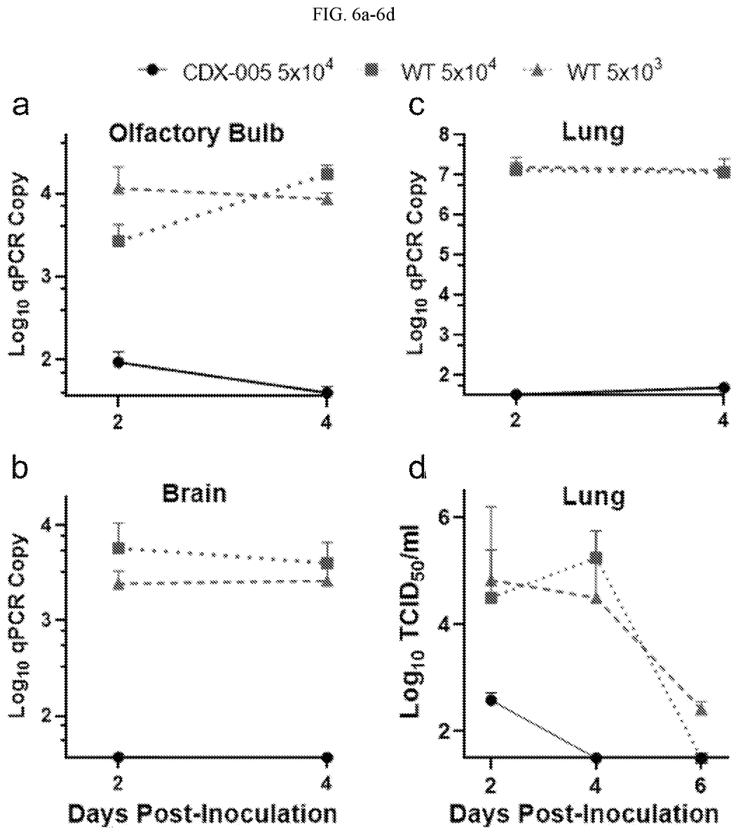

Because COVID-19 disease is associated with pulmonary, olfactory and neural dysfunction, we measured viral load in homogenized lungs, olfactory bulbs and brains. Total viral RNA measured by qPCR was near or below the limit of detection in lung, olfactory bulb, and brain on Days 2 and 4 post-inoculation (PI) in CDX-005 inoculated hamsters. In contrast, viral RNA was detected in wt WA1 infected hamsters in all three tissues at both times (FIG. 6a-c). To test if infectious virus was present, we performed TCID₅₀ assays on lung homogenates on Days 2, 4, and 6 PI (FIG. 6d). By Day 4, infectious virus loads were nearly 10,000-times lower in the lungs of animals inoculated with CDX-005 versus wt WA1-infected animals at either dose (N=3/group; P<0.01 by factorial ANOVA for independent samples) (FIG. 6d). Thus, CDX-005, is highly attenuated relative to wt WA1 in vivo.

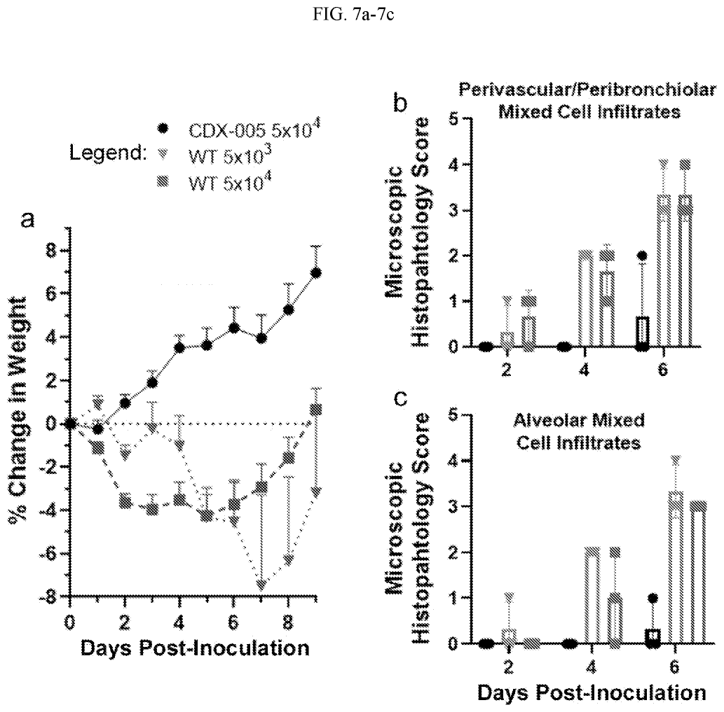

To evaluate the safety of CDX-005, the change in weight in hamsters was monitored for nine days after inoculation. Hamsters inoculated with CDX-005 experienced weight gain during the period, whereas those inoculated with wt WA1 at either dose experienced weight loss (FIG. 7a). Only at Day 9 PI did the wt WA1 treated animals return to their starting weight (FIG. 7a). A mixed model ANOVA indicated that the changes were significantly different between CDX-005 and wt WA1 treated groups (P<0.001).

We also performed histological examinations of the lungs, brain, and kidney. Formalin fixed paraffin sections were stained with hematoxylin and eosin and light microscopic evaluation was conducted by a blinded board-certified veterinary pathologist (N=3 per group). Multiple parameters were scored on a 0-5 pathology rating scale. No changes were noted in brain and kidney sections of hamsters administered wt WA1 or CDX-005. Consistent with the pathological cellular infiltration found in lungs of humans with COVID-19, however, alveolar and/or perivascular or peribronchiolar mixed cell infiltrates, necrosis of the bronchiolar or bronchial epithelium with neutrophilic infiltration into the lumen, and perivascular edema occasionally accompanied by hyperplasia of the bronchiolar or bronchial epithelium was seen in hamsters infected with wt WA1 (FIG. 7b-c). In contrast, lung pathology was limited to minimal alveolar to mild perivascular or peribronchiolar mixed cell infiltrates at Day 6 PI in CDX-005 inoculated hamsters (FIGS. 7b-c).

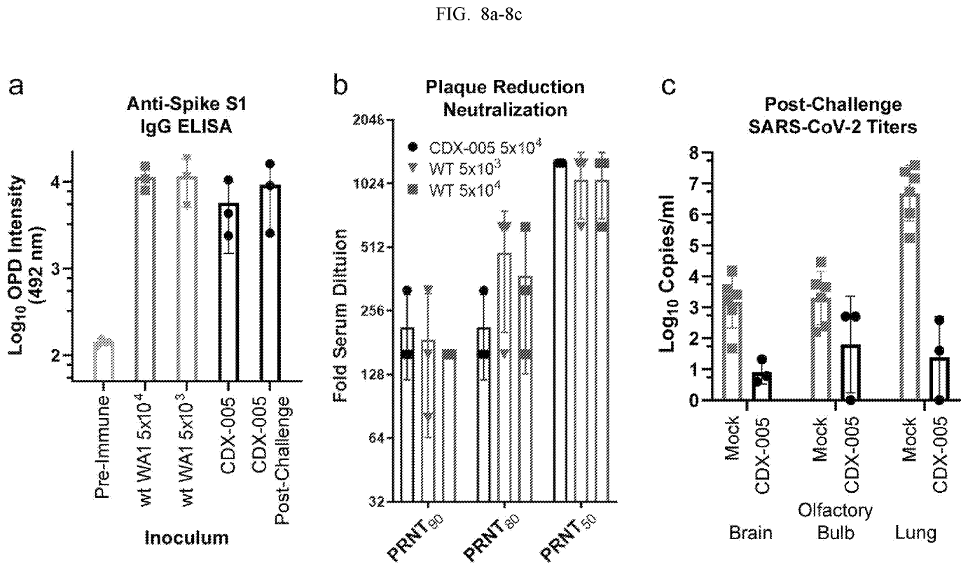

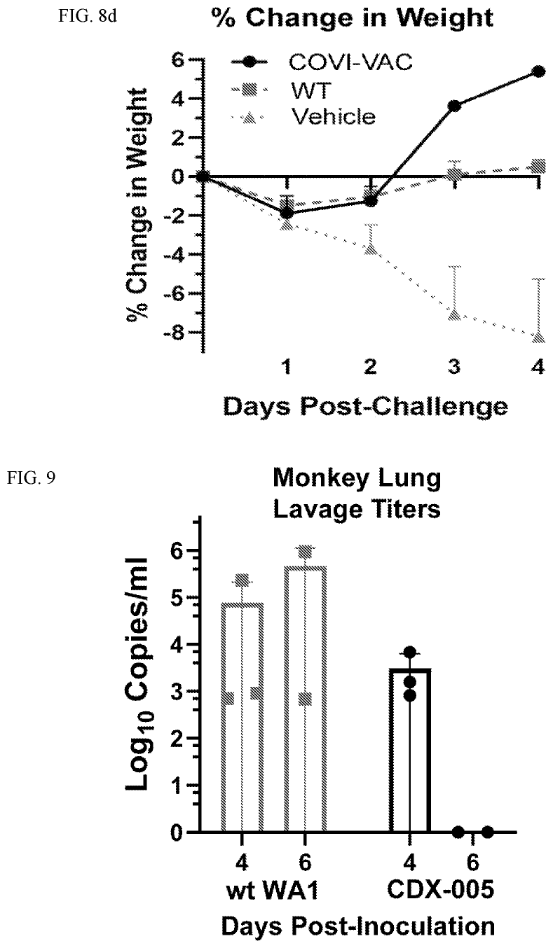

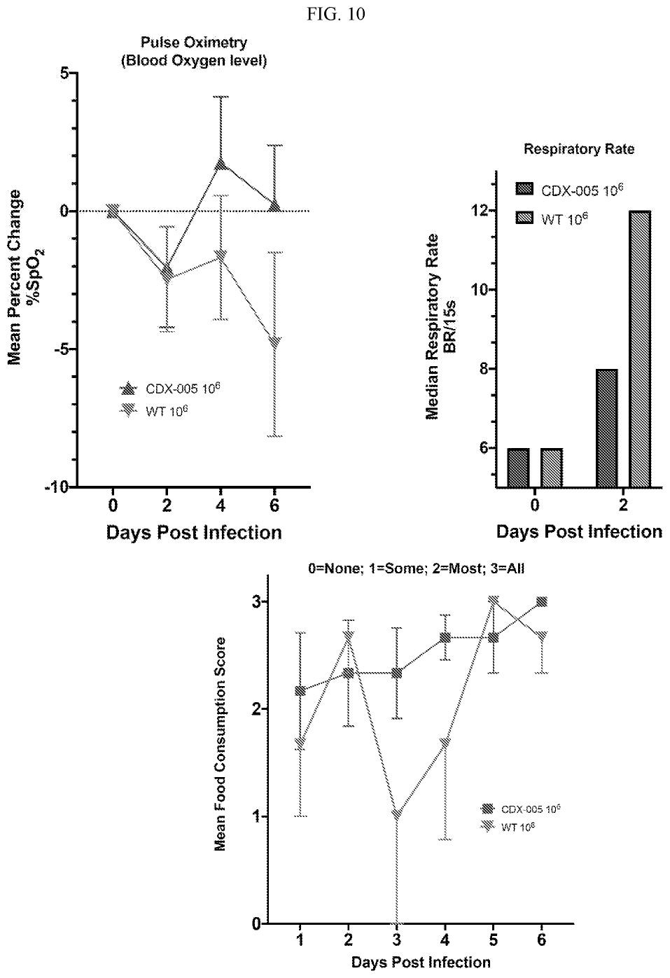

To assess efficacy of CDX-005 as a vaccine, we measured its ability to induce Abs against wt WA1. First, we performed an ELISA to determine IgG titers against SARS-CoV-2 Spike S1 in sera from naïve (mock) hamsters and those inoculated with wt WA1 or CDX-005. Like wt WA1, CDX-005 inoculation induced a strong anti-Spike S1 Abs response (FIG. 8a). We then tested for the presence of neutralizing Abs against wt WA1 virus in serum of inoculated hamsters by plaque reduction neutralization titers (PRNT) on Day 16 PI. We calculated 50, 80, and 90 percent neutralization (inhibition of plaque formation) with serum dilutions up to 1280-fold (FIG. 8a). All CDX-005 inoculated hamsters produced neutralizing Ab titers at levels similar to those induced by wt WA1 inoculation (FIG. 8b).

Finally, we measured the efficacy of CDX-005 in a challenge study. Hamsters were vaccinated intranasally (IN) a single dose of 5×10⁴ PFU CDX-005, and then challenged IN with 5×10⁴ PFU wt WA1 on Day 16 PI. Lungs were harvested on Day 18 (Day 2 post-challenge), and viral loads measured by qRT-PCR. Viral loads of the challenge wt WA1 virus were reduced by more than 10,000-fold in the lungs of CDX-005 vaccinated compared to un-vaccinated hamsters, attesting to the efficacy of the vaccine (FIG. 8c). Whereas SARS-CoV-2 virus was detected in the brains of challenged mock inoculated hamsters, none was detected in CDX-005 protected animals (FIG. 8c). Levels in the olfactory bulbs were not statistically different in the two groups (FIG. 8c). Thus, CDX-005 conferred protection and there was no evidence of vaccine-induced disease enhancement upon challenge. We also found that IgG Abs levels remained high in CDX-005 inoculated hamsters at Day 2 post-challenge (FIG. 8a).

### Example 5B

Hamster Studies

The WHO ad hoc Expert Working Group on COVID-19 modelling concluded that both rhesus macaques and ferrets appear to reproduce mild to moderate human disease, but more recent work suggests that Syrian Golden Hamsters may be a more useful model to replicate more severe pulmonary manifestations of this infection.1-3 Hence, Codagenix is currently evaluating attenuation of CDX-005 P1 (Lot #1-060820-9-1) compared to WA1 in the hamster model under BSL-3. All animal studies were performed according to IIT Research Institute IACUC approved protocols.

Thirty-six male Syrian hamsters (Charles Rivers) 5-6 weeks old were utilized on study. For challenge, hamsters were anaesthetized with ketamine (100 mg/kg) and xylazine (10 mg/kg) via intraperitoneal injection and inoculated intranasally on Day 0 (12 per group) with 0.05 ml of either nominal doses of 5×10⁴ PFU/ml or 5×10³ PFU/ml of wt WA1 SARS-CoV-2 or 5×10⁴ PFU/ml CDX-005 Animals were observed twice daily and body weights collected daily through Day 8 and then daily from Day 16-Day 18. On Day 16, three CDX-005 inoculated animals were challenged intranasally with 5×10⁴ PFU/ml wt WAL. Six naïve hamsters inoculated with either 5×10⁴ PFU/ml (N=3) or 5×10³ PFU/ml (N=3) of wt WA1 served as controls. We combined these two groups as titers overlapped at the two inoculation doses.

### Weight

These 36 hamsters and an additional 58 (half female/half male) 5-6 weeks old Syrian Golden hamsters (Charles Rivers) were used to study the effects of CDX-005 and wt WA1 inoculation on hamster health as assessed by weight loss. (These additional hamsters are currently being evaluated for other CDX-005 and wt WA1 mediated effects.) In total forty 5×10⁴ PFU CDX-005, forty 5×10⁴ PFU wt WA1, and twelve 5×10³ PFU wt WA1 were weighed daily for up to nine days. The N decreased over time for each group as animals were sacrificed for other endpoints on various days PI. The minimum N for 5×10⁴ PFU CDX-005 and 5×10⁴ PFU wt WA1 was 10 and 3 for 5×10³ PFU wt WA1.

### Tissue Harvesting

On days 2, 4, 6 post inoculation three hamsters from each group and on three hamsters on Day 18 from animals challenged on Day 16 were euthanized by intravenous injection of Beuthanasia at 150 mg/kg. The left lung was collected for viral load determination. To measure viral load, lung was homogenized in a 10% w/v in DMEM with antibiotics using a tissue homogenizer (Omni homogenizer) on Day 18 in animals challenged on Day 16 We attempted to perform nasal washes but were unsuccessful in obtaining reproducible washes in these small animals.

### Histopathology

Histopathology was performed by a blinded licensed veterinary pathologist. The lungs, brains, and kidneys were formalin fixed, dehydrated, embedded in paraffin, and stained with hematoxylin and eosin. Light microscopic evaluation was conducted by a blinded board-certified veterinary pathologist. Each tissue was graded on multiple pathological parameters and sections scored as 0=Normal, 1=Minimal, 2=Mild, 3=Moderate, 4=Marked, or 5=Severe. Evaluation of all tissues included assessment of cellular infiltration. At least five sections were examined for each organ and scores averaged.

### Viral Load

Viral load was measured by qPCR and TCID50 in harvested tissue. To measure viral load, tissue was homogenized in a 10% w/v in DMEM with antibiotics using a bead mill homogenizer (Omni). Infectious virus titers were determined by 50% tissue culture infectious dose (TCID50) assay titrating 10-fold serial dilutions of the lung homogenate on Vero E6 cells and are expressed in log 10 TCID50 units per ml. RNA was extracted from 100 μl of brain homogenate using the Quick-RNA Viral Kit (Zymo Research) according to the manufacturer's protocol. qRT-PCR was performed using the iTaq 1-step universal probe kit (Bio-Rad) using the following PCR cycling conditions: 40 cycles of 15 s at 95° C., 15 s at 60° C. and 20 s at 72° C.

### Antibodies—Plaque Reduction Neutralization Titer

Hamster sera collected at Day 16 PI were heat inactivated for 30′ at 56 C. 50 ul two-fold serial dilutions were performed in DMEM/1% FBS in 96-well U-bottom plates, starting with an initial dilution of 1:5. Approximately 30 PFU of SARS-CoV-2 Washington/1/2020 in 50 ul DMEM/1% FBS was added to the serum dilutions and mixed, bringing the final volume in the neutralization wells to 100 ul, and the total initial serum dilution to 1:10. Dilution plates were incubated for one hour at 37° C./5% CO2.

The cell growth medium on 24-well plates containing confluent monolayers of Vero E6 cells (seeded one day prior in DMEM/5% FBS), was removed, and 150 ul fresh DMEM/1% FBS was added, followed by 100 ul of each neutralization reaction. After one hour virus adsorption at 37° C./5% CO2, 0.75 ml semisolid overlay was added to the 24 well plates for a final concentration of 1×DMEM, 1.75% FBS, 0.3% Gum Tragacanth, 1× Penicillin+Streptomycin, in a total volume of 1 ml. 24 well plates were incubated 48 hours at 37° C. to allow for plaque formation. Plaques were visualized by fixing and staining the cell monolayers with 1% Crystal Violet in 50% Methanol/4% Formaldehyde. The plaque reduction neutralization titer (PRNT)50, 80, 90 was determined as the reciprocal of the last serum dilution that reduced plaque numbers by the pre-defined cutoff (50%, 80%, 90%) relative to the plaque numbers in non-neutralized wells (containing naïve hamster serum). Sera that failed to neutralize at the lowest dilution (1:10) was assigned a titer of 5, and sera that neutralized at the highest tested serum dilution (1:1280) were assigned a titer of ≥1280.

### Antibodies—IgG ELISA

Ninety-six well plates were coated with SARS-CoV-2(2019-nCoV) Spike S1-His (Sino Biological) at 30 ng/well in 50 ng/ml BSA/0.05M Carbonate/Bicarbonate Buffer pH9.6 overnight at 4° C. Plates were blocked with 10% goat serum in PBS 2 hr at 37° C., washed four times with washing buffer (0.1% Tween 20 in PBS) then incubated with a serially diluted serum (1:10 starting dilution and two folds thereafter) in 10% Goat serum/0.05% Tween-20 in PBS and incubated 1 hr at 37° C. Plates were washed four times with washing buffer then incubated with 1:10,000 horseradish peroxidase (HRP) conjugated affinity pure goat anti-Syrian hamster IgG (H & L) (Jackson ImmunoResearch Laboratories, Inc.) for 1 hr at 37° C. After the incubation, the plates were washed four times with washing buffer and Thermo Scientific OPD (o-phenylenediamine dihydrochloride) was added for colorimetric reaction. Following 10 min of incubation in the dark at 25° C., the reaction was stopped by adding 50 ml 2.5M sulfuric acid solution and the resultant absorbance was read on a microplate reader at 490 nm. Relative IgG levels among different groups were reported and compared as the log of the dilution at which the intensity of OPD colorimetric reaction product reached five time above the background (no serum) control intensity.

### Example 6

### CDX-005 Characteristics

CDX-005 contains 283 and CDX-007 contains 149 silent mutations in the Spike gene relative to wt WA1 virus. The resulting full-length wt WA1 and deoptimized cDNAs were transcribed in vitro to make full-length viral RNA that was electroporated into Vero E6 cells. Transfected cells were incubated for 6 days or until CPE appeared. Infection medium was collected on Days 2, 4, and 6. Virus titer was determined by plaque assay on Vero E6 cells. Plaques were visible as early as Day 2 post transfection, with peak virus generation on Days 4-6. Though plaques formed by CDX-005 and CDX-007 are smaller than wt, both grow robustly in Vero E6 cells, indicating their suitability for scale-up manufacturing. Thus, as with our other SAVE vaccines, we were able to rapidly generate multiple vaccine candidates with different degrees of attenuation.

Since CDX-005 is more deoptimized and more attenuated than CDX-007 but grows robustly enough to be produced at scale, to maximize in vivo safety, we selected it for further study. In CDX-005, 1,272 nucleotides of the Spike ORF were codon pair deoptimized for human cells, yielding 283 silent mutations. The polybasic furin cleavage site was removed from the Spike protein for added attenuation and safety.

We performed growth optimization studies for CDX-005 in our GMP characterized animal origin-free (AOF) Vero (WHO-10-87) cells so that we can begin large scale vaccine production by Q4 2020. Growth at 33° C. results in higher titers for both CDX-005 and wt WA1 than at 37° C. Virus peaks before the cytopathic effect (CPE) is observed, with 80-90% of virus being cell associated at the peak. Though the kinetics differ, similar virus titers can be achieved at 0.01 MOI and 0.0001 MOI.

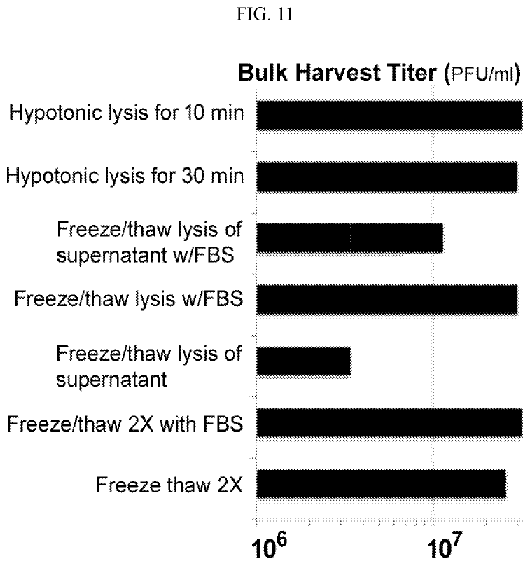

We also investigated optimal conditions for virus harvest. Vero WHO 10-87 cells were grown DMEM with 5% fetal bovine serum (FBS) at 37° C./5% CO₂. At 48 h post-infection with CDX-005 in culture at 33° C., cells and supernatant were harvested using the schemes described in FIG. 11.

The data demonstrate that 48 hr after 0.01 MOI infection at 33° C. most CDX-005 is cell-associated (˜80-90%) but that virus recovery from Vero cells is straight-forward. Hypotonic lysis is an effective means to harvest CDX-005, and the broad lysis window suggests this method will be feasible in scaled batches where some flexibility may be beneficial.

Freeze/thaw lysis is also effective and FBS is neither necessary nor beneficial. This is desirable both because FBS during infection can lead to Vero cell overgrowth, reducing virus yield, and the FDA prefers serum-free production. Also of note, CDX-005 appears to be stable when frozen in plain DMEM as FBS provided little or no stabilization at least after two freeze/thaw cycles. Thus, with optimal timing of harvest, whether grown at 33° C. or 37° C., crude bulk titers of 2-3×10⁷ PFU/ml of CDX-005 are routinely observed, or about 10⁶ PFU/cm² growth surface area.

Based on these studies we are currently growing CDX-005 by inoculating Vero (WHO-10-87) cells with 0.01 MOI at 33° C. We have selected and tested a vaccine formulation of DMEM with 5% sucrose and 5% glycine for our first-in-human studies in the UK. In this formulation, CDX-005 is stable for at least three freeze-thaw cycles and one month at −80° C. (the longest tested storage duration thus far).

Finally, as a first step in assessing the genomic stability of CDX-005, we have sequenced viral passages 1-6 after propagating the virus on Vero (WHO 10-87) cells. The data indicate that the virus is extremely stable. Sequencing of passage 6 revealed no subpopulations. We have grown and harvested nine passages and are currently sequencing passage 9. We will have completed harvesting and sequencing of passage 10 by the beginning of the work described here.

### Example 7

### Assess Immunogenicity of SARS-CoV-2 (Wt WA1) and CDX-005 by Serum IgG Antibody ELISAs and PRNT Assays

Th1/Th2: SARS-CoV-2-specific T cells are present relatively early and increase over time in infected individuals. The strongest T-cell responses appear to be directed to the Spike (S) surface glycoprotein, and SARS-CoV-2-specific T cells predominantly produce effector and Th1 cytokines, although Th2 and Th17 cytokines are detected. It has been suggested that Th1 and T-cytotoxic lymphocytes are the immune cells most affected by SARS-CoV-2, and that T-cell responses and the Th1/Th2 balance may in part dictate the severity of COVID-19. In older individuals who commonly have dampened Th1 responses, the immune system may be forced into a Th2 response to counteract the viral load, producing all the negative effects of the Th2 response, seriously aggravating the clinical picture. Interestingly, T cell responses to SARS-CoV-2 also differ in adults and children. Adults, but not children who developed COVID-19 show increased numbers of activated CD4 and CD8 cells expressing D related antigen (DR) and plasma IL-12, IL-10, and CXCL9 levels. These suggest prominent Th1 polarization of immune response against SARS-CoV-2 in infected adults as compared with children. Because CDX-005 is a live virus, which unlike other vaccine classes can boost immune response by inducing T cell responses, studying the Th1/Th2 balance as directed by the FDA is particularly relevant.

As specified by the FDA, we will measure mRNA levels in lung tissue for Th1/Th2 factors interferon gamma (IFNγ), interleukin 12 (IL-12), tumor necrosis factor alpha (TNFβ), IL4, IL10, and transforming growth factor beta (TGFβ). Tissue homogenates will be analyzed by qPCR using published hamster-specific primers and conditions. The assays will be performed at Bioqual using standard in-house procedures.

We expect that both wt WA1 and CDX-005 inoculation will induce a predominantly Th1 response since these will be young (but not juvenile animals). We expect that there will be a different response to initial inoculation compared to challenge and that those differences may provide insight into the nature of the immune response engendered by SARS-CoV-2.

### Example 8

### Assess the Effect of SARS-CoV-2 (Wt WA1) and CDX-005 on Th1/Th2 Balance by qPCR

Efficacy: We will perform a standard challenge study to assess late passage CDX-005 vaccine efficacy. Hamsters inoculated with either 5×10⁴ PFU CDX-005 or Vehicle will be challenged with ˜1.0×10⁵ TCID₅₀ wt WA1 on Day 27. Day 27 was selected based on other hamster studies and our own data. Three mock challenged animals will be included as contemporaneous negative controls, one for each of the three time points at which we will sacrifice challenged animals. Again the choice of sacrifice at Day 2 and 4 post-challenge is based on published and our own data. We have included a later time point at the suggestion of the FDA and to better understand SARS-CoV-2 induced immune responses. Analysis of hamster serum and tissue will include the same analyses using the same protocols as for hamsters that received only inoculum.

We expect that late passage CDX-005 inoculation will provide protection against challenge similar to that of early passage virus. Data suggest that CDX-005 vaccination is highly effective, reducing Day 2 qPCR lung titers by at least 5,000-fold. A more than 100-fold loss of efficacy or significant differences in measures of safety (i.e., biodistribution, histopathology, or attenuation) of late passage relative to early passage CDX-005 would warrant further examination. We would inform the FDA and work with them to decide on next steps.

### Example 9

As a prelude to moving CDX-005 to first-in-human clinical trials, we examined response of non-human primates to the vaccine. We have inoculated, intranasally, fifteen African green monkeys, six with 10⁶ PFU wt WA1, six with 10⁶ PFU CDX-005, and three with Dulbecco's PBS. Our findings show that while viral titers in lavage fluid of wt WA1 and CDX-005 inoculated animals were similar at Day 4 PI, viral titers remained high in wt WA1 but plummeted to undetectable in CDX-005 inoculated monkeys. These data further demonstrate CDX-005's potential as a SARS-CoV-2 vaccine.

### Example 10

### First-In-Human, Randomised, Double-Blind, Placebo-Controlled, Dose-Escalation Study in Healthy Yong Adults Evaluating the Safety and Immunogenicity of COVI-VAC

COVI-VAC is a live, attenuated vaccine having CDX-005 for prevention of COVID-19. The attenuated virus, carries 283 designed silent mutations with a human codon-pair deoptimized nucleic acid sequence in the gene encoding the viral spike protein and deletion of the furin cleavage site in the spike gene. In addition, it carries 2 silent and 5 nonsilent mutations that were selected for during the virus recovery process in Vero cells.

Primary Study Objective is to assess the safety and tolerability of COVI-VAC at 1 or 2 doses of approximately 5×10⁴, 5×10⁵, and 5×10⁶ plaque-forming units (PFU) administered by nose drops. The Endpoints are: reactogenicity events for 14 days after each dose; adverse events (AEs) from Day 1 to Day 57; medically attended AEs (MAAEs), new-onset chronic illnesses (NCIs), serious AEs (SAEs) from Day 1 to Day 400.

Secondary Study Objective is to assess the humoral immunogenicity of COVI-VAC administered by nose drops. Endpoints are: immunoglobulin G (IgG) titre measured by enzyme-linked immunosorbent assay (ELISA) in serum collected on Days 1, 15, 29, 43, 57, 120, 210, and 400; neutralizing antibody level measured by microneutralization assay in serum collected on Days 1, 15, 29, 43, 57, 120, 210, and 400.

Exploratory objectives and endpoints include:
to measure magnitude and duration of vaccine virus shedding after COVI-VAC administered by nose drops: copy number/mL as assessed by quantitative polymerase chain reaction (qPCR) assay in nasopharyngeal swab samples; Culture results from stool samples collected on Days 4/5 and 14/15;
to assess cellular immunogenicity of COVI-VAC administered by nose drops; to assess mucosal immunogenicity of COVI-VAC administered by nose drops: Spot-forming units (SFU)/10⁶ cells measured by interferon-gamma (IFN-γ) enzyme-linked immunosorbent spot assay (ELISpot) in whole-killed virus-stimulated peripheral blood mononuclear cells (PBMCs) collected on Days 1, 8, 29, and 36;
to assess mucosal immunogenicity of COVI-VAC administered by nose drops: Immunoglobulin A (IgA) titre measured by ELISA in nasal wick samples collected on Days 1, 15, 29, 43, and 57;
to measure incidence of respiratory illness, including that due to SARS-CoV-2 infection, after COVI-VAC administered by nose drops: Results of multiplex PCR respiratory panel (including SARS-CoV-2); Results of multiplex PCR respiratory panel (including SARS-CoV-2) in subjects with respiratory MAAEs; and
to assess the genetic stability of the vaccine virus: Sequence analysis of vaccine virus from nasopharyngeal swab samples

This study is a Phase 1, randomised, double-blind, placebo-controlled, dose-escalation, clinical trial to evaluate the safety and immunogenicity of COVI-VAC in healthy adults aged 18 to 30 years. Potential subjects will be screened using the site's generic screening process, and individuals who pass this screen will be admitted to the Quarantine Unit 1 to 2 days before dosing (Day −2/−1) and provide informed consent. They will then be screened for eligibility for this study before randomisation on Day 1. Approximately 48 subjects who meet all study inclusion and no exclusion criteria will be enrolled in 3 escalating-dose cohorts and randomised within each cohort in a 3:3:2 ratio to receive 2 doses of COVI-VAC/placebo (normal saline) as shown in the table below.

| | COVI-VAC | | | | |
|---|---|---|---|---|---|
| | Day 1 | COVI-VAC | COVI- | Placebo | |
| | Placebo | Days 1 | VAC | Days 1 | |
| Cohort | Day 29 | and 29 | Total | and 29 | Total |
| 1 (5 × 10⁴ PFU) | 6 | 6 | 12 | 4 | 16 |
| 2 (5 × 10⁵ PFU) | 6 | 6 | 12 | 4 | 16 |
| 3 (5 × 10⁶ PFU) | 6 | 6 | 12 | 4 | 16 |
| Total | 18 | 18 | 36 | 12 | 48 |

Abbreviations: PFU = plaque-forming units

Cohort 1 will include a sentinel group of 3 subjects (2 active, 1 placebo). The Safety Review Committee (SRC) will review blinded safety data for these 3 subjects through Day 8 before the remaining subject in Cohort 1 are dosed.

Subjects will remain in the Quarantine Unit until 14 days after Dose 1 (and Dose 2 if administered in the inpatient setting) and will be discharged on Day 15 (and 43, if applicable) unless the subject is experiencing clinically significant symptoms or evidence of ongoing viral infection or the Quarantine Unit has been informed by the laboratory unit that the subject is shedding vaccine virus at a level with more than a low transmission risk as documented in the Risk Management Plan (on the basis of Day 14/42 nasopharyngeal swab sample). These subjects will continue to be confined in the Quarantine Unit until qPCR assay results are consistent with low transmission risk (samples will be collected twice daily).

The SRC will also review blinded safety data through Day 15 and blinded nasopharyngeal swab shedding data through at least Day 8 to determine if the cohort of subjects will be confined in the Quarantine Unit for 14 days after Dose 2 or will be discharged on Day 29 and subsequently seen as outpatients. If subjects are to be seen as outpatients, the SRC will also decide using these data on which 2 days in the first week after Dose 2 the subjects will return to the unit for visits. If subjects are to be seen as inpatients, the SRC will also determine the frequency of nasopharyngeal swab sample collection (no greater than twice daily).

Each subject will record reactogenicity (local events, systemic events, and temperature) in a diary daily for 14 days after the COVI-VAC/placebo dose.

All AEs and concomitant medications will be recorded from signing of the informed consent form (ICF) to Day 57. Thereafter to the end of the study (Day 400), only MAAEs, NCIs, SAEs, immunosuppressive medications, blood products, and vaccines will be recorded. Samples for safety laboratory tests (haematology, biochemistry, coagulation, urinalysis) will be collected before Dose 1 and on Days 8, 36, and 57. A complete physical examination will be performed on Day 2/1, and targeted and symptom-driven physical examinations will be performed predose on Day 1 and Day 29; 2 hours after each dose; on Days 2/30, 4/32, 8/36, and 15/43 while in the Quarantine Unit, and at each outpatient visit in the Dosing Period. Peak expiratory flow (PEF) and vital signs (including oxygen saturation) will be measured predose on Days 1 and 29; 2 hours after each dose; on Days 2/30, 4/32, 8/36, and 15/43 while in the Quarantine Unit; and at each outpatient visit in the Dosing Period. An electrocardiogram (ECG) will be performed before Dose 1 and on Days 2, 8 and 57. A chest X-ray will be performed 14 to 22 days after Dose 1 (between Day 15 and Day 22).

A serum sample will be collected from each subject for evaluation of IgG titre measured by ELISA and neutralizing antibody level measured by microneutralization predose on Days 1 and 29 and on Days 15, 43, 57, 120, 210, and 400. A whole blood sample will be collected from each subject and processed to isolate PBMCs for evaluation of T-cell response by IFN-γ ELISpot predose on Days 1 and 29 and on Days 8 and 36.

A nasopharyngeal swab sample will be collected from each subject twice daily (frequency may be reduced after Dose 2) while in the Quarantine Unit (except postdose only on Day 1, predose only on Day 29 if subjects are admitted on an outpatient basis for Dose 2, and 1 sample only on Day 15/43) and at outpatient visits as determined after Dose 2 to measure concentration of vaccine virus for assessment of shedding by qPCR assay. Once a negative result for an individual subject is obtained, later samples for that subject may not be tested. Samples with evidence of vaccine virus shedding will be retained for potential viral sequencing. A swab sample from stool will be collected on Days 4 or 5 and 14 or 15 to measure vaccine virus for titre.

A nasal wick sample will be collected from each subject for measurement of IgA by ELISA for evaluation of mucosal immune response predose on Days 1 and 29 and on Days 15, 43, and 57.

If a subject experiences acute symptoms compatible with viral respiratory infection, nasopharyngeal swab samples will be collected for multiplex PCR respiratory panel (including SARS-CoV-2).

Any sample positive for SARS-CoV-2 will be retained for analysis to determine if it is wild-type SARS-CoV-2 or vaccine virus.

Each subject will participate in the study for approximately 13 months, including the screening period. The end of the study is defined as the date of the last visit of the last subject participating in the study. The expected duration of study conduct is approximately 17 months, assuming 4 months to enroll subjects.

COVI-VAC is administered by nose drops, up to 2 doses at a 28-day interval. The minimum infectious dose for SARS-CoV-2 is unknown, but animal models result in reproducible infection at doses of 10⁴ to 10⁶ PFU. Weight-based extrapolation of the COVI-VAC dose that was well tolerated in the Syrian hamster model results in a dose of approximately 3.5×10⁷ PFU in a 70 kg human. The dose levels chosen for this study are likely to be both well tolerated and sufficient for evaluation of the activity of COVI-VAC.

### Analyses

Safety: The number (percentage) of subjects with AEs (including MAAEs, NCIs, and SAEs) from Day 1 to Day 57 will be summarised for each Medical Dictionary for Regulatory Activities (MedDRA) system organ class and preferred term and by group. The number (percentage) of subjects with MAAEs, with NCIs, and with SAEs from Day 1 to Day 400 will be summarised in a similar fashion. The number (percentage) of subjects with AEs by severity and by relationship to investigational medicinal product (IMP) will also be summarised. Listings of AEs, MAAEs, NCIs, and SAEs will be provided. The number (percentage) of subjects with local and reactogenicity systemic events after each dose will be summarised by group. Reactogenicity events will also be summarised by severity.

Summary statistics for continuous parameters (safety laboratory tests, PEF, and vital signs) will be presented by group as follows: predose, postdose, and change from predose to postdose assessment. The number and percentage of subjects with postvaccination safety laboratory values or vital sign values recorded as newly abnormal (i.e., an event with an increase in the toxicity grade relative to the baseline value and with a severity grade of moderate or higher) after study vaccination will be tabulated. Shift tables which cross-tabulate the predose and postdose safety laboratory values of each subject by severity grade will be prepared.

Summaries of the number and percentage of subjects with normal, abnormal not clinically significant, and abnormal clinically significant interpretations for physical examinations, ECGs, and chest X-rays will be presented.

Immunogenicity: The primary variables of interest for assessment of humoral immune response to COVI-VAC are IgG titre and neutralizing antibody level. The following measures and their 95% CIs will be summarised by group:
Geometric mean at baseline and on Days 15, 29, 43, 57, 120, 210, and 400
Responder rate on Days 15, 29, 43, 57, 120, 210, and 400

Cellular and mucosal immune response data will be summarised in the same fashion at the relevant sample collection time points.

Shedding: Vaccine virus shedding data from nasopharyngeal and stool swab samples will be summarised by count and percent positive by time point along with median values. The median, interquartile range, minimum, and maximum duration of vaccine virus shedding will be presented by group for the nasopharyngeal swab results.

Respiratory Virus Incidence: Multiplex PCR respiratory panel (including SARS-CoV-2) results from symptomatic subjects and associated symptoms will be listed.

| | Study Visit | | | | | | | | | | | | | | | | | |
|---|---|---|---|---|---|---|---|---|---|---|---|---|---|---|---|---|---|---|
| | Dosing Period | | | | | | | | | | | | | | | | | |
| | Dose 1 & Dose 2 (if Inpatient^{a}) | | | | | | | | | | | | | | | | | |
| | Day −2 | | | | | | | | Dose 2 (if Outpatient^{a}) | | | | | | | Follow- | | |
| | to −1/ | | | Day | Day | Day | Day | Day | | | Post- | | | | Day | up Period | | |
| | Day 27 | Day | | 2/ | 4/ | 8/ | 15/ | 22/ | Day | | Dose | Day | Day | Day | 57/ | Day | Day | Day |
| | to 28 | 1/29 | | 30 | 32 | 36 | 43 | 50 | 29^{b} | | 2^{a} | 36 | 43 | 50 | ET^{c} | 120 | 210 | 400 |
| | Window (days) | | | | | | | | | | | | | | | | | |
| | | | | | | | | | ±3 | | | | | | | | | |
| | | Pre | Post | | | | | ± 1 | Pre | Post | ±1 | ±1 | ±1 | ±1 | ±3 | ±7 | ±7 | ±14 |
| Quarantine in clinical | | | | | | | | | | | | | | | | | | |
| research unit | | | | | | | | | | | | | | | | | | |
| Discharge from | | | | | | | X^{d} | | | X | | | | | | | | |
| clinical research unit | | | | | | | | | | | | | | | | | | |
| Informed consent | X^{e} | | | | | | | | | | | | | | | | | |
| Demographics | X^{e} | | | | | | | | | | | | | | | | | |
| Medical history | X^{e} | | | | | | | | | | | | | | | | | |
| Concomitant | X | X | | | | | | X | X | X | X | X | X | X | X | | | |
| medication | | | | | | | | | | | | | | | | | | |
| recording (all) | | | | | | | | | | | | | | | | | | |
| Immunosuppressive | | | | | | | | | | | | | | | | X | X | X |
| medications, blood | | | | | | | | | | | | | | | | | | |
| products, and vaccines | | | | | | | | | | | | | | | | | | |
| recording (only) | | | | | | | | | | | | | | | | | | |
| AE assessment (all) | X | X | | | | | | X | X | X | X | X | X | X | X | | | |
| MAAE/NCI/SAE | | | | | | | | | | | | | | | | X | X | X |
| assessment (only) | | | | | | | | | | | | | | | | | | |
| Complete PE | X^{e} | | | | | | | | | | | | | | | | | |
| Targeted and | | X | X^{f} | X | X | X | X | X | X | X^{f} | X | X | X | X | X | | | |
| symptom- | | | | | | | | | | | | | | | | | | |
| driven PE | | | | | | | | | | | | | | | | | | |
| Height and weight | X^{e} | | | | | | | | | | | | | | | | | |
| Chest X-ray | | | | | | | X^{g} | | | | | | | | | | | |
| ECG | X^{e} | | | X^{h} | | X^{h} | | | | | | | | | X | | | |
| Spirometry | X^{e} | | | | | | | | | | | | | | | | | |
| Peak expiratory flow | | X | X^{f} | X | X | X | X | X | X | X^{f} | X | X | X | X | X | | | |
| Vital signs (including | X^{e} | X | X^{f} | X | X | X | X | X | X | x^{f} | X | X | X | X | X | | | |
| oxygen saturation)ⁱ | | | | | | | | | | | | | | | | | | |
| Alcohol breath test | Xe | | | | | | | | | | | | | | | | | |
| Laboratory samples: | | | | | | | | | | | | | | | | | | |
| Hepatitis B, and C | X^{e} | | | | | | | | | | | | | | | | | |
| and HIV tests | | | | | | | | | | | | | | | | | | |
| hVIVO COVID Clear | X^{e} | | | | | | | | | | | | | | | | | |
| Test | | | | | | | | | | | | | | | | | | |
| Urine drug & cotinine | X^{e} | | | | | | | | | | | | | | | | | |
| screen | | | | | | | | | | | | | | | | | | |
| Serum (S)/urine (U) | S | | | | | | | | U | | | | | | S | | | |
| pregnancy test^{j} | | | | | | | | | | | | | | | | | | |
| Safety laboratory tests | X^{e} | | | | | X | | | | | | X | | | X | | | |
| Haemoglobin Ale | X^{e} | | | | | | | | | | | | | | | | | |
| Serum sample | | X | | | | | X | | X | | | | X | | X | X | X | X |
| (immunogenicity) | | | | | | | | | | | | | | | | | | |
| Whole blood sample | | X | | | | X | | | X | | | X | | | | | | |
| for PBMC isolation | | | | | | | | | | | | | | | | | | |
| Nasal wick sample | | X | | | | | X | | X | | | | X | | X | | | |
| for IgA analysis | | | | | | | | | | | | | | | | | | |
| Nasopharyngeal swab | X^{e} | If a subject experiences acute symptoms compatible with viral | | | | | | | | | | | | | | | | |
| for multiplex PCR | | respiratory infectionk | | | | | | | | | | | | | | | | |
| respiratory panel | | | | | | | | | | | | | | | | | | |
| Nasopharyngeal swab | | X¹ | X¹ | twice daily^{d, l} | | | | | X | | X | X | X | | | | | |
| for viral shedding | | | | | | | | | | | | | | | | | | |
| analysis | | | | | | | | | | | | | | | | | | |
| Stool swab for viral | | | | | X^{m} | | Xⁿ | | | | | | | | | | | |
| shedding analysis | | | | | | | | | | | | | | | | | | |
| Eligibility criteria | X^{e} | X^{o} | | | | | | | | | | | | | | | | |
| check | | | | | | | | | | | | | | | | | | |
| Temporary delay | | X | | | | | | | X | | | | | | | | | |
| criteria check | | | | | | | | | | | | | | | | | | |
| Randomization | | X^{o} | | | | | | | | | | | | | | | | |
| COVI-VAC/placebo | | X | | | | | | | X | | | | | | | | | |
| administration | | | | | | | | | | | | | | | | | | |
| Diary: distribute (D)/ | | | D | R | R | R | R | | | D | R | R | R | | | | | |
| review (R) | | | | | | | | | | | | | | | | | | |

AE = adverse event;
ECG = electrocardiogram;
ET = early termination;
HIV = human immunodeficiency virus;
IgA = immunoglobulin A;
MAAE = medically attended adverse event;
NCI = new-onset chronic illness;
PBMC = peripheral blood mononuclear cell;
PCR = polymerase chain reaction;
PE = physical examination;
Pre = predose;
Post = postdose;
SAE = serious adverse event;
SRC = Safety Review Committee.
^{a}The SRC will review blinded safety data (AE, reactogenicity, and safety laboratory data) through Day 15 and nasopharyngeal swab shedding data through at least Day 8 (blinded individual data, day of maximal shedding, and range of duration) for all subjects in each cohort to determine if the cohort of subjects will be confined in the Quarantine Unit for 14 days after Dose 2 or will be discharged on Day 29 and subsequently seen as outpatients. If subjects are to be seen as outpatients, the SRC will also decide using these data on which 2 days in the first week after Dose 2 the subjects will return to the unit for visits.
^{b}The Day 29 visit may be delayed up to 2 weeks to avoid dosing over major holiday periods and to optimise subject scheduling. If so, the subsequent visits will be shifted accordingly but will retain the original visit numbering system.
^{c}If a subject prematurely discontinues the study before Day 57, then the procedures listed for Day 57/ET visit should be performed.
^{d}Unless the subject is experiencing clinically significant symptoms or evidence of ongoing viral infection or the Quarantine Unit has been informed by the laboratory unit that the subject is shedding vaccine virus at a level with more than a low transmission risk (on the basis of Day 14/42 nasopharyngeal swab sample). Any subject whose PCR assay results show continued viral shedding at a level with more than a low transmission risk will continue to be confined in the Quarantine Unit until qPCR assay results are consistent with a low transmission risk (samples will be collected twice daily) and any clinically significant symptoms or evidence of ongoing viral infection has resolved.
^{e}Screening procedures - at first admission (Day −2 to Day −1) ONLY
^{f}2 hours postdose
^{g}After Dose 1 ONLY. To be performed between Day 15 and Day 22, inclusive.
^{h}Day 2 and Day 8 ONLY
ⁱMeasured before any blood sample collection
^{j}Required for all women who are not surgically sterilised
^{k}Grade 3 symptoms within 14 days after each dose (Days 1 to 15 and 29 to 43) and symptoms of any grade at other times
¹Predose on Day 29 ONLY; if subjects are to be seen as inpatients for Dose 2, the SRC will also determine the frequency of nasopharyngeal swab sample collection (no greater than twice daily).
^{m}Day 4 or 5 ONLY
ⁿDay 14 or 15 ONLY
^{o}Day 1 ONLY

### Assessments

### Safety

Evaluation of safety is the primary objective for this study. Safety assessments are standard for early-phase clinical trials and are in accordance with FDA's guidance on preventive vaccine clinical trials [FDA 2007]. In addition, because of the inflammatory sequellae and hypercoagulability seen in wild-type infection, safety laboratory studies will also include C-reactive protein, IL-6 and TNF, d-dimer and high sensitivity troponin-T.

Vital sign assessments will include pulse oximetry and peak flow will also be recorded to assess subclinical respiratory impairment.

In addition, a chest X-ray will be performed 2 to 3 weeks after the first dose of CodaVax-COVID/placebo to monitor subjects for subclinical pulmonary inflammation. Evidence is emerging of the utility of chest imaging in early detection of COVID-19. Chest X-ray screening of asymptomatic individuals and symptomatic individuals with low clinical suspicion for COVID-19 immediately after strict local quarantine in Italy showed a high rate of abnormal findings. In a recent meta-analysis of clinical studies evaluating the proportion of positive chest imaging findings in asymptomatic individuals with SARS-COV-2, the authors concluded that asymptomatic cases can have positive chest imaging and that close clinical monitoring of asymptomatic individuals with radiographic findings is necessary because a significant percentage of them develop symptoms.

### Immunogenicity

Binding and neutralizing serum antibodies are the most frequently assessed vaccine biomarkers, but cellular and mucosal immunity may play and equal or even more important role in preventing infection and disease and in reducing the risk of ongoing transmission. In this study serum binding and neutralizing antibodies will be measured as secondary endpoints and mucosal IgA and T-cell response as measured by IFN-γ ELISpot as exploratory endpoints.

### Shedding

Result from the hamster study indicate that carriage of the vaccine virus will be transient. Nasopharyngeal swab samples will be obtained for qPCR to determine how long the vector persists at the site of administration. Data published to date indicate that gastrointestinal shedding starts later and lasts longer than upper airway shedding although it is of unclear significance. Rectal swab samples will be collected in this study for plaque assay to evaluate gastrointestinal shedding of infectious virus.

### Subject Population

For purposes of this study, Inclusion Criteria and Exclusion Criteria are set for as follows. Actual criteria for administration to the population at large can differ. These inclusion and exclusion criteria are not to be interpreted as limiting to the claims unless specifically provided for in the claims.

### Inclusion Criteria

Subjects who meet all of the following criteria may be included in the study:
1. Men and women aged between 18 to 30 years of age, inclusive, on the day of signing the ICF
2. In good health with no history, or current evidence, of clinically significant medical conditions with particular reference to, but not restricted to, hypertension, diabetes, thromboembolic disorders, coronary heart disease, chronic obstructive lung disease, and no clinically significant test abnormalities that will interfere with subject safety, as defined by medical history, physical examination, vital signs (including oxygen saturation), ECG, spirometry, and safety laboratory tests as determined by the Investigator
3. Total body weight of ≥50 kg and body mass index (BMI) ≥18.0 kg/m² and ≤28.0 kg/m² (the upper limit of the BMI may be increased to ≤30 kg/m² at the Investigator's discretion in case of a muscular healthy subject for whom BMI may be biased upwards)
4. Negative drugs of abuse, cotinine, and alcohol screen (unless explained by prescribed medication)
5. Negative pregnancy test for women who have not been surgically sterilised
6. Negative COVID Clear test
7. Willingness to comply with conditions to mitigate risk of vaccine virus transmission (the following conditions are a minimum standard but may be increased if public health authorities recommend a higher standard for mitigation of SARS-CoV-2 transmission):
   Hygiene measures intended to prevent interpersonal transmission of study drug, including, but not limited to, frequent handwashing with soap or hand disinfectant, respiratory hygiene, and cough etiquette for at least 14 days after each IMP dose
   Wearing a surgical mask when in public areas or in contact with other people for at least 14 days after each IMP dose
   Sealing of all tissues and materials used to collect respiratory secretions in a primary container and placement within a secondary container not accessible to children or animals for at least 14 days after each IMP dose
   No close contact with vulnerable individuals for at least 14 days after each IMP dose. Vulnerable individuals include, but are not limited to, the following:
      Persons ≥65 years of age
      Children ≤1 year of age
      Residents and workers in nursing homes
      Persons of any age with significant chronic medical conditions such as:
         Chronic pulmonary disease (e.g., severe asthma, chronic obstructive pulmonary disease)
         Chronic cardiovascular disease (e.g., cardiomyopathy, congestive heart failure, cardiac surgery, ischemic heart disease, known anatomic defects)
         Morbid obesity
         Insulin-dependent or type 2 diabetes mellitus
         Medical follow-up or hospitalisation during the past 5 years because of chronic metabolic disease (e.g., renal dysfunction, haemoglobinopathies)
         Immunosuppression
         Cancer
         Neurological and neurodevelopmental conditions (e.g., cerebral palsy, epilepsy, stroke, seizures)
      Women who are pregnant or who are trying to become pregnant
      Any other person defined as vulnerable during the COVID-19 pandemic
8. Female subjects of childbearing potential must use one form of highly effective contraception. The contraception use must continue for at least 90 days after the last IMP dose. Highly effective contraception is as described below:
   Established use of hormonal methods of contraception described below (for ≥14 days before Day 1). When hormonal methods of contraception are used, male partners must use a condom with a spermicide:
      Combined (oestrogen and progestogen containing) hormonal contraception associated with inhibition of ovulation:
         Oral
         Intravaginal
         Transdermal
      Progestogen-only hormonal contraception associated with inhibition of ovulation:
         Oral
         Injectable
         Implantable
   Intrauterine device
   Intrauterine hormone-releasing system
   Bilateral tubal ligation
   Male sterilisation (with the appropriate postvasectomy documentation of the absence of sperm in the ejaculate) where the vasectomised male is the sole partner for that woman
   True abstinence—sexual abstinence is considered a highly effective method only if defined as refraining from heterosexual intercourse during the entire period of risk associated with the study treatments. The reliability of sexual abstinence needs to be evaluated in relation to the duration of the clinical trial and the preferred and usual lifestyle of the subject.
9. Male subjects must agree to the contraceptive requirements below with use continuing for at least 90 days after last IMP dose:
   Use of a condom with a spermicide to prevent pregnancy in a female partner or to prevent exposure of any partner (male and female) to the IMP
   Male sterilisation with the appropriate postvasectomy documentation of the absence of sperm in the ejaculate (please note that the use of condom with spermicide will still be required to prevent partner exposure). This applies only to males participating in the study.
   In addition, for female partners of childbearing potential, that partner must use another form of contraception such as one of the highly effective methods mentioned above for female subjects.
   True abstinence—sexual abstinence is considered a highly effective method only if defined as refraining from heterosexual intercourse during the entire period of risk associated with the study treatments. The reliability of sexual abstinence needs to be evaluated in relation to the duration of the clinical trial and the preferred and usual lifestyle of the subject.

10. In addition to the contraceptive requirements above, male subjects must agree not to donate sperm for at least 90 days after the last IMP dose.

11. Willingness to participate and comply with all aspects of the study through the entire study period, including all visits to the research unit

12. Provision of written informed consent

### Exclusion Criteria

Subjects who meet any of the following criteria will be excluded from the study:
1. Haemoglobin A1c≥6.0% or 42 mmol/mol
2. Forced expiratory volume in 1 second (FEV₁)<80% predicted value
3. Signs or symptoms suggestive of upper or lower respiratory tract infection (including fever or persistent cough) within 28 days of Day 1
4. Pregnant, possibly pregnant, or lactating women
5. Women who have been pregnant through the third trimester or given birth within the past 6 months
6. Planning a pregnancy (subject or partner) within 90 days after the last IMP dose
7. Inadequate venous access for repeated phlebotomy
8. History of confirmed or suspected SARS-CoV-2 infection
9. Contact with any individual subsequently confirmed to have SARS-CoV-2 within 14 days after contact
10. History of wheeze treated with inhaler(s)
11. Respiratory symptoms, including wheeze, that have ever resulted in hospitalisation
12. Known bronchial hyperreactivity to viruses
13. Any significant abnormality altering the anatomy of the nose in a substantial way or nasopharynx that may interfere with the aims of the study and in particular any of the nasal assessments or viral challenge (historical nasal polyps can be included, but large nasal polyps causing current and significant symptoms and/or requiring regular treatments in the last month are excluded)
14. Any clinically significant history of epistaxis (large nosebleeds) within the last 3 months before Day 1 or history of being hospitalised due to epistaxis on any previous occasion
15. Any nasal or sinus surgery within 3 months before Day 1
16. History of nasal surgery or nasal cauterisation
17. History of autoimmune or demyelinating illness
18. History of Bell's palsy
19. History of psychosis, hospitalisation for psychiatric illness, clinically relevant depression, or suicide attempt in the past 10 years
20. History of seizures (other than childhood febrile seizures), dementia, or progressive neurological disease
21. History of postinfectious or post vaccine neurological sequelae, including Guillain-Barré syndrome
22. History of severe reaction to immunisation
23. History of anaphylaxis or a history of severe allergic reaction or significant intolerance to any food or drug, as assessed by the Investigator
24. Known or suspected malignancy, except for non-melanoma skin cancers and other early stage surgically excised malignancies that the Investigator considers exceedingly unlikely to recur
25. Immunodeficiency, including the use of corticosteroids (including intranasal steroids), alkylating drugs, antimetabolites, radiation, immune-modulating biologics, or other immunomodulating therapies within 90 days before Day 1 or planned use of any of these during the study (topical steroid creams of mild to moderate potency used on the skin are allowed)
26. Any other chronic medical condition that is not stable or requiring medication changes within 30 days before Day 1
27. Receipt of any licensed or investigational coronavirus vaccine or inoculation with SARS-CoV-2
28. Receipt of ≥3 IMPs within 12 months before Day 1
29. Participation in a viral challenge study with a respiratory virus within 90 days or 5 half-lives (whichever is longer) before Day 1
30. Receipt of an IMP within 90 days or 5 half-lives (whichever is longer) before Day 1
31. Receipt of blood transfusion or blood products within 90 days before Day 1 or planned use during the study
32. Receipt of an intranasal vaccine within 90 days before Day 1
33. Receipt of any vaccine, tuberculosis skin test, or allergy antigen inoculation within 30 days before Day 1 or planned receipt through Day 57
34. Receipt of intranasal steroids within 30 days before Day 1 or of any other intranasal medications (including over-the-counter medications but excluding saline) within 7 days before Day 1
35. Current use of any medication for prophylaxis or treatment of COVID-19
36. Planned hospitalisation or surgical procedure through Day 57
37. Regular or current use of intranasal illicit drugs
38. History or presence of alcohol addiction, or excessive use of alcohol (weekly intake >28 units alcohol; 1 unit being a half glass of beer, a small glass of wine, or a measure of spirits)
39. Excessive consumption of xanthine containing substances (e.g., daily intake >5 cups of caffeinated drinks, e.g., coffee, tea, cola).
40. Current smokers of any type (e.g., cigarettes, electronic cigarettes, marijuana)
41. Smoking history of ≥5 pack-years or any smoking in the last 30 days before Day 1
42. Strenuous exercise for 48 hours before admission on Day −2 or Day −1
43. Blood donation of ≥470 mL within 90 days before Day 1
44. Clinically significant ECG abnormality as determined by the Investigator
45. Positive result for HIV, hepatitis B virus, or hepatitis C virus or active hepatitis A virus infection
46. Any medical, psychiatric, or social condition or occupational or other responsibility that, in the judgment of the Investigator, would interfere with or serve as a contraindication to protocol adherence, assessment of safety (including reactogenicity), or a subject's ability to give informed consent
47. Any reason that in the Investigator's opinion raises a concern that the subject will not be able to cope with quarantine requirements
48. Employee or immediate relative of employee of Codagenix, vendors, or research sites associated with the study

Subjects who fail to meet inclusion and exclusion criteria can be rescreened at the discretion of the Investigator, in consultation with the Sponsor, and may participate in the study if they meet inclusion and exclusion criteria at a later date.

### Temporary Delay Criteria

Administration of COVI-VAC/placebo will be delayed for any subject who meets any of the following criteria:
1. Epistaxis (nosebleed) within past 3 days
2. Symptoms of upper respiratory infection or fever (subjective or objective) or malaise within past 3 days

Any signs of symptoms that could inhibit the proper administration of the IMP or interpretation of diary data (e.g., temperature ≥38° C., nasal congestion, rhinorrhoea)

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

1. A polynucleotide encoding one or more viral proteins or one or more fragments thereof of a parent SARS-CoV-2 coronavirus:
wherein the polynucleotide is recoded compared to its parent SARS-CoV-2 coronavirus polynucleotide, and
wherein the amino acid sequence of the one or more viral proteins, or one or more fragments thereof of the parent SARS-CoV-2 coronavirus encoded by the polynucleotide remains the same, or
wherein the amino acid sequence of the one or more viral proteins or one or more fragments thereof of the parent SARS-CoV-2 coronavirus encoded by the polynucleotide comprises up to 20 amino acid substitutions, additions, or deletions.

2. A polynucleotide of claim 1, wherein the parent SARS-CoV-2 coronavirus is a wild-type SARS-CoV-2 or a natural isolate SARS-Co-V-2.

3. (canceled)

4. A polynucleotide of claim 1, wherein the parent SARS-CoV-2 coronavirus is Washington isolate of SARS-CoV-2 coronavirus having a nucleic acid sequence of GenBank accession no. MN985325.1, or wherein the parent SARS-CoV-2 coronavirus is BetaCoV/Wuhan/IVDC-HB-01/2019 isolate of SARS-CoV-2 coronavirus (SEQ ID NO:1).

5. (canceled)

6. (canceled)

7. (canceled)

8. A polynucleotide of claim 1, wherein the polynucleotide is recoded by reducing codon-pair bias (CPB) or reducing codon usage bias compared to its parent SARS-CoV-2 coronavirus polynucleotide, or increasing the number of CpG or UpA di-nucleotides compared to its parent SARS-CoV-2 coronavirus polynucleotide.

9. (canceled)

10. A polynucleotide of claim 1, wherein each of the recoded one or more viral proteins, or each of the recoded one or more fragments thereof has a codon pair bias less than, −0.05, less than −0.1, less than −0.2, less than −0.3, or less than −0.4.

11. A polynucleotide of claim 1, wherein the polynucleotide is CPB deoptimized compared to its parent SARS-CoV-2 coronavirus polynucleotide, or wherein the polynucleotide is codon deoptimized compared to its parent SARS-CoV-2 coronavirus polynucleotide.

12. (canceled)

13. A polynucleotide of claim 11, wherein the CPB deoptimized is based on CPB in humans.

14. A polynucleotide of claim 11, wherein the CPB deoptimized is based on CPB in a coronavirus.

15. (canceled)

16. (canceled)

17. A polynucleotide of claim 1, comprising a recoded nucleotide sequence selected from RNA-dependent RNA polymerase (RdRP), a fragment of RdRP, a spike protein, a fragment of spike protein, and combinations thereof.

18. A polynucleotide of claim 1, comprising at least one CPB deoptimized region selected from bp 11294-12709, bp 14641-15903, bp 21656-22306, bp 22505-23905, and bp 24110-25381 of SEQ ID NO:1 or SEQ ID NO:2.

19. A polynucleotide of claim 1, comprising a recoded spike protein or a fragment of spike protein wherein the furin cleavage site is eliminated.

20. A polynucleotide of claim 1, having SEQ ID NO:4, nucleotides 1-29,834 of SEQ ID NO:4, SEQ ID NO:7, or nucleotides 1-29,834 of SEQ ID NO:7.

21. (canceled)

22. A polynucleotide of claim 1, having SEQ ID NO:3.

23. (canceled)

24. A vector comprising a polynucleotide of claim 1.

25. A cell comprising a polynucleotide of claim 1.

26. The cell of claim 25, wherein the cell is Vero cell or baby hamster kidney (BHK) cell.

27. A polypeptide encoded by a polynucleotide of claim 1.

28. A modified SARS-CoV-2 coronavirus comprising a polynucleotide of claim 1, or a polypeptide encoded by a polynucleotide of claim 1.

29. (canceled)

30. A modified SARS-CoV-2 coronavirus of claim 28, wherein expression of one or more of its viral proteins is reduced compared to its parent SARS-CoV-2 coronavirus.

31. A modified SARS-CoV-2 coronavirus of claim 1, wherein the reduction in the expression of one or more of its viral proteins is reduced as the result of recoding a region selected from RdRP, spike protein and combinations thereof.

32. A modified SARS-CoV-2 coronavirus comprising a polynucleotide having SEQ ID NO:4, or nucleotides 1-29,834 of SEQ ID NO:4, or nucleotides 1-29,834 of SEQ ID NO:4 and one or more consecutive adenines on the 3′ end, or a polypeptide encoded by the polynucleotide having SEQ ID NO:4, or nucleotides 1-29,834 of SEQ ID NO:4, or nucleotides 1-29,834 of SEQ ID NO:4 and one or more consecutive adenines on the 3′ end.

33. (canceled)

34. A vaccine composition for inducing a protective an immune response in a subject, comprising:
a modified SARS-CoV-2 coronavirus of claim 28.

35. The vaccine composition of claim 34, further comprising a pharmaceutically acceptable carrier or excipient.

36. An immune composition for eliciting an immune response in a subject, comprising:
a modified SARS-CoV-2 coronavirus of claim 28.

37. The immune composition of claim 36, further comprising a pharmaceutically acceptable carrier or excipient.

38. A method of eliciting an immune response in a subject, comprising:
administering to the subject a dose of:
a modified SARS-CoV-2 coronavirus of claim 28.

39. A method of eliciting an immune response in a subject, comprising:
administering to the subject a prime dose of a modified SARS-CoV-2 coronavirus of claim 28; and
administering to the subject one or more boost doses of a modified SARS-CoV-2 coronavirus of claim 28.

40. A method of a claim 38, wherein the immune response is a protective immune response.

41. A method of claim 38, wherein the dose is a prophylactically effective or therapeutically effective dose.

42. A method of claim 38, wherein administering is via a nasal route.

43. A method of claim 38, wherein administering is via nasal drop or nasal spray.

44. (canceled)

45. A method of claim 38, wherein the dose is about 10⁴-10⁶ PFU, or the prime dose is about 10⁴-10⁶ PFU and the one or more boost dose is about 10⁴-10⁶ PFU.

46. A method of making a modified SARS-CoV-2 coronavirus, comprising:
obtaining a nucleotide sequence encoding one or more proteins of a parent SARS-CoV-2 coronavirus or one or more fragments thereof,
recoding the nucleotide sequence to reduce protein expression of the one or more proteins, or the one or more fragments thereof, and
substituting a nucleic acid having the recoded nucleotide sequence into the parent SARS-CoV-2 coronavirus genome to make the modified SARS-CoV-2 coronavirus genome,
wherein expression of the recoded nucleotide sequence is reduced compared to the parent virus.

47. (canceled)

48. (canceled)


SEQUENCE LISTING (164 sequences)

SEQ ID NO: 1 - DNA, 29891 bp, SARS-CoV-2
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaa

SEQ ID NO: 2 - DNA, 29891 bp, Artificial Sequence
  SARS CoV-2
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagatggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagaggcgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgaaacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagggacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagatctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaa

SEQ ID NO: 3 - DNA, 29891 bp, Artificial Sequence
  synthetic construct
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagatggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagaggcgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaaacttaaggattgtgttatgtacgctagtgccgttgtgttattgattcttatgactgctagaacagtttatgacgacggagctagacgcgtttggacacttatgaacgtacttacactcgtatataaggtatattatggtaacgcactcgatcaggctatatctatgtgggcattgattatatccgttacgtctaattattcaggcgtagttactacagttatgtttctcgctagaggtatcgtttttatgtgtgttgagtattgtcctatattttttataaccggtaatacattgcaatgtattatgttagtttattgttttctcggttatttttgtacatgttatttcggtctattttgtttgcttaataggtattttagattgacattaggcgtatacgattatctcgttagtacacaggaatttagatatatgaattcacagggtctattgcctcctaaaaactcaatcgacgcattcaaacttaatattaagctattaggcgtaggtggtaagccttgtattaaagtcgctacagtgcaatctaaaatgtctgacgttaagtgtacatcagtcgttctgttatcagtgttacagcaattgagagtcgaatctagttctaagttatgggctcaatgcgttcaattgcacaacgatatactgttagctaaggatactactgaggcattcgaaaaaatggttagtctattatcagtattgctatctatgcagggtgcagtcgatattaataagttatgcgaagagatgctcgataatagggctacattgcaggcaatcgctagcgaattttcatcactaccatcttatgccgcattcgcaactgctcaggaagcatacgaacaagccgtagctaacggtgattcagaggttgtgcttaaaaaacttaaaaaatcacttaacgttgctaaatccgaattcgatagggacgccgctatgcaacgtaaactcgaaaaaatggccgatcaggctatgacacaaatgtataaacaggctagatccgaagataagagagctaaggttactagtgcaatgcaaactatgctttttactatgttgcgtaaactcgataacgacgcacttaataatattattaataacgctagggacggttgtgtaccacttaacattataccacttactactgccgctaaacttatggtcgttatacccgattataatacttataaaaatacatgtgacggtactacttttacatacgctagtgcattatgggagatacaacaggtagtcgatgccgattctaaaatcgttcaattgtcagagatatctatggataattcacctaatctcgcatggcctcttatcgttaccgcacttagagctaattctgccgttaagttacagaataacgaattgtcacccgttgccctacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaataacgtcgcatttcaaaccgttaaaccaggtaattttaataaggatttttacgatttcgccgttagtaagggattttttaaagagggtagttcagtcgaacttaaacacttctttttcgcacaagacggtaacgccgctattagcgattacgattattatagatataatctacctactatgtgtgatattagacaattgttattcgtagtcgaggtagtcgataagtatttcgattgttatgacggaggttgtattaacgctaatcaggtaatcgttaataaccttgataagtctgccggttttccttttaataaatggggtaaggctagattgtattatgattctatgtcatacgaagaccaagacgctctattcgcatatactaaacgtaacgttatacctacaattacgcaaatgaatcttaagtatgctattagcgctaaaaatcgcgctagaacagtcgccggagtgtcaatctgtagtactatgactaatcgtcaatttcaccaaaaattgcttaaatcaatcgccgctactagaggcgctacagtcgttatcggtacatctaaattttatggcggatggcataatatgcttaaaacggtttattctgacgttgagaatccacaccttatgggttgggattatcctaaatgcgatcgcgctatgcctaatatgcttagaattatggctagtctcgtactcgcacgtaaacatacaacatgttgtagtctatcacatagattttatagattagctaacgaatgtgcacaagtgcttagcgaaatggttatgtgtggtggatcactatatgttaaacccggtggtacatctagcggtgacgctactaccgcatacgctaatagcgtattcaatatatgtcaagccgttaccgctaacgttaacgctctactatctactgacggtaataagatagccgataagtatgttagaaacttacaacatagattgtatgagtgtctttatcgtaatcgcgatgtcgatactgatttcgttaacgaattctatgcttatcttagaaaacactttagtatgatgatactatctgacgatgccgtagtgtgttttaactctacttatgctagtcagggattagtcgcatcaatcaaaaattttaaatcagtgttatattatcaaaataacgtttttatgtcagaagctaaatgttggactgagactgatcttactaagggtccacacgaattttgttcacaacatactatgttagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgaaacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagggacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattcttttactaggggggtttattatcctgataaggtttttcgtagttcagtcttgcatagtactcaggatctatttttacctttcttttcgaacgttacttggtttcacgctatacacgtttccggtactaacggtactaaaagattcgataatcctgtcttaccatttaacgacggagtgtatttcgctagtaccgaaaaatctaatattattaggggttggatattcggtactactcttgactctaaaacgcaatcattgttaatcgttaataacgctactaacgtcgttattaaggtatgcgaatttcagttttgtaacgatcctttcttaggcgtttattatcataaaaataataagtcttggatggaatccgaatttagagtgtatagttccgctaataattgtacattcgaatacgtttcgcaaccatttcttatggatcttgagggtaagcagggtaattttaaaaatttgcgtgaattcgtttttaaaaatatagacggttattttaagatatactctaaacatacccctattaatctcgttagggatctacctcagggtttttccgctcttgagccattagtcgatttgccaataggtattaatattactagatttcagactcttttagcgttacatagatcttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacatctaattttagagtgcaacctaccgaatcaatcgttagatttcctaatattactaacttatgtccatttggggaagtttttaacgctactagattcgctagcgtttacgcttggaatcgtaaacgtatatctaattgcgttgccgattattccgtactttataattccgctagtttttctacttttaagtgttacggagttagtccaactaagcttaacgatctatgttttactaacgtatacgctgattcattcgttattagaggtgacgaagttagacagattgcaccaggtcaaacaggtaagattgccgattataattataagttgcctgacgattttacaggttgcgttatcgcttggaattctaataatctcgattctaaagtcggaggtaattataattatctatatagattgtttcgtaagtctaaccttaaaccattcgaacgggatattagtactgagatttatcaggcaggtagtacaccttgtaacggtgtcgaaggttttaattgttattttccacttcaatcttacggttttcaacctactaacggagtcggatatcaaccatatagagtagtcgttctatcattcgaactgttacatgcacccgctacagtttgtggtcctaaaaaatctactaacttagttaagaataaatgcgttaattttaactttaacggtcttacaggtacaggcgtacttaccgaatctaacaaaaaatttttaccatttcaacaatttggtagggatatagccgatactactgacgcagttagggatccacaaacacttgagatactcgatattacaccttgttcttttggcggggttagcgtaattacacccggtactaatacatctaatcaggtcgcagtactttatcaagacgttaattgtactgaagtgccagtcgctatacatgctgatcaattgacacctacatggcgggtttattctaccggttctaacgtttttcagactagagccggttgtcttataggcgctgaacatgttaataactcatacgaatgcgatattcctataggcgcaggtatttgcgcatcatatcaaacacagactaactcacctagacgtgctagatccgttgctagtcaatctattatcgcttatactatgtctttaggcgctgaaaactcagtcgcatactctaataactcaatcgctatacctactaattttacaattagcgttactactgagattttacccgttagtatgactaagacttcagtcgattgtactatgtatatttgcggtgactctaccgaatgttctaatctgttattgcaatacggatctttttgtacacaattgaatcgtgcacttacaggtattgcagtcgaacaggataagaatacacaagaggtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagatctcatttgcgctcaaaaatttaacggacttacagttttaccacctttacttactgacgaaatgattgcgcaatatacatccgcattgttagccggaactattacatccggatggacttttggcgcaggcgcagcattacagattccattcgctatgcaaatggcttataggtttaacggtataggcgttacgcaaaacgtactttatgagaatcaaaaacttatcgctaaccaatttaattccgctatcggtaagattcaggattcattgtctagtactgctagtgcactcggtaagttgcaagacgtagtgaatcaaaacgctcaagcacttaatacactcgttaaacagcttagttctaattttggcgcaatttctagtgtgcttaacgatatactatctagactcgataaagtcgaagccgaagtgcaaatcgatagattgattaccggtaggttgcaatcattgcaaacatacgttacacagcaattgattagggccgcagagatacgcgctagcgctaatctcgcagctactaaaatgtctgaatgcgtactcggacaatctaaacgtgtcgatttttgcggtaagggatatcatcttatgtcttttccacaatctgcacctcacggagtcgtgtttttacacgttacttatgtgccagctcaagagaaaaattttacaaccgctcctgctatttgtcatgacggtaaggcacattttcctagagagggcgtattcgtttctaacggtacacattggttcgttacacaacgtaatttttacgaacctcaaattattactactgataatacattcgtatcaggtaattgtgacgtagtgataggtatcgttaataatacagtttacgatccacttcaacctgaactcgatagttttaaagaggaactcgataagtattttaaaaatcatacatcacctgacgtcgacttaggcgatatttcaggtattaacgctagtgtcgttaacattcaaaaagagattgatagacttaacgaagtcgctaaaaatcttaacgaatcacttatcgatctgcaagagttaggtaagtatgagcaatatattaaatggccttggtatatttggttaggctttatagccggattgatcgcaatcgttatggttacaattatgttatgttgtatgacatcatgttgttcatgtcttaagggatgttgttcatgcggatcatgttgtaaatttgacgaagacgattccgaaccagtgcttaaaggcgttaagttacattatacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaa

SEQ ID NO: 4 - DNA, 29855 bp, Artificial Sequence
  synthetic construct
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttatattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacctttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgttcttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcagtgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactttttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccattgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacgtagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagatctcatttgcgctcaaaaatttaacggacttacagttttaccacctttacttactgacgaaatgattgcgcaatatacatccgcattgttagccggaactattacatccggatggacttttggcgcaggcgtagcattacagattccattcgctatgcaaatggcttataggtttaacggtataggcgttacgcaaaacgtactttatgagaatcaaaaacttatcgctaaccaatttaattccgctatcggtaagattcaggattcattgtctagtactgctagtgcactcggtaagttgcaagacgtagtgaatcaaaacgctcaagcacttaatacactcgttaaacagcttagttctaattttggcgcaatttctagtgtgcttaacgatatactatctagactcgataaagtcgaagccgaagtgcaaatcgatagattgattaccggtaggttgcaatcattgcaaacatacgttacacagcaattgattagggccgcagagatacgcgctagcgctaatctcgcagctactaaaatgtctgaatgcgtactcggacaatctaaacgtgtcgatttttgcggtaagggatatcatcttatgtcttttccacaatctgcacctcacggagtcgtgtttttacacgttacttatgtgccagctcaagagaaaaattttacaaccgctcctgctatttgtcatgacggtaaggcacattttcctagagagggcgtattcgtttctaacggtacacattggttcgttacacaacgtaatttttacgaacctcaaattattactactgataatacattcgtatcaggtaattgtgacgtagtgataggtatcgttaataatacagtttacgatccacttcaacctgaactcgatagttttaaagaggaactcgataagtattttaaaaatcatacatcacctgacgtcgacttaggcgatatttcaggtattaacgctagtgtcgttaacattcaaaaagagattgatagacttaacgaagtcgctaaaaatcttaacgaatcacttatcgatctgcaagagttaggtaagtatgagcaatatattaaatggccttggtatatttggttaggctttatagccggattgatcgcaatcgttatggttacaattatgttatgttgtatgacatcatgttgttcatgtcttaagggatgttgttcatgcggatcatgttgtaaatttgacgaagacgattccgaaccagtgcttaaaggcgttaagttacattatacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgttcaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaa

SEQ ID NO: 5 - DNA, 36 bp, SARS-CoV-2
actaattctcctcggcgggcacgtagtgtagctagt

SEQ ID NO: 6 - protein, 1261 aa, Artificial Sequence
  synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALRRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGVALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 7 - DNA, 29853 bp, Artificial Sequence
  synthetic construct
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactttttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattcttttactaggggggtttattatcctgataaggtttttcgtagttcagtcttgcatagtactcaggatctatttttacctttcttttcgaacgttacttggtttcacgctatacacgtttccggtactaacggtactaaaagattcgataatcctgtcttaccatttaacgacggagtgtatttcgctagtaccgaaaaatctaatattattaggggttggatattcggtactactcttgactctaaaacgcaatcattgttaatcgttaataacgctactaacgtcgttattaaggtatgcgaatttcagttttgtaacgatcctttcttaggcgtttattatcataaaaataataagtcttggatggaatccgaatttagagtgtatagttccgctaataattgtacattcgaatacgtttcgcaaccatttcttatggatcttgagggtaagcagggtaattttaaaaatttgcgtgaattcgtttttaaaaatatagacggttattttaagatatactctaaacatacccctattaatctcgttagggatctacctcagggtttttccgctcttgagccattagtcgatttgccaataggtattaatattactagatttcagactcttttagcgttacatagatcttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgttcaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaa

SEQ ID NO: 8 - protein, 12 aa, SARS-CoV-2
TNSPRRARSVAS

SEQ ID NO: 9 - DNA, 45 bp, Artificial Sequence
  synthetic construct
taatacgactcactattattaaaggtttataccttcccaggtaac

SEQ ID NO: 10 - DNA, 23 bp, Artificial Sequence
  synthetic construct
gatgccaaaataatggcgatctc

SEQ ID NO: 11 - DNA, 22 bp, Artificial Sequence
  synthetic construct
gttggttgccataacaagtgtg

SEQ ID NO: 12 - DNA, 27 bp, Artificial Sequence
  synthetic construct
ctaattgaggttgaacctcaacaattg

SEQ ID NO: 13 - DNA, 28 bp, Artificial Sequence
  synthetic construct
gagtatggtactgaagatgattaccaag

SEQ ID NO: 14 - DNA, 24 bp, Artificial Sequence
  synthetic construct
ctaggtggaatgtggtaggattac

SEQ ID NO: 15 - DNA, 29 bp, Artificial Sequence
  synthetic construct
gctgttacagcgtataatggttatcttac

SEQ ID NO: 16 - DNA, 29 bp, Artificial Sequence
  synthetic construct
gctggtttaagtataatgtctcctacaac

SEQ ID NO: 17 - DNA, 28 bp, Artificial Sequence
  synthetic construct
gcacaaaaccagttgaaacatcaaattc

SEQ ID NO: 18 - DNA, 28 bp, Artificial Sequence
  synthetic construct
gcaactagtgttttgagtttttccattg

SEQ ID NO: 19 - DNA, 25 bp, Artificial Sequence
  synthetic construct
gtgaagaatcatctgcaaaatcagc

SEQ ID NO: 20 - DNA, 32 bp, Artificial Sequence
  synthetic construct
caaatgatataagcaattgttatccagaaagg

SEQ ID NO: 21 - DNA, 35 bp, Artificial Sequence
  synthetic construct
gcctttaatactttactattccttatgtcattcac

SEQ ID NO: 22 - DNA, 27 bp, Artificial Sequence
  synthetic construct
ccagacaaactagtatcaaccatatcc

SEQ ID NO: 23 - DNA, 25 bp, Artificial Sequence
  synthetic construct
gctatgggtattattgctatgtctg

SEQ ID NO: 24 - DNA, 21 bp, Artificial Sequence
  synthetic construct
cctacaaggtggttccagttc

SEQ ID NO: 25 - DNA, 19 bp, Artificial Sequence
  synthetic construct
cgacagatgtcttgtgctg

SEQ ID NO: 26 - DNA, 25 bp, Artificial Sequence
  synthetic construct
ggtatccagttgaaactacaaatgg

SEQ ID NO: 27 - DNA, 23 bp, Artificial Sequence
  synthetic construct
gatcagacataccacccaaattg

SEQ ID NO: 28 - DNA, 34 bp, Artificial Sequence
  synthetic construct
cttatgtattgtaagtacaaatgaaagacatcag

SEQ ID NO: 29 - DNA, 27 bp, Artificial Sequence
  synthetic construct
ggtgatgattatgtgtaccttccttac

SEQ ID NO: 30 - DNA, 27 bp, Artificial Sequence
  synthetic construct
ctgttaattgcagatgaaacatcatgc

SEQ ID NO: 31 - DNA, 29 bp, Artificial Sequence
  synthetic construct
gtgtgtagacttatgaaaactataggtcc

SEQ ID NO: 32 - DNA, 21 bp, Artificial Sequence
  synthetic construct
catacaaactgccaccatcac

SEQ ID NO: 33 - DNA, 31 bp, Artificial Sequence
  synthetic construct
ccttgtagtgacaaagcttataaaatagaag

SEQ ID NO: 34 - DNA, 21 bp, Artificial Sequence
  synthetic construct
ctggtgcaactcctttatcag

SEQ ID NO: 35 - DNA, 28 bp, Artificial Sequence
  synthetic construct
gcaaagaatgctattagaaaagtgtgac

SEQ ID NO: 36 - DNA, 32 bp, Artificial Sequence
  synthetic construct
gatagattcctttttctacagtgaaggatttc

SEQ ID NO: 37 - DNA, 23 bp, Artificial Sequence
  synthetic construct
gactcctggtgattcttcttcag

SEQ ID NO: 38 - DNA, 23 bp, Artificial Sequence
  synthetic construct
ctctagcagcaatatcaccaagg

SEQ ID NO: 39 - DNA, 28 bp, Artificial Sequence
  synthetic construct
gcacaagtcaaacaaatttacaaaacac

SEQ ID NO: 40 - DNA, 28 bp, Artificial Sequence
  synthetic construct
caaaaggtgtgagtaaactgttacaaac

SEQ ID NO: 41 - DNA, 19 bp, Artificial Sequence
  synthetic construct
ctcactccctttcggatgg

SEQ ID NO: 42 - DNA, 32 bp, Artificial Sequence
  synthetic construct
gaggtttatgatgtaatcaagattccaaatgg

SEQ ID NO: 43 - DNA, 29 bp, Artificial Sequence
  synthetic construct
gctacaggattggcaactataaattaaac

SEQ ID NO: 44 - DNA, 22 bp, Artificial Sequence
  synthetic construct
ccattctagcaggagaagttcc

SEQ ID NO: 45 - DNA, 21 bp, Artificial Sequence
  synthetic construct
gcaatcctgctaacaatgctg

SEQ ID NO: 46 - DNA, 53 bp, Artificial Sequence
  synthetic construct
tttttttttttttttttttttttttgtcattctcctaagaagctattaaaatc

SEQ ID NO: 47 - DNA, 23 bp, Artificial Sequence
  synthetic construct
gaagacttggtacacttgagacg

SEQ ID NO: 48 - DNA, 37 bp, Artificial Sequence
  synthetic construct
aattttgacgtcgatcccgttgacattgattattgac

SEQ ID NO: 49 - DNA, 28 bp, Artificial Sequence
  synthetic construct
ccacacagattttaaagttcgtttagag

SEQ ID NO: 50 - DNA, 16 bp, Artificial Sequence
  synthetic construct
gcaaatgggcggtagg

SEQ ID NO: 51 - DNA, 32 bp, Artificial Sequence
  synthetic construct
atcgcaggtctcgcgctttcccgaattcgagc

SEQ ID NO: 52 - DNA, 37 bp, Artificial Sequence
  synthetic construct
atcgcaggtctcggaggatatagttcctcctttcagc

SEQ ID NO: 53 - DNA, 44 bp, Artificial Sequence
  synthetic construct
tatgcgggtctccggagtaatacgactcactattattaaaggtt

SEQ ID NO: 54 - DNA, 33 bp, Artificial Sequence
  synthetic construct
tatgcgggtctccagaccttcggaaccttctcc

SEQ ID NO: 55 - DNA, 40 bp, Artificial Sequence
  synthetic construct
tatgcgggtctcggtcttaatgacaaccttcttgaaatac

SEQ ID NO: 56 - DNA, 35 bp, Artificial Sequence
  synthetic construct
tatgcgggtctcgaatcttcttcttgctcttcttc

SEQ ID NO: 57 - DNA, 40 bp, Artificial Sequence
  synthetic construct
tatgcgggtctcggattggttagatgatgatagtcaacaa

SEQ ID NO: 58 - DNA, 35 bp, Artificial Sequence
  synthetic construct
tatgcgggtctcgctttataggaaccagcaagtga

SEQ ID NO: 59 - DNA, 35 bp, Artificial Sequence
  synthetic construct
tatgcgggtctcgaaagattggtcctattctggac

SEQ ID NO: 60 - DNA, 36 bp, Artificial Sequence
  synthetic construct
tatgcgggtctcgatggttttagatcttcgcaggca

SEQ ID NO: 61 - DNA, 38 bp, Artificial Sequence
  synthetic construct
tgagtcggtctccggagccagtctctgaagaagtagtg

SEQ ID NO: 62 - DNA, 35 bp, Artificial Sequence
  synthetic construct
tgagtcggtctccatcagacactaatgcctgatct

SEQ ID NO: 63 - DNA, 33 bp, Artificial Sequence
  synthetic construct
tgagtcggtctcctgatgttggtgatagtgcgg

SEQ ID NO: 64 - DNA, 42 bp, Artificial Sequence
  synthetic construct
tgagtcggtctcccaagtacaagtaaataacagaataaacac

SEQ ID NO: 65 - DNA, 44 bp, Artificial Sequence
  synthetic construct
tgagtcggtctcccttgacattttatcttactaatgatgtttct

SEQ ID NO: 66 - DNA, 36 bp, Artificial Sequence
  synthetic construct
tgagtcggtctccagtggcaagagaaggtaacaaaa

SEQ ID NO: 67 - DNA, 41 bp, Artificial Sequence
  synthetic construct
tgagtcggtctcccactgtagcttattttaatatggtctat

SEQ ID NO: 68 - DNA, 34 bp, Artificial Sequence
  synthetic construct
tgagtcggtctcccctacctccctttgttgtgtt

SEQ ID NO: 69 - DNA, 34 bp, Artificial Sequence
  synthetic construct
tgagtcggtctcgtaggtttgtacttgcactgtt

SEQ ID NO: 70 - DNA, 44 bp, Artificial Sequence
  synthetic construct
tgagtcggtctcgatggcactgtagagaataaaacattaaagtt

SEQ ID NO: 71 - DNA, 37 bp, Artificial Sequence
  synthetic construct
ctaaggggtctcgggagttcccacctacaagttttgg

SEQ ID NO: 72 - DNA, 34 bp, Artificial Sequence
  synthetic construct
ctaaggggtctcggtgtaccatctgtttttacga

SEQ ID NO: 73 - DNA, 37 bp, Artificial Sequence
  synthetic construct
ctaaggggtctcgacacttatgattgaacggttcgtg

SEQ ID NO: 74 - DNA, 31 bp, Artificial Sequence
  synthetic construct
ctaaggggtctcgaaagcactcacagtgtca

SEQ ID NO: 75 - DNA, 43 bp, Artificial Sequence
  synthetic construct
ctaaggggtctcgctttggtttatgataataagcttaaagcac

SEQ ID NO: 76 - DNA, 37 bp, Artificial Sequence
  synthetic construct
ctaaggggtctcgttgcaattccaaaataggcataca

SEQ ID NO: 77 - DNA, 38 bp, Artificial Sequence
  synthetic construct
ctaaggggtctccgcaatgtcgatagatatcctgctaa

SEQ ID NO: 78 - DNA, 41 bp, Artificial Sequence
  synthetic construct
ctaaggggtctccagtatattttgcgacattcatcattatg

SEQ ID NO: 79 - DNA, 40 bp, Artificial Sequence
  synthetic construct
ctaaggggtctcctactcaactgtgtcaatatttaaacac

SEQ ID NO: 80 - DNA, 46 bp, Artificial Sequence
  synthetic construct
ctaaggggtctccatggtatattttaatagaaaagtcctaggttga

SEQ ID NO: 81 - DNA, 40 bp, Artificial Sequence
  synthetic construct
aagcacggtctccggagatgaaaatggaaccattacagat

SEQ ID NO: 82 - DNA, 33 bp, Artificial Sequence
  synthetic construct
aagcacggtctcccttggttttgatggatctgg

SEQ ID NO: 83 - DNA, 41 bp, Artificial Sequence
  synthetic construct
aagcacggtctcccaagcaagaggtcatttattgaagatct

SEQ ID NO: 84 - DNA, 36 bp, Artificial Sequence
  synthetic construct
aagcacggtctccagttgccatctctttttgagggt

SEQ ID NO: 85 - DNA, 32 bp, Artificial Sequence
  synthetic construct
aagcacggtctcgaactagcactctccaaggg

SEQ ID NO: 86 - DNA, 37 bp, Artificial Sequence
  synthetic construct
aagcacggtctcgtctgttgtcacttactgtacaagc

SEQ ID NO: 87 - DNA, 36 bp, Artificial Sequence
  synthetic construct
aagcacggtctcccagatgtttcatctcgttgactt

SEQ ID NO: 88 - DNA, 33 bp, Artificial Sequence
  synthetic construct
aagcacggtctcctgctcccttctgcgtagaag

SEQ ID NO: 89 - DNA, 33 bp, Artificial Sequence
  synthetic construct
aagcacggtctcgagcagaggcggcagtcaagc

SEQ ID NO: 90 - DNA, 39 bp, Artificial Sequence
  synthetic construct
aagcacggtctcgatggggatatagttcctcctttcagc

SEQ ID NO: 91 - DNA, 35 bp, Artificial Sequence
  synthetic construct
ccatgtggtctccggaggctttcccgaattcgagc

SEQ ID NO: 92 - DNA, 36 bp, Artificial Sequence
  synthetic construct
ccatgtggtctcctctgcttatatagacctcccacc

SEQ ID NO: 93 - DNA, 44 bp, Artificial Sequence
  synthetic construct
ccatgtggtctcgcagataatacgactcactattattaaaggtt

SEQ ID NO: 94 - DNA, 32 bp, Artificial Sequence
  synthetic construct
ccatgtggtctcggaccttcggaaccttctcc

SEQ ID NO: 95 - DNA, 41 bp, Artificial Sequence
  synthetic construct
ccatgtggtctccggtcttaatgacaaccttcttgaaatac

SEQ ID NO: 96 - DNA, 35 bp, Artificial Sequence
  synthetic construct
atcgcaggtctcgcctcgaccaattctcatgtttg

SEQ ID NO: 97 - DNA, 35 bp, Artificial Sequence
  synthetic construct
atcgcaggtctcgagcgttattagcgatgagctcg

SEQ ID NO: 98 - DNA, 34 bp, Artificial Sequence
  synthetic construct
atcgcaggtctcgggttttagatcttcgcaggca

SEQ ID NO: 99 - DNA, 36 bp, Artificial Sequence
  synthetic construct
atcgcaggtctccaaccagtctctgaagaagtagtg

SEQ ID NO: 100 - DNA, 41 bp, Artificial Sequence
  synthetic construct
atcgcaggtctccacactgtagagaataaaacattaaagtt

SEQ ID NO: 101 - DNA, 36 bp, Artificial Sequence
  synthetic construct
atcgcaggtctcggtgttcccacctacaagttttgg

SEQ ID NO: 102 - DNA, 45 bp, Artificial Sequence
  synthetic construct
atcgcaggtctcgcattatattttaatagaaaagtcctaggttga

SEQ ID NO: 103 - DNA, 37 bp, Artificial Sequence
  synthetic construct
atcgcaggtctcgaatgaaaatggaaccattacagat

SEQ ID NO: 104 - DNA, 34 bp, Artificial Sequence
  synthetic construct
ctcatcggtctcgtagttattagcgatgagctcg

SEQ ID NO: 105 - DNA, 42 bp, Artificial Sequence
  synthetic construct
ctcatcggtctcgactaatacgactcactattattaaaggtt

SEQ ID NO: 106 - DNA, 46 bp, Artificial Sequence
  synthetic construct
tcaggtggtctcgaggaactatatcctcgaccaattctcatgtttg

SEQ ID NO: 107 - DNA, 31 bp, Artificial Sequence
  synthetic construct
tcaggtggtctcggcgatgagctcggacttc

SEQ ID NO: 108 - DNA, 42 bp, Artificial Sequence
  synthetic construct
tcaggtggtctcgtcgctaataacgctttcccgaattcgagc

SEQ ID NO: 109 - DNA, 33 bp, Artificial Sequence
  synthetic construct
tcaggtggtctcggcttatatagacctcccacc

SEQ ID NO: 110 - DNA, 47 bp, Artificial Sequence
  synthetic construct
tcaggtggtctcgaagcagataatacgactcactattattaaaggtt

SEQ ID NO: 111 - DNA, 32 bp, Artificial Sequence
  synthetic construct
tcaggtggtctcgaaccttctccaacaacacc

SEQ ID NO: 112 - DNA, 50 bp, Artificial Sequence
  synthetic construct
tcaggtggtctccggttccgaaggtcttaatgacaaccttcttgaaatac

SEQ ID NO: 113 - DNA, 32 bp, Artificial Sequence
  synthetic construct
tcaggtggtctcccttgctcttcttcaggttg

SEQ ID NO: 114 - DNA, 49 bp, Artificial Sequence
  synthetic construct
tcaggtggtctcccaagaagaagattggttagatgatgatagtcaacaa

SEQ ID NO: 115 - DNA, 32 bp, Artificial Sequence
  synthetic construct
tcaggtggtctccataggaaccagcaagtgag

SEQ ID NO: 116 - DNA, 39 bp, Artificial Sequence
  synthetic construct
tcaggtggtctcgctataaagattggtcctattctggac

SEQ ID NO: 117 - DNA, 31 bp, Artificial Sequence
  synthetic construct
tcaggtggtctcgtttagatcttcgcaggca

SEQ ID NO: 118 - DNA, 39 bp, Artificial Sequence
  synthetic construct
tcaggtggtctcctaaaaccagtctctgaagaagtagtg

SEQ ID NO: 119 - DNA, 36 bp, Artificial Sequence
  synthetic construct
tcaggtggtctcccatcagacactaatgcctgatct

SEQ ID NO: 120 - DNA, 32 bp, Artificial Sequence
  synthetic construct
tcaggtggtctccgatgttggtgatagtgcgg

SEQ ID NO: 121 - DNA, 37 bp, Artificial Sequence
  synthetic construct
tcaggtggtctcctaaataacagaataaacaccaggt

SEQ ID NO: 122 - DNA, 54 bp, Artificial Sequence
  synthetic construct
tcaggtggtctcgtttacttgtacttgacattttatcttactaatgatgtttct

SEQ ID NO: 123 - DNA, 36 bp, Artificial Sequence
  synthetic construct
tcaggtggtctcgagagaaggtaacaaaaacaaaca

SEQ ID NO: 124 - DNA, 48 bp, Artificial Sequence
  synthetic construct
tcaggtggtctcgctcttgccactgtagcttattttaatatggtctat

SEQ ID NO: 125 - DNA, 42 bp, Artificial Sequence
  synthetic construct
tcaggtggtctcgagtacaaacctacctccctttgttgtgtt

SEQ ID NO: 126 - DNA, 32 bp, Artificial Sequence
  synthetic construct
tcaggtggtctcctacttgcactgttatccga

SEQ ID NO: 127 - DNA, 48 bp, Artificial Sequence
  synthetic construct
tcaggtggtctccggtgggaacactgtagagaataaaacattaaagtt

SEQ ID NO: 128 - DNA, 32 bp, Artificial Sequence
  synthetic construct
tcaggtggtctcgcacctacaagttttggacc

SEQ ID NO: 129 - DNA, 40 bp, Artificial Sequence
  synthetic construct
tcaggtggtctcgtcataagtgtaccatctgtttttacga

SEQ ID NO: 130 - DNA, 32 bp, Artificial Sequence
  synthetic construct
tcaggtggtctccatgattgaacggttcgtgt

SEQ ID NO: 131 - DNA, 34 bp, Artificial Sequence
  synthetic construct
tcaggtggtctccaccaaagcactcacagtgtca

SEQ ID NO: 132 - DNA, 40 bp, Artificial Sequence
  synthetic construct
tcaggtggtctcgtggtttatgataataagcttaaagcac

SEQ ID NO: 133 - DNA, 35 bp, Artificial Sequence
  synthetic construct
tcaggtggtctcgcaattccaaaataggcatacac

SEQ ID NO: 134 - DNA, 41 bp, Artificial Sequence
  synthetic construct
tcaggtggtctccattgcaatgtcgatagatatcctgctaa

SEQ ID NO: 135 - DNA, 33 bp, Artificial Sequence
  synthetic construct
tcaggtggtctccgcgacattcatcattatgcc

SEQ ID NO: 136 - DNA, 50 bp, Artificial Sequence
  synthetic construct
tcaggtggtctcgtcgcaaaatatactcaactgtgtcaatatttaaacac

SEQ ID NO: 137 - DNA, 46 bp, Artificial Sequence
  synthetic construct
tcaggtggtctcgtcattatattttaatagaaaagtcctaggttga

SEQ ID NO: 138 - DNA, 36 bp, Artificial Sequence
  synthetic construct
tcaggtggtctcgatgaaaatggaaccattacagat

SEQ ID NO: 139 - DNA, 35 bp, Artificial Sequence
  synthetic construct
tcaggtggtctcgtgcttggttttgatggatctgg

SEQ ID NO: 140 - DNA, 39 bp, Artificial Sequence
  synthetic construct
tcaggtggtctcgagcaagaggtcatttattgaagatct

SEQ ID NO: 141 - DNA, 36 bp, Artificial Sequence
  synthetic construct
tcaggtggtctcgtttttgagggttatgattttgga

SEQ ID NO: 142 - DNA, 44 bp, Artificial Sequence
  synthetic construct
tcaggtggtctccaaaagagatggcaactagcactctccaaggg

SEQ ID NO: 143 - DNA, 37 bp, Artificial Sequence
  synthetic construct
tcaggtggtctcctgtcacttactgtacaagcaaagc

SEQ ID NO: 144 - DNA, 41 bp, Artificial Sequence
  synthetic construct
tcaggtggtctcggacaacagatgtttcatctcgttgactt

SEQ ID NO: 145 - DNA, 35 bp, Artificial Sequence
  synthetic construct
tcaggtggtctcgtctgctcccttctgcgtagaag

SEQ ID NO: 146 - DNA, 31 bp, Artificial Sequence
  synthetic construct
tcaggtggtctcgcagaggcggcagtcaagc

SEQ ID NO: 147 - DNA, 34 bp, Artificial Sequence
  synthetic construct
tcaggtggtctcgtcctcctttcagcaaaaaacc

SEQ ID NO: 148 - DNA, 40 bp, Artificial Sequence
  synthetic construct
gtccgagctcatcgctaataacgctttcccgaattcgagc

SEQ ID NO: 149 - DNA, 40 bp, Artificial Sequence
  synthetic construct
gctcgaattcgggaaagcgttattagcgatgagctcggac

SEQ ID NO: 150 - DNA, 47 bp, Artificial Sequence
  synthetic construct
cgtctcaagtgtaccaagtcttctcgaccaattctcatgtttgacag

SEQ ID NO: 151 - DNA, 26 bp, Artificial Sequence
  synthetic construct
aaggaaaaaagcggccgcccgggccg

SEQ ID NO: 152 - DNA, 43 bp, Artificial Sequence
  synthetic construct
aaggaaaaaagcggccgctcgaccaattctcatgtttgacagc

SEQ ID NO: 153 - DNA, 38 bp, Artificial Sequence
  synthetic construct
ctcacccagggattggctgatacgaaaaacatattctc

SEQ ID NO: 154 - DNA, 38 bp, Artificial Sequence
  synthetic construct
gagaatatgtttttcgtatcagccaatccctgggtgag

SEQ ID NO: 155 - DNA, 40 bp, Artificial Sequence
  synthetic construct
cttacgtgccgatcaacgactcattttcgccaaaagttgg

SEQ ID NO: 156 - DNA, 40 bp, Artificial Sequence
  synthetic construct
ccaacttttggcgaaaatgagtcgttgatcggcacgtaag

SEQ ID NO: 157 - DNA, 40 bp, Artificial Sequence
  synthetic construct
ggatggctcaggcatcgtgtctctgaaaatcgactggatc

SEQ ID NO: 158 - DNA, 40 bp, Artificial Sequence
  synthetic construct
gatccagtcgattttcagagacacgatgcctgagccatcc

SEQ ID NO: 159 - DNA, 40 bp, Artificial Sequence
  synthetic construct
cttaacctggacaggtcacgtgttccaactgagtgtatag

SEQ ID NO: 160 - DNA, 40 bp, Artificial Sequence
  synthetic construct
ctatacactcagttggaacacgtgacctgtccaggttaag

SEQ ID NO: 161 - DNA, 38 bp, Artificial Sequence
  synthetic construct
aattttgacgtcgttattagcgatgagctcggacttcc

SEQ ID NO: 162 - DNA, 55 bp, Artificial Sequence
  synthetic construct
gaataatacgactcactatagggacgttcgtgttgttttagatttcatctaaacg

SEQ ID NO: 163 - DNA, 55 bp, Artificial Sequence
  synthetic construct
tttttttttttttttttttttgtcattctcctaagaagctattaaaatcacatgg

SEQ ID NO: 164 - DNA, 48 bp, Artificial Sequence
  synthetic construct
gataatacgactcactatagattaaaggtttataccttcccaggtaac